(19) United States
(12) Patent Application Publication
(10) Pub. No.: US 2024/0051937 A1
(43) Pub. Date: Feb. 15, 2024

(54) ISOINDOLINONE AMIDE COMPOUNDS USEFUL TO TREAT DISEASES ASSOCIATED WITH GSPT1

(71) Applicant: Monte Rosa Therapeutics, Inc., Boston, MA (US)

(72) Inventors: Bernhard Fasching, Basel (CH); Thomas Ryckmans, Basel (CH); Alexander Flohr, Basel (CH)

(21) Appl. No.: 18/485,780

(22) Filed: Oct. 12, 2023

Related U.S. Application Data
(63) Continuation of application No. PCT/IB2022/000223, filed on Apr. 14, 2022.

(30) Foreign Application Priority Data
Apr. 14, 2021 (CH) 00384/21

(51) Int. Cl. C07D 401/04; C07D 401/14; C07D 405/14; C07D 491/08; C07D 417/14; A61K 45/06

(52) U.S. Cl. CPC C07D 401/04; C07D 401/14; C07D 405/14; C07D 491/08; C07D 417/14; A61K 45/06

(57) ABSTRACT

Disclosed herein, in part, is a compound or pharmaceutically acceptable salts or stereoisomers thereof of formula I:

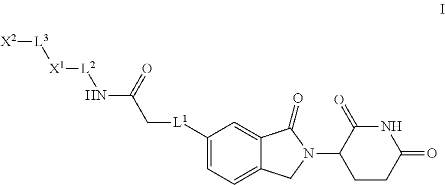

wherein X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, and —C₁₋₆ alkylhydroxy; or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, and C₁₋₄ alkylhydroxy; X² is selected from the group consisting of hydrogen, C₃₋₆ cycloalkyl, —C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy; L¹ is selected from —CH₂—, O and NH; L² is selected from a covalent bond, and linear or branched C₁₋₆ alkyl; L³ is selected from a covalent bond, linear or branched C₁₋₆ alkyl, —O—, and —C₁₋₄ alkoxy.

### CROSS-REFERENCE

This application is a continuation of International Patent Application No. PCT/IB2022/000223, filed Apr. 14, 2022, which claims priority to Swiss Patent Application Number 00384/21, filed Apr. 14, 2021, the contents of each of which are incorporated herein by reference in its entirety.

### FIELD OF DISCLOSURE

The present disclosure relates to new compounds as modulators of cereblon. The disclosure also relates to methods of preparation of these compounds, compositions comprising these compounds, and methods of using them in the treatment of abnormal cell growth in mammals, especially humans.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues (Tan et al. Nature 2007, 446, 640-645 and Sheard et al. Nature 2010, 468, 400-405) to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein. Particularly relevant compounds for the E3 ligase cereblon are the IMiD (immunomodulatory imide drugs) class including Thalidomide, Lenalidomide and Pomalidomide. These IMiDs have been approved by the FDA for use in hematological cancers. However, compounds for efficiently targeting other diseases, in particular other types of cancers, are still required.

### SUMMARY OF DISCLOSURE

It is therefore an object of the present disclosure to advance the state of the art of cereblon modulators and provide modulators for novel use in different diseases, in particular in different cancers.

In some embodiments, compounds are provided for use in therapy of solid tumors, such as for use in the therapy of lung cancer for example, non-small cell lung cancer (e.g., squamous cell lung cancer) and small cell lung cancer, breast cancer, and neuroendocrine cancer, e.g., neuroendocrine prostate cancer such as castration-resistant neuroendocrine prostate cancer (NEPC) and lung neuroendocrine tumors (Lu-NETs). In some embodiments, compounds are provided for use in therapy of blood-borne (or haematological) cancers such as for use in the therapy of leukemias (e.g. acute myelogenous leukemia (AML)) and myelomas (e.g. multiple myeloma (MM)).

The present disclosure is in a first aspect directed towards a compound or pharmaceutically acceptable salts or stereoisomers thereof of formula I.

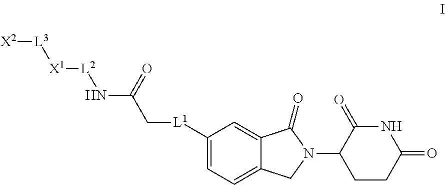

wherein X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, —C₁₋₆ alkylhydroxy;
or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, C₁₋₄ alkylhydroxy;
X² is hydrogen, C₃₋₆ cycloalkyl, —C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
L¹ is selected from —CH₂—, O and NH;
L² is a covalent bond, linear or branched C₁₋₆ alkyl;
L³ is a covalent bond, linear or branched C₁₋₆ alkyl, —O—, —C₁₋₄ alkoxy.

In some embodiments of a compound of formula I, X¹ is linear or branched C₁₋₆ alkyl, C₃₋₆ cycloalkyl, C₆ aryl, 5-10 membered heteroaryl, 4-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₆ alkoxy.

In some embodiments of a compound of formula I, X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula I, L¹ is —CH₂— and/or L² is a covalent bond or —CH₂— and/or L³ is a covalent bond, —CH₂—, —O—CH₂—, —O—CH₂—CH₂— or —O—.

In some embodiments, the present disclosure is directed towards a compound or pharmaceutically acceptable salts or stereoisomers thereof of formula II.

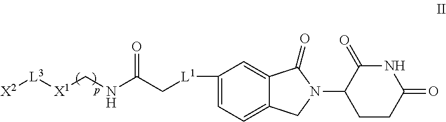

wherein X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, CF₃, CHF₂, —O—CHF₂, —O—(CH₂)₂—OMe, OCF₃, —CN, NH₂, C₁₋₆ alkoxy or C₁₋₆ alkylhydroxy;
or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
L¹ is selected from —CH₂—, O and NH;
L³ is a covalent bond, linear or branched C₁₋₆ alkyl, —O—, —C₁₋₄ alkoxy;
p is 0, 1.

In some embodiments, the present disclosure is directed towards a compound or pharmaceutically acceptable salts or stereoisomers thereof of formula III, IV, V:

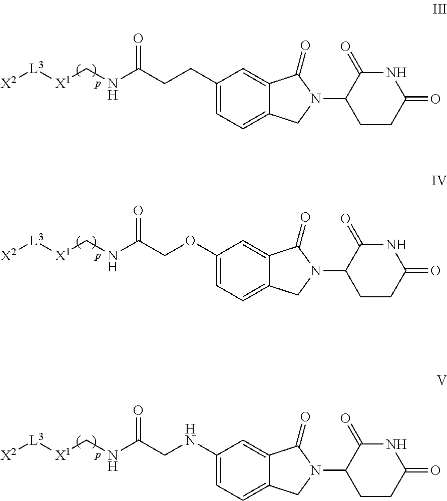

wherein X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, CF₃, CHF₂, —O—CHF₂, —O—(CH₂)₂—OMe, OCF₃, —CN, NH₂, C₁₋₆ alkoxy or C₁₋₆ alkylhydroxy;
or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
L³ is a covalent bond, linear or branched C₁₋₆ alkyl, —O—, —C₁₋₄ alkoxy;
p is 0 or 1.

In some embodiments, a compound of formula I is a compound or pharmaceutically acceptable salts or stereoisomers thereof of formula VI, VII or VIII:

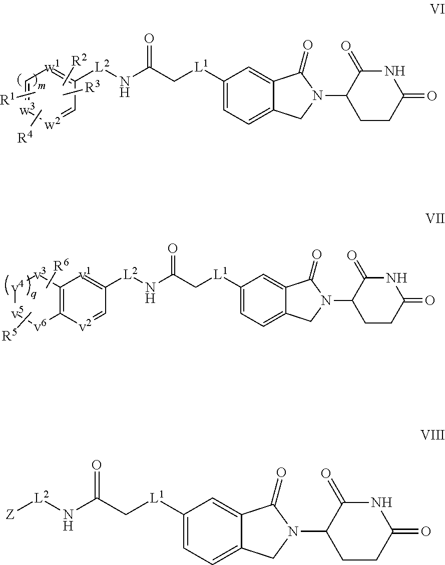

wherein
w¹, w², w³ are independently of each other selected from C, N, S, O, with the proviso that at least one of w¹, w², w³ is C;
one or two of v³, v⁴, v⁵, v⁶ are independently of each other selected from C and O and the remaining of v³, v⁴, v⁵, v⁶ are C;
v¹, v² are independently of each other selected from C and N;
L¹ is selected from —CH₂—, O and NH; L² is a covalent bond, linear or branched C₁₋₆ alkyl;
R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein
X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
R⁵, R⁶ are independently of each other selected from H, linear or branched C₁₋₄ alkyl, halogen, preferably F, Cl, more preferably F;
Z is linear or branched —C₁₋₆ alkyl, —C₃₋₆ cycloalkyl, —C₁₋₄ alkoxy, 4-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the amide forms a 4-6 membered heterocycloalkyl, which is unsubstituted or substituted with C₁₋₄ alkyl, C₆ aryl, C₆ aryloxy, 6 membered heteroaryl or CF₃;
q is 0 or 1; m is 0 or 1.

In some embodiments, the present disclosure is directed towards a compound or pharmaceutically acceptable salts or stereoisomers thereof of formula X

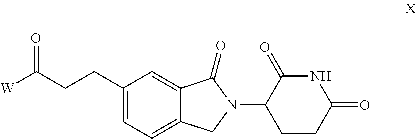

wherein W is selected from

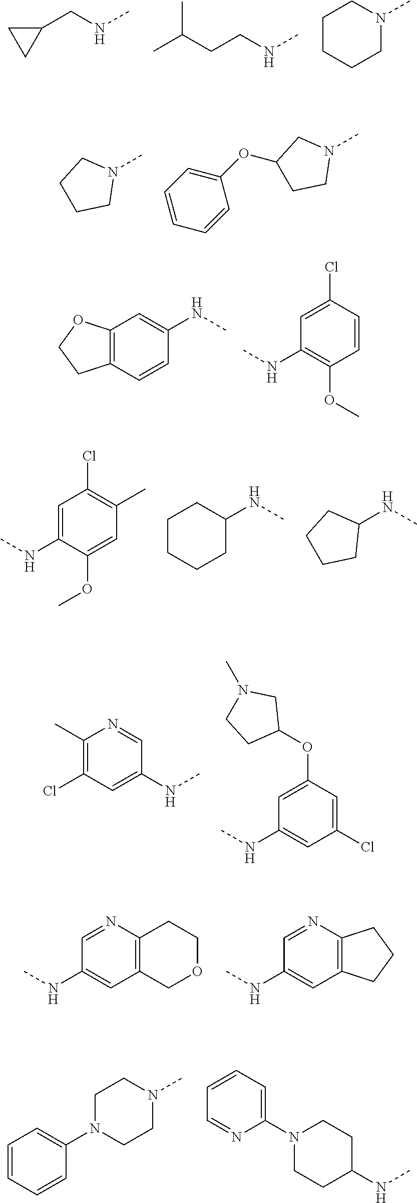

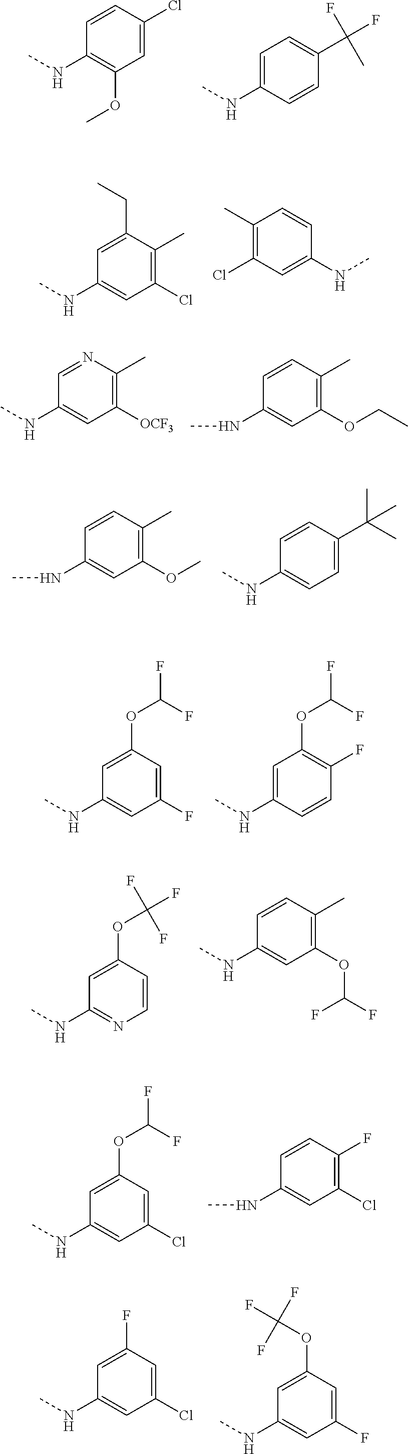

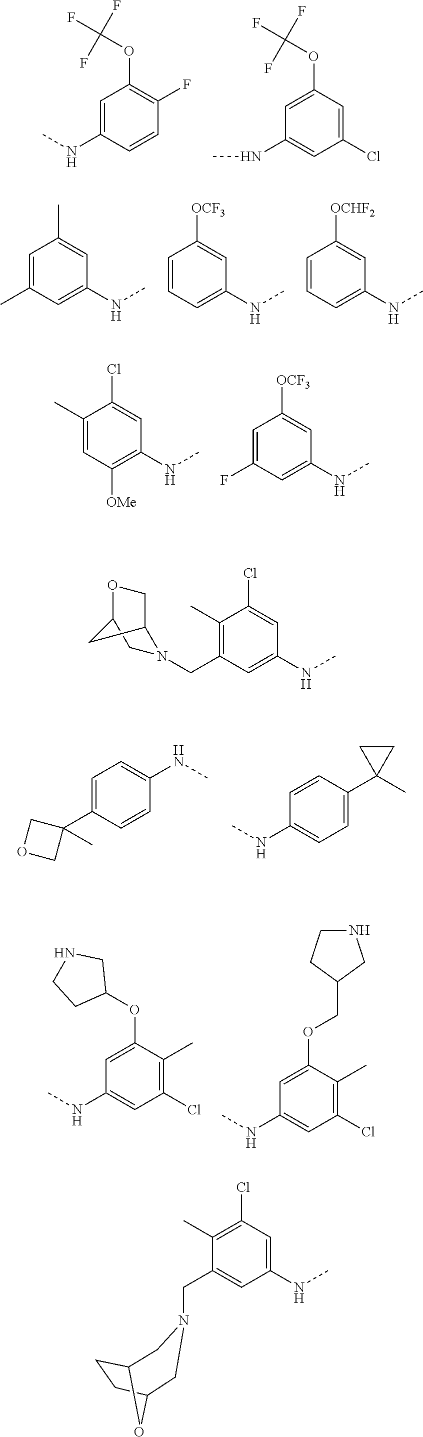

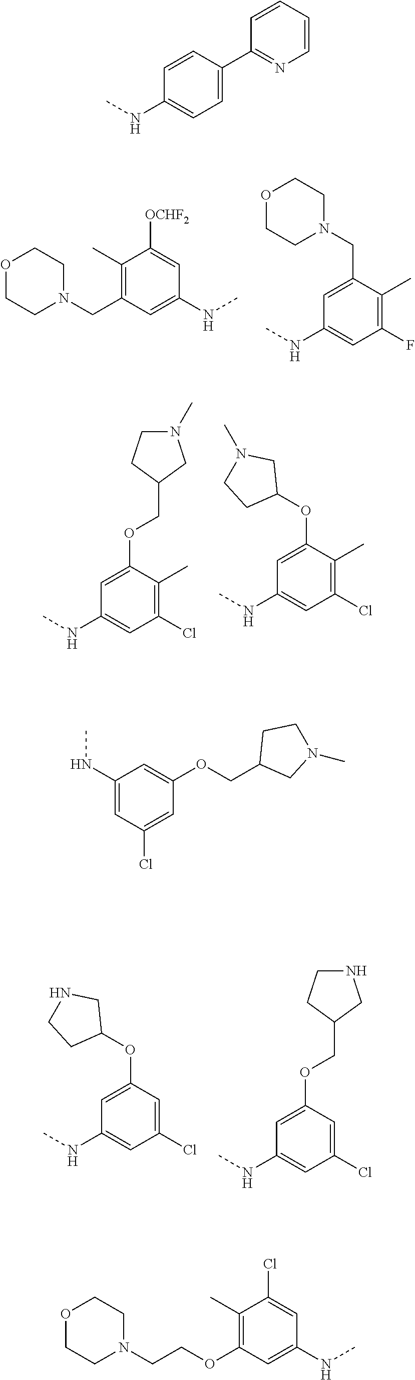

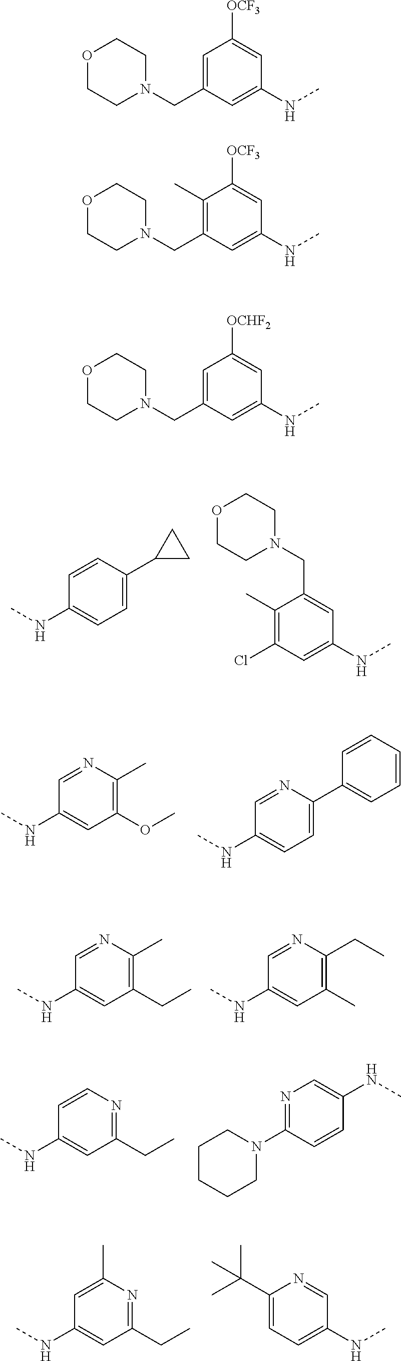

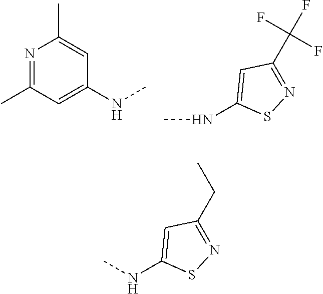

In some embodiments, provided herein is a compound of formula I

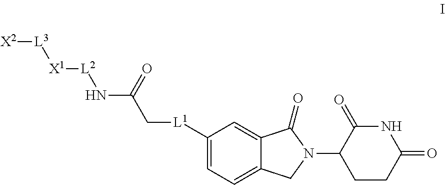

or a pharmaceutically acceptable salt or stereoisomer thereof,
wherein X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, and —C₁₋₆ alkylhydroxy;
or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, and C₁₋₄ alkylhydroxy;
X² is selected from the group consisting of hydrogen, C₃₋₆ cycloalkyl, —C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is selected from the group consisting of —CH₂—, O and NH;
L² is selected from the group consisting of covalent bond, and linear or branched C₁₋₆ alkyl; and
L³ is selected from the group consisting of covalent bond, linear or branched C₁₋₆ alkyl, —O—, and —C₁₋₄ alkoxy.

In some embodiments, X¹ is selected from the group consisting of linear or branched C₁₋₆ alkyl, C₃₋₆ cycloalkyl, C₆ aryl, 5-10 membered heteroaryl, and 4-6 membered heterocycloalkyl, wherein X¹ is unsubstituted or substituted with one or more substituents independently selected from the group consisting of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, and C₁₋₄ alkoxy; or X¹ together with the N atom of the amide forms a 4-8 membered heterocycloalkyl, which is unsubstituted or substituted with one or more substituents independently selected from the group consisting of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, and C₁₋₆ alkoxy.

In some embodiments, X² is selected from the group consisting of H, C₃₋₆ cycloalkyl, C₆ aryl, 5-10 membered heteroaryl, and 4-8 membered heterocycloalkyl, wherein X² is unsubstituted or substituted with one or more substituents independently selected from the group consisting of linear or branched C₁₋₄ alkyl, —C₁₋₄ alkoxy, NMe₂, halogen, CF₃, CHF₂, CMeF₂, OCF₃, and OCHF₂.

In some embodiments, L¹ is —CH₂— and/or L² is selected from a covalent bond and —CH₂— and/or L³ is selected from the group consisting of a covalent bond, —CH₂— —O—CH₂—, —O—CH₂—CH₂— and —O—.

In some embodiments, the compound is of formula II

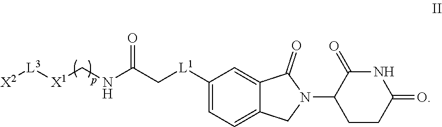

or a pharmaceutically acceptable salt or stereoisomer thereof,
wherein X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, CF₃, CHF₂, —O—CHF₂, —O—(CH₂)₂—OMe, OCF₃, —CN, NH₂, C₁₋₆ alkoxy and C₁₋₆ alkylhydroxy; or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₄ alkylhydroxy, and C₁₋₆ alkoxy;
X² is selected from the group consisting of hydrogen, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is selected from the group consisting of —CH₂—, O and NH;
L³ is selected from the group consisting of a covalent bond, linear or branched C₁₋₆ alkyl, —O—, and —C₁₋₄ alkoxy; and
p is 0 or 1.

In some embodiments, the compound is, of formula III, IV, or V:

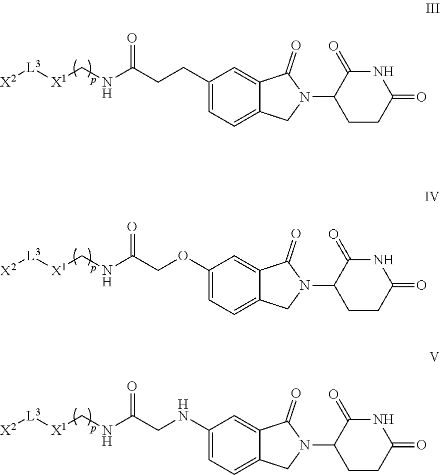

or a pharmaceutically acceptable salt or stereoisomer thereof,
wherein X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, CF₃, CHF₂, —O—CHF₂, —O—(CH₂)₂—OMe, OCF₃, —CN, NH₂, C₁₋₆ alkoxy and C₁₋₆ alkylhydroxy; or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₄ alkylhydroxy, and C₁₋₆ alkoxy;
X² is selected from the group consisting of hydrogen, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L³ is selected from the group consisting of a covalent bond, linear or branched C₁₋₆ alkyl, —O—, and —C₁₋₄ alkoxy; and
p is 0 or 1.

In some embodiments, the compound is of formula VI, VII or VIII:

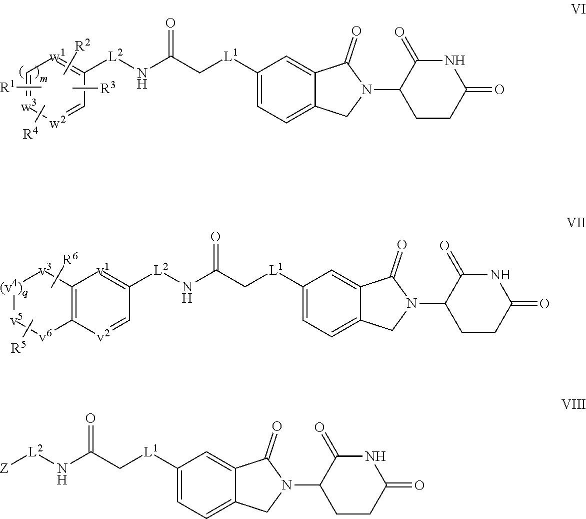

or a pharmaceutically acceptable salt or stereoisomer thereof,
wherein
each of w¹, w², and w³ is independently selected from the group consisting of C, N, S, and O, with the proviso that at least one of w¹, w², and w³ is C;
one or two of v³, v⁴, v⁵, and v⁶ is independently selected from C and O and the remaining of v³, v⁴, v⁵, and v⁶ are C;
each of v¹, and v² is independently selected from C and N;
L¹ is selected from —CH₂—, O and NH;
L² is selected from the group consisting of a covalent bond, and linear or branched C₁₋₆ alkyl;
each of R¹, R², R³, and R⁴ is independently selected from the group consisting of hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, and a group of formula -L³-X², wherein L³ is selected from the group consisting of a covalent bond, linear or branched C₁₋₆ alkyl, —O—, and —C₁₋₄ alkoxy, and X² is selected from the group consisting of C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
each of R⁵, and R⁶ is independently selected from the group consisting of H, linear or branched C₁₋₄ alkyl, and halogen, preferably F, or Cl, or more preferably F;
Z is selected from the group consisting of linear or branched —C₁₋₆ alkyl, —C₃₋₆ cycloalkyl, —C₁₋₄ alkoxy, and 4-6 membered heterocycloalkyl, wherein Z is unsubstituted or substituted with a substituent selected from the group consisting of with C₁₋₄ alkyl, C₆ aryl, C₆ aryloxy, 6 membered heteroaryl and CF₃; or Z together with the N atom of the amide forms a 4-6 membered heterocycloalkyl, which is unsubstituted or substituted with a substituent selected from the group consisting of C₁₋₄ alkyl, C₆ aryl, C₆ aryloxy, 6 membered heteroaryl and CF₃;
q is 0 or 1; and
m is 0 or 1.

In some embodiments, the compound is of formula X

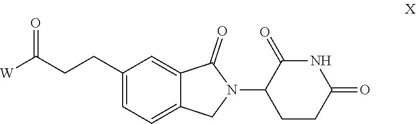

or a pharmaceutically acceptable salt or stereoisomer thereof,
wherein
W is selected from the group consisting of:

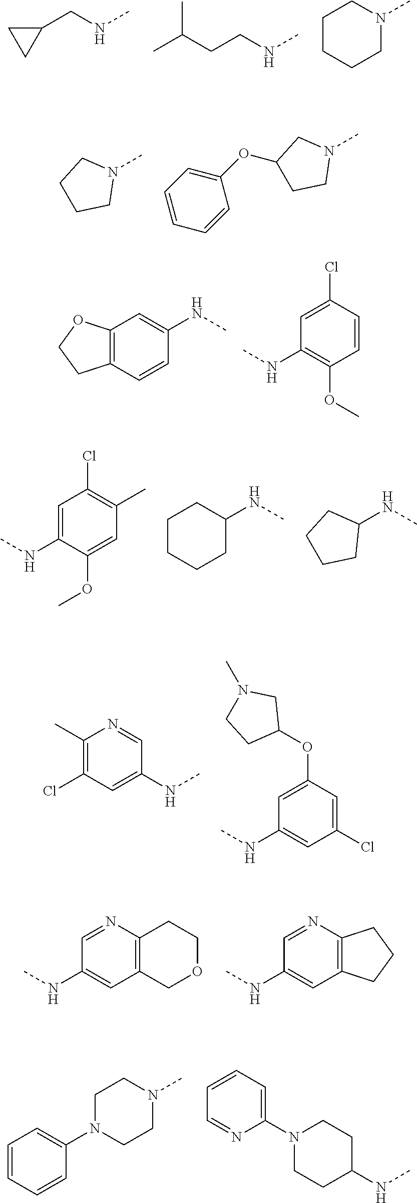

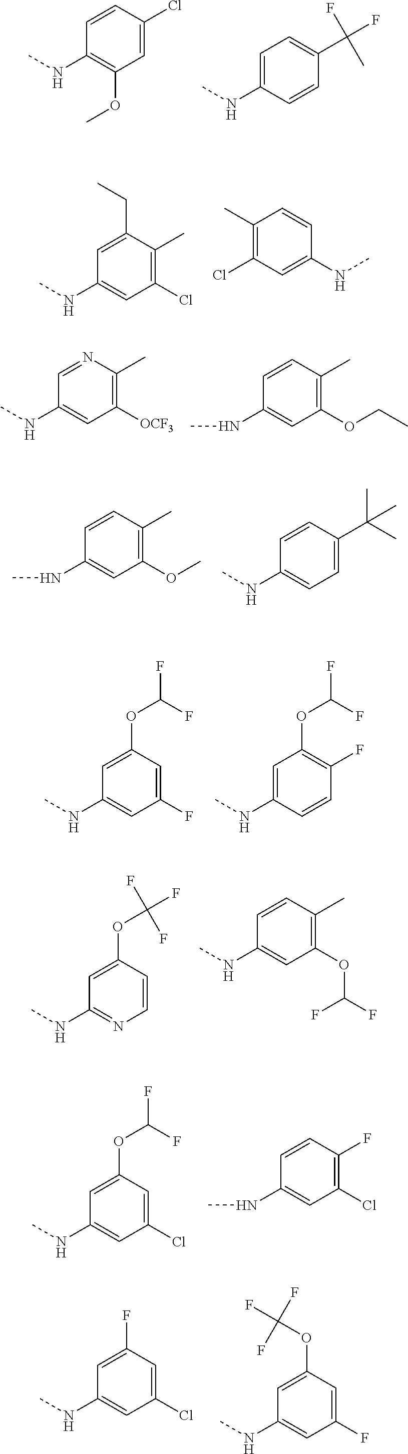

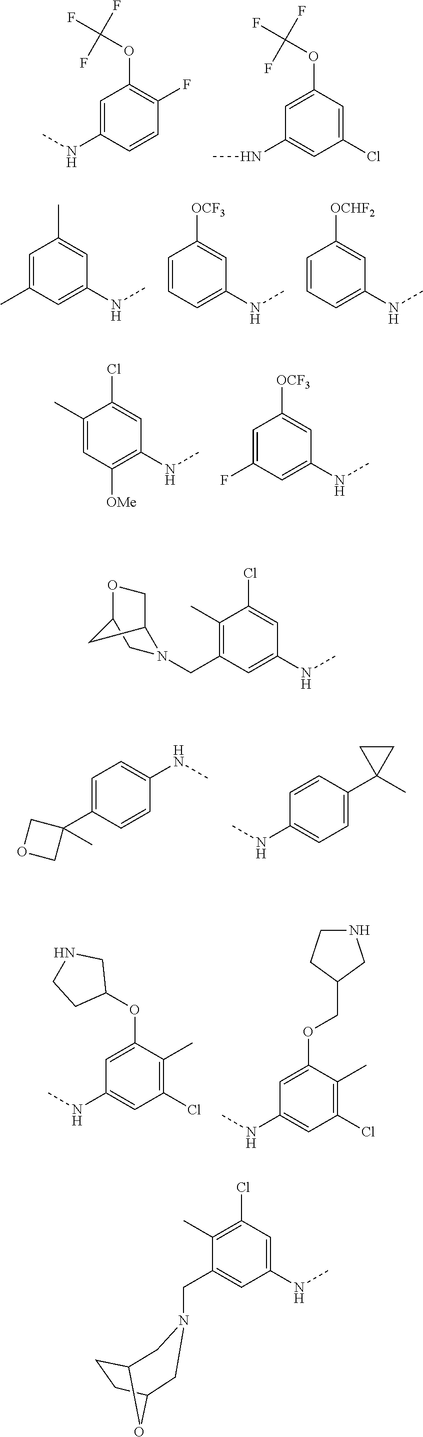

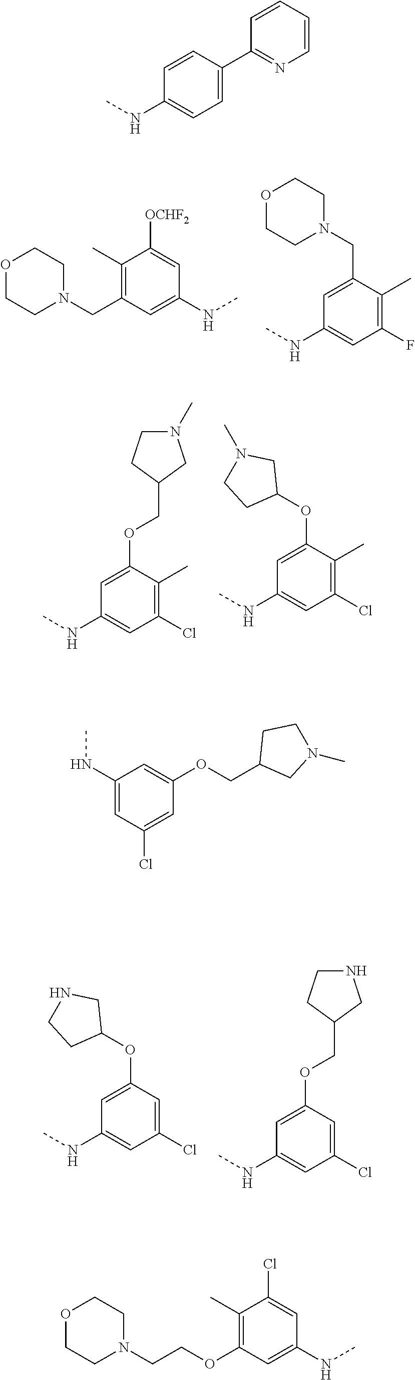

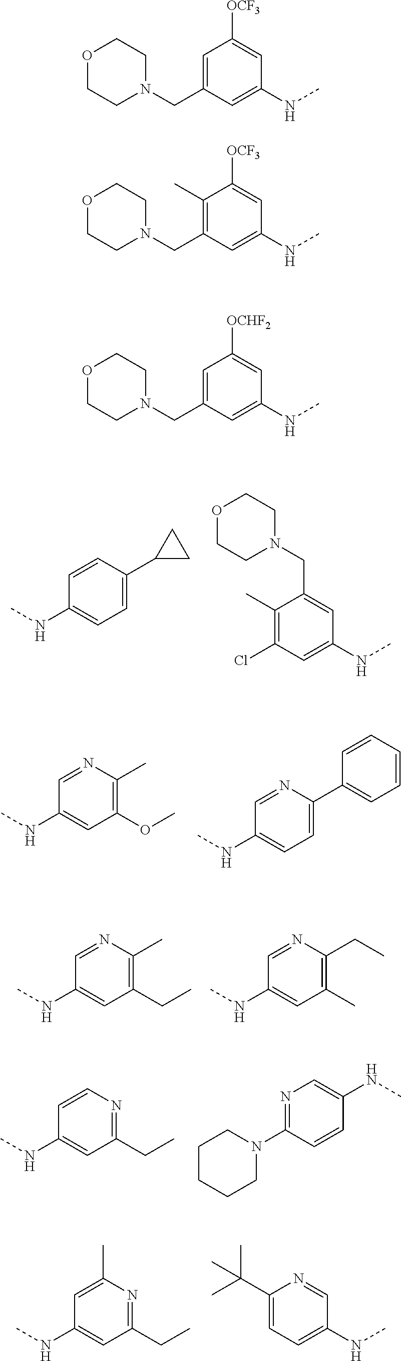

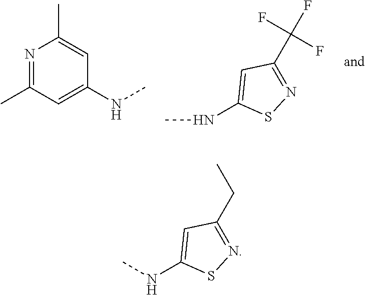

In a second aspect, the disclosure is directed to a composition comprising a compound according to any one of the embodiments or pharmaceutically acceptable salts or stereoisomers thereof described herein.

In some embodiments, the composition further comprises a pharmaceutically acceptable carrier.

In some embodiments, the composition further comprises a second therapeutically active agent.

In a third aspect, the disclosure is directed to a composition according to any of the embodiments described herein, for use in therapy.

In a fourth aspect, some embodiments comprise a compound or pharmaceutically acceptable salts or stereoisomers thereof of formula I-X, a pharmaceutical acceptable salt thereof or a composition described herein for use in the treatment of diseases associated with or caused by GSPT1, in particular the treatment of cancer associated with GSPT1, such as solid cancers including but not limited to cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, uterus, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

Some embodiments comprise the compound or the composition according to any of the embodiments described herein for use in the treatment of breast cancer.

Some embodiments comprise the compound or the composition according to any of the embodiments described herein for use in the treatment of lung cancer, for example, non-small cell lung cancer (e.g., squamous cell lung cancer) and small cell lung cancer.

Some embodiments comprise the use of a compound or a composition according to any of the embodiments described herein for treating neuroendocrine prostate cancer, for example, castration-resistant neuroendocrine prostate cancer (NEPC).

Some embodiments comprise the use of a compound or a composition according to any of the embodiments described herein for treating lung neuroendocrine tumors (Lu-NETs).

Some embodiments comprise the compound or the composition according to any of the embodiments described herein for use in the treatment of acute myelogenous leukemia (AML) and multiple myeloma (MM).

In a fifth aspect, the disclosure is directed to a use of a compound or the composition according to any of the embodiments described herein for binding to cereblon comprising administering to a subject a therapeutically-effective amount of the composition.

Some embodiments comprise the use of a composition according to any of the embodiments described herein for treating diseases associated or caused by GSPT1, in particular for treating cancer associated with or caused by GSPT1, such as solid cancers and blood bourne (liquid) or hematological cancers as defined herein.

Some embodiments comprise the use of a compound or a composition according to any of the embodiments described herein for treating breast cancer.

Some embodiments comprise the use of a compound or a composition according to any of the embodiments described herein for treating lung cancer, for example, non-small cell lung cancer (e.g., squamous cell lung cancer) and small cell lung cancer.

Some embodiments comprise the use of a compound or a composition according to any of the embodiments described herein for treating neuroendocrine prostate cancer, for example, castration-resistant neuroendocrine prostate cancer (NEPC).

Some embodiments comprise the use of a compound or a composition according to any of the embodiments described herein for treating lung neuroendocrine tumors (Lu-NETs).

Some embodiments comprise the use of a compound or a composition according to any of the embodiments described herein for treating acute myelogenous leukemia (AML) and multiple myeloma (MM).

In a sixth aspect, the disclosure is directed to a method of treating cancer in a subject, comprising administering to a subject a therapeutically effective amount of the compound or the composition of any of the embodiments as described herein.

In some embodiments, the method comprises a compound according to any of the embodiments as described herein or pharmaceutically acceptable salts or stereoisomers thereof that binds to cereblon.

In a seventh aspect, the disclosure is directed to a method of treating a Myc-driven cancer in a subject in need thereof, comprising administering the subject a therapeutically effective amount of the compound or a composition as described herein. In an eighth aspect, the disclosure is directed to a method of degrading GSPT1 in a subject suffering from cancer, comprising administering the subject a therapeutically effective amount of a compound or a composition as described herein.

In a ninth aspect, the disclosure is directed to a method of reducing the level of GSPT1 in a subject suffering from cancer, comprising administering the subject a therapeutically effective amount of a compound or a composition as described herein.

### DETAILED DESCRIPTION OF THE DISCLOSURE

Unless specified otherwise the following general definitions apply to all compounds of the disclosure according to the description.

The term “compound of the disclosure” as used herein, refers to compounds represented by formulae I to X (including salts and stereoisomers thereof) and any of the specific examples disclosed herein.

It is understood that “independently of each other” means that when a group is occurring more than one time in any compound, its definition on each occurrence is independent from any other occurrence.

It is further understood that a dashed line (or a wave being transverse to a bond) or a solid line without attachment, such as —C₁₋₄ alkyl, depicts the site of attachment of a residue (i.e. a partial formula).

It is further understood that the abbreviations “C” and “N” are representative for all possible degrees of saturation, which typically do not result in radicals, nitrenes or carbenes, i.e. N includes —NH— and —N═, C includes —CH₂— and ═CH—. In addition, “C” as an atom in an aromatic or heteroaromatic ring which has a substituent R^{x} at any suitable position, includes ═CH— as well as ═CR^{x}—.

The term “saturated” in reference to ring systems refers to a ring having no double or triple bonds. The term “partially unsaturated” in reference to ring systems refers to a ring that includes at least one double or triple bond, but does not include aromatic systems.

The term “aromatic” refers to monocyclic or multicyclic (e.g. bicyclic) ring systems, which show some or complete conjugation or delocalization of their electrons. Aromatic monocyclic rings, such as aryl or heteroaryl rings as defined herein, include phenyl, pyridinyl, furyl and the like. Aromatic multicyclic rings, such as aryl or heteroaryl rings as defined herein, refer to ring systems, wherein at least one ring is an aromatic ring, and thus include (i) aromatic ring systems, wherein an aromatic ring is fused to one or more aromatic rings, such as in e.g. naphthyl, indolyl, benzimidazolyl, and the like (also referred to as fully aromatic ring systems), and (ii) aromatic ring systems, wherein an aromatic ring is fused to one or more non-aromatic rings, such as in e.g. indanyl, indenyl, phthalimidyl, naphthimidyl, phenanthridinyl, tetrahydronaphthyl, 1,4-dihydronapthyl, and the like (also referred to as partially aromatic ring systems).

The term “non-aromatic” refers to (i) fully saturated rings such as monocyclic rings, e.g. cyclohexyl, and bicyclic rings, e.g. tetrahydronaphthyl, and (ii) partially unsaturated rings such as monocyclic rings, e.g. cyclohexenyl, and bicyclic rings, e.g. 1,4-dihydronapthyl.

The term “C₆₋₁₀ aryl” refers to a fully or partially aromatic ring system having 6, 7, 8, 9, 10 ring atoms and includes monocycles and fused bicycles. Examples of fully aromatic C₆₋₁₀ aryl include e.g. phenyl, naphthyl. Examples of partially aromatic C₆₋₁₀ aryl include e.g. 2,3-dihydroindenyl, 1, 2, 3, 4-tetrahydronaphthyl. In some embodiments for group X¹ C₆₋₁₀ aryl is phenyl, 2,3-dihydroindenyl. In some embodiments for group X² C₆₋₁₀ aryl is phenyl. The term “—C₁₋₆ alkyl-C₆₋₁₀ aryl” refers to a C₆₋₁₀ aryl which is linked through a C₁₋₆ alkyl group as defined herein. The term “—C₁₋₆ alkoxy-C₆₋₁₀ aryl” refers to a C₆₋₁₀ aryl which is linked through a C₁₋₆ alkoxy group as defined herein. The term “—O—C₆₋₁₀ aryl” or “C₆₋₁₀ aryloxy” refers to a C₆₋₁₀ aryl which is linked through a —O— group. The C₆₋₁₀ aryl group may be unsubstituted or substituted with C₁₋₄ alkyl, such as methyl, ethyl, t-butyl, fluorinated C₁₋₄ alkyl, such as —CF₃, —C(CH₃)F₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl.

In some embodiments for X¹ a C₆₋₁₀ aryl group refers to a fully aromatic ring system, e.g. phenyl, which is unsubstituted or substituted with C₁₋₄ alkyl, such as methyl, ethyl, t-butyl, fluorinated C₁₋₄ alkyl, such as —C(CH₃)F₂, C₁₋₄ alkoxy, such as methoxy, ethoxy, fluorinated C₁₋₄ alkoxy, such as —OCF₃, —OCHF₂, halogen, such as F or Cl, In some embodiments for X² a C₆₋₁₀ aryl group refers to a fully aromatic ring system, e.g. phenyl, which is unsubstituted or substituted with C₁₋₄ alkyl, such as methyl, ethyl, C₁₋₄ alkoxy, such as methoxy, ethoxy, halogen, such as F, Cl, or Br, such as F or Cl, e.g. F.

The term “5-10 membered heteroaryl” refers to a fully or partially aromatic ring system in form of monocycles or fused bicycles having 5, 6, 7, 8, 9, 10 ring atoms selected from C, N, O, and S, such as C, N, and O, or C, N, and S, with the number of N atoms being e.g. 0, 1, 2 or 3 and the number of O and S atoms each independently being 0, 1 or 2. In some embodiments a 5-10 membered heteroaryl refers to a fully aromatic ring system having 5, 6, 7, 8, 9, 10, such as 5 or 6, e.g. 6 ring atoms selected from C and N, with the number of N atoms being 1, 2 or 3, such as 1 or 2, e.g. 1. In some embodiments a 5-10 membered heteroaryl refers to a fully aromatic ring system having 6 ring atoms selected from C and N, with the number of N atoms being 1 or 2, e.g. 1. In some embodiments a 5-10 membered heteroaryl refers to a ring system having 5 ring atoms selected from C, N, O, S with the number of N, S and O atoms each being independently 0, 1 or 2. In some embodiments the total number of N, S and O atoms is 2. In other embodiments a 5-10 membered heteroaryl refers to a partially aromatic ring system having 9 or 10 ring atoms selected from C, N and O, with the number of 0 atoms being 1, 2 or 3, such as 1 or 2, e.g. 1, and the number of N atoms being 1 or 2, such as 1. In some embodiments, examples of “5-10 membered heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiophenyl, thiazolyl, thienyl, indolyl, quinazolinyl, oxazolinyl, isoxazolinyl, indazolinyl, isothiazolyl, 1,3-benzodioxolyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, benzodihydropyrane, 1,2,3,4-tetrahydronaphthyl, 2,3-dihydroindenyl and the like. In some embodiments, examples of “5-10 membered heteroaryl” include 5-membered heteroaryl, such as isothiazole, 6-membered heteroaryl, such as pyridinyl, 9-membered heteroaryl, such as 2,3-dihydrobenzofuryl, cyclopentenopyridine, and 10-membered heteroaryl, such as dihydropyrano-pyridine. The term “—C₁₋₆ alkyl 5-10 membered heteroaryl” refers to a 5-10 membered heteroaryl, which is linked through a C₁₋₆ alkyl group as defined herein to its neighbouring group. The term “—C₁₋₆ alkoxy 5-10 membered heteroaryl” refers to a 5-10 membered heteroaryl, which is linked through a C₁₋₆ alkoxy group as defined herein to its neighbouring group. The term “—O—5-10 membered heteroaryl” refers to a 5-10 membered heteroaryl, which is linked through a —O— group to its neighbouring group. The term “—O—C₆₋₁₀ aryl” refers to a C₆₋₁₀ aryl which is linked through a —O— group. The 5-10 membered heteroaryl group may be unsubstituted or substituted C₁₋₄ alkyl, such as methyl, ethyl, t-butyl, fluorinated C₁₋₄ alkyl, such as —CF₃, —C(CH₃)F₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl. In some embodiments, the 5-10 membered heteroaryl group may be unsubstituted or substituted with C₁₋₄ alkyl, such as methyl, ethyl, t-butyl, C₁₋₄ alkoxy, such as methoxy, fluorinated C₁₋₄ alkoxy, such as —OCF₃, halogen, such as F or Cl.

In some embodiments for X¹ a 5-10 membered heteroaryl refers to a fully aromatic ring system having either 5 ring atoms selected from C, N and S with the number of N and S atoms being independently of each other 1 or 2, e.g. 1 or 6 ring atoms selected from C and N, with the number of N atoms being 1 or 2, and a partially aromatic ring system having 9 or 10 ring atoms selected from C, N and O, with the number of O atoms being 0 or 1 and the number of N atoms being 0 or 1. In some embodiments for X¹ a 5-10 membered heteroaryl refers to pyridinyl, isothiazole, 2,3-dihydrobenzofuryl, cyclopentenopyridine, dihydropyrano-pyridine.

In some embodiments for X² a 5-10 membered heteroaryl refers to a fully aromatic ring system having 6 ring atoms selected from C and N, with the number of N atoms being 1 or 2, such as 1. In some embodiments for X² a 5-10 membered heteroaryl refers to pyridinyl.

It is understood and known to the skilled person that the exact position of the double bond(s) in a partially or fully aromatic heteroaryl are positioned such that the rules of valency are fulfilled.

The term “C₃₋₆ cycloalkyl” refers to a non-aromatic, i.e. saturated or partially unsaturated alkyl ring system containing 3, 4, 5 or 6 carbon atoms, such as cyclopropyl, cyclobutyl, cyclopentyl, cyclohexyl, unsubstituted or substituted by e.g. one or more of C₁₋₄ alkyl, such as methyl and halogen, such as F. The term “—C₁₋₄ alkyl-C₃₋₆ cycloalkyl” refers to -L²-X¹- or L³-X²- with L², L³ being a C₁₋₄ alkyl group and X¹, X² being C₃₋₆ cycloalkyl as defined herein and refers to a C₃₋₆ cycloalkyl, which is linked through a C₁₋₆ alkyl group as defined herein to its neighbouring group. The term “—O—C₃₋₆ cycloalkyl” refers to -L²-X¹- or L³-X²- with L², L³ being —O— and X¹, X² being C₃₋₆ cycloalkyl as defined herein and refers to a C₃₋₆ cycloalkyl, which is linked through —O— to its neighbouring group. The term “—C₁₋₄ alkoxy-C₃₋₆ cycloalkyl” refers to -L²-X¹- or L³-X²- with L², L³ being a C₁₋₄ alkoxy group and X¹, X² being C₃₋₆ cycloalkyl as defined herein and refers to a C₃₋₆ cycloalkyl, which is linked through a C₁₋₆ alkoxy group as defined herein to its neighbouring group. In some embodiments for X¹ a C₃₋₆ cycloalkyl refers to cyclopropyl, cyclopentyl, cyclohexyl. In some embodiments for X² a C₃₋₆ cycloalkyl refers to cyclopropyl.

The term “4-8 membered heterocycloalkyl” refers to a non-aromatic, i.e. saturated or partially unsaturated ring system having 4, 5, 6, 7 or 8 ring atoms (of which at least one is a heteroatom), which ring atoms are selected from C, N, O, and S, such as C, N, and O, the number of N atoms being 0, 1, or 2 and the number of O and S atoms each independently being 0, 1, or 2, such as 0 or 1. In some embodiments the term “4-8 membered heterocycloalkyl” comprises saturated or partially unsaturated monocycles, fused bicycles, bridged bicycles or spirobicycles. In some embodiments the term “4-8 membered heterocycloalkyl” comprises fully saturated or partially unsaturated monocycles and bridged bicycles. Examples of 4-8 membered heterocycloalkyl groups include azetidinyl, oxetanyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl, 2-oxa-5-azabicyclo[2.2.1]heptan-5-yl, 8-oxa-3-azabicyclo[3.2.1]octan-3-yl and the like. The 4-8 membered heterocycloalkyl group may be unsubstituted. The 4-8 membered heterocycloalkyl group may be substituted with C₁₋₄ alkyl, such as methyl, ethyl, C₁₋₄ alkoxy, such as methoxy, ethoxy, halogen, such as F, Cl or Br, e.g. F or Cl.

In some embodiments, the 4-8 membered heterocycloalkyl representing group X¹ is a non-aromatic ring system having 5 or 6 ring atoms of which at least one is a heteroatom selected from N and O, the number of N atoms being 1 or 2 and the number of O being 0, 1, or 2, such as a non-aromatic 6 membered ring system having 1 or 2 N-atoms, such as piperidine.

In some embodiments, the 4-8 membered heterocycloalkyl formed by groups X¹ together with the N atom of the amide forms is a non-aromatic ring system having 5 or 6 ring atoms of which at least one is a heteroatom selected from N and O, the number of N atoms being 1 or 2 and the number of O being 0, 1, or 2, e.g. a non-aromatic ring system having 5 or 6 ring atoms comprising one or two N-atoms. Examples include pyrrolidinyl, piperdinyl, morpholinyl, piperazinyl, N-methyl piperazinyl.

In some embodiments, the 4-8 membered heterocycloalkyl representing X² is a non-aromatic ring system having 4, 5, 6, 7 or 8 ring atoms of which at least one is a heteroatom selected from N and O, the number of N atoms being 0, 1 or 2 and the number of O being 0, 1, or 2. The 4-8 membered heterocycloalkyl representing X² may be unsubstituted or substituted by e.g. one or more of C₁₋₄ alkyl, such as methyl, halogen, e.g. F.

In some embodiments, 4-8 membered heterocycloalkyl representing X² include 4-membered heterocycloalkyl, such as oxetanyl, methyl-oxetanyl; 5-membered heterocycloalkyl, such as pyrrolidinyl, N-methyl-pyrrolidinyl; 6-membered heterocycloalkyl, such as morpholinyl, piperidinyl; 7-membered heterocycloalkyl, such as 2-oxa-5-azabicyclo[2.2.1]heptan-5-yl; 8-membered heterocycloalkyl, such as 8-oxa-3-azabicyclo[3.2.1]octan-3-yl.

The term “C₁₋₄ alkyl 4-8 membered heterocycloalkyl” refers to -L²-X¹- or L¹-X²- with L², L³ being C₁₋₄ alkyl and X¹, X² being 4-8 membered heterocycloalkyl as defined herein. Thus, the 4-8 membered hetereocycloalkyl is linked through a C₁₋₄ alkyl group as defined to the neighbouring group. In some embodiments, the alkyl may be C₁, resulting in —(CH₂)-(4-8 membered heterocycloalkyl) or C₂, resulting in —(CH₂)₂-(4-8 membered heterocycloalkyl) or C₃, resulting in —(CH₂)₃-(4-8 membered heterocycloalkyl) or C₄, resulting in —(CH₂)₄-(4-8 membered heterocycloalkyl). Examples include —(CH₂)-morpholinyl, —(CH₂)₂-morpholinyl, —(CH₂)₃-morpholinyl, —(CH₂)₄-morpholinyl, —(CH₂)-piperazinyl, —(CH₂)₂—N-methyl-piperazinyl, —(CH₂)₃-piperazinyl or —(CH₂)₄-piperazinyl.

The term “—C₁₋₄ alkoxy 4-8 membered heterocycloalkyl” refers to -L²-X¹- or L³-X²- with L² L³ being C₁₋₄ alkoxy and X¹, X² being 4-8 membered heterocycloalkyl as defined herein. Thus, the 4-8 membered hetereocycloalkyl is linked via a C₁₋₄ alkoxy group as defined herein to its neighbouring group. In some embodiments, the C₁₋₄ alkoxy may be C₁, resulting in —(O—CH₂)-(4-8 membered heterocycloalkyl) or C₂, resulting in —(O—CH₂)₂-(4-8 membered heterocycloalkyl) or C₃, resulting in —(O—CH₂)₃-(4-8 membered heterocycloalkyl). Examples include —(O—CH₂)-(N-morpholinyl), —(O—CH₂)₂-(N-morpholinyl).

The term “—O-(4-8 membered heterocycloalkyl)” refers to -L²-X¹- or L³-X²- with L², L³ being —O— and X¹, X² being 4-8 membered heterocycloalkyl as defined herein. Thus, the 4-8 membered hetereocycloalkyl is linked through an —O-atom to the neighbouring group. Examples include —O-morpholinyl, —O-piperazinyl, —O-pyrrolidinyl and the like.

The term “halogen” or “hal” as used herein may be fluoro, chloro, bromo or iodo such as fluoro, chloro or bromo, e.g. fluoro or chloro.

The term “C₁₋₄ alkyl” and “C₁₋₆alkyl” refer to a fully saturated branched or unbranched hydrocarbon moiety having 1, 2, 3 or 4 and 1, 2, 3, 4, 5 or 6 carbon atoms, respectively. Representative examples of alkyl include, but are not limited to, methyl, ethyl, n-propyl, iso-propyl, n-butyl, sec-butyl, iso-butyl, tert-butyl, n-pentyl, iso-pentyl, neopentyl, n-hexyl, iso-hexyl or neohexyl.

The terms “C₁₋₆ alkylhydroxy” and “C₁₋₄ alkylhydroxy” refer to a fully saturated branched or unbranched C₁₋₆ alkyl and C₁₋₄ alkyl, which are substituted with at least one, such as only one, hydroxy group. Examples include but are not limited to hydroxymethyl, hydroxyethyl, hydroxypropyl, hydroxy-iso-propyl, hydroxy-n-butyl, hydroxyl-iso-butyl, hydroxyl-tert-butyl, hydroxypentyl, hydroxyhexyl.

Based on the definitions given throughout the application the skilled person knows which combinations are synthetically feasible and realistic, e.g. typically combinations of groups leading to some heteroatoms directly linked to each other, e.g. —O—O—, are not contemplated, however synthetically feasible combinations, such as —S—N═ in an isothiazole are contemplated.

In a first aspect the disclosure provides a compound or pharmaceutically acceptable salts or stereoisomers thereof of formula I:

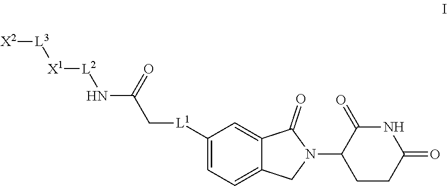

wherein X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, —C₁₋₆ alkylhydroxy;
or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, C₁₋₄ alkylhydroxy;
X² is hydrogen, C₃₋₆ cycloalkyl, —C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
L¹ is selected from —CH₂—, O and NH; L² is a covalent bond, linear or branched C₁₋₆ alkyl;
L³ is a covalent bond, linear or branched C₁₋₆ alkyl, —O—, —C₁₋₄ alkoxy.

In some embodiments of a compound of formula I, L¹ is —CH₂—. In some embodiments of a compound of formula I, L¹ is 0. In some embodiments of a compound of formula I, L¹ is NH.

In some embodiments of a compound of formula I, L² is a covalent bond or C₁₋₄ alkyl, e.g. —CH₂—. In some embodiments of a compound of formula I, L² is a covalent bond. In some embodiments of a compound of formula I, L² is linear or branched C₁₋₆ alkyl, such as C₁₋₄ alkyl, e.g. —CH₂—.

In some embodiments of a compound of formula I, L³ is a covalent bond. In some embodiments of a compound of formula I, L³ is linear or branched C₁₋₆ alkyl. In some embodiments of a compound of formula I, L³ is linear or branched C₁₋₄ alkyl, such as —CH₂—.

In some embodiments of a compound of formula I, L³ is —O—. In some embodiments of a compound of formula I, L³ is —C₁₋₄ alkoxy, such as —O—CH₂— or —O—CH₂—CH₂.

In some embodiments of a compound of formula I, L¹ is —CH₂— and L² is a covalent bond or C₁₋₄ alkyl, e.g. —CH₂—.

In some embodiments of a compound of formula I, L¹ is —CH₂—, L² is a covalent bond or C₁-4 alkyl, e.g. —CH₂—, and L³ is a covalent bond, linear or branched C₁₋₄ alkyl, such as —CH₂—, —O—, —C₁₋₄ alkoxy, such as —OCH₂— or —OCH₂CH₂—. In some embodiments of a compound of formula I, L¹ is —CH₂—, L² is a covalent bond or —CH₂—, and L³ is a covalent bond, —CH₂—, —O—, —OCH₂— or —OCH₂CH₂—.

In some embodiments of a compound of formula I, X¹ is linear or branched C₁₋₆ alkyl, C₃₋₆ cycloalkyl, C₆ aryl, 5-10 membered heteroaryl, 5-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, X¹ is linear or branched C₁₋₆ alkyl, C₃₋₆ cycloalkyl, C₆ aryl, 5-10 membered heteroaryl, 6 membered heterocycloalkyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula I, X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula I, X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula I, X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula I, L¹ is —CH₂— and X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, —C₁₋₆ alkylhydroxy; or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, C₁₋₄ alkylhydroxy.

In some embodiments of a compound of formula I, L¹ is —CH₂— and X¹ is linear or branched C₁₋₆ alkyl, C₃₋₆ cycloalkyl, C₆ aryl, 5-10 membered heteroaryl, 5-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L¹ is —CH₂— and X¹ is linear or branched C₁₋₆ alkyl, C₃₋₆ cycloalkyl, C₆ aryl, 5-10 membered heteroaryl, 6 membered heterocycloalkyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L¹ is —CH₂— and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L¹ is —CH₂— and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L² is a covalent bond and X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, —C₁₋₆ alkylhydroxy; or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, C₁₋₄ alkylhydroxy.

In some embodiments of a compound of formula I, L² is a covalent bond and X¹ is linear or branched C₁₋₆ alkyl, C₃₋₆ cycloalkyl, C₆ aryl, 5-10 membered heteroaryl, 5-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L² is a covalent bond and X¹ is linear or branched C₁₋₆ alkyl, C₃₋₆ cycloalkyl, C₆ aryl, 5-10 membered heteroaryl, 6 membered heterocycloalkyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L² is a covalent bond and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L² is a covalent bond and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L² is —CH₂— and X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, —C₁₋₆ alkylhydroxy; or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, C₁₋₄ alkylhydroxy.

In some embodiments of a compound of formula I, L² is —CH₂— and X¹ is linear or branched C₁₋₆ alkyl, C₃₋₆ cycloalkyl, C₆ aryl, 5-10 membered heteroaryl, 5-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L² is —CH₂— and X¹ is linear or branched C₁₋₆ alkyl, C₃₋₆ cycloalkyl, C₆ aryl, 5-10 membered heteroaryl, 6 membered heterocycloalkyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L² is —CH₂— and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L² is —CH₂— and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L² is —CH₂— and X¹ is cyclopropyl, wherein X¹ is unsubstituted.

In some embodiments of a compound of formula I, L¹ is —CH₂—, L² is a covalent bond and X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, —C₁₋₆ alkylhydroxy; or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, C₁₋₄ alkylhydroxy.

In some embodiments of a compound of formula I, L¹ is —CH₂—, L² is a covalent bond and X¹ is linear or branched C₁₋₆ alkyl, C₃₋₆ cycloalkyl, C₆ aryl, 5-10 membered heteroaryl, 5-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L¹ is —CH₂—, L² is a covalent bond and X¹ is linear or branched C₁₋₆ alkyl, C₃₋₆ cycloalkyl, C₆ aryl, 5-10 membered heteroaryl, 6 membered heterocycloalkyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L¹ is —CH₂—, L² is a covalent bond and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄. alkylhydroxy, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L¹ is —CH₂—, L² is a covalent bond and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L¹ is —CH₂—, L² is —CH₂— and X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, —C₁₋₆ alkylhydroxy; or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, C₁₋₄ alkylhydroxy.

In some embodiments of a compound of formula I, L¹ is —CH₂—, L² is —CH₂— and X¹ is linear or branched C₁₋₆ alkyl, C₃₋₆ cycloalkyl, C₆ aryl, 5-10 membered heteroaryl, 5-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L¹ is —CH₂—, L² is —CH₂— and X¹ is linear or branched C₁₋₆ alkyl, C₃₋₆ cycloalkyl, C₆ aryl, 5-10 membered heteroaryl, 6 membered heterocycloalkyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L¹ is —CH₂—, L² is —CH₂— and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula I, L¹ is —CH₂—, L² is —CH₂— and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula I, L¹ is —CH₂—, L² is —CH₂— and X¹ is cyclopropyl, wherein X¹ is unsubstituted.

In some embodiments of a compound of formula I, L³ is a covalent bond and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula I, L³ is a covalent bond and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula I, L³ is a covalent bond and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula I, L³ is a covalent bond and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula I, L³ is a covalent bond and X² is H, cyclopropyl, C₆ aryl, pyridinyl, oxetanyl, piperidinyl, wherein X² is unsubstituted or substituted with one or more of linear or branched C₁₋₄ alkyl, e.g. Me.

In some embodiments of a compound of formula I, L³ is —O—CH₂— and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula I, L³ is —O—CH₂— and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula I, L³ is —O—CH₂— and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula I, L³ is —O—CH₂— and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula I, L³ is —O—CH₂— and X² is H, pyrrolidinyl, wherein X² is unsubstituted or substituted with one or more of linear or branched C₁₋₄ alkyl, e.g. Me.

In some embodiments of a compound of formula I, L³ is —O—CH₂CH₂— and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula I, L³ is —O—CH₂CH₂— and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula I, L³ is —O—CH₂CH₂— and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula I, L³ is —O—CH₂CH₂— and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula I, L³ is —O—CH₂CH₂— and X² is H or morpholinyl, wherein X² is unsubstituted or substituted with one or more of linear or branched C₁₋₄ alkyl, e.g. Me.

In some embodiments of a compound of formula I, L³ is —CH₂— and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula I, L³ is —CH₂— and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula I, L³ is —CH₂— and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula I, L³ is —CH₂— and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula I, L³ is —CH₂— and X² is H, morpholinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula I, L³ is —O— and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula I, L³ is —O— and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula I, L³ is —O— and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula I, L³ is —O— and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula I, L³ is —O— and X² is H, C₆ aryl, pyrrolidinyl, wherein X² is unsubstituted or substituted with one or more of linear or branched C₁₋₄ alkyl, e.g. Me.

In specific embodiments the compound of formula I is a compound or pharmaceutically acceptable salts or stereoisomers thereof of formula II, such as III, IV or V:

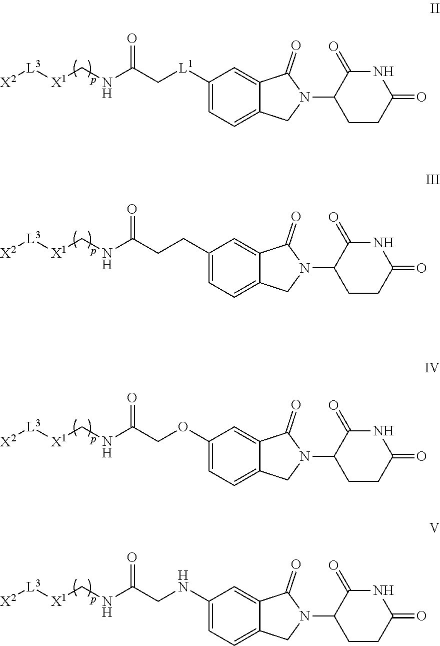

wherein X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, CF₃, CHF₂, —O—CHF₂, —O—(CH₂)₂—OMe, OCF₃, —CN, NH₂, C₁₋₆ alkoxy or C₁₋₆ alkylhydroxy;
or X¹ together with the N atom of the amide forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, —CN, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
L¹ is selected from —CH₂—, O and NH; L³ is a covalent bond, linear or branched C₁₋₆ alkyl, —O—, —C₁₋₄ alkoxy;
p is 0 or 1.

In some embodiments of a compound of formula II, L¹ is —CH₂—. In some embodiments of a compound of formula II, L¹ is O. In some embodiments of a compound of formula II, L¹ is NH.

In some embodiments of a compound of formula II, III, IV or V, L³ is a covalent bond. In some embodiments of a compound of formula II, III, IV or V, L³ is linear or branched C₁₋₆ alkyl. In some embodiments of a compound of formula II, III, IV or V, L³ is —O—. In some embodiments L³ is linear or branched C₁₋₄ alkoxy, such as —O—CH₂—, —O—CH₂—CH₂—. In some embodiments of a compound of formula II, III, IV or V, p is 0. In some embodiments of a compound of formula II, III, IV or V, p is 1.

In some embodiments of a compound of formula II, L¹ is —CH₂— and L³ is a covalent bond. In some embodiments of a compound of formula II, L¹ is —CH₂— and L³ is —CH₂—. In some embodiments of a compound of formula II, L¹ is —CH₂— and L³ is —O—. In some embodiments of a compound of formula II, L¹ is —CH₂— and L³ is —O—CH₂—. In some embodiments of a compound of formula II, L¹ is —CH₂— and L³ is —O—CH₂—CH₂—.

In some embodiments of a compound of formula II, L¹ is —CH₂—, p is 0 and L³ is a covalent bond. In some embodiments of a compound of formula II, L¹ is —CH₂—, p is 0 and L³ is —CH₂—. In some embodiments of a compound of formula II, L¹ is —CH₂—, p is 0 and L³ is —O—. In some embodiments of a compound of formula II, L¹ is —CH₂—, p is 0 and L³ is —O—CH₂—. In some embodiments of a compound of formula II, L¹ is —CH₂—, p is 0 and L³ is —O—CH₂—CH₂—.

In some embodiments of a compound of formula II, L¹ is —CH₂—, p is 1 and L³ is a covalent bond. In some embodiments of a compound of formula II, L¹ is —CH₂—, p is 1 and L³ is —CH₂—. In some embodiments of a compound of formula II, L¹ is —CH₂—, p is 1 and L³ is —O—. In some embodiments of a compound of formula II, L¹ is —CH₂—, p is 1 and L³ is —O—CH₂—. In some embodiments of a compound of formula II, L¹ is —CH₂—, p is 1 and L³ is —O—CH₂—CH₂—.

In some embodiments of a compound of formula III, IV or V, p is 0 and L³ is a covalent bond. In some embodiments of a compound of formula III, IV or V, p is 0 and L³ is —CH₂—. In some embodiments of a compound of formula III, IV or V, p is 0 and L³ is —O—. In some embodiments of a compound of formula III, IV or V, p is 0 and L³ is —O—CH₂—. In some embodiments of a compound of formula III, IV or V, p is 0 and L³ is —O—CH₂—CH₂—.

In some embodiments of a compound of formula III, IV or V, p is 1 and L³ is a covalent bond. In some embodiments of a compound of formula III, IV or V, p is 1 and L³ is —CH₂—. In some embodiments of a compound of formula III, IV or V, p is 1 and L³ is —O—. In some embodiments of a compound of formula III, IV or V, p is 1 and L³ is —O—CH₂—. In some embodiments of a compound of formula III, IV or V, p is 1 and L³ is —O—CH₂—CH₂—.

In some embodiments of a compound of formula II, III, IV or V, X¹ is linear or branched C₁-6 alkyl, C₃₋₆ cycloalkyl, C₆ aryl, 5-10 membered heteroaryl, 5-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula II, III, IV or V, X¹ is linear or branched C₁. 6 alkyl, C₃₋₆ cycloalkyl, C₆ aryl, 5-10 membered heteroaryl, 6 membered heterocycloalkyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula II, III, IV or V, X¹ is linear or branched C₁. 6 alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula II, III, IV or V, X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula II, III, IV or V, X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula II, III, IV or V, X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula II, III, IV or V, X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula II, III, IV or V, X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula II, III, IV or V, p is 0 and X¹ is linear or branched C₁₋₆ alkyl, C₃₋₆ cycloalkyl, C₆ aryl, 5-10 membered heteroaryl, 5-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula II, III, IV or V, p is 0 and X¹ is linear or branched C₁₋₆ alkyl, C₃₋₆ cycloalkyl, C₆ aryl, 5-10 membered heteroaryl, 6 membered heterocycloalkyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula II, III, IV or V, p is 0 and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula II, III, IV or V, p is 0 and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula II, III, IV or V, p is 1 and X¹ is linear or branched C₁₋₆ alkyl, C₃₋₆ cycloalkyl, C₆ aryl, 5-10 membered heteroaryl, 5-6 membered heterocycloalkyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula II, III, IV or V, p is 1 and X¹ is linear or branched C₁₋₆ alkyl, C₃₋₆ cycloalkyl, C₆ aryl, 5-10 membered heteroaryl, 6 membered heterocycloalkyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a 4-8 membered heterocycloalkyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula II, III, IV or V, p is 1 and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, or C₁₋₄ alkoxy.

In some embodiments of a compound of formula II, III, IV or V, p is 1 and X¹ is linear or branched C₁₋₆ alkyl, cyclopropyl, cyclopentyl, cyclohexyl, phenyl, pyridinyl, isothiazole, 2,3-dihydrobenzofuryl, cyclopentenopyridine, dihydropyrano-pyridine, piperidinyl, wherein X¹ is unsubstituted or substituted with one or more of halogen, linear or branched C₁₋₄ alkyl, CF₃, CHF₂, CMeF₂, OCF₃, OCHF₂, C₁₋₄ alkoxy; or X¹ together with the N atom of the amide forms a pyrrolidinyl, piperdinyl, morpholinyl, piperazinyl, N-methyl piperazinyl, which is unsubstituted or substituted with one or more of halogen, linear or branched C₁₋₄ alkyl, CF₃, CHF₂, CMeF₂, OCF₃, OCHF₂ and C₁₋₄ alkoxy.

In some embodiments of a compound of formula II, III, IV or V, p is 1 and X¹ is linear or branched C₁₋₆ alkyl or cyclopropyl.

In some embodiments of a compound of formula II, III, IV or V, L³ is a covalent bond and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula II, III, IV or V, L³ is a covalent bond and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula II, III, IV or V, L³ is a covalent bond and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula II, III, IV or V, L³ is a covalent bond and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula II, III, IV or V, L³ is a covalent bond and X² is H, cyclopropyl, C₆ aryl, pyridinyl, oxetanyl, piperidinyl, wherein X² is unsubstituted or substituted with one or more of linear or branched C₁₋₄ alkyl, e.g. Me.

In some embodiments of a compound of formula II, III, IV or V, L³ is —O—CH₂— and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula II, III, IV or V, L³ is —O—CH₂— and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula II, III, IV or V, L³ is —O—CH₂— and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula II, III, IV or V, L³ is —O—CH₂— and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula II, III, IV or V, L³ is —O—CH₂— and X² is H, pyrrolidinyl, wherein X² is unsubstituted or substituted with one or more of linear or branched C₁₋₄ alkyl, e.g. Me.

In some embodiments of a compound of formula II, III, IV or V, L³ is —O—CH₂CH₂— and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁-4 alkylhydroxy.

In some embodiments of a compound of formula II, III, IV or V, L³ is —O—CH₂CH₂— and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula II, III, IV or V, L³ is —O—CH₂CH₂— and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula II, III, IV or V, L³ is —O—CH₂CH₂— and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula II, III, IV or V, L³ is —O—CH₂CH₂— and X² is H or morpholinyl, wherein X² is unsubstituted or substituted with one or more of linear or branched C₁₋₄ alkyl, e.g. Me.

In some embodiments of a compound of formula II, III, IV or V, L³ is —CH₂— and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula II, III, IV or V, L³ is —CH₂— and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula II, III, IV or V, L³ is —CH₂— and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula II, III, IV or V, L³ is —CH₂— and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula II, III, IV or V, L³ is —CH₂— and X² is H, morpholinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula II, III, IV or V, L³ is —O— and X² is H, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula II, III, IV or V, L³ is —O— and X² is H, C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂.

In some embodiments of a compound of formula II, III, IV or V, L³ is —O— and X² is H, C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me.

In some embodiments of a compound of formula II, III, IV or V, L³ is —O— and X² is H, cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me.

In some embodiments of a compound of formula II, III, IV or V, L³ is —O— and X² is H, C₆ aryl, pyrrolidinyl, wherein X² is unsubstituted or substituted with one or more of linear or branched C₁₋₄ alkyl, e.g. Me.

In specific embodiments the compound of formula I is a compound or pharmaceutically acceptable salts or stereoisomers thereof of formula VI, such as VIa, VIb or VIc:

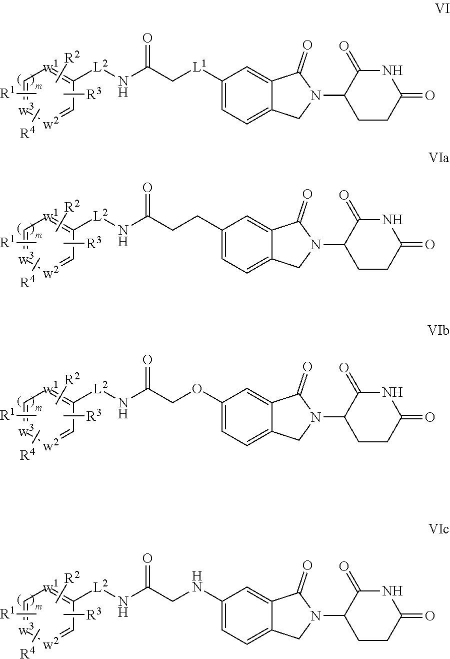

wherein
w¹, w², w³ are independently of each other selected from C, N, S, O, with the proviso that at least one of w¹, w², w³ is C;
L¹ is selected from —CH₂—, O and NH; L² is a covalent bond, linear or branched C₁₋₆ alkyl;
R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
m is 0 or 1.

In some embodiments of a compound of formula VI, L¹ is —CH₂—. In some embodiments of a compound of formula I, L¹ is O. In some embodiments of a compound of formula I, L¹ is NH.

In some embodiments of a compound of formula VI, such as VIa, VIb or VIc, L² is a covalent bond or C₁₋₄ alkyl, e.g. —CH₂—. In some embodiments of a compound of formula VI, such as VIa, VIb or VIc, L² is a covalent bond. In some embodiments of a compound of formula VI, such as VIa, VIb or VIc, L² is linear or branched C₁₋₆ alkyl, such as C₁₋₄ alkyl, e.g. —CH₂—.

In some embodiments of a compound of formula VI, such as VIa, VIb or VIc, m is 0. In some embodiments of a compound of formula VI, such as VIa, VIb or VIc, m is 1.

In some embodiments of a compound of formula VI, L¹ is —CH₂— and L² is a covalent bond or C₁₋₄ alkyl, e.g. —CH₂—. In some embodiments of a compound of formula VI, L¹ is —CH₂— and L² is a covalent bond.

In some embodiments of a compound of formula VI, such as VIa, VIb or VIc, w¹, w², w³ are independently of each other selected from C, N, S, with the proviso that at least one of w¹, w², w³ is C.

In some embodiments of a compound of formula VI, such as VIa, VIb or VIc, w¹, w², w³ are independently of each other selected from C, N, S, with the proviso that, for m=1, at least one of w¹, w², w³ is C and for m=0, one of w¹, w², w³ is C.

In some embodiments of a compound of formula VI, such as VIa, VIb or VIc, w¹, w², w³ are independently of each other selected from C, N, S, m=1, with the proviso that one or two of w¹, w², w³ are C. In some embodiments of a compound of formula VI, such as VIa, VIb or VIc, w¹, w², w³ are independently of each other selected from C, N, S, m=0, with the proviso that one of w¹, w², w³ is C.

In some embodiments of a compound of formula VI, such as VIa, VIb or VIc, w¹, w², w³ are independently of each other selected from C, N, S, with the proviso that, for m=1, at least two of w¹, w², w³ are C and for m=0, w² is C, one of w¹, w³ is S and one of w¹, w³ is N.

In some embodiments of a compound of formula VI, such as VIa, VIb or VIc, w¹, w², w³ are independently of each other selected from C, N, S, m=1, with the proviso that two of w¹, w², w³ are C. In some embodiments of a compound of formula VI, such as VIa, VIb or VIc, w¹, w², w³ are independently of each other selected from C, N, S, m=0, with the proviso that w² is C, one of w¹, w³ is S and one of w¹, w³ is N.

In some embodiments of a compound of formula VI, such as VIa, VIb or VIc, R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VI, such as VIa, VIb or VIc, R¹ is H; and R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VI, such as VIa, VIb or VIc, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂; R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VI, such as VIa, VIb or VIc, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VI, such as VIa, VIb or VIc, R¹ is a group of formula -L³-X², wherein L³ is a covalent bond, linear or branched C₁₋₆ alkyl, —O—, —C₁₋₄ alkoxy and X² is cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

More specific embodiments of the compound or pharmaceutically acceptable salts or stereoisomers thereof of formula VI are provided by formula VIa-1, VIb-1 or VIc-1, wherein 5 L² is —(CH₂)ₚ— and m is 1.

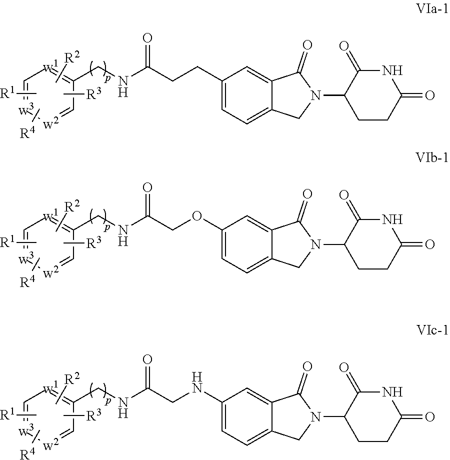

wherein
w¹, w², w³ are independently of each other selected from C and N with the proviso that at least one of w¹, w², w³ is C;
R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein
X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
p is 0 or 1.

In some embodiments of a compound of formula VIa-1, VIb-1 or VIc-1, p is 0. In some embodiments of a compound of formula VIa-1, VIb-1 or VIc-1, p is 1.

In some embodiments of a compound of formula VIa-1, VIb-1 or VIc-1, w¹, w² and w³ are independently of each other selected from C and N with the proviso that at least two of w¹, w², w³ are C.

In some embodiments of a compound of formula VIa-1, VIb-1 or VIc-1, w¹, w² and w³ are C. In some embodiments of a compound of formula VIa-1, VIb-1 or VIc-1, w¹, w² are C and w³ is N. In some embodiments of a compound of formula VIa-1, VIb-1 or VIc-1, w¹, w³ are C and w² is N. In some embodiments of a compound of formula VIa-1, VIb-1 or VIc-1, w², w³ are C and w¹ is N.

In some embodiments of a compound of formula VIa-1, VIb-1 or VIc-1, R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIa-1, VIb-1 or VIc-1, R¹ is H; and R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIa-1, VIb-1 or VIc-1, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂; R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIa-1, VIb-1 or VIc-1, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VIa-1, VIb-1 or VIc-1, R¹ is a group of formula -L³-X², wherein L³ is a covalent bond, linear or branched C₁₋₆ alkyl, —O—, —C₁₋₄ alkoxy and X² is cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments the compound or pharmaceutically acceptable salts or stereoisomers thereof of formula VI is defined by formula VI-1a, such as VIa-1a, VIb-1a, VIc-1a, wherein L² is a covalent bond, m is 1 and w¹, w², w³ are C

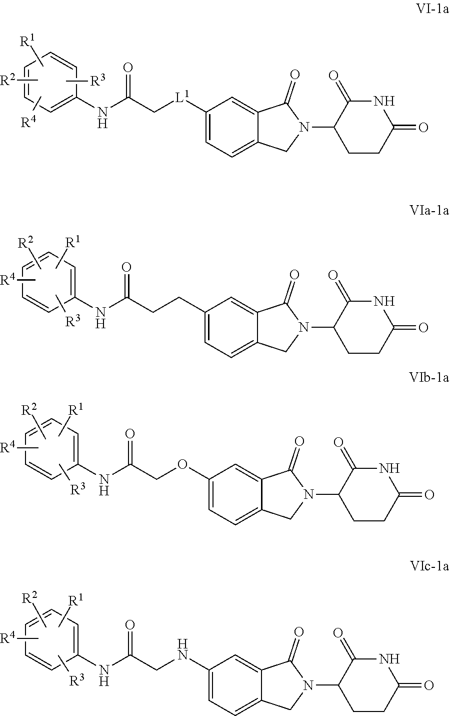

wherein
R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, or a group of formula -L³-X², wherein L³ is a covalent bond, linear or branched C₁₋₆ alkyl, —O—, —C₁₋₄ alkoxy and X² is C₃-6 cycloalkyl, C₆₋₁₀ aryl, 5-10 membered heteroaryl, 4-8 membered heterocycloalkyl, wherein
X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula VI-1a, L¹ is —CH₂—. In some embodiments of a compound of formula VI-1a, L¹ is O. In some embodiments of a compound of formula VI-1a, L¹ is NH.

In some embodiments of a compound of formula VI-1a, such as VIa-1a, VIb-1a, VIc-1a, R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VI-1a, such as VIa-1a, VIb-1a, VIc-1a, R¹ is H; and R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VI-1a, such as VIa-1a, VIb-1a, VIc-1a, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂; R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VI-1a, such as VIa-1a, VIb-1a, VIc-1a, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VI-1a, such as VIa-1a, VIb-1a, VIc-1a, R¹ is a group of formula -L³-X², wherein L³ is a covalent bond, linear or branched C₁₋₆ alkyl, —O—, —C₁₋₄ alkoxy and X² is cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VI-1a, such as VIa-1a, VIb-1a, VIc-1a, R¹ is a group of formula -L³-X², wherein L³ is one of a covalent bond, —CH₂—, —OCH₂—, —OCH₂CH₂—, —O— and X² is H, cyclopropyl, pyridinyl, oxetanyl, pyrrolidinyl, morpholinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl; with the proviso that a least one of R¹, R², R³, R⁴ is H.

In some embodiments the compound or pharmaceutically acceptable salts or stereoisomers thereof of formula VI is defined by formula VIa-1b, VIa-1c, VIa-1d, wherein L² is a covalent bond, m is 1 and one of w¹, w², w³ is N and the other two of w¹, w², w³ are C

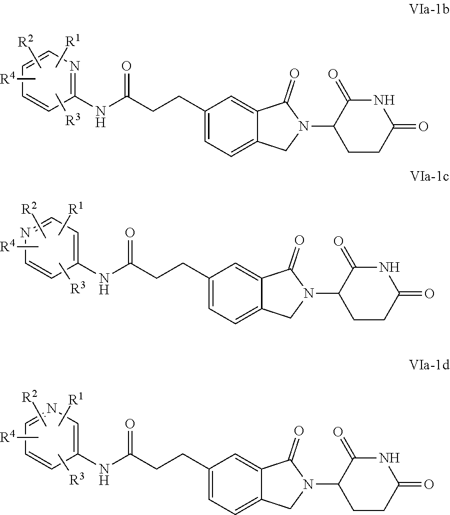

wherein
R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, or a group of formula -L³-X², wherein L³ is a covalent bond, linear or branched C₁₋₆ alkyl, —O—, —C₁₋₄ alkoxy and X² is C₃-6 cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula VIa-1b, VIa-1c or VIa-1d, R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIa-1b, VIa-1c or VIa-1d, R¹ is H; and R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIa-1b, VIa-1c or VIa-1d, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂; R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIa-1b, VIa-1c or VIa-1d, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VIa-1b, VIa-1c or VIa-1d, R¹ is a group of formula -L³-X², wherein L³ is a covalent bond, linear or branched C₁₋₆ alkyl, —O—, —C₁₋₄ alkoxy and X² is cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VIa-1b, VIa-1c or VIa-1d, R¹ is a group of formula -L³-X², wherein L³ is one of a covalent bond, —CH₂—, —OCH₂—, —OCH₂CH₂—, —O— and X² is H, cyclopropyl, pyridinyl, oxetanyl, pyrrolidinyl, morpholinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl; with the proviso that a least one of R¹, R², R³, R⁴ is H.

In some embodiments the compound or pharmaceutically acceptable salts or stereoisomers thereof of formula VI is defined by formula VI-2a, VI-2b or VI-2c, such as VIa-2a, VIa-2b, VIa-2c, VIb-2a, VIb-2b, VIb-2c, VIc-2a, VIc-2b or VIc-2c, wherein L² is a covalent bond, m is 1 and w¹, w², w³ are C

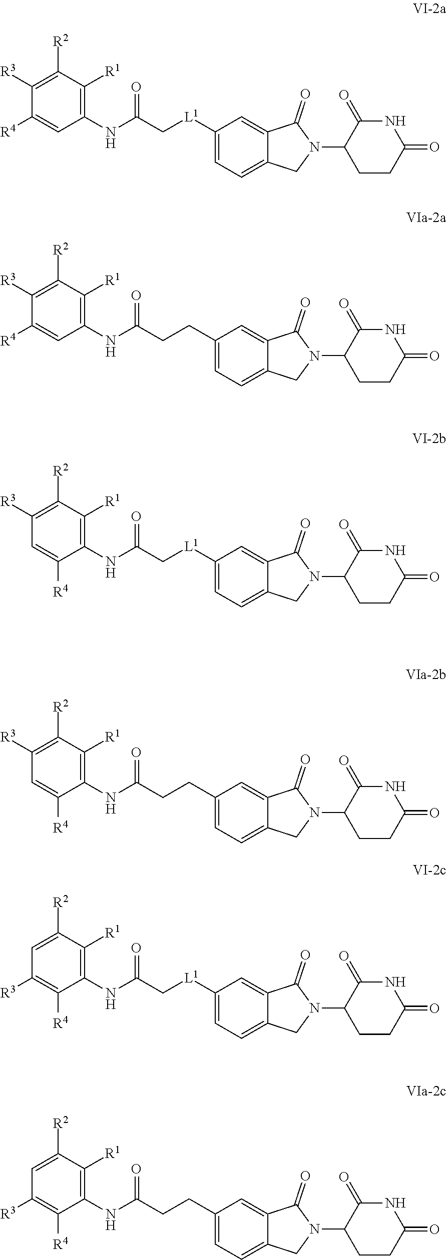

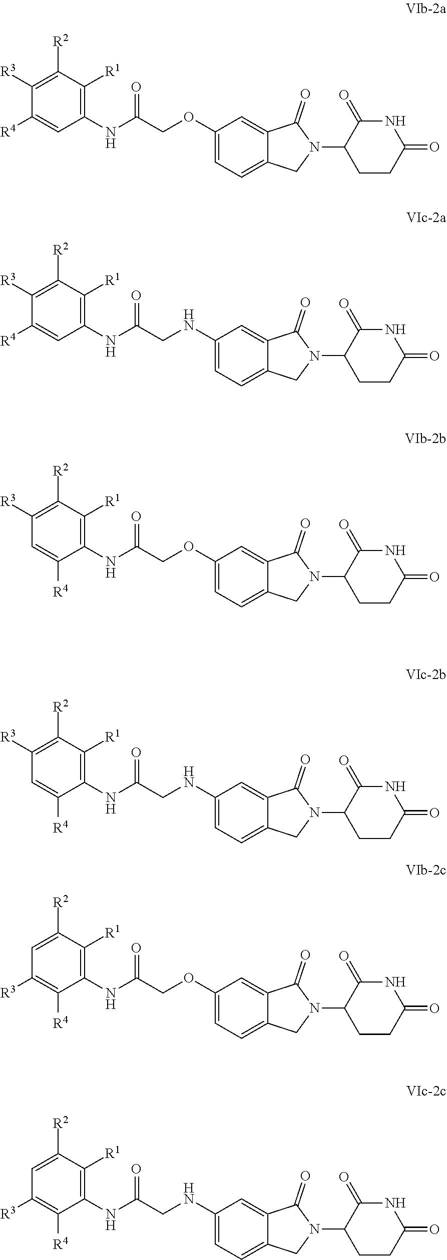

wherein
R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula VI-2a, VI-2b or VI-2c, L¹ is —CH₂—. In some embodiments of a compound of formula VI-2a, VI-2b or VI-2c, L¹ is 0. In some embodiments of a compound of formula VI-2a, VI-2b or VI-2c, L¹ is NH.

In some embodiments of a compound of formula VI-2a, VI-2b or VI-2c, such as VIa-2a, VIa-2b, VIa-2c, VIb-2a, VIb-2b, VIb-2c, VIc-2a, VIc-2b or VIc-2c, R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VI-2a, VI-2b or VI-2c, such as VIa-2a, VIa-2b, VIa-2c, VIb-2a, VIb-2b, VIb-2c, VIc-2a, VIc-2b or VIc-2c, R¹ is H; and R², R³, R⁴ are independently of each other selected from hydrogen, linear or branched —C₁₋₆ alkyl, —C₁. 6 alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VI-2a, VI-2b or VI-2c, such as VIa-2a, VIa-2b, VIa-2c, VIb-2a, VIb-2b, VIb-2c, VIc-2a, VIc-2b or VIc-2c, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂; R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VI-2a, VI-2b or VI-2c, such as VIa-2a, VIa-2b, VIa-2c, VIb-2a, VIb-2b, VIb-2c, VIc-2a, VIc-2b or VIc-2c, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VI-2a, VI-2b or VI-2c, such as VIa-2a, VIa-2b, VIa-2c, VIb-2a, VIb-2b, VIb-2c, VIc-2a, VIc-2b or VIc-2c, R¹ is a group of formula -L³-X², wherein L³ is a covalent bond, linear or branched C₁₋₆ alkyl, —O—, —C₁₋₄ alkoxy and X² is cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VI-2a, VI-2b or VI-2c, such as VIa-2a, VIa-2b, VIa-2c, VIb-2a, VIb-2b, VIb-2c, VIc-2a, VIc-2b or VIc-2c, one of R², R³ is a group of formula -L³-X², wherein L³ is one of a covalent bond, —CH₂—, —OCH₂—, —OCH₂CH₂—, —O— and X² is H, cyclopropyl, pyridinyl, oxetanyl, pyrrolidinyl, morpholinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R¹, R⁴ and the other one of R², R³ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl; with the proviso that a least one of R¹, R³, R⁴ is H.

In some embodiments the compound or pharmaceutically acceptable salts or stereoisomers thereof of formula VI is defined by formula VIa-2d, VIa-2e or VIa-2f, wherein L² is a covalent bond, m is 1 and one of w¹, w², w³ is N and the other two of w¹, w², w³ are C

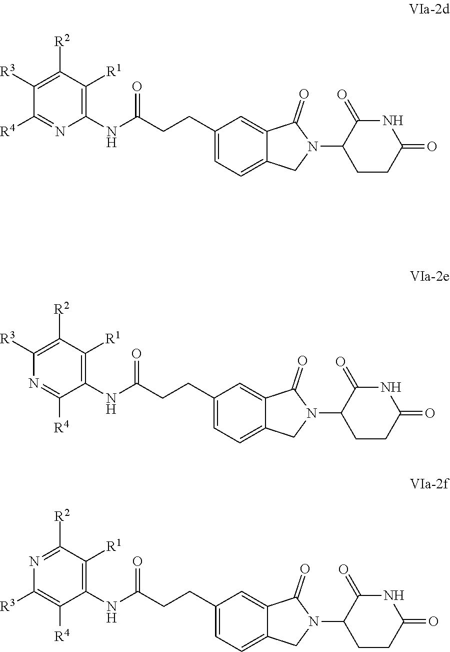

wherein
R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy.

In some embodiments of a compound of formula VIa-2d, VIa-2e, VIa-2f, R¹, R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIa-2d, VIa-2e, VIa-2f, R¹ is H; and R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIa-2d, VIa-2e, VIa-2f, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-6 membered heteroaryl, 4-8 membered heterocycloalkyl, wherein X² is unsubstituted or substituted with one or more of linear or branched C₁₋₄ alkyl, —C₁₋₄ alkoxy, NMe₂, halogen, CF₃, CHF₂, CMeF₂, OCF₃, OCHF₂; R², R³, R⁴ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIa-2d, VIa-2e, VIa-2f, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 6 membered heteroaryl, 4-8 membered heterocycloalkyl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VIa-2d, VIa-2e, VIa-2f, R¹ is a group of formula -L³-X², wherein L³ is a covalent bond, linear or branched C₁₋₆ alkyl, —O—, —C₁₋₄ alkoxy and X² is cyclopropyl, C₆ aryl, pyridinyl, oxetanyl, pyrrolidinyl, morpholinyl, piperidinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl.

In some embodiments of a compound of formula VIa-2d, VIa-2e, VIa-2f, R³ is a group of formula -L³-X², wherein L³ is one of a covalent bond, —CH₂—, —OCH₂—, —OCH₂CH₂—, —O— and X² is H, cyclopropyl, pyridinyl, oxetanyl, pyrrolidinyl, morpholinyl, 2-oxa-5-aza-bicyclo[2.2.1]heptane, 8-oxa-3-azabicyclo[3.2.1]octan-3-yl, wherein X² is unsubstituted or substituted with one or more of linear or branched C₁₋₄ alkyl, e.g. Me; R², R³, R⁴ are independently of each other selected from hydrogen, linear or branched —C₁₋₄ alkyl, such as methyl, ethyl and t-butyl, —C₁₋₄ alkoxy, such as methoxy and ethoxy, CF₃, CHF₂, CMeF₂, OCF₃, OCHF₂, halogen, such as F, Cl, Br, e.g. F or Cl; with the proviso that a least one of R¹, R³, R⁴ is H.

In some embodiments, the compound or pharmaceutically acceptable salts or stereoisomers thereof of formula VI is defined by formula VIa-3, VIb-3 or VIc-3, wherein L² is —(CH₂)ₚ— and m is 0

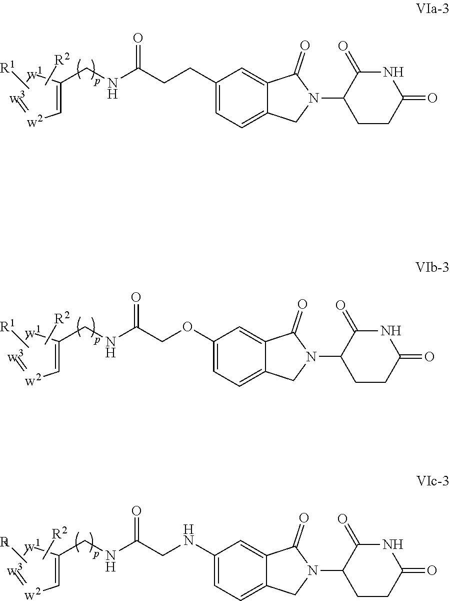

wherein
w¹, w², w³ are independently of each other selected from C, N, S, O, with the proviso that one or two of w¹, w², w³ is C;
R¹, R² are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein
X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
p is 0 or 1.

In some embodiments of a compound of formula VIa-3, VIb-3 or VIc-3, p is 0. In some embodiments of a compound of formula VIa-3, VIb-3 or VIc-3 p is 1.

In some embodiments of a compound of formula VIa-3, VIb-3 or VIc-3, w¹, w² and w³ are independently of each other selected from C, N, S, with the proviso that at least one of w¹, w², w³ is C. In some embodiments of a compound of formula VIa-3, VIb-3 or VIc-3, w¹, w² and w³ are independently of each other selected from C, N, S, with the proviso that one of w¹, w², w³ is C. In some embodiments of a compound of formula VIa-3, VIb-3 or VIc-3, w² is C, one of w¹, w³ is S and the other of w¹, w³ is N.

In some embodiments of a compound of formula VIa-3, VIb-3 or VIc-3, p is 0 and w¹, w² and w³ are independently of each other selected from C, N, S, with the proviso that at least one of w¹, w², w³ is C. In some embodiments of a compound of formula VIa-3, VIb-3 or VIc-3, p is 0 and w¹, w² and w³ are independently of each other selected from C, N, S, with the proviso that one of w¹, w², w³ is C. In some embodiments of a compound of formula VIa-3, VIb-3 or VIc-3, p is 0 and w² is C, one of w¹, w³ is S and the other of w¹, w³ is N.

In some embodiments of a compound of formula VIa-3, VIb-3 or VIc-3, R¹, R² are independently selected from linear or branched —C₁₋₆ alkyl, such as ethyl, CF₃, CHF₂, CMeF₂, halogen, such as F, Cl.

In some embodiments of a compound of formula VIa-3, VIb-3 or VIc-3, R¹, R² are independently selected from linear or branched —C₁₋₆ alkyl, such as ethyl, CF₃.

In some embodiments of a compound of formula VIa-3, VIb-3 or VIc-3, R¹, R² are independently selected from linear or branched —C₁₋₆ alkyl, such as ethyl, CF₃, CHF₂, CMeF₂, halogen, such as F, Cl and w¹, w² and w³ are independently of each other selected from C, N, S, with the proviso that at least one of w¹, w², w³ is C. In some embodiments of a compound of formula VIa-3, VIb-3 or VIc-3, R¹, R² are independently selected from linear or branched —C₁₋₆ alkyl, such as ethyl, CF₃, CHF₂, CMeF₂, halogen, such as F, Cl and w¹, w² and w³ are independently of each other selected from C, N, S, with the proviso that one of w¹, w², w³ is C. In some embodiments of a compound of formula VIa-3, VIb-3 or VIc-3, R¹, R² are independently selected from linear or branched —C₁₋₆ alkyl, such as ethyl, CF₃, CHF₂, CMeF₂, halogen, such as F, Cl; w² is C, one of w¹, w³ is S and the other of w¹, w³ is N.

In some embodiments the compound of formula I is a compound or pharmaceutically acceptable salts or stereoisomers thereof of formula VII, such as VIIa, VIIb or VIIc:

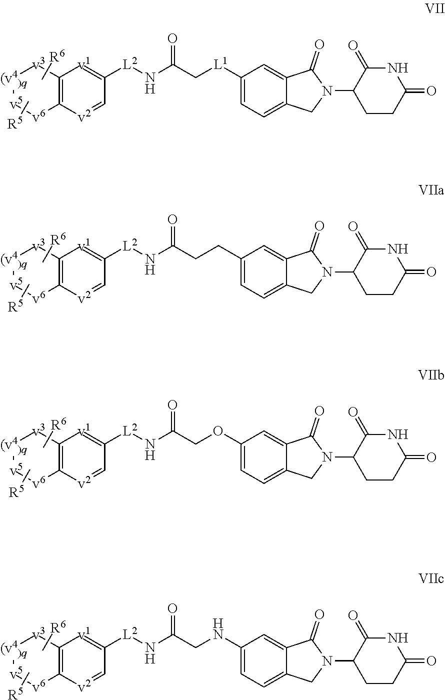

wherein
v¹, v², v³, v⁴, v⁵, v⁶ are independently of each other selected from C, N, O, with the proviso that at least three of v¹, v², V³, V⁴, v⁵, v⁶ are C;
L¹ is selected from —CH₂—, O and NH;
L² is a covalent bond, linear or branched C₁₋₆ alkyl;
R⁵, R⁶ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl;
q is 0 or 1.

In some embodiments of a compound of formula VII, L¹ is —CH₂—. In some embodiments of a compound of formula I, L¹ is O. In some embodiments of a compound of formula I, L¹ is NH.

In some embodiments of a compound of formula VII, such as VIIa, VIIb, VIIc, L² is a covalent bond. In some embodiments of a compound of formula VII, such as VIIa, VIIb, VIIc, L² is linear or branched C₁₋₆ alkyl, such as C₁₋₄ alkyl, e.g. —CH₂—.

In some embodiments of a compound of formula VII, such as VIIa, VIIb, VIIc, q is 0. In some embodiments of a compound of formula VII, such as VIIa, VIIb, VIIc, q is 1.

In some embodiments of a compound of formula VII, L¹ is —CH₂— and L² is a covalent.

In some embodiments of a compound of formula VII, such as VIIa, VIIb or VIIc, v¹, v² are independently of each other selected from C, N. In some embodiments of a compound of formula VII, such as VIIa, VIIb or VIIc, one or two of v³, v⁴, v⁵, v⁶ are independently of each other selected from C and O and the remaining of v³, v⁴, v⁵, v⁶ are C.

In some embodiments of a compound of formula VII, such as VIIa, VIIb or VIIc, v¹, v² are independently of each other selected from C, N, with the proviso that at least one of v¹, v² is C. In some embodiments of a compound of formula VII, such as VIIa, VIIb or VIIc, one of v¹, v² is N, the other of v¹, v² is C. In some embodiments of a compound of formula VII, such as VIIa, VIIb or VIIc, v¹, v² are C.

In some embodiments of a compound of formula VII, such as VIIa, VIIb or VIIc, v³, v⁴, v⁵, v⁶ are independently of each other selected from C, O, with the proviso that at least two of v³, v⁴, v⁵, v⁶ are C.

In some embodiments of a compound of formula VII, such as VIIa, VIIb or VIIc, v¹, v², v³, v⁴, v⁵, v⁶ are independently of each other selected from C, N, S, O, q=0, with the proviso that four of v¹, v², v³, v⁴, v⁵, v⁶ are C.

In some embodiments of a compound of formula VII, such as VIIa, VIIb or VIIc, q=0, v¹, v⁵, v⁶ are C and v², v³, are independently of each other selected from C, N, O, e.g. N, O.

In some embodiments of a compound of formula VII, such as VIIa, VIIb or VIIc, q=0, v¹, v⁵, v⁶ are C, v² is selected from C, N and v³ is selected from C, O.

In some embodiments of a compound of formula VII, such as VIIa, VIIb or VIIc, q is 0, v³, v⁵, v⁶ are independently of each other selected from C, O, with the proviso that at least one of v³, v⁵, v⁶ are C.

In some embodiments of a compound of formula VII, such as VIIa, VIIb or VIIc, q is 0, v³, v⁵, v⁶ are independently of each other selected from C, O, with the proviso that at least two of v³, v⁵, v⁶ are C.

In some embodiments of a compound of formula VII, such as VIIa, VIIb or VIIc, q is 1, v³, v⁴, v⁵, v⁶ are independently of each other selected from C, O, with the proviso that at least three of v³, v⁴, v⁵, v⁶ are C.

In some embodiments of a compound of formula VII, such as VIIa, VIIb or VIIc, q is 1, one of v³, v⁴, v⁵, v⁶ is 0, and the remaining of v³, v⁴, v⁵, v⁶ are C.

In some embodiments of a compound of formula VII, such as VIIa, VIIb, VIIc, R⁵, R⁶ are independently of each other selected from hydrogen, linear or branched —C₁₋₄ alkyl, —C₁₋₄ alkoxy, CF₃, CHF₂, CMeF₂, OCF₃, OCHF₂, halogen, such as F or Cl.

In some embodiments of a compound of formula VII, such as VIIa, VIIb, VIIc, R⁵, R⁶ are independently of each other selected from hydrogen, linear or branched —C₁₋₄ alkyl, such as methyl; halogen, such as F.

In some embodiments of a compound of formula VII, such as VIIa, VIIb, VIIc, R⁵ is H and R⁶ is selected from hydrogen, linear or branched —C₁₋₄ alkyl, such as methyl; halogen, such as F.

More specific embodiments of the compound or pharmaceutically acceptable salts or stereoisomers thereof of formula VII are provided by formula VIIa-1a, VIIa-1b or VIIa-1c, wherein L² is a covalent bond, q is 1 and v¹, v² are either both C or one of v¹, v² is C and the other of v¹, v² is N

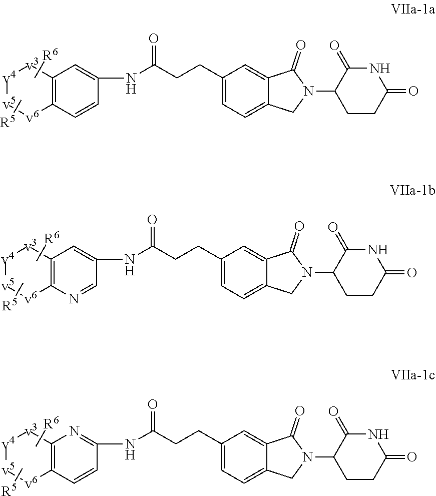

wherein
v³, v⁴, v⁵, v⁶ are independently of each other selected from C, O, with the proviso that at least three of v³, v⁴, v⁵, v⁶ are C;
R⁵, R⁶ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIIa-1a, VIIa-1b, VIIa-1c, one of v³, v⁴, v⁵, v⁶ is O, and the remaining of v³, v⁴, v⁵, v⁶ are C.

In some embodiments of a compound of formula VIIa-1a, VIIa-1b, VIIa-1c, v³, v⁵, v⁶ are C and v⁴ is selected from C, O.

In some embodiments of a compound of formula VIIa-1a, VIIa-1b, VIIa-1c, v³, v⁴, v⁵, v⁶ are C.

In some embodiments of a compound of formula VIIa-1a, VIIa-1b, VIIa-1c, v³, v⁵, v⁶ are C and v⁴ is O.

In some embodiments of a compound of formula VIIa-1a, VIIa-1b, VIIa-1c, R⁵, R⁶ are independently of each other selected from hydrogen, linear or branched —C₁₋₄ alkyl, —C₁₋₄ alkoxy, CF₃, CHF₂, CMeF₂, OCF₃, OCHF₂, halogen, such as F or Cl.

In some embodiments of a compound of formula VIIa-1a, VIIa-1b, VIIa-1c, R⁵, R⁶ are independently of each other selected from hydrogen, linear or branched —C₁₋₄ alkyl, such as methyl; halogen, such as F.

In some embodiments of a compound of formula VIIa-1a, VIIa-1b, VIIa-1c, R⁵ is H and R⁶ is selected from hydrogen, linear or branched —C₁₋₄ alkyl, such as methyl; halogen, such as F.

In some embodiments of a compound of formula VIIa-1a, v³, v⁶ are C, one of v⁴, v⁵ is C, the other one of v⁴, v⁵ is O and R⁶ is H.

In some embodiments of a compound of formula VIIa-1a, v⁴, v⁵ are C, one of v³, v⁶ is C, the other one of v³, v⁶ is O and R⁶ is H.

In some embodiments of a compound of formula VIIa-1b, v³, v⁶ are C, one of v⁴, v⁵ is C, the other one of v⁴, v⁵ is O and R⁶ is H.

In some embodiments of a compound of formula VIIa-1b, v⁴, v⁵ are C, one of v³, v⁶ is C, the other one of v³, v⁶ is O and R⁶ is H.

In some embodiments of a compound of formula VIIa-1c, v³, v⁶ are C, one of v⁴, v⁵ is C, the other one of v⁴, v⁵ is O and R⁶ is H.

In some embodiments of a compound of formula VIIa-1c, v⁴, v⁵ are C, one of v³, v⁶ is C, the other one of v³, v⁶ is O and R⁶ is H.

More specific embodiments of the compound or pharmaceutically acceptable salts or stereoisomers thereof of formula VII are provided by formula VIIa-3a, VIIa-3b or VIIa-3c, wherein L² is a covalent bond, q is 0 and v¹, v² are either both C or one of v¹, v² is C and the other of v¹, v² is N

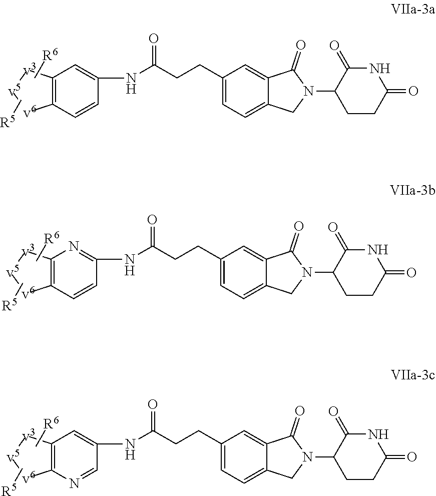

wherein
v³, v⁵, v⁶, are independently of each other selected from C, O, with the proviso that at least one of v³, v⁵, v⁶ are C;
R⁵, R⁶ are independently of each other selected from hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl.

In some embodiments of a compound of formula VIIa-3a, VIIa-3b, VIIa-3c, v³, v⁵, v⁶ are independently of each other selected from C, O, with the proviso that at least two of v³, v⁵, v⁶ are C.

In some embodiments of a compound of formula VIIa-3a, VIIa-3b, VIIa-3c, v⁵, v⁶ are C and v³ is selected from C, O.

In some embodiments of a compound of formula VIIa-3a, one of v³, v⁵, v⁶ is selected from C, O, the other two of v³, v⁵, v⁶ is C and R⁵ is H.

In some embodiments of a compound of formula VIIa-3a, v⁵ is O, v³, v⁶ are C and R⁵ is H.

In some embodiments of a compound of formula VIIa-3a, v⁵ is C, one of v³, v⁶, such as v³ is O, the other one of v³, v⁶, such as v⁶ is C and R⁵ is H.

In some embodiments of a compound of formula VIIa-3b, VIIa-3c, v³, v⁵, v⁶ are C and R⁵ is H.

In some embodiments of a compound of formula VIIa-3b, VIIa-3c, one of v³, v⁵, v⁶ is N, the other two of v³, v⁵, v⁶ are C and R⁵ is H.

In some embodiments of a compound of formula VIIa-3a, VIIa-3b, VIIa-3c, R⁵, R⁶ are independently of each other selected from hydrogen, linear or branched —C₁₋₄ alkyl, —C₁₋₄ alkoxy, CF₃, CHF₂, CMeF₂, OCF₃, OCHF₂, halogen, such as F or Cl.

In some embodiments of a compound of formula VIIa-3a, VIIa-3b, VIIa-3c, R⁵, R⁶ are independently of each other selected from hydrogen, linear or branched —C₁₋₄ alkyl, such as methyl; halogen, such as F.

In some embodiments of a compound of formula VIIa-3a, VIIa-3b, VIIa-3c, R⁵ is H and R⁶ is selected from hydrogen, linear or branched —C₁₋₄ alkyl, such as methyl; halogen, such as F.

In some embodiments the compound of formula I is a compound or pharmaceutically acceptable salts or stereoisomers thereof of formula VIII, such as VIIIa, VIIIb or VIIIc:

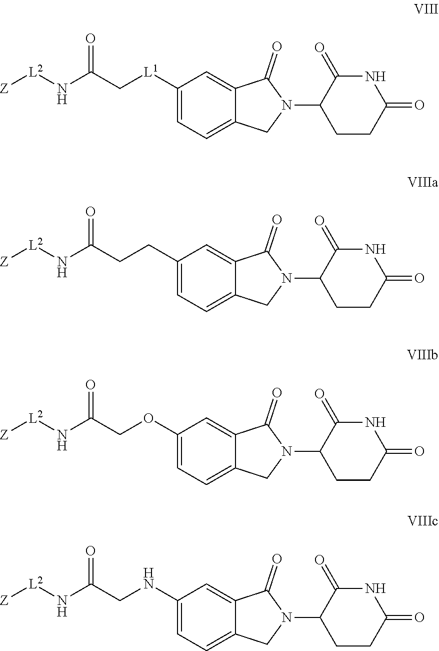

wherein
L¹ is selected from —CH₂—, O and NH;
L² is a covalent bond, linear or branched C₁₋₆ alkyl;
Z is linear or branched —C₁₋₆ alkyl, —C₃₋₆ cycloalkyl, —C₁₋₄ alkoxy, 4-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the amide forms a 4-6 membered heterocycloalkyl, which is unsubstituted or substituted with C₁₋₄ alkyl, C₆ aryl, C₆ aryloxy, 6 membered heteroaryl or CF₃.

In some embodiments of a compound of formula VIII, L¹ is —CH₂—. In some embodiments of a compound of formula VIII, L¹ is O. In some embodiments of a compound of formula VIII, L¹ is NH.

In some embodiments of a compound of formula VIII, VIIIa, VIIIb or VIIIc, L² is a covalent bond. In some embodiments of a compound of formula VIII, L² is linear or branched C₁₋₆ alkyl, such as C₁₋₄ alkyl, e.g. —CH₂—.

In some embodiments of a compound of formula VIII, VIIIa, VIIIb or VIIIc, Z is linear or branched —C₁₋₆ alkyl, —C₃₋₆ cycloalkyl, 5-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the amide forms a 5-6 membered heterocycloalkyl, which is unsubstituted or substituted with C₁₋₄ alkyl, C₆ aryl, C₆ aryloxy, 6 membered heteroaryl or CF₃.

In some embodiments of a compound of formula VIII, VIIIa, VIIIb or VIIIc, Z is linear or branched —C₁₋₆ alkyl, —C₃₋₆ cycloalkyl, 5-6 membered heterocycloalkyl, wherein Z is unsubstituted or substituted with C₁₋₄ alkyl or 6 membered heteroaryl; or Z together with the N atom of the amide forms a 5-6 membered heterocycloalkyl, which is unsubstituted or substituted with C₁₋₄ alkyl, C₆ aryl or C₆ aryloxy.

In some embodiments of a compound of formula VIII, VIIIa, VIIIb or VIIIc, L² is a covalent bond and Z is linear or branched —C₁₋₆ alkyl, —C₅₋₆ cycloalkyl, 5-6 membered heterocycloalkyl, wherein Z is unsubstituted or substituted with C₁₋₄ alkyl or 6 membered heteroaryl.

In some embodiments of a compound of formula VIII, VIIIa, VIIIb or VIIIc, L² is —CH₂— and Z is —C₃₋₆ cycloalkyl, such as cyclopropyl, or Z together with the N atom of the amide forms a 5-6 membered heterocycloalkyl, which is unsubstituted or substituted with C₁₋₄ alkyl, C₆ aryl or C₆ aryloxy.

More specific embodiments of the compound or pharmaceutically acceptable salts or stereoisomers thereof of formula VIII are provided by formula VIIIa-1, VIIIb-1 or VIIIc-1, wherein L² is —(CH₂)ₚ—

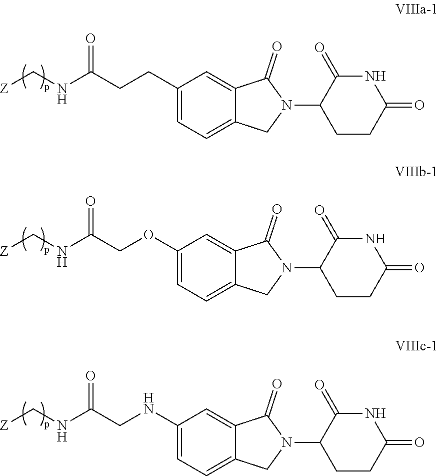

wherein
p is 0 or 1;
Z is linear or branched —C₁₋₆ alkyl, —C₃₋₆ cycloalkyl, —C₁₋₄ alkoxy, 4-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the amide forms a 4-6 membered heterocycloalkyl, which is unsubstituted or substituted with C₁₋₄ alkyl, C₆ aryl, C₆ aryloxy, 6 membered heteroaryl or CF₃.

In some embodiments of a compound of formula VIIIa-1, VIIIb-1, VIIIc-1, p is 0. In some embodiments of a compound of formula VIIIa-1, VIIIb-1, VIIIc-1, p is 1.

In some embodiments of a compound of formula VIIIa-1, VIIIb-1, VIIIc-1, Z is linear or branched —C₁₋₆ alkyl, —C₃₋₆ cycloalkyl, 5-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the amide forms a 5-6 membered heterocycloalkyl, which is unsubstituted or substituted with C₁₋₄ alkyl, C₆ aryl, C₆ aryloxy, 6 membered heteroaryl or CF₃.

In some embodiments of a compound of formula VIIIa-1, VIIIb-1, VIIIc-1, Z is linear or branched —C₁₋₆ alkyl, —C₃₋₆ cycloalkyl, 5-6 membered heterocycloalkyl, wherein Z is unsubstituted or substituted with C₁₋₄ alkyl or 6 membered heteroaryl; or Z together with the N atom of the amide forms a 5-6 membered heterocycloalkyl, which is unsubstituted or substituted with C₁₋₄ alkyl, C₆ aryl or C₆ aryloxy.

In some embodiments of a compound of formula VIIIa-1, VIIIb-1, VIIIc-1, p is 0 and Z is linear or branched —C₁₋₆ alkyl, —C₅₋₆ cycloalkyl, 5-6 membered heterocycloalkyl, wherein Z is unsubstituted or substituted with C₁₋₄ alkyl or 6 membered heteroaryl.

In some embodiments of a compound of formula VIIIa-1, VIIIb-1, VIIIc-1, p is 1 and Z is —C₃₋₆ cycloalkyl, such as cyclopropyl, or Z together with the N atom of the amide forms a 5-6 membered heterocycloalkyl, which is unsubstituted or substituted with C₁₋₄ alkyl, C₆ aryl or C₆ aryloxy.

In more specific embodiments, the present disclosure is directed towards a compound or pharmaceutically acceptable salts or stereoisomers thereof of formula IX

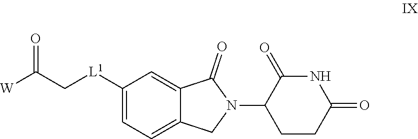

wherein L¹ is selected from —CH₂—, O and NH; and
W is selected from

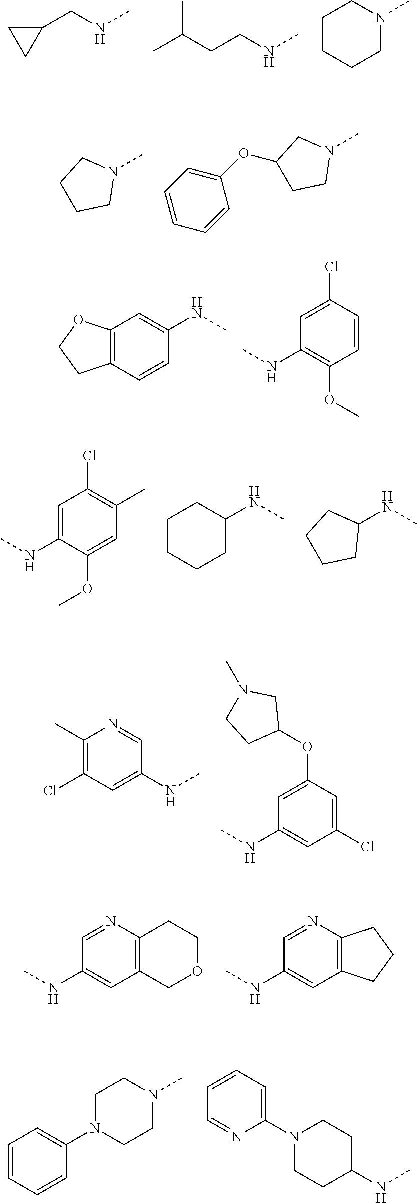

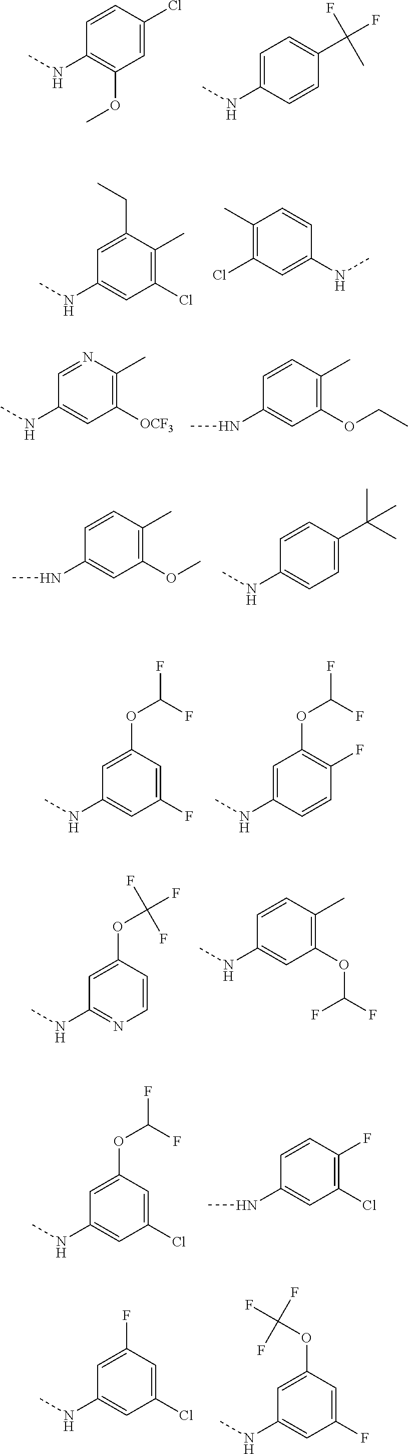

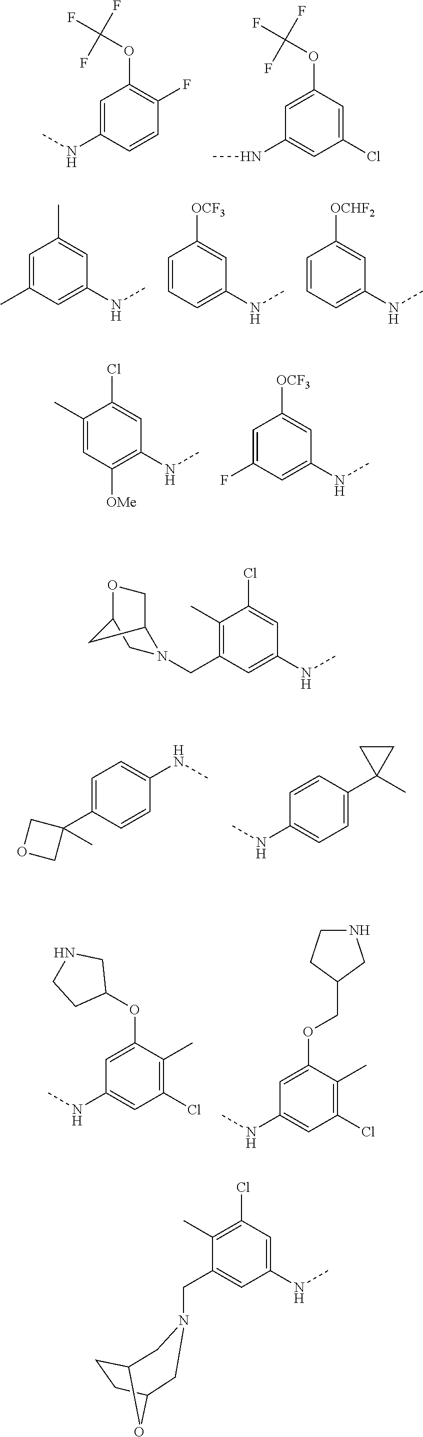

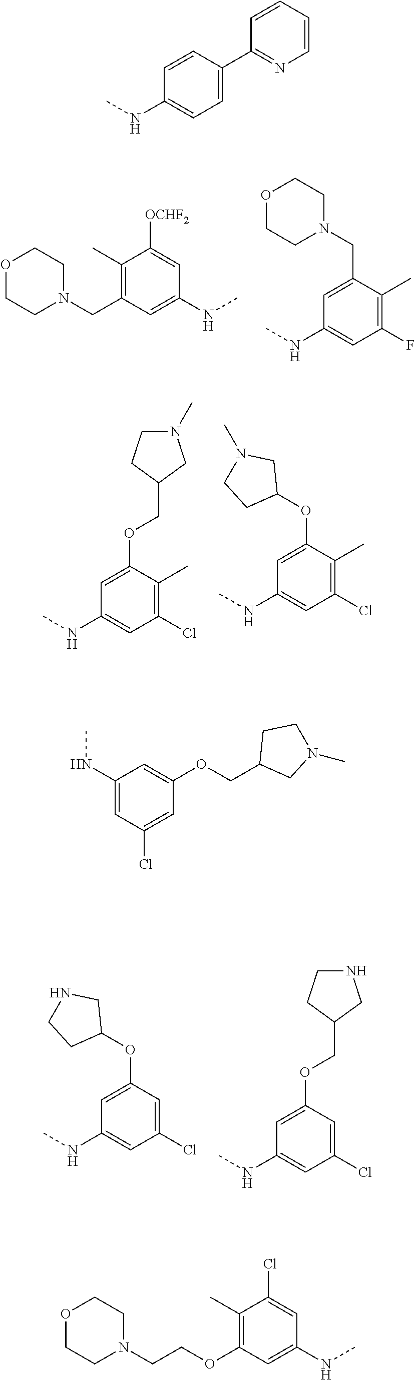

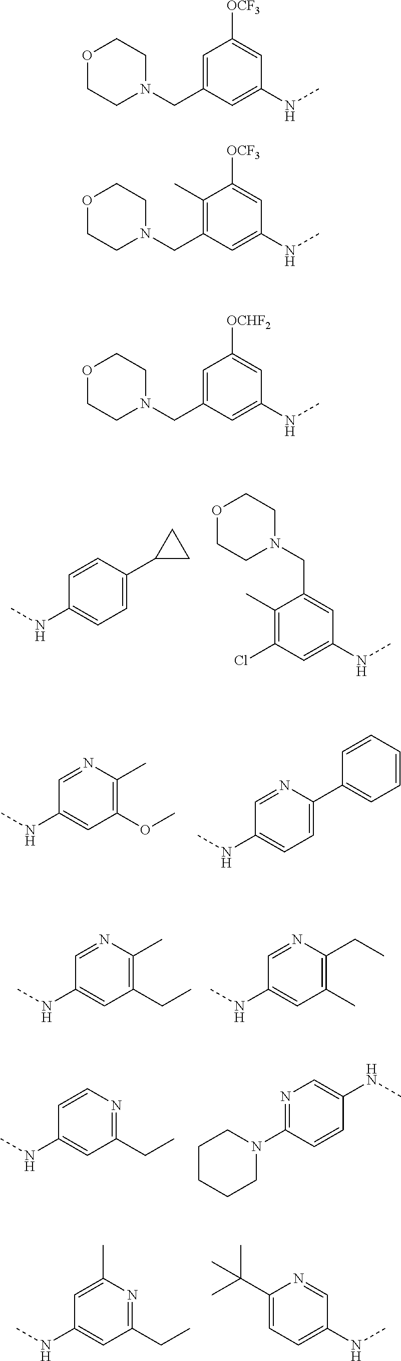

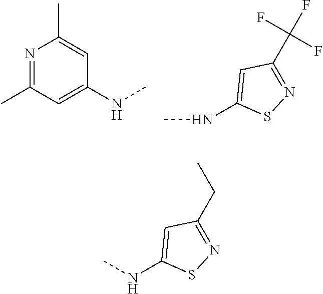

In some embodiments of a compound of formula IX, L¹ is —CH₂—. In some embodiments of a compound of formula IX, L¹ is O. In some embodiments of a compound of formula IX, L¹ is NH.

In some embodiments of a compound of formula IX, L¹ is O or NH and w is selected from

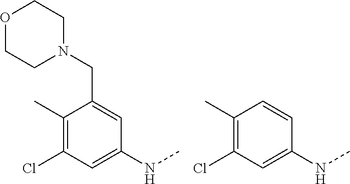

In more specific embodiments, the present disclosure is directed towards a compound or pharmaceutically acceptable salts or stereoisomers thereof of formula X

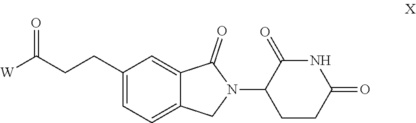

wherein
W is selected from

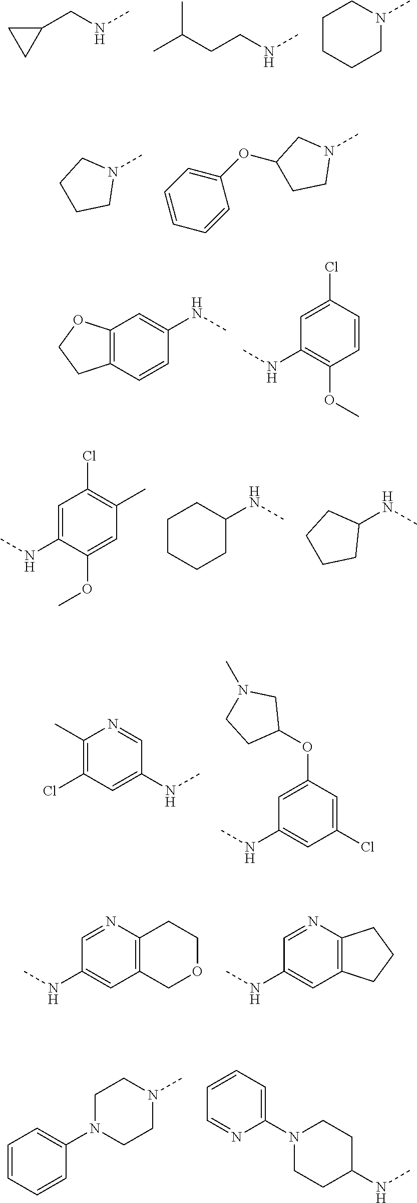

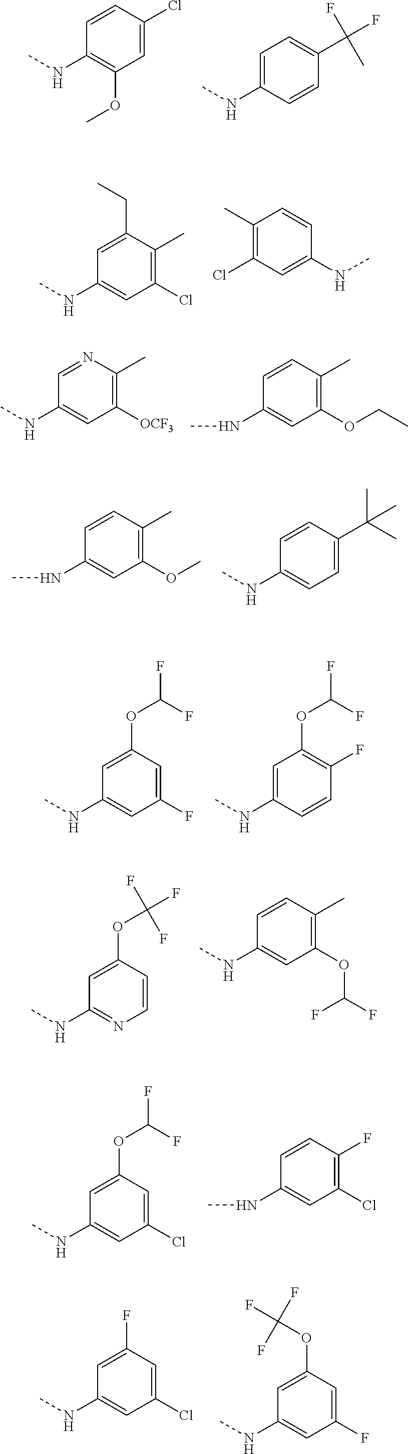

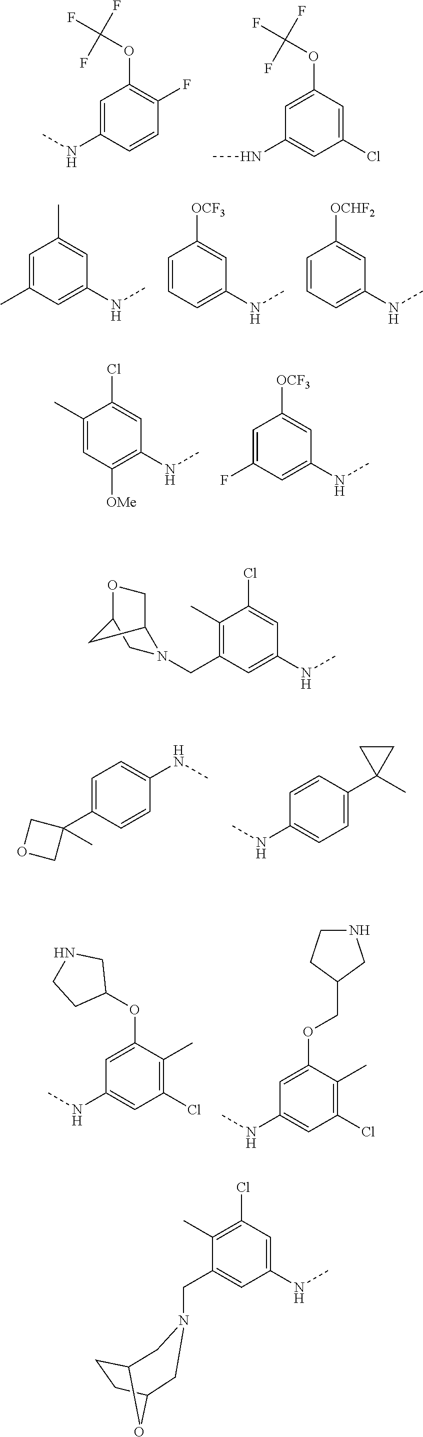

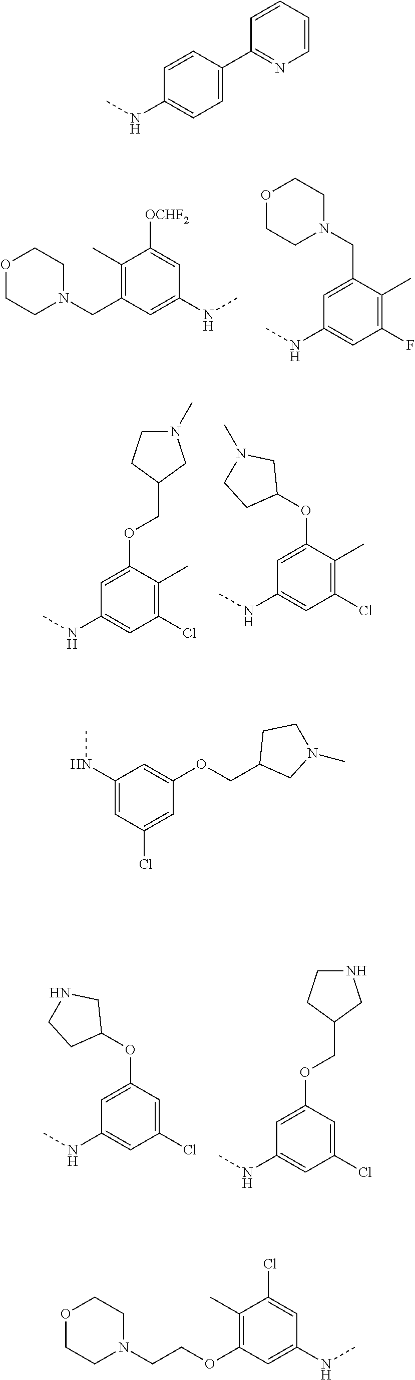

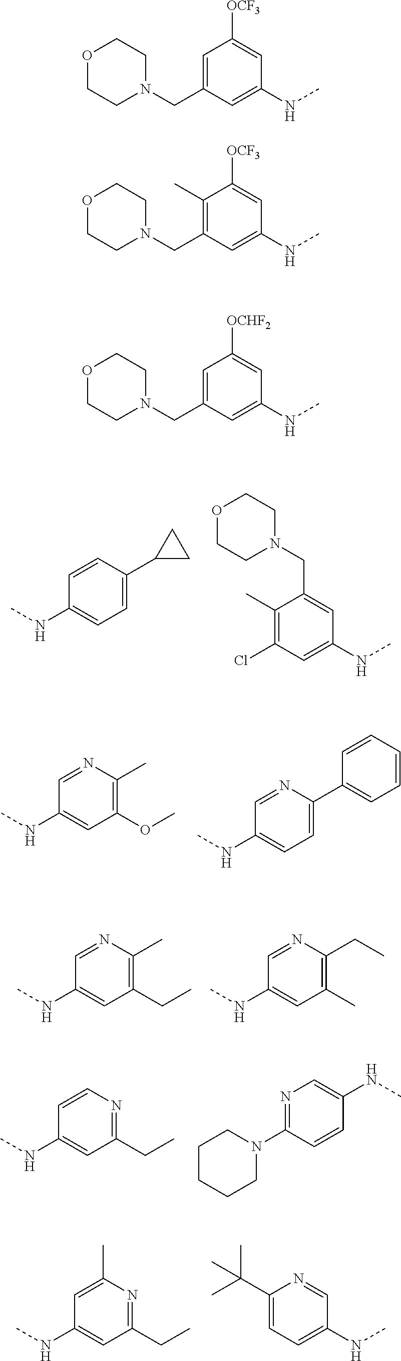

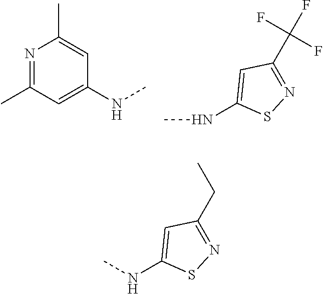

In some embodiments, provided herein is a compound of formula I

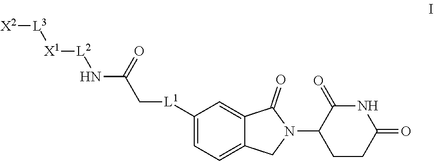

or a pharmaceutically acceptable salt or stereoisomer thereof,
wherein
X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, and —C₁₋₆ alkylhydroxy;
or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, and C₁₋₄ alkylhydroxy;
X² is selected from the group consisting of hydrogen, C₃₋₆ cycloalkyl, —C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is selected from —CH₂—, O and NH;
L² is selected from covalent bond, and linear or branched C₁₋₆ alkyl; and
L³ is selected from covalent bond, linear or branched C₁₋₆ alkyl, —O—, and —C₁₋₄ alkoxy.

In some embodiments, X¹ is selected from the group consisting of linear or branched C₁₋₆ alkyl, C₃₋₆ cycloalkyl, C₆ aryl, 5-10 membered heteroaryl, and 4-6 membered heterocycloalkyl, wherein X¹ is unsubstituted or substituted with one or more substituents independently selected from the group consisting of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, and C₁₋₄ alkoxy; or X¹ together with the N atom of the amide forms a 4-8 membered heterocycloalkyl, which is unsubstituted or substituted with one or more substituents independently selected from the group consisting of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, and C₁₋₆ alkoxy.

In some embodiments, X² is selected from the group consisting of H, C₃₋₆ cycloalkyl, C₆ aryl, 5-10 membered heteroaryl, and 4-8 membered heterocycloalkyl, wherein X² is unsubstituted or substituted with one or more substituents independently selected from the group consisting of linear or branched C₁₋₄ alkyl, —C₁₋₄ alkoxy, NMe₂, halogen, CF₃, CHF₂, CMeF₂, OCF₃, and OCHF₂.

In some embodiments, L¹ is —CH₂— and/or L² is selected from a covalent bond and —CH₂— and/or L³ is selected from the group consisting of a covalent bond, —CH₂— —O—CH₂—, —O—CH₂—CH₂— and —O—.

In some embodiments, the compound is of formula II

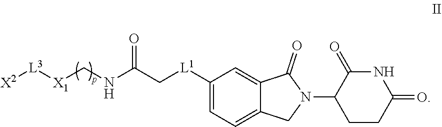

or a pharmaceutically acceptable salt or stereoisomer thereof,
wherein
X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, CF₃, CHF₂, —O—CHF₂, —O—(CH₂)₂—OMe, OCF₃, —CN, NH₂, C₁₋₆ alkoxy and C₁₋₆ alkylhydroxy; or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₄ alkylhydroxy, and C₁₋₆ alkoxy;
X² is selected from the group consisting of hydrogen, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is selected from —CH₂—, O and NH;
L³ is selected from the group consisting of a covalent bond, linear or branched C₁₋₆ alkyl, —O—, and —C₁₋₄ alkoxy; and
p is 0, or 1.

In some embodiments, the compound is, of formula III, IV, or V:

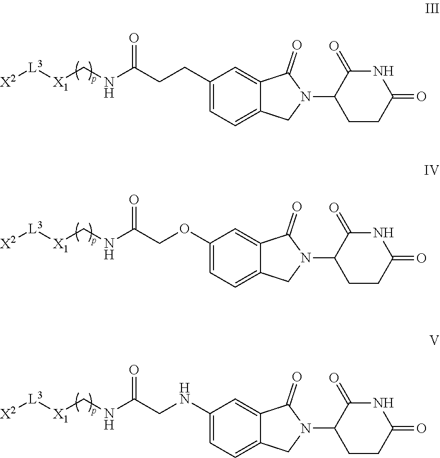

or a pharmaceutically acceptable salt or stereoisomer thereof,
wherein
X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, CF₃, CHF₂, —O—CHF₂, —O—(CH₂)₂—OMe, OCF₃, —CN, NH₂, C₁₋₆ alkoxy and C₁₋₆ alkylhydroxy;
or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₄ alkylhydroxy, and C₁₋₆ alkoxy;
X² is selected from the group consisting of hydrogen, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L³ is selected from the group consisting of a covalent bond, linear or branched C₁₋₆ alkyl, —O—, and —C₁₋₄ alkoxy; and
p is 0 or 1.

In some embodiments, the compound is of formula VI, VII or VIII:

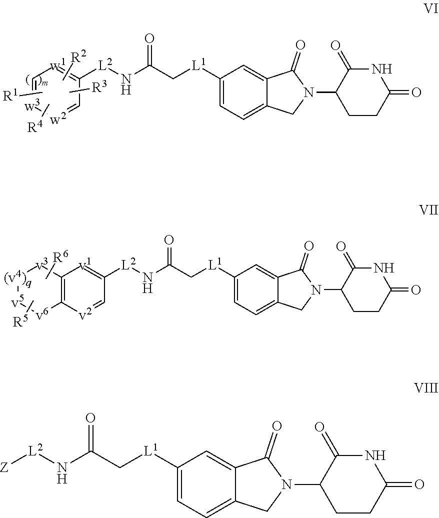

or a pharmaceutically acceptable salt or stereoisomer thereof,
wherein
each of w¹, w², and w³ is independently selected from the group consisting of C, N, S, and O, with the proviso that at least one of w¹, w², and w³ is C;
one or two of v³, v⁴, v⁵, and v⁶ is independently selected from C and O and the remaining of v³, v⁴, v⁵, and v⁶ are C;
each of v¹, and v² is independently selected from C and N;
L¹ is selected from —CH₂—, O and NH;
L² is selected from the group consisting of a covalent bond, and linear or branched C₁₋₆ alkyl;
each of R¹, R², R³, and R⁴ is independently selected from the group consisting of hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, and a group of formula -L³-X², wherein L³ is selected from the group consisting of a covalent bond, linear or branched C₁₋₆ alkyl, —O—, and —C₁₋₄ alkoxy, and X² is selected from the group consisting of C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
each of R⁵, and R⁶ is independently selected from the group consisting of H, linear or branched C₁₋₄ alkyl, and halogen, preferably F, or Cl, or more preferably F;
Z is selected from the group consisting of linear or branched —C₁₋₆ alkyl, —C₃₋₆ cycloalkyl, —C₁₋₄ alkoxy, and 4-6 membered heterocycloalkyl, wherein Z is unsubstituted or substituted with a substituent selected from the group consisting of with C₁₋₄ alkyl, C₆ aryl, C₆ aryloxy, 6 membered heteroaryl and CF₃; or Z together with the N atom of the amide forms a 4-6 membered heterocycloalkyl, which is unsubstituted or substituted with a substituent selected from the group consisting of C₁₋₄ alkyl, C₆ aryl, C₆ aryloxy, 6 membered heteroaryl and CF₃;
q is 0 or 1; and
m is 0 or 1.

In some embodiments, the compound is of formula X

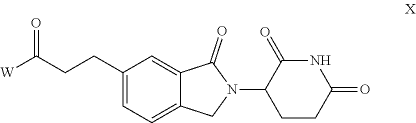

or a pharmaceutically acceptable salt or stereoisomer thereof,
wherein
W is selected from the group consisting of:

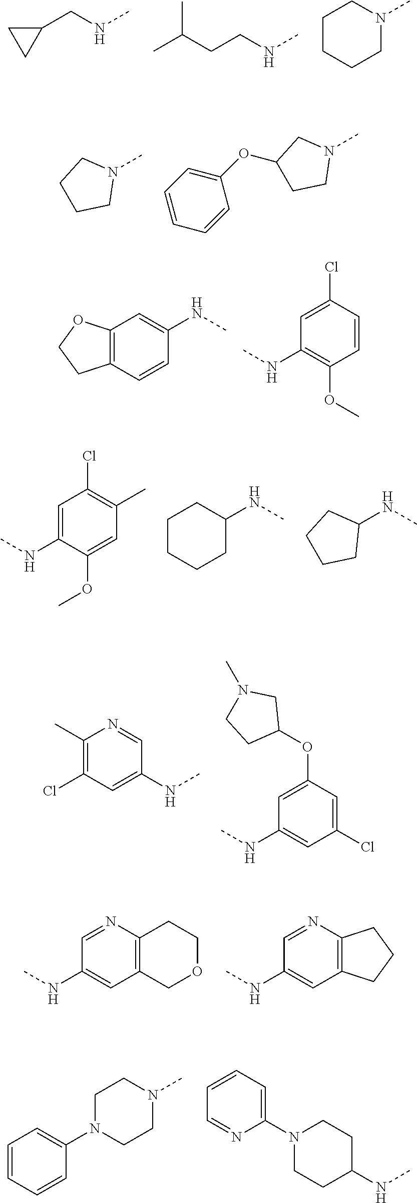

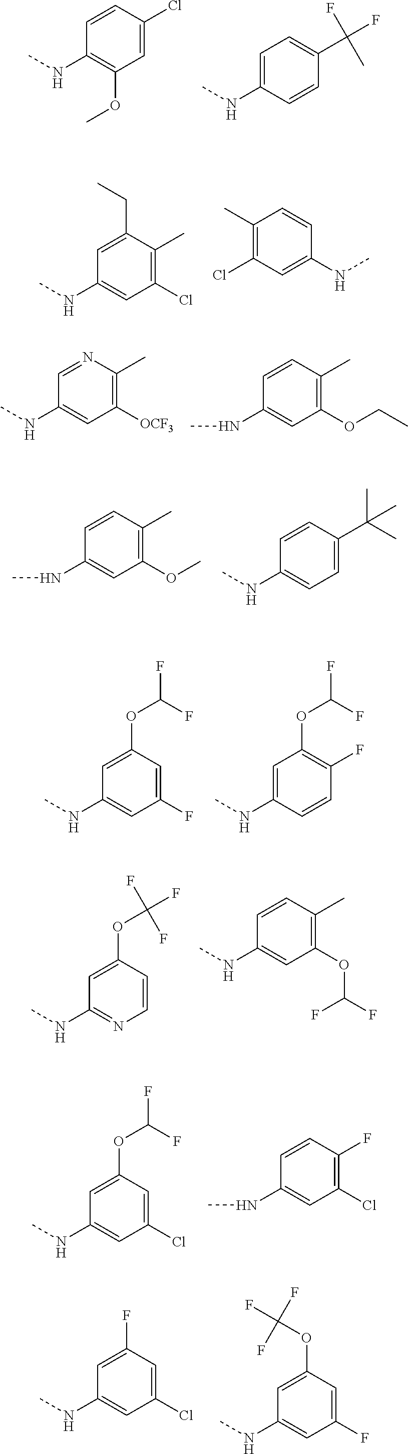

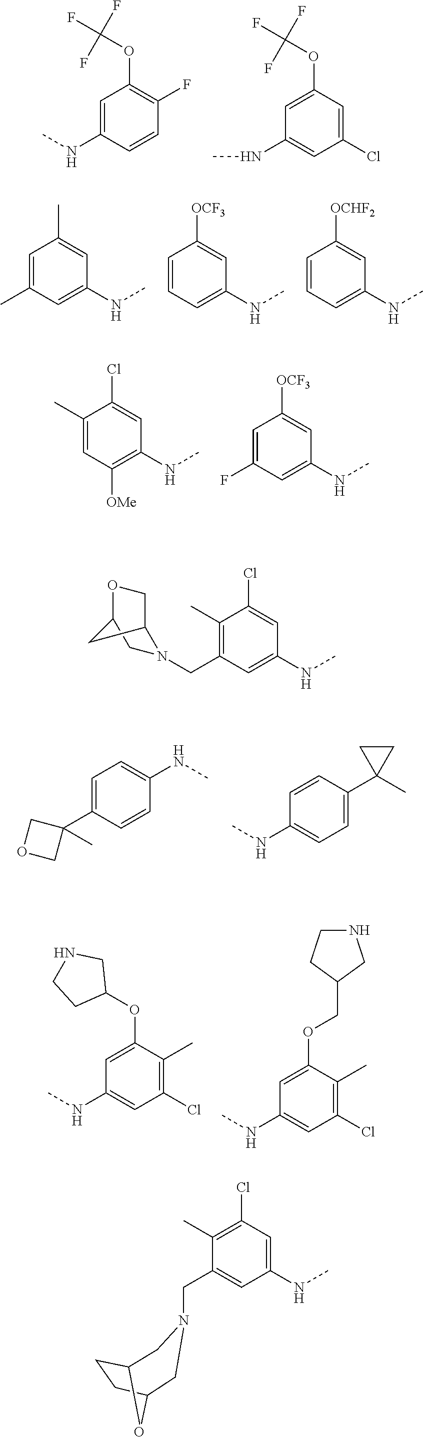

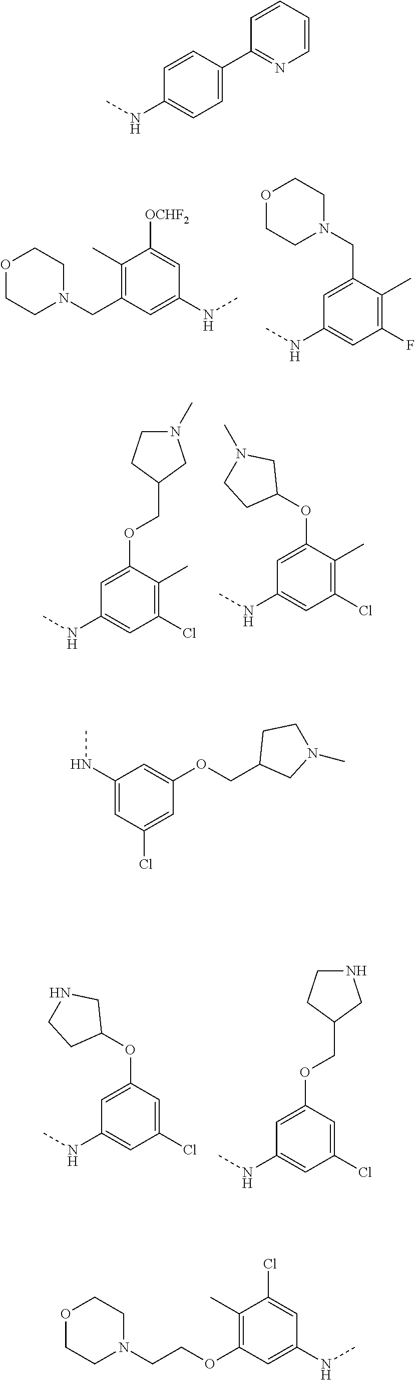

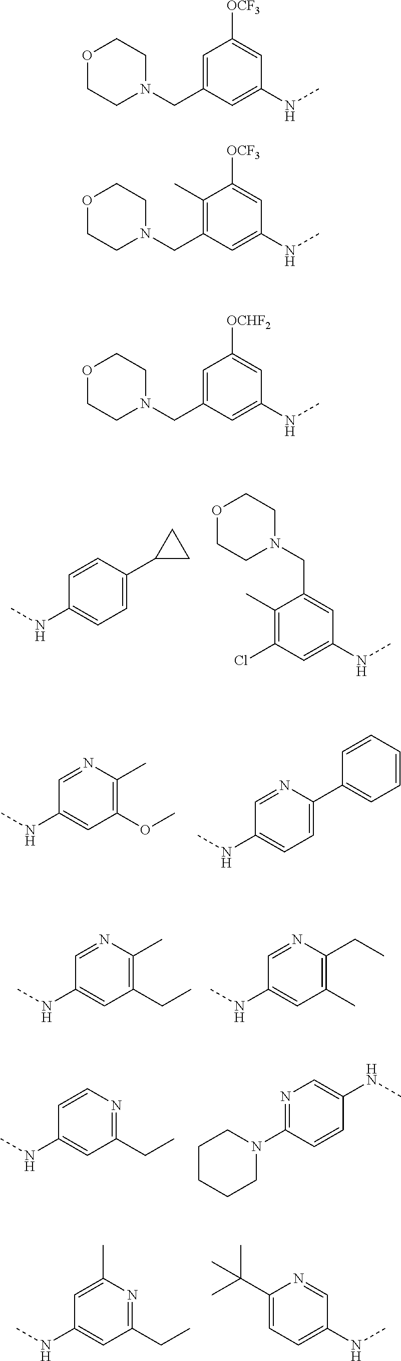

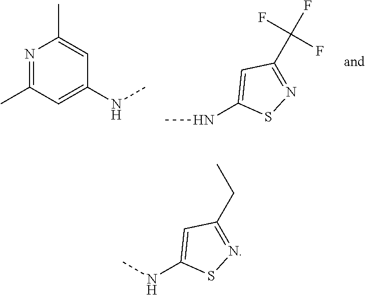

In further specific embodiments, the disclosure is directed to the specific examples disclosed in Table 1.

In some embodiments, the disclosure is directed to the (S) enantiomer of the compounds of any of formula I-X. In some embodiments, the disclosure is directed to the (R) enantiomer of the compounds of any of formula I-X. In some embodiments, the disclosure is directed to the racemate of the compounds of any of formula I-X.

The compounds of the disclosure may contain one or more asymmetric centers in the molecule. A compound without designation of the stereochemistry is to be understood to include all the optical isomers (e.g., diastereomers, enantiomers, etc.) in pure or substantially pure form, as well as mixtures thereof (e.g. a racemic mixture, or an enantiomerically enriched mixture). It is well known in the art how to prepare such optically active forms (e.g. by resolution of the racemic form by recrystallization techniques, by synthesis from optically-active starting materials, by chiral synthesis, by chromatographic separation using a chiral stationary phase, and other methods).

The compounds may be isotopically-labeled compounds, for example, compounds including various isotopes of hydrogen, carbon, nitrogen, oxygen, phosphorous, fluorine, iodine, or chlorine. The disclosed compounds may exist in tautomeric forms and mixtures and separate individual tautomers are contemplated. In addition, some compounds may exhibit polymorphism.

The compounds of the disclosure include the free form as well as the pharmaceutically acceptable salts and stereoisomers thereof. The pharmaceutically acceptable salts include all the typical pharmaceutically acceptable salts. The pharmaceutically acceptable salts of the present compounds can be synthesized from the compounds of this disclosure which contain a basic or acidic moiety by conventional chemical methods, see e.g. Berge et al, “Pharmaceutical Salts”, J. Pharm. ScL, 1977:66:1-19. Furthermore, the compounds of the disclosure also include lyophilized and polymorphs of the free form.

For example, conventional pharmaceutically acceptable salts for a basic compound include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. Conventional pharmaceutically acceptable salts for an acidic compound include those derived from inorganic bases include aluminum, ammonium, calcium, copper, ferric, ferrous, lithium, magnesium, manganic salts, manganous, potassium, sodium, zinc and the like. Salts derived from pharmaceutically acceptable organic bases include salts of primary, secondary and tertiary amines, substituted amines including naturally occurring substituted amines, cyclic amines and basic ion exchange resins, such as arginine, betaine caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The compounds of the disclosure may exist in solid, i.e. crystalline (e.g., polymorphs, i.e., different crystalline structures that have the same chemical composition but differ in packing, geometrical arrangement) or noncrystalline form (optionally as solvates) or liquid form. In the solid state, it may exist in, or as a mixture thereof. In crystalline solvates, solvent molecules are incorporated into the crystalline lattice during crystallization. The formation of solvates may include non-aqueous solvents such as, but not limited to, ethanol, isopropanol, DMSO, acetic acid, ethanolamine, or ethyl acetate, or aqueous solvents such as water (also called “hydrates”). Different polymorphs may be produced, for example, by changing or adjusting the reaction conditions or reagents.

In a further aspect, the disclosure also provides methods of preparation of the compounds of formula I-X of the disclosure. In some embodiments, they are prepared according to the general procedure A.

In yet another aspect, the disclosure further provides a pharmaceutical composition comprising a therapeutically-effective amount of one or more of the compounds of the disclosure or pharmaceutically acceptable salt thereof and one or more pharmaceutically acceptable carriers and/or excipients (also referred to as diluents). The excipients are acceptable in the sense of being compatible with the other ingredients of the formulation and not deleterious to the recipient thereof (i.e., the patient). The term “therapeutically-effective amount” as used herein refers to the amount of a compound (as such or in form of a pharmaceutical composition) of the present disclosure which is effective for producing some desired therapeutic effect.

Pharmaceutical compositions may be in unit dose form containing a predetermined amount of a compound of the disclosure per unit dose. Such a unit may contain a therapeutically effective dose of a compound of the disclosure or salt thereof or a fraction of a therapeutically effective dose such that multiple unit dosage forms might be administered at a given time to achieve the desired therapeutically effective dose. Preferred unit dosage formulations are those containing a daily dose or sub-dose, or an appropriate fraction thereof, of a compound of the disclosure or salt thereof.

The compounds of the disclosure may be administered by any acceptable means in solid or liquid form, including (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) nasally; (9) pulmonary; or (10) intrathecally.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical compositions.

Such compositions may contain further components conventional in pharmaceutical preparations, e.g. wetting agents, emulsifiers and lubricants, such as sodium lauryl sulfate and magnesium stearate, as well as coloring agents, release agents, coating agents, sweetening, flavoring and perfuming agents, preservatives and antioxidants, pH modifiers, bulking agents, and further active agents. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Such compositions may be prepared by any method known in the art, for example, by bringing into association the active ingredient with one or more carriers and/or excipients. Different compositions and examples of carriers and/or excipients are well known to the skilled person and are described in detail in, e.g., Remington: The Science and Practice of Pharmacy. Pharmaceutical Press, 2013; Rowe, Sheskey, Quinn: Handbook of Pharmaceutical Excipients. Pharmaceutical Press, 2009. Excipients that may be used in the preparation of the pharmaceutical compositions may include one or more of buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide a composition suitable for an administration of choice.

As indicated above, the compounds of the present disclosure may be in solid or liquid form and administered by various routes in any convenient administrative form, e.g., tablets, powders, capsules, solutions, dispersions, suspensions, syrups, sprays, suppositories, gels, emulsions, patches, etc.

In solid dosage forms of the disclosure for oral administration (capsules, tablets, pills, dragees, powders, granules, trouches and the like), a compound is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like. A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. An oral composition can also include adjuvants such as wetting agents, emulsifying and suspending agents, sweetening, flavoring, coloring, perfuming and preservative agents.

In form of suspensions, a compound may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for rectal or vaginal administration of a compound of the disclosure include a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Other suitable forms include pessaries, tampons, creams, gels, pastes, foams or spray formulations containing such carriers as are known in the art to be appropriate.

Dosage forms for the topical or transdermal administration of a compound of the disclosure include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required. Such ointments, pastes, creams and gels may contain, in addition to a compound of the disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Dosage forms such as powders and sprays for administration of a compound of the disclosure, may contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Dosage forms such as transdermal patches for administration of a compound of the disclosure may include absorption enhancers or retarders to increase or decrease the flux of the compound across the skin. The rate of such flux can be controlled by either providing a rate controlling membrane or dispersing the compound in a polymer matrix or gel. Other dosage forms contemplated include ophthalmic formulations, eye ointments, powders, solutions and the like. It is understood that all contemplated compositions must be stable under the conditions of manufacture and storage, and preserved against the contaminating action of microorganisms, such as bacteria and fungi.

The dosage levels of a compound of the disclosure in the pharmaceutical compositions of the disclosure may be adjusted in order to obtain an amount of a compound of the disclosure which is effective to achieve the desired therapeutic response for a particular patient, composition, and mode of administration, without being deleterious to the patient. The dosage of choice will depend upon a variety of factors including the nature of the particular compound of the present disclosure used, the route of administration, the time of administration, the rate of excretion or metabolism of the particular compound used, the rate and extent of absorption, the duration of the treatment, other drugs, compounds and/or materials used in combination with the particular compound, the age, sex, weight, condition, general health and prior medical history of the patient being treated, and like factors well known in the medical arts. A medical practitioner having ordinary skill in the art can readily determine and prescribe the effective amount of the pharmaceutical composition required.

Typically, a suitable daily dose of a compound of the disclosure will be that amount of the compound, which is the lowest dose effective to produce a therapeutic effect. Such an effective dose will generally depend upon the factors described above. Generally, oral, intravenous, intracerebroventricular and subcutaneous doses of the compounds of this disclosure for a patient, when used for the indicated analgesic effects, will range from about 0.0001 to about 100 mg, more usual 0.1 to 100 mg/kg per kilogram of body weight of recipient (patient, mammal) per day. Acceptable daily dosages may be from about 1 to about 1000 mg/day, and for example, from about 1 to about 100 mg/day.

A compound of the disclosure, or a pharmaceutically acceptable salt, or stereoisomer, thereof, may be administered once daily (QD) or divided into multiple daily doses such as twice daily (BID), three times daily (TID), and four times daily (QID) or may be administered in regular intervals of more than one day, such as every two days (Q2D). Administration may be continuous (i.e., daily for consecutive days or every day) or intermittent, e.g., in cycles (i.e., including days, weeks, or months of rest without drug). In some embodiments, a compound of the disclosure, or a pharmaceutically acceptable salt, or stereoisomer thereof, is administered every day for at least 21 days. In some embodiments, a compound of the disclosure, or a pharmaceutically acceptable salt or stereoisomer thereof, is administered every two days for at least 21 days.

The term “intermittent” or “intermittently” as used herein is intended to mean stopping and starting at either regular or irregular intervals. For example, intermittent administration of a compound of the disclosure, or a pharmaceutically acceptable salt, or stereoisomer, thereof, is administration for one to six days per week, administration in cycles (e.g., daily administration for two to eight consecutive weeks, then a rest (or holiday) period with no administration for up to one week), or administration on alternate days. The term “cycling” as used herein is intended to mean that a compound of the disclosure, or a pharmaceutically acceptable salt, or stereoisomer, thereof, is administered daily or continuously but with a rest period. In some embodiments, the rest period is the same length as the treatment period. In other embodiments, the rest period has different length from the treatment period. In some embodiments, a compound of the disclosure, or a pharmaceutically acceptable salt, or stereoisomer, thereof, is administered intermittently once per day for 5 days followed by a rest of 3 days (i.e. 5 days on/3 days off). This intermittent administration is repeated for 3 to 4 cycles. In some embodiments, a compound of the disclosure, or a pharmaceutically acceptable salt, or stereoisomer thereof, is administered intermittently once per day for 5 days followed by a rest of 9 days (i.e. 5 days on/9 days off). This intermittent administration is repeated for 2 cycles. It is understood that dosing regimen also depend on factors as indicated above, e.g. on the administration, and can be readily arrived at by one skilled in medicine or the pharmacy art.

The compounds of the disclosure modulate the activity of cereblon. Thus, the compounds and compositions of the disclosure can be useful as a medicament, i.e. as a medicament in therapy, more specifically for the treatment of cancer, as detailed below. Therefore, in a further aspect, the present disclosure provides a method of treatment of a mammal, for example, a human, suffering from cancer, as detailed below. The term “treatment” is intended to encompass prophylaxis, therapy and cure. Such treatment comprises the step of administering a therapeutically effective amount of a compound of Formula I or salt thereof (or of a pharmaceutical composition containing a compound of Formula I or salt thereof) to said mammal, for example, a human.

Thus, the disclosure is directed towards the use of the compounds of the disclosure or pharmaceutically acceptable salts or stereoisomers thereof or a pharmaceutical composition thereof for the treatment of a disease associated or caused with GSPT1, in particular the treatment of cancer, as detailed below, in a mammal, for example a human.

Myc-Driven Cancers

Described herein, in some embodiments, are cancers exhibiting increased expression of one or more of c-Myc, L-Myc, N-Myc, EIF4EBP1, and EIF4EBP2 as well as ones with increase phosphorylation of one or both of EIF4EBP1 and EIF4EBP2.

Myc-driven cancers refer to cancers where there is abnormal activation of Myc oncogene, either due to transcriptional overexpression (e.g., caused by gene amplification, translocation, alterations in upstream signaling pathways) and/or protein stabilization. A myc-driven cancer cell includes a cancer cell that has an increased expression or overexpression (and/or increased activity) of at least one myc transcription factor such as N-myc and/or L-myc and/or C-myc, or a surrogate marker thereof, relative to a control cell such as a normal (e.g., non-cancerous) cell of the same or corresponding cell type. The term “cancerous” when referring to a sample such as a cell or tissue, generally refers to any sample, such as cells or tissues that exhibit, or are predisposed to exhibiting, unregulated growth, including, for example, a neoplastic cell/tissue such as a premalignant cell/tissue or a cancer cell (e.g., carcinoma cell or sarcoma cell).

In some embodiments the Myc-driven cancer or tumor as defined herein refers to a blood borne tumor cancer, such as a hematological cancer, preferably a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In some embodiments, the Myc-driven cancer or tumour is a solid tumor cancer, such as breast cancer, colorectal cancer, lung cancer, e.g. SCLC, NSCLC, neuroendocrine cancer, e.g., neuroendocrine prostate cancer (for example, NEPC (castration-resistant neuroendocrine prostate cancer)) and lung neuroendocrine tumors (Lu-NETs), liver cancer, stomach cancer, pancreatic cancer, gastric cancer, esophageal cancer, bladder cancer, skin cancer, brain cancer, cervical cancer, ovarian cancer, melanoma and head and neck cancer.

In some embodiments the Myc-driven cancer as used herein refers in particular to breast cancer and SCLC. In some embodiments the myc-driven cancer as used herein refers in particular to NSCLC. In some embodiments, the cancer is solid tumor cancer exhibiting amplification of the N-Myc gene and/or the L-Myc gene. In some embodiments the Myc-driven cancer as used herein refers to neuroendocrine cancer, for example, neuroendocrine prostate cancer (for example, NEPC (castration-resistant neuroendocrine prostate cancer)) and lung neuroendocrine tumors (Lu-NETs), acute myelogenous leukemia (AML), lymphoma, and multiple myeloma (MM).

Solid and Liquid Cancers

The term “solid cancer” or “solid tumor” refers to disease of tissues or organs, such as to malignant, neoplastic, or cancerous solid tumors, i.e. sarcomas, carcinomas. The tissue structure of solid tumors includes interdependent tissue compartments and usually does not contain cysts or fluid areas. A solid cancer or solid tumor includes cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer (for example, NEPC (castration-resistant neuroendocrine prostate cancer)) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, cervical cancer, ovarian cancer, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In some embodiments, a solid cancer or solid tumor is a cancer of the breast, lung, stomach, colon, bladder, brain, pancreas, liver, head and neck, prostate, ovaries, upper aerodigestive tract and the like.

The term “blood borne cancer” or “blood borne tumor” (also typically referred to as “hematological cancer”) refers to cancer of the body's blood-forming and immune system—the bone marrow and lymphatic tissue. The tissue structure of blood-borne cancers or tumors includes an abnormal mass of cells that is fluid in nature. Such cancers include leukemias (malignant neoplasms of the blood-forming tissues), lymphomas (Non-Hodgkin's Lymphoma), Hodgkin's disease (Hodgkin's Lymphoma) and myeloma. In one embodiment, the myeloma is multiple myeloma (MM). In some embodiments, the leukemia is, for example, acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, or B-cell acute lymphoblastic leukemia. The leukemia can be relapsed, refractory or resistant to conventional therapy. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma. In some embodiments blood-borne cancers or hematological cancers include acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In particular embodiments, the compounds of the disclosure or pharmaceutically acceptable salts or stereoisomers thereof or a pharmaceutical composition thereof are used for the treatment of cancer associated with GSPT1, such as solid cancers including but not limited to cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, uterus, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

Such a use (or method of treatment) of a subject comprises administering to a subject in need of such treatment a therapeutically effective amount of a compound of the disclosure or pharmaceutically acceptable salts thereof or a pharmaceutical composition thereof by targeting cereblon.

Disclosed herein, in part, is a method of treating a Myc-driven cancer in a subject in need thereof, comprising administering the subject a therapeutically effective amount of a compound described herein or a composition as described herein.

In some embodiments, the Myc-driven cancer is a Myc-driven lung cancer.

In some embodiments, the Myc-driven cancer is characterized by a high driven Myc tumor.

In some embodiments, the Myc-driven cancer is a Myc-driven small cell lung cancer.

In some embodiments, the Myc-driven small cell lung cancer is a high L-Myc small cell lung cancer.

In some embodiments, the Myc-driven cancer is a Myc-driven non-small cell lung cancer.

In some embodiments, the Myc-driven non-small cell lung cancer is a high N-Myc non-small cell lung cancer.

In some embodiments, the method comprises orally administering the compound to the subject.

In another aspect, provided herein is a method of degrading GSPT1 in a subject suffering from cancer, comprising administering to the subject a therapeutically effective amount of a compound described herein or a composition described herein.

In some embodiments, the cancer is a Myc-driven cancer.

In some embodiments, the Myc-driven cancer is a Myc-driven lung cancer.

In some embodiments, the Myc-driven cancer is a Myc-driven small cell lung cancer.

In some embodiments, the Myc-driven small cell lung cancer is a high L-Myc small cell lung cancer.

In some embodiments, the Myc-driven cancer is a Myc-driven non-small cell lung cancer.

In some embodiments, the Myc-driven non-small cell lung cancer is a high N-Myc non-small cell lung cancer.

In some embodiments, the method comprises orally administering the compound to the subject.

In another aspect, the disclosure is directed to a method of reducing the level of GSPT1 in a subject suffering from cancer, comprising administering the subject a therapeutically effective amount of a compound or a composition as described herein.

In some embodiments, the cancer is a Myc-driven cancer.

In some embodiments, the Myc-driven cancer is a Myc-driven lung cancer.

In some embodiments, the Myc-driven cancer is a Myc-driven small cell lung cancer.

In some embodiments, the Myc-driven small cell lung cancer is a high L-Myc small cell lung cancer.

In some embodiments, the Myc-driven cancer is a Myc-driven non-small cell lung cancer.

In some embodiments, the Myc-driven non-small cell lung cancer is a high N-Myc non-small cell lung cancer.

In some embodiments, the method comprises orally administering the compound to the subject.

The present disclosure contemplates administration of a compound of the disclosure alone or in combination with one or more additional therapeutic agents, such as other Tyrosine kinase inhibitors: Erlotinib hydrochloride (e.g. Tarceva® by Genentech/Roche), Linifanib (or ABT 869, by Genentech), sunitinib malate (e.g. Sutent® by Pfizer), bosutinib (or SKI-606, described in U.S. Pat. No. 6,780,996), dasatinib (e.g. Sprycel® by Bristol-Myers Squibb), armala (e.g. pazopanib, e.g. Votrient® by GlaxoSmithKline), imatinib and imatinib mesylate (e.g. Gilvec® and Gleevec® by Novartis); Vascular Endothelial Growth Factor (VEG) receptor inhibitors (Bevacizumab, or Avastin® by Genentech/Roche), axitinib, (or AG013736, described in WO 01/002369), Brivanib Alaninate (or BMS-582664), motesanib (or AMG-706, described in PCT WO 02/066470), pasireotide (e.g. SOM230, described in WO 02/010192), sorafenib (e.g. Nexavar®); HER2 receptor inhibitors: Trastuzumab (e.g. Herceptin® by Genentech/Roche), neratinib (or HKI-272, described WO 05/028443), lapatinib or lapatinib ditosylate (e.g. Tykerb® by GlaxoSmithKline); CD20 antibodies: Rituximab (e.g. Riuxan® and MabThera® by Genentech/Roche), tositumomab (e.g. Bexxar® by GlaxoSmithKline), ofatumumab (e.g. Arzerra® by GlaxoSmithKline); Bcr/Abl kinase inhibitors: nilotinib hydrochloride (e.g. Tasigna® by Novartis); DNA Synthesis inhibitors: Capecitabine (e.g. Xeloda® by Roche), gemcitabine hydrochloride (e.g. Gemzar® by Eli Lilly and Company), nelarabine (or Arranon® and Atriance® by GlaxoSmithKline); Antineoplastic agents: oxaliplatin (e.g. Eloxatin® ay Sanofi-Aventis described in U.S. Pat. No. 4,169,846); Epidermal growth factor receptor (EGFR) inhibitors: Gefitinib (or Iressa®), Afatinib (or Tovok® by Boehringer Ingelheim), cetuximab (e.g. Erbitux® by Bristol-Myers Squibb), panitumumab (e.g. Vectibix® by Amgen); HER dimerization inhibitors: Pertuzumab (e.g. Omnitarg®, by Genentech); Human Granulocyte colony-stimulatingfactor (G-CSF) modulators: Filgrastim (e.g. Neupogen® by Amgen); Immunomodulators: Afutuzumab (by Roche®), pegfilgrastim (e.g. Neulasta® by Amgen), lenalidomide (e.g. CC-5013, e.g. Revlimid®), thalidomide (e.g. Thalomid®); CD40 inhibitors: Dacetuzumab (e.g. SGN-40 or huS2C6, by Seattle Genetics, Inc); Pro-apoptotic receptor agonists (PARAs): Dulanermin (e.g. AMG-951, by Amgen/Genentech); Hedgehog antagonists: Vismodegib (or GDC-0449, described in WO 06/028958); Phospholipase A2 inhibitors: Anagrelide (e.g. Agrylin®); BCL-2 inhibitors: Navitoclax (or ABT-263, described in WO 09/155386); Mitogen-activated protein kinase kinase (MEK) inhibitors: XL-518 (Cas No. 1029872-29-4, by ACC Corp.); Aromatase inhibitors: Exemestane (e.g. Aromasin® by Pfizer), letrozole (e.g. Femara® by Novartis), anastrozole (e.g. Arimidex®); Topoisomerase I inhibitors: Irinotecan (e.g. Camptosar® by Pfizer), topotecan hydrochloride (e.g. Hycamtin® by GlaxoSmithKline); Topoisomerase II inhibitors: etoposide (e.g. VP-16 and Etoposide phosphate, e.g. Toposar®, VePesid® and Etopophos®), teniposide (e.g. VM-26, e.g. Vumon®); mTOR inhibitors: Temsirolimus (e.g. Torisel® by Pfizer), ridaforolimus (formally known as deferolimus, (or AP23573 and MK8669, described in WO 03/064383), everolimus (e.g. Afinitor® by Novartis); Osteoclastic bone resorption inhibitors: zoledronic acid (or Zometa® by Novartis); CD33 Antibody Drug Conjugates: Gemtuzumab ozogamicin (e.g. Mylotarg® by Pfizer/Wyeth); CD22 Antibody Drug Conjugates: Inotuzumab ozogamicin (also referred to as CMC-544 and WAY-207294, by Hangzhou Sage Chemical Co., Ltd.); CD20 Antibody Drug Conjugates: Ibritumomab tiuxetan (e.g. Zevalin®); Somatostain analogs: octreotide (e.g. octreotide acetate, e.g. Sandostatin® and Sandostatin LAR®); Synthetic Interleukin-11 (IL-11): oprelvekin (e.g. Neumega® by Pfizer/Wyeth); Synthetic erythropoietin: Darbepoetin alfa (e.g. Aranesp® by Amgen); Receptor Activator for Nuclear Factor kappa B (RANK) inhibitors: Denosumab (e.g. Prolia® by Amgen); Thrombopoietin mimetic peptibodies: Romiplostim (e.g. Nplate® by Amgen; Cell growth stimulators: Palifermin (e.g. Kepivance® by Amgen); Anti-Insulin-like Growth Factor-1 receptor (IGF-1R) antibodies: Figitumumab (e.g. CP-751,871, by ACC Corp), robatumumab (CAS No. 934235-44-6); Anti-CS1 antibodies: Elotuzumab (HuLuc63, CAS No. 915296-00-3); CD52 antibodies: Alemtuzumab (e.g. Campath®); CTLA-4 inhibitors: Tremelimumab (IgG2 monoclonal antibody by Pfizer, formerly known as ticilimumab, CP-675,206), ipilimumab (CTLA-4 antibody, e.g. MDX-010, CAS No. 477202-00-9); Histone deacetylase inhibitors (HDI): Voninostat (e.g. Zolinza® by Merck); Alkylating agents: Temozolomide (e.g. Temodar® and Temodal® by Schering-Plough/Merck), dactinomycin (e.g. actinomycin-D and e.g. Cosmegen®), melphalan (e.g. L-PAM, L-sarcolysin, and phenylalanine mustard, e.g. Alkeran®), altretamine (e.g. hexamethylmelamine (HMM), e.g. Hexalen®), carmustine (e.g. BiCNU®), bendamustine (e.g. Treanda®), busulfan (e.g. Busulfex® and Myleran®), carboplatin (e.g. Paraplatin®), lomustine (e.g. CCNU, e.g. CeeNU®), cisplatin (e.g. CDDP, e.g. Platinol® and Platinol®-AQ), chlorambucil (e.g. Leukeran®), cyclophosphamide (e.g. Cytoxan® and Neosar®), dacarbazine (e.g. DTIC, DIC and imidazole carboxamide, e.g. DTIC-Dome®), altretamine (e.g. hexamethylmelamine (HMM) e.g. Hexalen®), ifosfamide (e.g. Ifex®), procarbazine (e.g. Matulane®), mechlorethamine (e.g. nitrogen mustard, mustine and mechloroethamine hydrochloride, e.g. Mustargen®), streptozocin (e.g. Zanosar®), thiotepa (e.g. thiophosphoamide, TESPA and TSPA, e.g. Thioplex®; Biologic response modifiers: Bacillus calmette-guerin (e.g. theraCys® and TICE® BCG), denileukin diftitox (e.g. Ontak®); Anti-tumor antibiotics: doxorubicin (e.g. Adriamycin® and Rubex®), bleomycin (e.g. Lenoxane®), daunorubicin (e.g. dauorubicin hydrochloride, daunomycin, and rubidomycin hydrochloride, e.g. Cerubidine®), daunorubicin liposomal (daunorubicin citrate liposome, e.g. DaunoXome®), mitoxantrone (e.g. DHAD, e.g. Novantrone®), epirubicin (e.g. Ellence™), idarubicin (e.g. Idamycin®, Idamycin PFS®), mitomycin C (e.g. Mutamycin®); Anti-microtubule agents: Estramustine (e.g. Emcyl®); Cathepsin K inhibitors: Odanacatib (or MK-0822, by Lanzhou Chon Chemicals, ACC Corp., and ChemieTek, described in WO 03/075836); Epothilone B analogs: Ixabepilone (e.g. Lxempra® by Bristol-Myers Squibb); Heat Shock Protein (HSP) inhibitors: Tanespimycin (17-allylamino-17-demethoxygeldanamycin, e.g. KOS-953 and 17-AAG, by SIGMA, described in U.S. Pat. No. 4,261,989); TpoR agonists: Eltrombopag (e.g. Promacta® and Revolade®) by GlaxoSmithKline); Anti-mitotic agents: Docetaxel (e.g. Taxotere® by Sanofi-Aventis); Adrenal steroid inhibitors: aminoglutethimide (e.g. Cytadren®); Anti-androgens: Nilutamide (e.g. Nilandron® and Anandron®), bicalutamide (sold under tradename Casodex®), flutamide (e.g. Fulexin™); Androgens: Fluoxymesterone (e.g. Halotestin®); Proteasome inhibitors: Bortezomib (e.g. Velcade®); CDK1 inhibitors: Alvocidib (e.g. flovopirdol or HMR-1275, described in U.S. Pat. No. 5,621,002); Gonadotropin-releasing hormone (GnRH) receptor agonists: Leuprolide or leuprolide acetate (e.g. Viadure® by Bayer AG, Eligard® by Sanofi-Aventis and Lupron® by Abbott Lab); Taxane anti-neoplastic agents: Cabazitaxel, larotaxel; 5HT1a receptor agonists: Xaliproden (or SR57746, described in U.S. Pat. No. 5,266,573); HPC vaccines: Cervarix® sold by GlaxoSmithKline, Gardasil® sold by Merck; Iron Chelating agents: Deferasinox (e.g. Exjade® by Novartis); Anti-metabolites: Claribine (2-chlorodeoxyadenosine, e.g. Leustatin®), 5-fluorouracil (e.g. Adrucil®), 6-thioguanine (e.g. Purinethol®), pemetrexed (e.g. Alimta®), cytarabine (e.g. arabinosylcytosine (Ara-C), e.g. Cytosar-U®), cytarabine liposomal (e.g. Liposomal Ara-C, e.g. DepoCyt™) decitabine (e.g. Dacogen®), hydroxyurea (e.g. Hydrea®, Droxia™ and Mylocel™) fludarabine (e.g. Fludara®), floxuridine (e.g. FUDR®), cladribine (e.g. 2-chlorodeoxyadenosine (2-CdA) e.g. Leustatin™), methotrexate (e.g. amethopterin, methotrexate sodim (MTX), e.g. Rheumatrex® and Trexall™), pentostatin (e.g. Nipent®); Bisphosphonates: Pamidronate (e.g. Aredia®), zoledronic acid (e.g. Zometa®); Demethylating agents: 5-azacitidine (e.g. Vidaza®), decitabine (e.g. Dacogen®); Plant Alkaloids: Paclitaxel protein-bound (e.g. Abraxane®), vinblastine (e.g. vinblastine sulfate, vincaleukoblastine and VLB, e.g. Alkaban-AQ® and Velban®), vincristine (e.g. vincristine sulfate, LCR, and VCR, e.g. Oncovin® and Vincasar Pfs®), vinorelbine (e.g. Navelbine®), paclitaxel (e.g. Taxol and Onxal™); Retinoids: Alitretinoin (e.g. Panretin®), tretinoin (all-trans retinoic acid, e.g. ATRA, e.g. Vesanoid®), Isotretinoin (13-cis-retinoic acid, e.g. Accutane®, Amnesteem®, Claravis®, Clarus®, Decutan®, Isotane®, Izotech®, Oratane®, Isotret®, and Sotret®), bexarotene (e.g. Targretin®); Glucocorticosteroids: Hydrocortisone (e.g. cortisone, hydrocortisone sodium succinate, hydrocortisone sodium phosphate, and e.g. Ala-Cort®, Hydrocortisone Phosphate, Solu-Cortef®, Hydrocort Acetate® and Lanacort®), dexamethasone, prednisolone (e.g. Delta-Cortel®, Orapred®, Pediapred® and Prelone®), prednisone (e.g. Deltasone®, Liquid Red®, Meticorten® and Orasone®), methylprednisolone (e.g. 6-Methylprednisolone, Methylprednisolone Acetate, Methylprednisolone Sodium Succinate, e.g. Duralone®, Medralone®, Medrol®, M-Prednisol® and Solu-Medrol®); Cytokines: interleukin-2 (e.g. aldesleukin and IL-2, e.g. Proleukin®), interleukin-11 (e.g. oprevelkin, e.g. Neumega®), alpha interferon alfa (e.g. IFN-alpha, e.g. Intron® A, and Roferon-A®); Lutinizing hormone releasing hormone (LHRH) agonists: Goserelin (e.g. Zoladex®); Progesterones: megestrol (e.g. megestrol acetate, e.g. Megace®); Miscellaneous cytotoxic agents: Arsenic trioxide (e.g. Trisenox®), asparaginase (e.g. L-asparaginase, Erwinia L-asparaginase, e.g. Elspar® and Kidrolase®); Anti-nausea drugs: NK-1 receptor antagonists: Casopitant (e.g. Rezonic® and Zunrisa® by GlaxoSmithKline); and Cytoprotective agents: Amifostine (e.g. Ethyol®), leucovorin (e.g. calcium leucovorin, citrovorum factor and folinic acid).

### EXAMPLES

### Example 1: General Procedure A

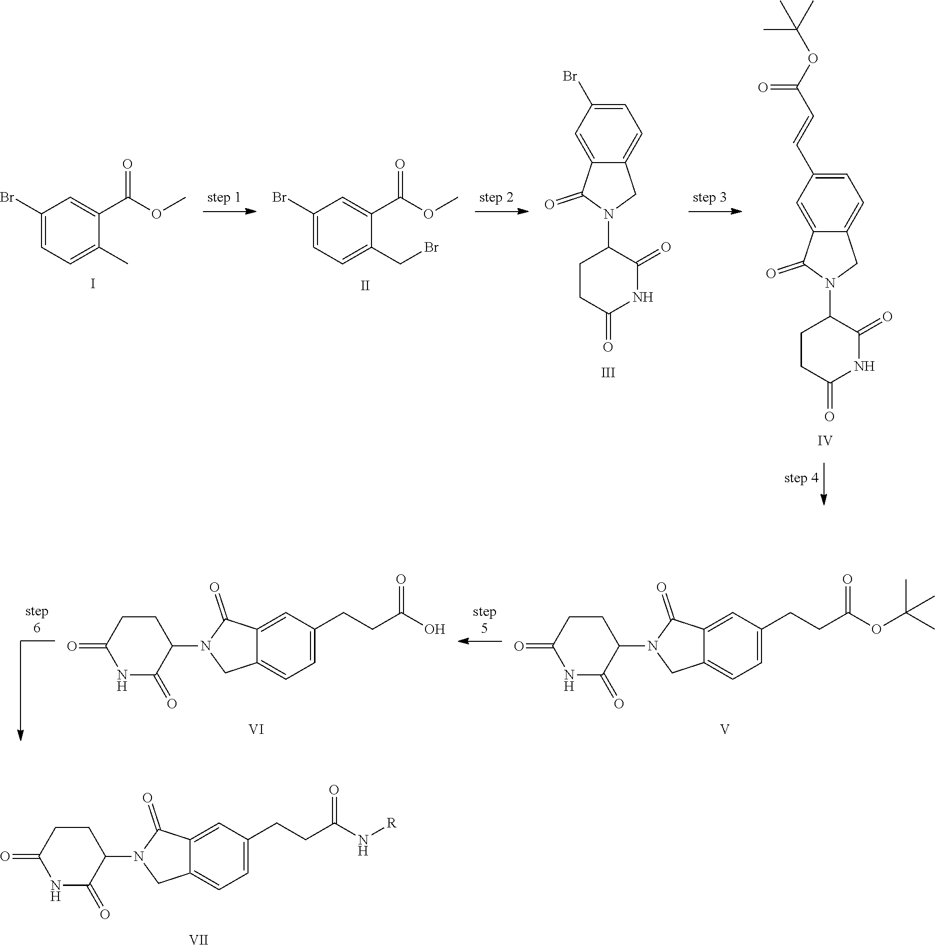

II: To a solution of methyl 5-bromo-2-methylbenzoate I (100 g, 436 mmol, 1.00 eq) in trichloromethane (800 mL) was added N-bromosuccinimide (77.6 g, 436 mmol, 1.00 eq) and (E)-2,2′-(diazene-1,2-diyl)bis(2-methylpropanenitrile) (10.5 g, 43.3 mmol, 0.10 eq). The solution was degassed by purging with nitrogen, and the reaction was stirred at 80° C. for 12 h under nitrogen atmosphere. The mixture was concentrated under reduced pressure to give a residue. The residue was purified by silica gel column chromatography (petroleum ether/ethyl acetate=1/0 to 20/1) to afford methyl 5-bromo-2-(bromomethyl)benzoate II. ¹H NMR (400 MHz, DMSO-d₆) δ=7.98 (d, J=2.0 Hz, 1H), 7.81-7.79 (m, 1H), 7.57-7.55 (m, 1H), 4.97 (s, 2H), 3.87 (m, 3H).

III: To a solution of methyl 5-bromo-2-(bromomethyl)benzoate II (72.5 g, 235 mmol 1.00 eq) and 3-aminopiperidine-2,6-dione hydrochloride (46.6 g, 283 mmol, 1.20 eq, hydrochloride) in acetonitrile (600 mL) was added diisopropylethylamine (123 mL, 706 mmol, 3.00 eq) in one portion under nitrogen. The reaction was stirred at 80° C. for 4 h. After cooling to room temperature, the mixture was concentrated under reduced pressure to give a residue. The residue was triturated with hydrochloric acid (1 M)/ethyl acetate (300 mL/200 mL) at 25° C. for 30 min. The mixture was filtered, and the filter cake was washed with ethyl acetate (100 mL) and dried under reduced pressure to afford 3-(6-bromo-1-oxoisoindolin-2-yl)piperidine-2,6-dione III. ¹H NMR (400 MHz, DMSO-d₆) δ=11.04 (s, 1H), 7.86 (d, J=1.6 Hz, 1H), 7.82 (dd, J=1.8, 8.1 Hz, 1H), 7.60 (d, J=8.1 Hz, 1H), 5.12 (dd, J=5.1, 13.3 Hz, 1H), 4.49-4.28 (m, 2H), 2.99-2.84 (m, 1H), 2.60 (br d, J=17.5 Hz, 1H), 2.39 (dq, J=4.4, 13.2 Hz, 1H), 2.13-1.91 (m, 1H).

IV: To a solution of tri-tert-butylphosphonium tetrafluoroborate (1.43 g, 4.91 mmol, 0.53 eq), N-cyclohexyl-N-methylcyclohexanamine (3.26 mL, 15.3 mmol, 1.65 eq) and tris(dibenzylideneacetone)dipalladium(0) (3.00 g, 3.28 mmol, 0.35 eq) in dioxane (150 mL) were added 3-(6-bromo-1-oxoisoindolin-2-yl)piperidine-2,6-dione III (3.00 g, 9.28 mmol, 1.00 eq) and tert-butyl acrylate (3.40 mL, 23.4 mmol, 2.52 eq). The reaction was stirred at 60° C. for 12 h under nitrogen. The mixture was diluted with ethyl acetate (500 mL) and water (150 mL). The organic layer was separated and concentrated under reduced pressure to give a residue. The residue was purified by silica gel column chromatography (petroleum ether/ethyl acetate=1/1 to 0/1) to afford tert-butyl 3-(2-(2,6-dioxopiperidin-3-yl)-3-oxoisoindolin-5-yl)acrylate IV. ¹H NMR (400 MHz, DMSO-d₆) δ=11.01 (s, 1H), 8.02 (s, 1H), 8.00 (dd, J=1.5, 7.9 Hz, 1H), 7.71 (s, 1H), 7.73-7.61 (m, 1H), 6.66 (d, J=16.0 Hz, 1H), 5.14 (dd, J=5.1, 13.3 Hz, 1H), 4.58-4.45 (m, 1H), 4.43-4.29 (m, 1H), 2.99-2.83 (m, 1H), 2.71-2.60 (m, 1H), 2.41 (dt, J=8.9, 13.2 Hz, 1H), 2.07-1.97 (m, 1H), 1.50 (s, 9H). MS (ESI) m/z 371.2 [M+H]⁺.

V: To a solution of tert-butyl 3-(2-(2,6-dioxopiperidin-3-yl)-3-oxoisoindolin-5-yl)acrylate IV (2.00 g, 5.40 mmol, 1.00 eq) in tetrahydrofuran (20.0 mL) was added palladium on carbon (200 mg, 10% weight on C). The reaction was stirred at 25° C. for 12 h under hydrogen (15 Psi). The mixture was filtered, and the filtrate was concentrated under reduced pressure to give a residue. The residue was triturated with ethyl acetate (20.0 mL) at 25° C. for 10 min.

The resulting solid was collected by filtration and dried under vacuum to afford tert-butyl 3-(2-(2,6-dioxopiperidin-3-yl)-3-oxoisoindolin-5-yl)propanoate V. ¹H NMR (400 MHz, DMSO-d₆) δ=10.99 (s, 1H), 7.59 (s, 1H), 7.51 (d, J=1.1 Hz, 2H), 5.11 (dd, J=5.1, 13.3 Hz, 1H), 4.49-4.37 (m, 1H), 4.35-4.24 (m, 1H), 2.98-2.85 (m, 3H), 2.62 (br d, J=2.1 Hz, 1H), 2.59-2.55 (m, 2H), 2.45-2.30 (m, 1H), 2.07-1.94 (m, 1H), 1.37 (s, 9H). MS (ESI) m/z 317.1 [M+H−56]⁺

VI: To a solution of tert-butyl 3-(2-(2,6-dioxopiperidin-3-yl)-3-oxoisoindolin-5-yl)propanoate V (1.70 g, 4.56 mmol, 1.00 eq) in dichloromethane (20.0 mL) was added trifluoroacetic acid (4.00 mL, 54.0 mmol, 11.8 eq). The reaction was stirred at 25° C. for 2 h. The mixture was concentrated under reduced pressure to give a residue. The residue was triturated with methanol (20.0 mL) at 60° C. for 1 h. The resulting solid was collected by filtration and dried under vacuum to afford 3-(2-(2,6-dioxopiperidin-3-yl)-3-oxoisoindolin-5-yl)propanoic acid VI. ¹H NMR (400 MHz, DMSO-d₆) δ=12.54-11.74 (m, 1H), 10.98 (s, 1H), 7.59 (s, 1H), 7.51 (s, 2H), 5.11 (dd, J=5.1, 13.3 Hz, 1H), 4.47-4.37 (m, 1H), 4.34-4.24 (m, 1H), 2.98-2.85 (m, 3H), 2.65-2.55 (m, 3H), 2.40 (dq, J=4.4, 13.2 Hz, 1H), 2.00 (dtd, J=2.1, 5.2, 12.5 Hz, 1H). MS (ESI) m/z 317.1 [M+H]⁺.

VII: To a solution of 3-(2-(2,6-dioxopiperidin-3-yl)-3-oxoisoindolin-5-yl)propanoic acid VI (1.00 eq) and amine (1.00-2.00 eq) in dimethylformamide (0.14-0.63 M reaction) were added O-(7-azabenzotriazol-1-yl)-N,N,N′,N′-tetramethyluronium hexafluorophosphate (1.00-1.50 eq), and diisopropylethylamine (1.00-3.00 eq). The reaction was stirred at 25° C. for a time frame of 1-16 h. Upon reaction completion, the desired amide products were isolated as white solids according to one of the following variants.

Variant i): The reaction mixture was filtered, and the filtrate was diluted with dimethylformamide (if necessary) and purified by reversed phase preparative HPLC to afford the desired amide products as white solids.

Variant ii): Water was added to the reaction mixture, and the resulting solid was collected by filtration. The filter cake was purified by reversed phase preparative HPLC to afford the desired amide products as white solids.

Variant iii): Water was added to the reaction mixture, and the resulting solid was collected by filtration. The filter cake was triturated with one of the following: water, HCl 1M, methanol, or a mixture of ethyl acetate/methanol=3/1. The resulting solid was collected by filtration to afford the desired amide products as white solids.

Variant iv): The reaction mixture was diluted with ethyl acetate and water. The organic layer was separated, and the aqueous layer was extracted 3× with ethyl acetate. The combined organic layers were dried over sodium sulfate, filtered, and concentrated under reduced pressure to give a residue. The residue was purified by reversed phase preparative HPLC to afford the desired amide products as white solids.

TABLE 1
Specific examples

| Compound | No. | Compound | No. |
|---|---|---|---|
| 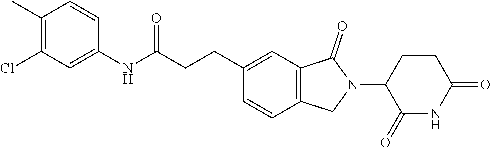 [SMILES: Cc1ccc(NC(=O)CCc2ccc3c(c2)C(=O)N(C2CCC(=O)NC2=O)C3)cc1Cl] | 1 | 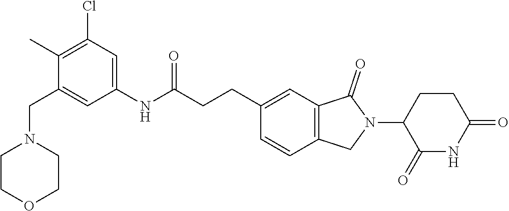 [SMILES: Cc1c(Cl)cc(NC(=O)CCc2ccc3c(c2)C(=O)N(C2CCC(=O)NC2=O)C3)cc1CN1CCOCC1] | 2 |
| 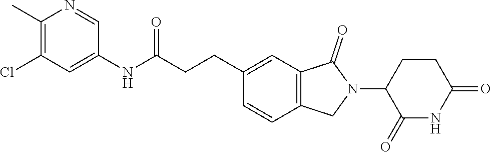 [SMILES: Cc1ncc(NC(=O)CCc2ccc3c(c2)C(=O)N(C2CCC(=O)NC2=O)C3)cc1Cl] | 3 | 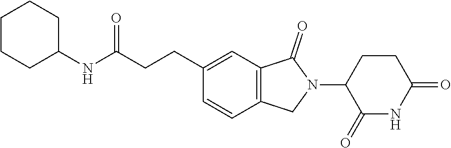 [SMILES: O=C1CCC(N2Cc3ccc(CCC(=O)NC4CCCCC4)cc3C2=O)C(=O)N1] | 4 |
| 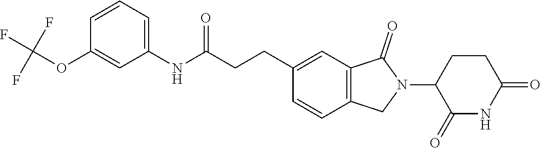 [SMILES: O=C1CCC(N2Cc3ccc(CCC(=O)Nc4cccc(OC(F)(F)F)c4)cc3C2=O)C(=O)N1] | 5 | 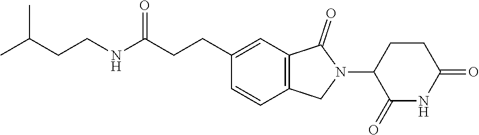 [SMILES: CC(C)CCNC(=O)CCc1ccc2c(c1)C(=O)N(C1CCC(=O)NC1=O)C2] | 6 |
| 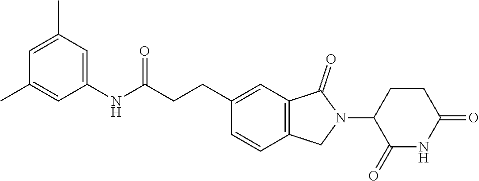 [SMILES: Cc1cc(C)cc(NC(=O)CCc2ccc3c(c2)C(=O)N(C2CCC(=O)NC2=O)C3)c1] | 7 | 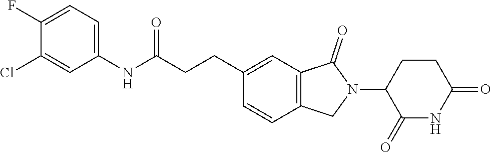 [SMILES: O=C1CCC(N2Cc3ccc(CCC(=O)Nc4ccc(F)c(Cl)c4)cc3C2=O)C(=O)N1] | 8 |
| 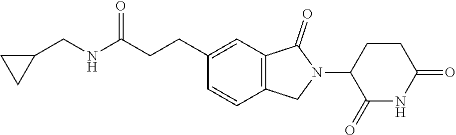 [SMILES: O=C(CCc1ccc2c(c1)C(=O)N(C1CCC(=O)NC1=O)C2)NCC1CC1] | 9 | 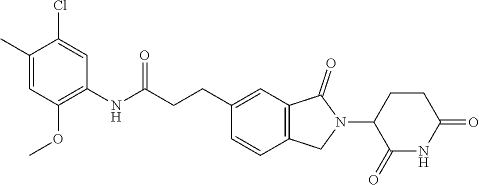 [SMILES: COc1cc(C)c(Cl)cc1NC(=O)CCc1ccc2c(c1)C(=O)N(C1CCC(=O)NC1=O)C2] | 10 |
| 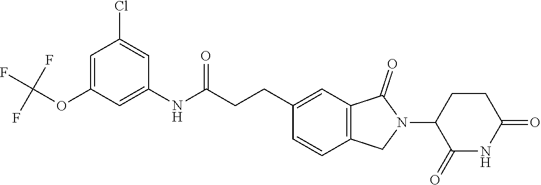 [SMILES: O=C1CCC(N2Cc3ccc(CCC(=O)Nc4cc(Cl)cc(OC(F)(F)F)c4)cc3C2=O)C(=O)N1] | 11 | 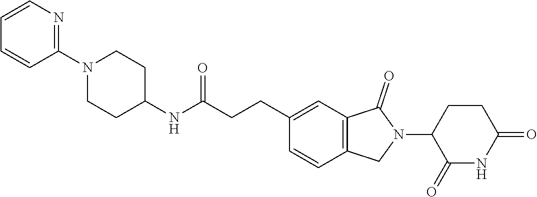 [SMILES: O=C1CCC(N2Cc3ccc(CCC(=O)NC4CCN(c5ccccn5)CC4)cc3C2=O)C(=O)N1] | 12 |
| 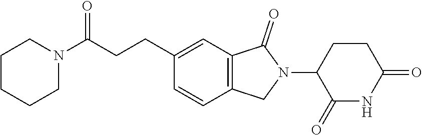 [SMILES: O=C1CCC(N2Cc3ccc(CCC(=O)N4CCCCC4)cc3C2=O)C(=O)N1] | 13 | 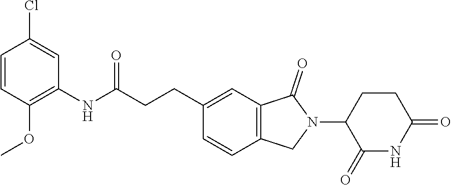 [SMILES: COc1ccc(Cl)cc1NC(=O)CCc1ccc2c(c1)C(=O)N(C1CCC(=O)NC1=O)C2] | 14 |
| 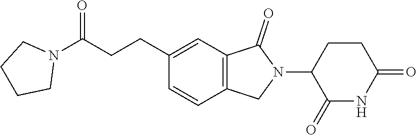 [SMILES: O=C1CCC(N2Cc3ccc(CCC(=O)N4CCCC4)cc3C2=O)C(=O)N1] | 15 | 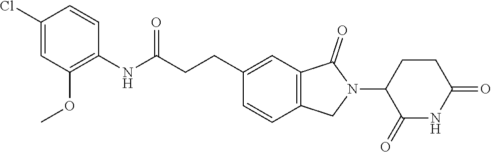 [SMILES: COc1cc(Cl)ccc1NC(=O)CCc1ccc2c(c1)C(=O)N(C1CCC(=O)NC1=O)C2] | 16 |
| 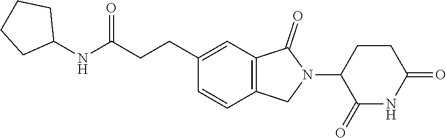 [SMILES: O=C1CCC(N2Cc3ccc(CCC(=O)NC4CCCC4)cc3C2=O)C(=O)N1] | 17 | 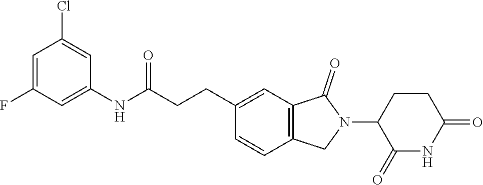 [SMILES: O=C1CCC(N2Cc3ccc(CCC(=O)Nc4cc(F)cc(Cl)c4)cc3C2=O)C(=O)N1] | 18 |
| 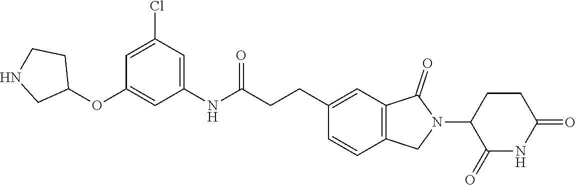 [SMILES: O=C1CCC(N2Cc3ccc(CCC(=O)Nc4cc(Cl)cc(OC5CCNC5)c4)cc3C2=O)C(=O)N1] | 19 | 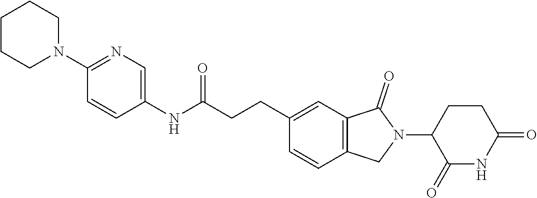 [SMILES: O=C1CCC(N2Cc3ccc(CCC(=O)Nc4ccc(N5CCCCC5)nc4)cc3C2=O)C(=O)N1] | 20 |
| 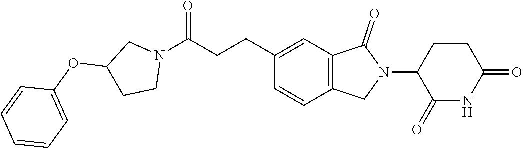 [SMILES: O=C1CCC(N2Cc3ccc(CCC(=O)N4CCC(Oc5ccccc5)C4)cc3C2=O)C(=O)N1] | 21 | 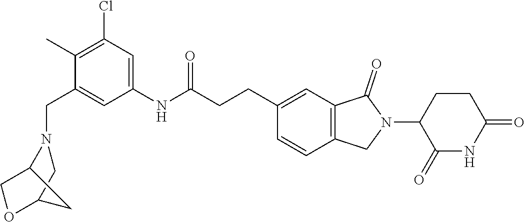 [SMILES: Cc1c(Cl)cc(NC(=O)CCc2ccc3c(c2)C(=O)N(C2CCC(=O)NC2=O)C3)cc1CN1CC2CC1CO2] | 22 |
| 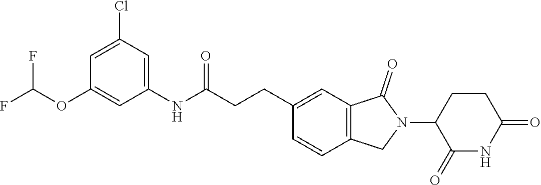 [SMILES: O=C1CCC(N2Cc3ccc(CCC(=O)Nc4cc(Cl)cc(OC(F)F)c4)cc3C2=O)C(=O)N1] | 23 | 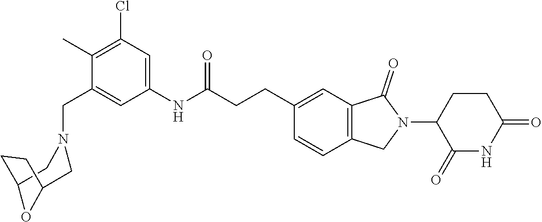 [SMILES: Cc1c(Cl)cc(NC(=O)CCc2ccc3c(c2)C(=O)N(C2CCC(=O)NC2=O)C3)cc1CN1CC2CCC(C1)O2] | 24 |
| 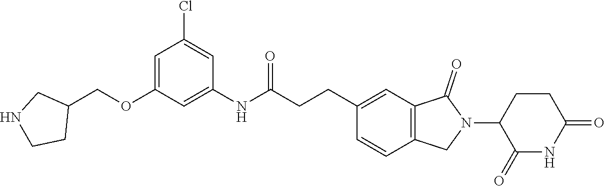 [SMILES: O=C1CCC(N2Cc3ccc(CCC(=O)Nc4cc(Cl)cc(OCC5CCNC5)c4)cc3C2=O)C(=O)N1] | 25 | 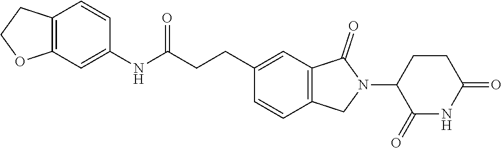 [SMILES: O=C1CCC(N2Cc3ccc(CCC(=O)Nc4ccc5c(c4)OCC5)cc3C2=O)C(=O)N1] | 26 |
| 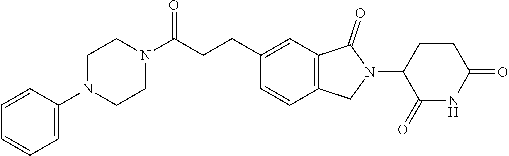 [SMILES: O=C1CCC(N2Cc3ccc(CCC(=O)N4CCN(c5ccccc5)CC4)cc3C2=O)C(=O)N1] | 27 | 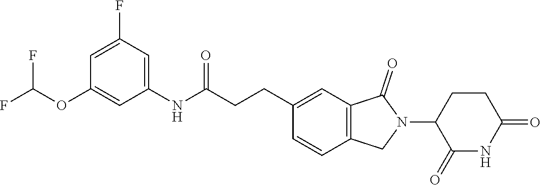 [SMILES: O=C1CCC(N2Cc3ccc(CCC(=O)Nc4cc(F)cc(OC(F)F)c4)cc3C2=O)C(=O)N1] | 28 |
| 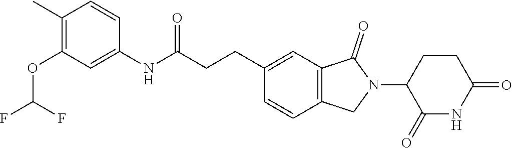 [SMILES: Cc1ccc(NC(=O)CCc2ccc3c(c2)C(=O)N(C2CCC(=O)NC2=O)C3)cc1OC(F)F] | 29 | 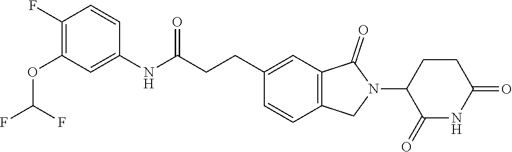 [SMILES: O=C1CCC(N2Cc3ccc(CCC(=O)Nc4ccc(F)c(OC(F)F)c4)cc3C2=O)C(=O)N1] | 30 |
| 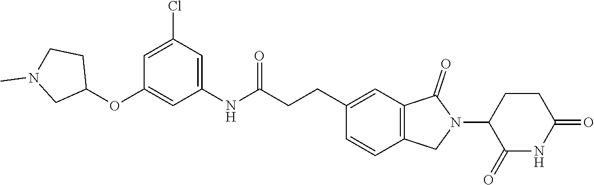 [SMILES: CN1CCC(Oc2cc(Cl)cc(NC(=O)CCc3ccc4c(c3)C(=O)N(C3CCC(=O)NC3=O)C4)c2)C1] | 31 | 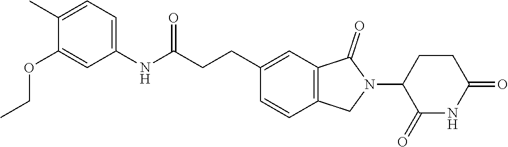 [SMILES: CCOc1cc(NC(=O)CCc2ccc3c(c2)C(=O)N(C2CCC(=O)NC2=O)C3)ccc1C] | 32 |
| 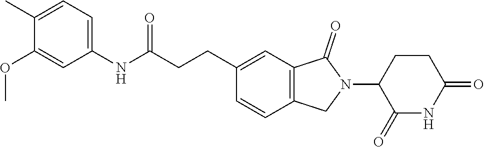 [SMILES: COc1cc(NC(=O)CCc2ccc3c(c2)C(=O)N(C2CCC(=O)NC2=O)C3)ccc1C] | 33 | 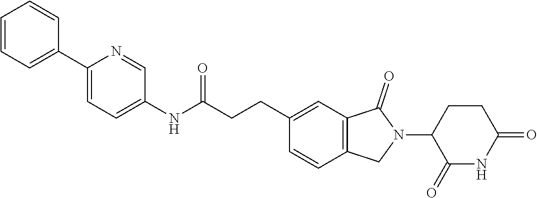 [SMILES: O=C1CCC(N2Cc3ccc(CCC(=O)Nc4ccc(-c5ccccc5)nc4)cc3C2=O)C(=O)N1] | 34 |
| 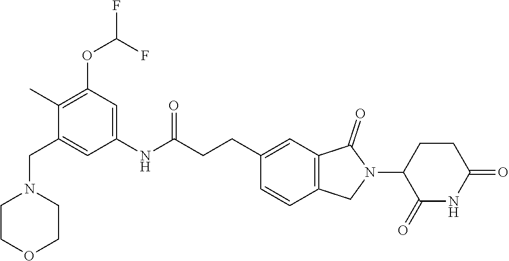 [SMILES: Cc1c(CN2CCOCC2)cc(NC(=O)CCc2ccc3c(c2)C(=O)N(C2CCC(=O)NC2=O)C3)cc1OC(F)F] | 35 | 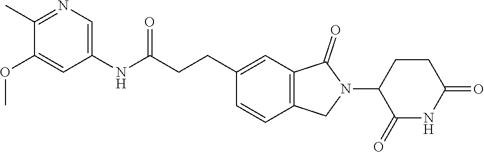 [SMILES: COc1cc(NC(=O)CCc2ccc3c(c2)C(=O)N(C2CCC(=O)NC2=O)C3)cnc1C] | 36 |
| 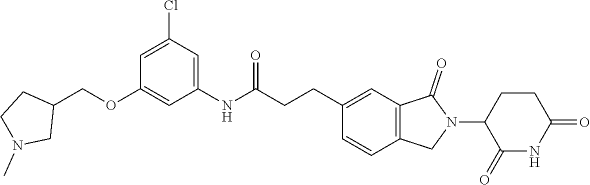 [SMILES: CN1CCC(COc2cc(Cl)cc(NC(=O)CCc3ccc4c(c3)C(=O)N(C3CCC(=O)NC3=O)C4)c2)C1] | 37 | 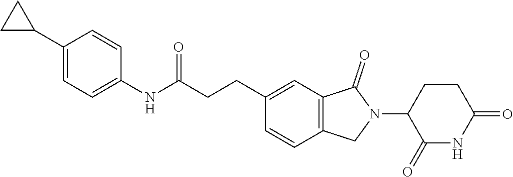 [SMILES: O=C1CCC(N2Cc3ccc(CCC(=O)Nc4ccc(C5CC5)cc4)cc3C2=O)C(=O)N1] | 38 |
| 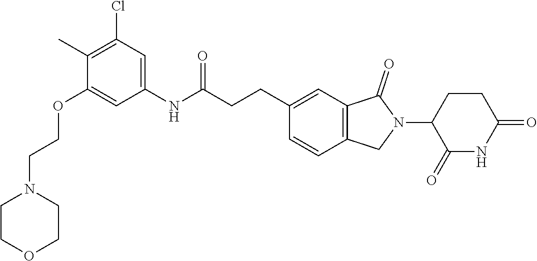 [SMILES: Cc1c(Cl)cc(NC(=O)CCc2ccc3c(c2)C(=O)N(C2CCC(=O)NC2=O)C3)cc1OCCN1CCOCC1] | 39 | 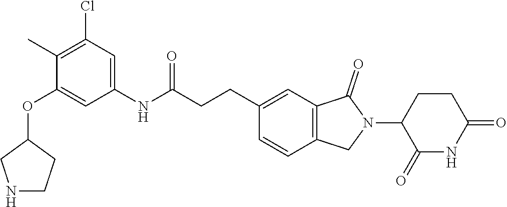 [SMILES: Cc1c(Cl)cc(NC(=O)CCc2ccc3c(c2)C(=O)N(C2CCC(=O)NC2=O)C3)cc1OC1CCNC1] | 40 |
| 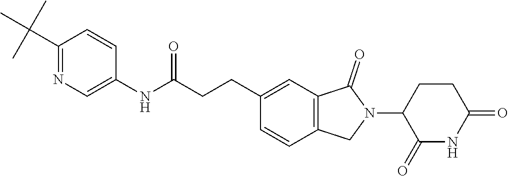 [SMILES: CC(C)(C)c1ccc(NC(=O)CCc2ccc3c(c2)C(=O)N(C2CCC(=O)NC2=O)C3)cn1] | 41 | 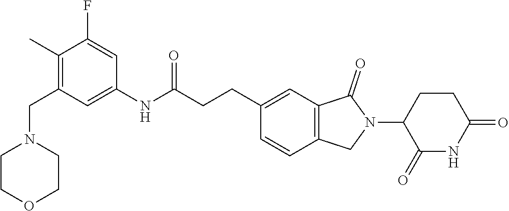 [SMILES: Cc1c(F)cc(NC(=O)CCc2ccc3c(c2)C(=O)N(C2CCC(=O)NC2=O)C3)cc1CN1CCOCC1] | 42 |
| 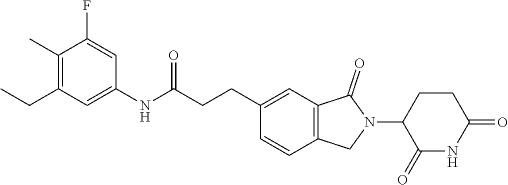 [SMILES: CCc1cc(NC(=O)CCc2ccc3c(c2)C(=O)N(C2CCC(=O)NC2=O)C3)cc(F)c1C] | 43 | 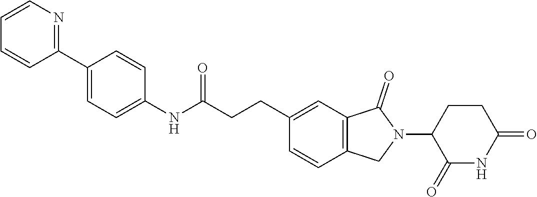 [SMILES: O=C1CCC(N2Cc3ccc(CCC(=O)Nc4ccc(-c5ccccn5)cc4)cc3C2=O)C(=O)N1] | 44 |
| 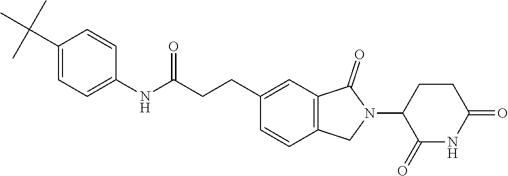 [SMILES: CC(C)(C)c1ccc(NC(=O)CCc2ccc3c(c2)C(=O)N(C2CCC(=O)NC2=O)C3)cc1] | 45 | 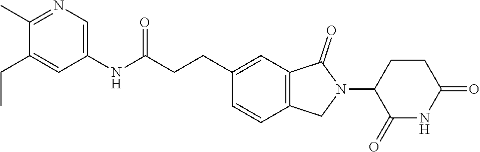 [SMILES: CCc1cc(NC(=O)CCc2ccc3c(c2)C(=O)N(C2CCC(=O)NC2=O)C3)cnc1C] | 46 |
| 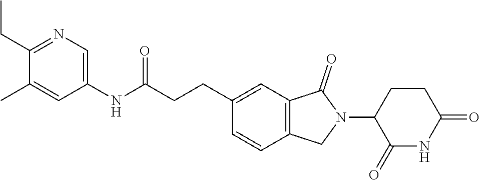 [SMILES: CCc1ncc(NC(=O)CCc2ccc3c(c2)C(=O)N(C2CCC(=O)NC2=O)C3)cc1C] | 47 | 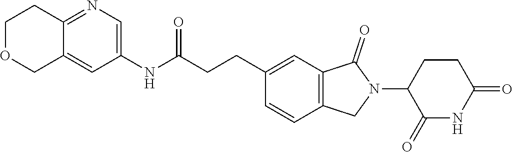 [SMILES: O=C1CCC(N2Cc3ccc(CCC(=O)Nc4cnc5c(c4)COCC5)cc3C2=O)C(=O)N1] | 48 |
| 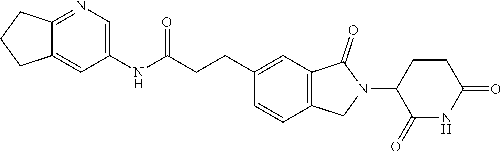 [SMILES: O=C1CCC(N2Cc3ccc(CCC(=O)Nc4cnc5c(c4)CCC5)cc3C2=O)C(=O)N1] | 49 | 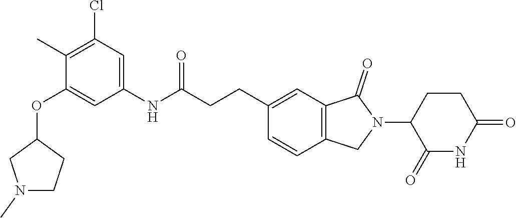 [SMILES: Cc1c(Cl)cc(NC(=O)CCc2ccc3c(c2)C(=O)N(C2CCC(=O)NC2=O)C3)cc1OC1CCN(C)C1] | 50 |
| 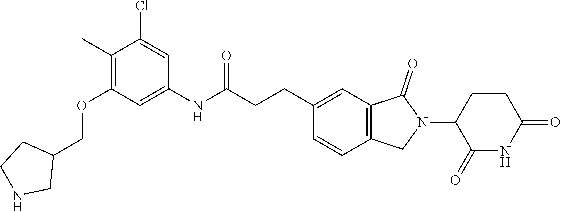 [SMILES: Cc1c(Cl)cc(NC(=O)CCc2ccc3c(c2)C(=O)N(C2CCC(=O)NC2=O)C3)cc1OCC1CCNC1] | 51 | 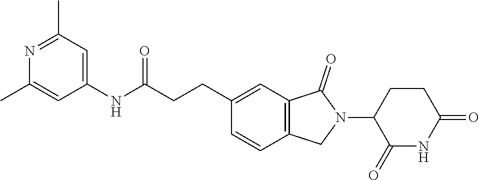 [SMILES: Cc1cc(NC(=O)CCc2ccc3c(c2)C(=O)N(C2CCC(=O)NC2=O)C3)cc(C)n1] | 52 |
| 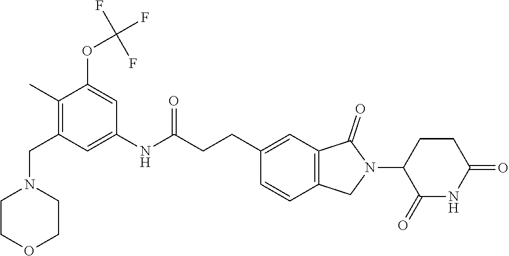 [SMILES: Cc1c(CN2CCOCC2)cc(NC(=O)CCc2ccc3c(c2)C(=O)N(C2CCC(=O)NC2=O)C3)cc1OC(F)(F)F] | 53 | 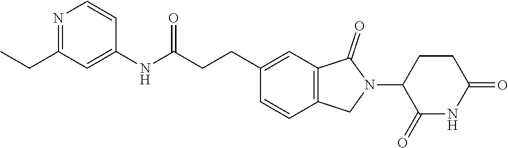 [SMILES: CCc1cc(NC(=O)CCc2ccc3c(c2)C(=O)N(C2CCC(=O)NC2=O)C3)ccn1] | 54 |
| 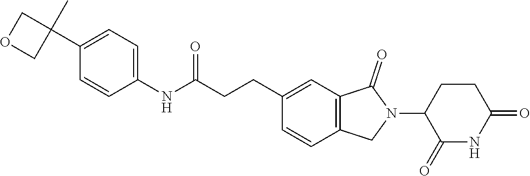 [SMILES: CC1(c2ccc(NC(=O)CCc3ccc4c(c3)C(=O)N(C3CCC(=O)NC3=O)C4)cc2)COC1] | 55 | 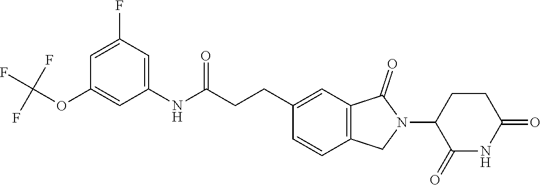 [SMILES: O=C1CCC(N2Cc3ccc(CCC(=O)Nc4cc(F)cc(OC(F)(F)F)c4)cc3C2=O)C(=O)N1] | 56 |
| 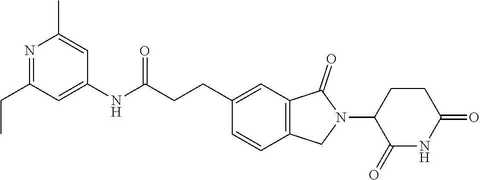 [SMILES: CCc1cc(NC(=O)CCc2ccc3c(c2)C(=O)N(C2CCC(=O)NC2=O)C3)cc(C)n1] | 57 | 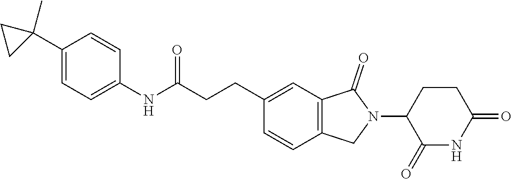 [SMILES: CC1(c2ccc(NC(=O)CCc3ccc4c(c3)C(=O)N(C3CCC(=O)NC3=O)C4)cc2)CC1] | 58 |
| 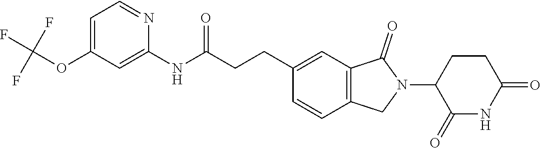 [SMILES: O=C1CCC(N2Cc3ccc(CCC(=O)Nc4cc(OC(F)(F)F)ccn4)cc3C2=O)C(=O)N1] | 59 | 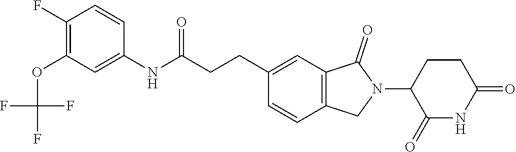 [SMILES: O=C1CCC(N2Cc3ccc(CCC(=O)Nc4ccc(F)c(OC(F)(F)F)c4)cc3C2=O)C(=O)N1] | 60 |
| 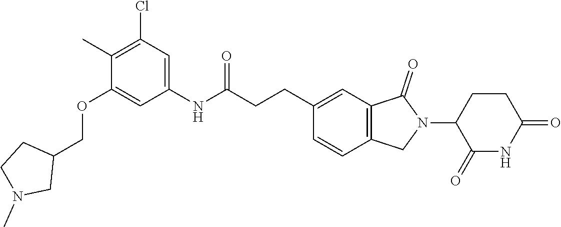 [SMILES: Cc1c(Cl)cc(NC(=O)CCc2ccc3c(c2)C(=O)N(C2CCC(=O)NC2=O)C3)cc1OCC1CCN(C)C1] | 61 | 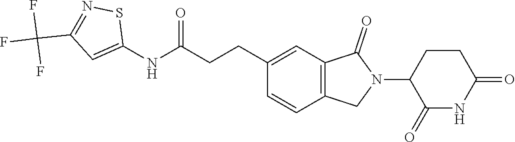 [SMILES: O=C1CCC(N2Cc3ccc(CCC(=O)Nc4cc(C(F)(F)F)ns4)cc3C2=O)C(=O)N1] | 62 |
| 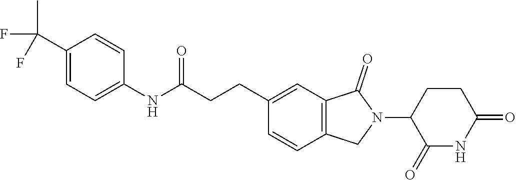 [SMILES: CC(F)(F)c1ccc(NC(=O)CCc2ccc3c(c2)C(=O)N(C2CCC(=O)NC2=O)C3)cc1] | 63 | 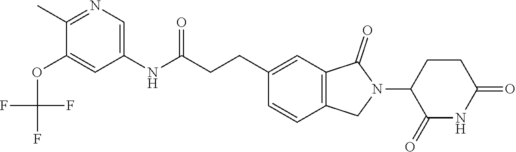 [SMILES: Cc1ncc(NC(=O)CCc2ccc3c(c2)C(=O)N(C2CCC(=O)NC2=O)C3)cc1OC(F)(F)F] | 64 |
| 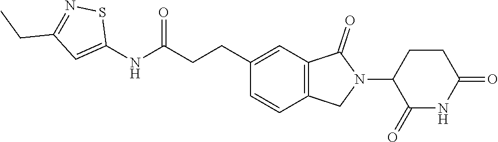 [SMILES: CCc1cc(NC(=O)CCc2ccc3c(c2)C(=O)N(C2CCC(=O)NC2=O)C3)sn1] | 65 | 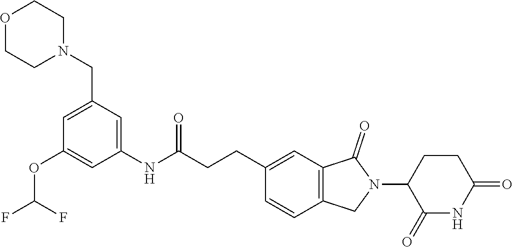 [SMILES: O=C1CCC(N2Cc3ccc(CCC(=O)Nc4cc(CN5CCOCC5)cc(OC(F)F)c4)cc3C2=O)C(=O)N1] | 66 |
| 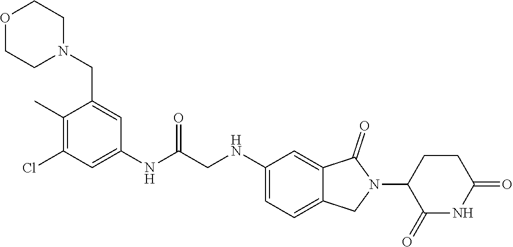 [SMILES: Cc1c(Cl)cc(NC(=O)CNc2ccc3c(c2)C(=O)N(C2CCC(=O)NC2=O)C3)cc1CN1CCOCC1] | 67 | 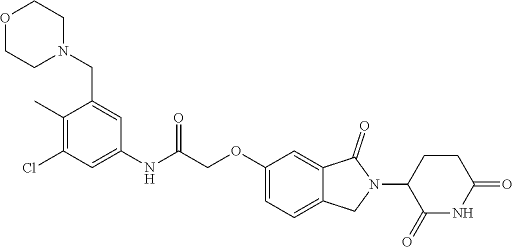 [SMILES: Cc1c(Cl)cc(NC(=O)COc2ccc3c(c2)C(=O)N(C2CCC(=O)NC2=O)C3)cc1CN1CCOCC1] | 68 |
| 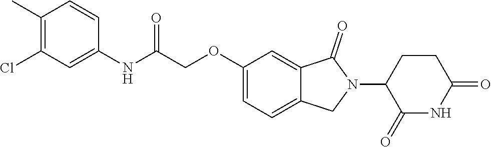 [SMILES: Cc1ccc(NC(=O)COc2ccc3c(c2)C(=O)N(C2CCC(=O)NC2=O)C3)cc1Cl] | 69 | | |

Compound 1: General procedure A with variant iii) was used for the preparation from compound VI employing 3-chloro-4-methylaniline.

¹H NMR (400 MHz, DMSO-d₆) δ=10.98 (s, 1H), 10.01 (s, 1H), 7.78 (d, J=2.0 Hz, 1H), 7.62 (s, 1H), 7.51 (d, J=0.9 Hz, 2H), 7.35-7.29 (m, 1H), 7.27-7.21 (m, 1H), 5.10 (dd, J=5.0, 13.3 Hz, 1H), 4.44-4.37 (m, 1H), 4.32-4.24 (m, 1H), 3.02 (t, J=7.5 Hz, 2H), 2.96-2.85 (m, 1H), 2.67 (t, J=7.5 Hz, 2H), 2.59 (br dd, J=2.1, 15.5 Hz, 1H), 2.44-2.31 (m, 1H), 2.25 (s, 3H), 2.04-1.93 (m, 1H). MS (ESI) m/z 440.1 [M+H]⁺

Compound 2: General procedure A with variant iii) was used for the preparation from compound VI employing 3-chloro-4-methyl-5-(morpholinomethyl)aniline.

¹H NMR (400 MHz, DMSO-d₆) δ=10.98 (s, 1H), 10.00 (s, 1H), 8.15 (s, 1H), 7.79 (d, J=2.0 Hz, 1H), 7.63 (s, 1H), 7.52 (s, 2H), 7.31 (d, J=1.9 Hz, 1H), 5.11 (dd, J=5.1, 13.3 Hz, 1H), 4.44-4.38 (m, 1H), 4.32-4.26 (m, 1H), 3.58-3.55 (m, 4H), 3.41 (s, 2H), 3.02 (br t, J=7.5 Hz, 2H), 2.94-2.89 (m, 1H), 2.67 (s, 2H), 2.62 (br s, 1H), 2.36 (br s, 5H), 2.29 (s, 3H), 2.02-1.97 (m, 1H). MS (ESI) m/z 539.2 [M+H]⁺

Step 1: To a solution of 2-methyl-5-nitro-benzoic acid (10.0 g, 55.2 mmol, 1.00 eq) in concentrated sulfuric acid (30.0 mL, 98% purity) was added 1,3-dichloro-5,5-dimethyl-imidazolidine-2,4-dione (13.0 g, 66.0 mmol, 1.20 eq). The mixture was stirred at 80° C. for 12 h. After cooling to room temperature, the mixture was poured into ice-water. The resulting white precipitate was collected by filtration and dried under reduced pressure to afford 3-chloro-2-methyl-5-nitro-benzoic acid (15.0 g, crude) as a white solid.

Step 2: To a solution of 3-chloro-2-methyl-5-nitro-benzoic acid (15.0 g, 69.6 mmol, 1.00 eq) in tetrahydrofuran (100 mL) was added borane dimethyl sulfide complex (10 M, 14.0 mL, 140.0 mmol, 2.01 eq). The mixture was stirred at 20° C. for 12 h. The mixture was quenched with methanol (10.0 mL) at 0° C. and then concentrated under reduced pressure to give a residue. The residue was diluted with ethyl acetate (200 mL) and adjusted pH=8 with aqueous sodium bicarbonate (10%, 200 mL). The organic layer was separated and concentrated under reduced pressure to afford (3-chloro-2-methyl-5-nitrophenyl)methanol (11.0 g, crude) as a yellow solid.

Step 3: To a solution of (3-chloro-2-methyl-5-nitro-phenyl)methanol (11.0 g, 54.5 mmol, 1 eq) (crude) in dichloromethane (200 mL) was added carbon tetrabromide (21 g, 63.32 mmol, 1.16 eq) and triphenylphosphine (18.6 g, 70.9 mmol, 1.30 eq) at 0° C. The mixture was stirred at 20° C. for 1 h. The mixture was concentrated under reduced pressure to give a residue. The residue was triturated with petroleum ether/ethyl acetate=1/1 (200 mL) and filtered. The filtrate was concentrated under reduced pressure to give a residue. The residue was purified by silica gel column chromatography (petroleum ether/ethyl acetate=8/1) to afford 1-(bromomethyl)-3-chloro-2-methyl-5-nitrobenzene (16.0 g, crude) as a yellow oil.

Step 4: To a solution of 1-(bromomethyl)-3-chloro-2-methyl-5-nitro-benzene (16.0 g, 60.5 mmol, 1.00 eq) in acetonitrile (150 mL) was added morpholine (7.98 mL, 90.7 mmol, 1.50 eq), potassium carbonate (25.0 g, 181 mmol, 2.99 eq) and potassium iodide (1.00 g, 6.05 mmol, 0.100 eq). The reaction was stirred at 80° C. for 12 h. After cooling to room temperature, the mixture was filtered, and the filtrate was concentrated under reduced pressure to give a residue. The residue was purified by silica gel column chromatography (petroleum ether/ethyl acetate=8/1) to give 4-(3-chloro-2-methyl-5-nitrobenzyl)morpholine.

Step 5: To a solution of 4-(3-chloro-2-methyl-5-nitrobenzyl)morpholine (9.70 g, 35.8 mmol, 1.00 eq) in methanol (100 mL) and water (30.0 mL) was added ammonium chloride (15.0 g, 280 mmol, 7.83 eq) and ferrous powder (10.0 g, 179 mmol, 5.00 eq). The reaction was stirred at 80° C. for 2 h. The mixture was filtered, and methanol was removed under reduced pressure. The remaining aqueous solution was extracted with ethyl acetate (2×100 mL). The combined organic layers were washed with brine (20.0 mL), dried over sodium sulfate, filtered, and concentrated under reduced pressure to afford 3-chloro-4-methyl-5-(morpholinomethyl)aniline.

Compound 3: General procedure A with variant i) was used for the preparation from compound VI employing 5-chloro-6-methylpyridin-3-amine.

¹H NMR (400 MHz, DMSO-d₆) δ=10.99 (s, 1H), 10.28 (s, 1H), 8.48 (d, J=2.3 Hz, 1H), 8.19 (d, J=2.1 Hz, 1H), 7.63 (s, 1H), 7.52 (d, J=1.0 Hz, 2H), 5.11 (dd, J=5.1, 13.3 Hz, 1H), 4.46-4.38 (m, 1H), 4.32-4.25 (m, 1H), 3.04 (t, J=7.4 Hz, 2H), 2.96-2.87 (m, 1H), 2.75-2.69 (m, 2H), 2.64-2.57 (m, 1H), 2.48 (s, 3H), 2.46-2.37 (m, 1H), 2.05-1.96 (m, 1H). MS (ESI) m/z 441.1 [M+H]⁺

Step 1: To a solution of 3-chloro-2-methyl-5-nitropyridine (580 mg, 3.36 mmol, 1.00 eq) in methanol (30.0 mL) were added iron power (938 mg, 16.8 mmol, 5.00 eq), ammonium chloride (1.44 g, 26.9 mmol, 8.00 eq) and water (10.0 mL). The reaction was stirred at 80° C. for 2 h. The mixture was filtered, and the filtrate was concentrated under reduced pressure to give a concentrated solution. The concentrated solution was extracted with water/ethyl acetate (20.0 ml/40.0 ml). The organic layer was collected and concentrated under reduced pressure to afford 5-chloro-6-methylpyridin-3-amine.

Compound 4: General procedure A with variant iii) was used for the preparation from compound VI employing cyclohexanamine.

¹H NMR (400 MHz, DMSO-d₆) δ=10.98 (s, 1H), 7.68 (br d, J=8.0 Hz, 1H), 7.57 (s, 1H), 7.53-7.42 (m, 2H), 5.11 (dd, J=5.2, 13.2 Hz, 1H), 4.46-4.35 (m, 1H), 4.33-4.23 (m, 1H), 3.54-3.46 (m, 1H), 2.96-2.86 (m, 3H), 2.60 (br dd, J=2.0, 15.6 Hz, 1H), 2.42-2.35 (m, 3H), 2.05-1.94 (m, 1H), 1.73-1.59 (m, 4H), 1.56-1.50 (m, 1H), 1.29-1.18 (m, 2H), 1.14-1.02 (m, 3H). MS (ESI) m/z 398.3 [M+1]⁺

Compound 5: General procedure A with variant i) was used for the preparation from compound VI employing 3-(trifluoromethoxy)aniline.

¹H NMR (400 MHz, DMSO-d₆) δ=10.14 (s, 1H), 9.40 (s, 1H), 6.94 (s, 1H), 6.80 (s, 1H), 6.72-6.56 (m, 4H), 6.18 (br d, J=7.8 Hz, 1H), 4.28 (dd, J=5.2, 13.4 Hz, 1H), 3.58 (d, J=17.0 Hz, 1H), 3.46 (d, J=17.0 Hz, 1H), 2.20 (t, J=7.6 Hz, 2H), 2.14-2.02 (m, 1H), 1.90-1.72 (m, 3H), 1.72-1.70 (m, 1H), 1.64-1.50 (m, 1H), 1.20-1.12 (m, 1H). MS (ESI) m/z 476.2 [M+H]⁺

Compound 6: General procedure A with variant iii) was used for the preparation from compound VI employing 3-methylbutan-1-amine.

¹H NMR (400 MHz, DMSO-d₆) δ=10.98 (s, 1H), 7.73 (br t, J=5.5 Hz, 1H), 7.56 (s, 1H), 7.54-7.42 (m, 2H), 5.11 (dd, J=5.1, 13.3 Hz, 1H), 4.45-4.37 (m, 1H), 4.33-4.23 (m, 1H), 3.06-2.98 (m, 2H), 2.97-2.87 (m, 3H), 2.60 (br d, J=17.7 Hz, 1H), 2.45-2.33 (m, 3H), 2.05-1.94 (m, 1H), 1.49-1.37 (m, 1H), 1.21 (q, J=7.0 Hz, 2H), 0.81 (d, J=6.6 Hz, 6H). MS (ESI) m/z 386.2 [M+H]⁺

Compound 7: General procedure A with variant i) was used for the preparation from compound VI employing 3,5-dimethylaniline.

¹H NMR (400 MHz, DMSO-d₆) δ=10.98 (br s, 1H), 9.74 (s, 1H), 7.62 (s, 1H), 7.52 (s, 2H), 7.18 (s, 2H), 6.66 (s, 1H), 5.10 (dd, J=5.2, 13.4 Hz, 1H), 4.42 (d, J=17.2 Hz, 1H), 4.28 (d, J=17.0 Hz, 1H), 3.02 (t, J=7.6 Hz, 2H), 2.90 (ddd, J=5.4, 13.6, 17.6 Hz, 1H), 2.68-2.56 (m, 3H), 2.44-2.34 (m, 1H), 2.22 (s, 6H), 2.04-1.94 (m, 1H). MS (ESI) m/z 420.3 [M+H]⁺

Compound 8: General procedure A with variant i) was used for the preparation from compound VI employing 3-chloro-4-fluoroaniline.

¹H NMR (400 MHz, DMSO-d₆) δ=10.98 (s, 1H), 10.14 (s, 1H), 7.90 (dd, J=2.4, 6.8 Hz, 1H), 7.62 (s, 1H), 7.51 (s, 2H), 7.42 (m, 1H), 7.37-7.31 (m, 1H), 5.10 (dd, J=5.2, 13.2 Hz, 1H), 4.45-4.24 (m, 2H), 3.02 (t, J=7.2 Hz, 2H), 2.97-2.84 (m, 1H), 2.73-2.64 (m, 2H), 2.63-2.56 (m, 1H), 2.45-2.32 (m, 1H), 2.05-1.94 (m, 1H). MS (ESI) m/z 444.1 [M+H]⁺.

Compound 9: General procedure A with variant ii) was used for the preparation from compound VI employing cyclopropylmethanamine.

¹H NMR (400 MHz, DMSO-d₆) δ=10.88 (s, 1H), 7.79 (t, J=5.6 Hz, 1H), 7.47 (s, 1H), 7.43-7.34 (m, 2H), 5.00 (dd, J=5.2, 13.2 Hz, 1H), 4.34-4.27 (m, 1H), 4.22-4.14 (m, 1H), 2.86-2.77 (m, 5H), 2.50 (br dd, J=2.4, 16.4 Hz, 1H), 2.36-2.25 (m, 3H), 1.94-1.84 (m, 1H), 0.79-0.68 (m, 1H), 0.31-0.23 (m, 2H), 0.04-−0.04 (m, 2H). MS (ESI) m/z 370.2 [M+1]⁺

Compound 10: General procedure A with variant i) was used for the preparation from compound VI employing 5-chloro-2-methoxy-4-methylaniline.

¹H NMR (400 MHz, DMSO-d₆) δ=10.98 (s, 1H), 9.22 (s, 1H), 8.04 (s, 1H), 7.64 (s, 1H), 7.52 (s, 2H), 7.02 (s, 1H), 5.12 (dd, J=5.2, 13.4 Hz, 1H), 4.42 (d, J=17.2 Hz, 1H), 4.28 (d, J=17.0 Hz, 1H), 3.80 (s, 3H), 3.00 (br t, J=7.5 Hz, 2H), 2.96-2.84 (m, 1H), 2.82-2.74 (m, 2H), 2.68-2.52 (m, 2H), 2.44-2.32 (m, 1H), 2.28 (s, 3H), 2.04-1.96 (m, 1H). MS (ESI) m/z 470.2 [M+H]⁺

Compound 11: General procedure A with variant i) was used for the preparation from compound VI employing 3-chloro-5-(trifluoromethoxy)aniline.

¹H NMR (400 MHz, DMSO-d₆) δ=10.98 (br s, 1H), 10.38 (s, 1H), 7.70 (t, J=1.8 Hz, 1H), 7.62 (br s, 2H), 7.54-7.50 (m, 2H), 7.20 (s, 1H), 5.10 (dd, J=5.2, 13.4 Hz, 1H), 4.42 (d, J=17.0 Hz, 1H), 4.28 (d, J=17.2 Hz, 1H), 3.04 (t, J=7.4 Hz, 2H), 2.96-2.86 (m, 1H), 2.76-2.70 (m, 2H), 2.62-2.58 (m, 1H), 2.40 (dd, J=4.4, 13.0, 1H), 2.04-1.96 (m, 1H). MS (ESI) m/z 510.2 [M+H]⁺

Compound 12: General procedure A with variant ii) was used for the preparation from compound VI employing 1-(pyridin-2-yl)piperidin-4-amine.

¹H NMR (400 MHz, DMSO-d₆) δ=10.99 (br s, 1H), 8.42 (br s, 1H), 8.09 (td, J=1.2, 4.8 Hz, 1H), 7.78 (d, J=7.6 Hz, 1H), 7.57 (s, 1H), 7.54-7.44 (m, 3H), 6.82 (d, J=8.8 Hz, 1H), 6.59 (dd, J=5.2, 6.8 Hz, 1H), 5.11 (dd, J=5.2, 13.2 Hz, 1H), 4.47-4.38 (m, 1H), 4.32-4.25 (m, 1H), 4.13 (br d, J=13.2 Hz, 2H), 3.85-3.75 (m, 1H), 2.94 (br t, J=7.6 Hz, 4H), 2.91-2.86 (m, 1H), 2.63-2.57 (m, 1H), 2.46-2.37 (m, 3H), 2.04-1.97 (m, 1H), 1.72 (br dd, J=2.8, 12.8 Hz, 2H), 1.34-1.24 (m, 2H). MS (ESI) m/z 476.3 [M+1]⁺

Step 1: A solution of 2-fluoropyridine (1.79 mL, 20.8 mmol, 1.00 eq), tert-butyl piperidin-4-ylcarbamate (5.00 g, 25.0 mmol, 1.20 eq) and potassium carbonate (5.75 g, 41.6 mmol, 2.00 eq) in dimethylacetamide (20.0 mL) was stirred at 120° C. for 12 h. The mixture was poured into water (20.0 mL) and extracted with ethyl acetate (3×20.0 mL). The combined organic layers were washed with brine (10.0 mL), dried over anhydrous sodium sulfate, filtered, and concentrated under reduced pressure to give a residue. The residue was purified by silica gel column chromatography (petroleum ether/ethyl acetate=5/1 to 3/1) to afford tert-butyl (1-(pyridin-2-yl)piperidin-4-yl)carbamate.

Step 2: A solution of tert-butyl (1-(pyridin-2-yl)piperidin-4-yl)carbamate (1.27 g, 4.58 mmol, 1.00 eq) and hydrochloric acid/ethyl acetate (8.00 mL) in ethyl acetate (24.0 mL) was stirred at 25° C. for 2 h. The mixture was concentrated under reduced pressure to give a residue. The residue was triturated with ethyl acetate (10.0 mL) at 25° C. for 10 min and filtered to afford 1-(pyridin-2-yl)piperidin-4-amine (1.10 g, crude).

Compound 13: General procedure A with variant i) was used for the preparation from compound VI employing piperidine.

¹H NMR (400 MHz, DMSO-d₆) δ=10.97 (s, 1H), 7.60 (s, 1H), 7.50 (s, 2H), 5.11 (dd, J=5.2, 13.2 Hz, 1H), 4.45-4.24 (m, 2H), 3.43-3.38 (m, 4H), 2.97-2.85 (m, 3H), 2.65 (t, J=7.6 Hz, 2H), 2.58 (s, 1H), 2.46-2.35 (m, 1H), 2.05-1.95 (m, 1H), 1.59-1.51 (m, 2H), 1.45-1.36 (m, 4H). MS (ESI) m/z 384.2 [M+H]⁺

Compound 14: General procedure A with variant ii) was used for the preparation from compound VI employing 5-chloro-2-methoxyaniline.

¹H NMR (400 MHz, DMSO-d₆) δ=10.98 (s, 1H), 9.30 (s, 1H), 8.10 (br d, J=2.0 Hz, 1H), 7.66 (s, 1H), 7.52 (s, 2H), 7.12-7.06 (m, 1H), 7.06-6.99 (m, 1H), 5.12 (dd, J=5.2, 13.2 Hz, 1H), 4.46-4.37 (m, 1H), 4.34-4.25 (m, 1H), 3.82 (s, 3H), 3.06-2.98 (m, 2H), 2.96-2.87 (m, 1H), 2.80 (br t, J=7.6 Hz, 2H), 2.60 (br d, J=18.2 Hz, 1H), 2.45-2.37 (m, 1H), 2.04-1.96 (m, 1H). MS (ESI) m/z 456.2 [M+H]⁺

Compound 15: General procedure A with variant i) was used for the preparation from compound VI employing pyrrolidine.

¹H NMR (400 MHz, DMSO-d₆) δ=10.97 (s, 1H), 7.60 (s, 1H), 7.54-7.47 (m, 2H), 5.10 (dd, J=5.2, 13.2 Hz, 1H), 4.46-4.24 (m, 2H), 3.37 (s, 2H), 3.27 (t, J=6.8 Hz, 2H), 2.96-2.86 (m, 3H), 2.63-2.56 (m, 3H), 2.44-2.36 (m, 1H), 2.02-1.97 (m, 1H), 1.87-1.79 (m, 2H), 1.77-1.70 (m, 2H). MS (ESI) m/z 370.1 [M+H]⁺

Compound 16: General procedure A with variant i) was used for the preparation from compound VI employing 4-chloro-2-methoxy-aniline.

¹H NMR (400 MHz, DMSO-d₆) δ=10.99 (s, 1H), 9.22 (s, 1H), 7.95 (d, J=8.6 Hz, 1H), 7.65 (s, 1H), 7.52 (s, 2H), 7.09 (d, J=2.3 Hz, 1H), 6.95 (dd, J=2.3, 8.6 Hz, 1H), 5.12 (dd, J=5.1, 13.3 Hz, 1H), 4.46-4.38 (m, 1H), 4.33-4.26 (m, 1H), 3.83 (s, 3H), 3.01 (br t, J=7.4 Hz, 2H), 2.96-2.86 (m, 1H), 2.81-2.73 (m, 2H), 2.64-2.56 (m, 1H), 2.46-2.34 (m, 1H), 2.04-1.96 (m, 1H). MS (ESI) m/z 455.9[M+H]⁺

Compound 17: General procedure A with variant i) was used for the preparation from compound VI employing cyclopentanamine.

¹H NMR (400 MHz, DMSO-d₆) δ=10.98 (s, 1H), 7.82-7.68 (m, 1H), 7.57 (s, 1H), 7.52-7.48 (m, 1H), 7.48-7.44 (m, 1H), 5.11 (dd, J=5.1, 13.3 Hz, 1H), 4.47-4.35 (m, 1H), 4.34-4.23 (m, 1H), 4.05-3.92 (m, 1H), 3.02-2.87 (m, 3H), 2.60 (br dd, J=1.8, 15.9 Hz, 1H), 2.44-2.32 (m, 3H), 2.04-1.94 (m, 1H), 1.83-1.70 (m, 2H), 1.63-1.54 (m, 2H), 1.49-1.41 (m, 2H), 1.36-1.23 (m, 2H). MS (ESI) m/z 384.0 [M+H]⁺

Compound 18: General procedure A with variant i) was used for the preparation from compound VI employing 3-chloro-5-fluoro-aniline.

¹H NMR (400 MHz, DMSO-d₆) δ=10.16 (s, 1H), 9.48 (s, 1H), 6.80 (s, 1H), 6.72-6.66 (m, 3H), 6.64-6.60 (m, 1H), 6.26 (td, J=2.2, 8.8 Hz, 1H), 4.28 (dd, J=5.2, 13.4 Hz, 1H), 3.60 (d, J=17.0 Hz, 1H), 3.46 (d, J=17.2 Hz, 1H), 2.22 (t, J=7.6 Hz, 2H), 2.14-2.04 (m, 1H), 1.88 (br t, J=7.6 Hz, 2H), 1.80 (br d, J=1.6 Hz, 1H), 1.62-1.50 (m, 1H), 1.22-1.14 (m, 1H). MS (ESI) m/z 444.2 [M+H]⁺

### Compound 19

Step 1: To a solution of tert-butyl 3-(3-amino-5-chloro-phenoxy)pyrrolidine-1-carboxylate (445 mg, 1.42 mmol, 1.50 eq), 0-(7-azabenzotriazol-1-yl)-N,N,N,N-tetramethyluronium hexafluorophosphate (540 mg, 1.42 mmol, 1.50 eq) and N,N-diisopropylethylamine (0.50 mL, 2.85 mmol, 3.00 eq) in dimethylformamide (3.00 mL) was added 3-(2-(2,6-dioxopiperidin-3-yl)-3-oxoisoindolin-5-yl)propanoic acid (300 mg, 0.95 mmol, 1.00 eq). The reaction was stirred at 25° C. for 12 h. The mixture was filtered, and the filtrate was concentrated under reduced pressure to give a residue. The residue was purified by reversed phase preparative HPLC to afford tert-butyl 3-(3-chloro-5-(3-(2-(2,6-dioxopiperidin-3-yl)-3-oxoisoindolin-5-yl)propanamido) phenoxy)pyrrolidine-1-carboxylate.

Step 2: A solution of tert-butyl 3-(3-chloro-5-(3-(2-(2,6-dioxopiperidin-3-yl)-3-oxoisoindolin-5-yl)propanamido)phenoxy)pyrrolidine-1-carboxylate (500 mg, 818 μmol, 1.00 eq) in hydrochloric acid/dioxane (4 M, 16.6 mL, 81.5 eq) was stirred at 25° C. for 2 h. The mixture was filtered, and the filtrate was concentrated under reduced pressure to give a residue. The residue was purified by reversed phase preparative HPLC to afford Compound 19.

¹H NMR (400 MHz, DMSO-d₆) δ=11.09-10.92 (m, 1H), 10.36-10.17 (m, 1H), 9.65-9.22 (m, 2H), 7.62 (s, 1H), 7.52 (d, J=0.8 Hz, 2H), 7.31 (br d, J=1.5 Hz, 1H), 7.27 (t, J=1.8 Hz, 1H), 6.76 (t, J=1.9 Hz, 1H), 5.20-5.04 (m, 2H), 4.47-4.38 (m, 1H), 4.35-4.26 (m, 1H), 3.48-3.24 (m, 4H), 3.03 (br t, J=7.4 Hz, 2H), 2.96-2.86 (m, 1H), 2.77-2.67 (m, 2H), 2.66-2.57 (m, 1H), 2.46-2.33 (m, 1H), 2.26-2.07 (m, 2H), 2.04-1.94 (m, 1H). MS (ESI) m/z 511.0 [M+H]⁺

Step 1: To a solution of 3-chloro-5-nitro-phenol (5.00 g, 28.8 mmol, 1.00 eq), tert-butyl 3-hydroxypyrrolidine-1-carboxylate (5.93 g, 31.7 mmol, 1.10 eq) and triphenylphosphine (8.31 g, 31.7 mmol, 1.10 eq) in tetrahydrofuran (2.00 mL) was added diisopropyl azodiformate (6.16 mL, 31.7 mmol, 1.10 eq) at 0° C. The reaction was stirred at 25° C. for 12 h. The mixture was concentrated under reduced pressure to give a residue. The residue was purified by reversed phase preparative HPLC to afford tert-butyl 3-(3-chloro-5-nitrophenoxy)pyrrolidine-1-carboxylate.

Step 2: A mixture of tert-butyl 3-(3-chloro-5-nitro-phenoxy)pyrrolidine-1-carboxylate (3.00 g, 8.75 mmol, 100 eq), iron powder (1.47 g, 26.2 mmol, 3.00 eq) and ammonium chloride (2.34 g, 43.8 mmol, 5.00 eq) in methanol (20.0 mL) and water (10.0 mL) was stirred at 80° C. for 2 h. The mixture was filtered, and the filtrate was concentrated under reduced pressure to give a residue. It was added to water (80.0 mL) and saturated sodium bicarbonate (40.0 mL) and stirred for 10 min. The aqueous layer was extracted with ethyl acetate (3×60.0 mL). The combined organic layers were washed with brine (100 mL), dried over anhydrous sodium sulfate, filtered, and concentrated under reduced pressure to afford tert-butyl 3-(3-amino-5-chlorophenoxy)pyrrolidine-1-carboxylate.

### Compound 20

To a solution of 3-(2-(2,6-dioxopiperidin-3-yl)-3-oxoisoindolin-5-yl)propanoic acid (150 mg, 474 μmol, 1.00 eq) and 6-(1-piperidyl)pyridin-3-amine (92.5 mg, 522 μmol, 1.10 eq) in dimethylformamide (2.00 mL) was added 1-hydroxybenzotriazole (128 mg, 0.95 mmol, 2.00 eq), N,N-diisopropylethylamine (0.25 mL, 1.42 mmol, 3.00 eq) and 1-(3-dimethylaminopropyl)-3-ethylcarbodiimide hydrochloride (182 mg, 0.95 mmol, 2.00 eq). The mixture was stirred at 25° C. for 2 h. The residue was diluted with ethyl acetate (40.0 mL) and water (40.0 mL). The organic layer was separated, and the aqueous layer was extracted with ethyl acetate (3×40.0 mL). The combined organic layers were dried over sodium sulfate, filtered, and concentrated under reduced pressure to give a residue. The residue was purified by reversed phase preparative HPLC to afford Compound 20.

¹H NMR (400 MHz, DMSO-d₆) δ=10.98 (s, 1H), 9.72 (s, 1H), 8.27-8.16 (m, 1H), 7.75-7.66 (m, 1H), 7.62 (s, 1H), 7.51 (s, 2H), 6.76 (d, J=9.1 Hz, 1H), 5.15-5.05 (m, 1H), 4.48-4.22 (m, 2H), 3.45-3.38 (m, 5H), 3.01 (br t, J=7.5 Hz, 2H), 2.95-2.84 (m, 1H), 2.69-2.61 (m, 2H), 2.61-2.55 (m, 1H), 2.45-2.31 (m, 1H), 2.04-1.94 (m, 1H), 1.61-1.48 (m, 6H). MS (ESI) m/z 476.4 [M+H]⁺

Step 1: To a solution of 2-fluoro-5-nitro-pyridine (5.00 g, 35.2 mmol, 1.00 eq) in acetonitrile (50.0 mL) were added potassium carbonate (9.73 g, 70.4 mmol, 2.00 eq) and piperidine (4.17 mL, 42.2 mmol, 1.20 eq). The reaction was stirred at 25° C. for 1 h. The mixture was filtered and concentrated under reduced pressure to afford 5-nitro-2-(1-piperidyl)pyridine.

Step 2: To a solution of 5-nitro-2-(piperidin-1-yl)pyridine (5.20 g, 25.1 mmol, 1.00 eq) and ammonium chloride (6.71 g, 125 mmol, 5.00 eq) in methanol (40.0 mL) and water (10.0 mL) was added iron powder (7.01 g, 126 mmol, 5.00 eq) in portions. The reaction was stirred at 80° C. for 2 h. The mixture was filtered, and the filtrate was concentrated under reduced pressure to give a residue. The residue was diluted with water (150 mL) and extracted with ethyl acetate (3×60.0 mL). The combined organic layers were washed with brine (60.0 mL), dried over sodium sulfate, filtered, and concentrated under reduced pressure to give a residue. The residue was purified by reversed phase preparative HPLC. The desired fraction was collected, diluted with saturated sodium bicarbonate (150 mL), and extracted with ethyl acetate (3×60.0 mL). The combined organic layers were washed with brine (60.0 mL), dried over sodium sulfate, filtered, and concentrated under reduced pressure to afford 6-(piperidin-1-yl)pyridin-3-amine.

Compound 21: General procedure A with variant i) was used for the preparation from compound VI employing 3-phenoxypyrrolidine.

¹H NMR (400 MHz, DMSO-d₆) δ=10.97 (s, 1H), 7.61 (d, J=6.7 Hz, 1H), 7.54-7.45 (m, 2H), 7.29 (t, J=7.6 Hz, 2H), 6.98-6.90 (m, 3H), 5.11 (br dd, J=4.7, 13.0 Hz, 1H), 5.07-4.97 (m, 1H), 4.46-4.34 (m, 1H), 4.33-4.23 (m, 1H), 3.79-3.60 (m, 1H), 3.59-3.52 (m, 2H), 3.52-3.29 (m, 1H), 2.98-2.85 (m, 3H), 2.67-2.56 (m, 3H), 2.45-2.33 (m, 1H), 2.20-1.96 (m, 3H). MS (ESI) m/z 462.1 [M+H]⁺

Step 1: To a solution of tert-butyl 3-hydroxypyrrolidine-1-carboxylate (3.00 g, 16.0 mmol, 1.00 eq) and triethylamine (5.58 mL, 40.0 mmol, 2.50 eq) in dichloromethane (30.0 mL) was added methanesulfonyl chloride (1.74 mL, 22.4 mmol, 1.40 eq) dropwise at 0° C. The reaction was stirred at 25° C. for 2 h. The mixture was diluted with water (150 mL) and extracted with ethyl acetate (3×80.0 mL). The combined organic layers were washed with brine (60.0 mL), dried over sodium sulfate, filtered, and concentrated under reduced pressure to afford tert-butyl 3-((methylsulfonyl)oxy)pyrrolidine-1-carboxylate.

Step 2: To a solution of tert-butyl 3-((methylsulfonyl)oxy)pyrrolidine-1-carboxylate (1.00 g, 3.77 mmol, 1.00 eq) and phenol (0.40 mL, 4.52 mmol, 1.20 eq) in dimethylformamide (10.0 mL) was added caesium carbonate (3.68 g, 11.3 mmol, 3.00 eq) in one portion. The reaction was stirred at 80° C. for 12 h. The mixture was diluted with water (50.0 mL) and extracted with ethyl acetate (3×60.0 mL). The combined organic layers were washed with brine (20.0 mL), dried over sodium sulfate, filtered, and concentrated under reduced pressure to give a residue. The residue was purified by silica gel column chromatography (petroleum ether/ethyl acetate=20/1 to 5/1) and then by reversed phase preparative HPLC to afford tert-butyl 3-phenoxypyrrolidine-1-carboxylate.

Step 3: A solution of tert-butyl 3-phenoxypyrrolidine-1-carboxylate (460 mg, 1.75 mmol, 1.00 eq) in 4 M hydrochloric acid/dioxane (4.00 mL) was stirred at 25° C. for 1 h. The mixture was concentrated under reduced pressure to afford 3-phenoxypyrrolidine (430 mg, crude).

Compound 22: General procedure A with variant ii) was used for the preparation from compound VI employing 3-(2-oxa-5-azabicyclo[2.2.1]heptan-5-ylmethyl)-5-chloro-4-methylaniline.

¹H NMR (400 MHz, CDCl₃) δ=10.99 (s, 1H), 10.86-10.39 (m, 1H), 10.38-10.27 (m, 1H), 7.81 (d, J=1.6 Hz, 1H), 7.77-7.67 (m, 1H), 7.63 (s, 1H), 7.53 (s, 2H), 5.10 (dd, J=5.2, 13.2 Hz, 1H), 4.76-4.62 (m, 1H), 4.60-4.43 (m, 2H), 4.42-4.35 (m, 2H), 4.33-4.25 (m, 1H), 4.21 (br d, J=9.6 Hz, 1H), 3.83-3.65 (m, 2H), 3.61-3.47 (m, 1H), 3.25-3.10 (m, 1H), 3.04 (br t, J=7.6 Hz, 2H), 2.97-2.86 (m, 1H), 2.76-2.69 (m, 2H), 2.60 (br d, J=16.8 Hz, 1H), 2.41 (s, 1H), 2.40-2.29 (m, 3H), 2.11-1.96 (m, 2H). MS (ESI) m/z 551.1 [M+H]⁺

Step 1: To a solution of 2-methyl-5-nitro-benzoic acid (10.0 g, 55.2 mmol, 1.00 eq) in concentrated sulfuric acid (30.0 mL, 98% purity) was added 1,3-dichloro-5,5-dimethyl-imidazolidine-2,4-dione (13.0 g, 66.0 mmol, 1.20 eq). The reaction was stirred at 80° C. for 12 h. After cooling to room temperature, the mixture was poured into ice-water. The resulting white precipitate was collected by filtration and dried under vacuum to afford 3-chloro-2-methyl-5-nitro-benzoic acid.

Step 2: To a solution of 3-chloro-2-methyl-5-nitro-benzoic acid (15.0 g, 69.6 mmol, 1.00 eq) in tetrahydrofuran (100 mL) was added borane dimethyl sulfide complex (10 M, 14.0 mL, 2.01 eq) at 20° C. The reaction was stirred at 20° C. for 12 h. The mixture was quenched with methanol (10.0 mL) at 0° C. and concentrated under reduced pressure to give a residue. Ethyl acetate (200 mL) was added, and the pH was adjusted pH=8 with aqueous sodium bicarbonate (10%, 200 mL). The organic layer was separated and concentrated under reduced pressure to afford (3-chloro-2-methyl-5-nitrophenyl)methanol.

Step 3: To a solution of (3-chloro-2-methyl-5-nitro-phenyl)methanol (2.00 g, 9.92 mmol, 1.00 eq) in dichloromethane (30.0 mL) was added thionyl chloride (3.60 mL, 49.6 mmol, 5.00 eq) at 25° C. The reaction was stirred at 25° C. for 12 h. The reaction was quenched by addition of ice water (20.0 mL) at 0° C., and the aqueous layer was extracted with ethyl acetate (3×20.0 mL). The combined organic layers were washed with brine (30.0 mL), dried over anhydrous sodium sulfate, filtered, and concentrated under reduced pressure to afford 1-chloro-3-(chloromethyl)-2-methyl-5-nitrobenzene.

Step 4: To a solution of 1-chloro-3-(chloromethyl)-2-methyl-5-nitro-benzene (2.40 g, 10.9 mmol, 1.00 eq), 2-oxa-5-azabicyclo[2.2.1]heptane (1.77 g, 13.1 mmol, 1.20 eq) and potassium carbonate (4.52 g, 32.7 mmol, 3.00 eq) in acetonitrile (2.00 mL) was added potassium iodide (181 mg, 1.09 mmol, 0.10 eq) at 25° C. The reaction was stirred at 80° C. for 12 h. The mixture was filtered, and the filtrate was concentrated under reduced pressure to give a residue. The residue was purified by reversed phase preparative HPLC to afford 5-(3-chloro-2-methyl-5-nitrobenzyl)-2-oxa-5-azabicyclo[2.2.1]heptane.

Step 5: To a mixture of 5-(3-chloro-2-methyl-5-nitrobenzyl)-2-oxa-5-azabicyclo[2.2.1]heptane (1.45 g, 5.13 mmol, 1.00 eq), iron powder (1.43 g, 25.6 mmol, 5.00 eq) and ammonium chloride (1.37 g, 25.6 mmol, 5.00 eq) in methanol (50.0 mL) was added water (50.0 mL) at 25° C. The reaction was stirred at 80° C. for 2 h. The mixture was concentrated under reduced pressure to give a residue. The residue was poured into saturated sodium bicarbonate (20.0 mL) and extracted with ethyl acetate (3×20.0 mL). The combined organic layers were washed with brine (20.0 mL), dried over anhydrous sodium sulfate, filtered, and concentrated under reduced pressure to afford 3-(2-oxa-5-azabicyclo[2.2.1]heptan-5-ylmethyl)-5-chloro-4-methylaniline.

Compound 23: General procedure A with variant ii) was used for the preparation from compound VI employing 3-chloro-5-(difluoromethoxy)aniline.

¹H NMR (400 MHz, DMSO-d₆) δ=10.98 (s, 1H), 10.28 (s, 1H), 7.63 (s, 1H), 7.58 (t, J=2.0 Hz, 1H), 7.52 (d, J=0.8 Hz, 2H), 7.38 (d, J=1.6 Hz, 1H), 7.26 (t, J=80 Hz, 1H), 6.98 (t, J=2.0 Hz, 1H), 5.11 (dd, J=5.2, 13.2 Hz, 1H), 4.48-4.37 (m, 1H), 4.34-4.25 (m, 1H), 3.03 (br t, J=7.2 Hz, 2H), 2.96-2.87 (m, 1H), 2.71 (br t, J=7.6 Hz, 2H), 2.60 (br d, J=18.4 Hz, 1H), 2.40 (br dd, J=4.4, 13.2 Hz, 1H), 2.04-1.95 (m, 1H). MS (ESI) m/z 492.2 [M+1]⁺

Step 1: To a solution of 3-chloro-5-nitro-phenol (1.50 g, 8.64 mmol, 1.00 eq) and sodium 2-chloro-2,2-difluoroacetate (5.27 g, 34.6 mmol, 4.00 eq) in dimethylformamide (17.0 mL) and water (2.00 mL) was added potassium carbonate (2.39 g, 17.3 mmol, 2.00 eq). The reaction was stirred at 100° C. for 12 h. The mixture was filtered and concentrated under reduced pressure to give a residue. The residue was diluted with water (100 mL) and extracted with ethyl acetate (3×20.0 mL). The combined organic layers were washed with saturated sodium carbonate solution (50.0 mL) and brine (20.0 mL), dried over anhydrous sodium sulfate, filtered, and concentrated under reduced pressure to afford 1-chloro-3-(difluoromethoxy)-5-nitrobenzene.

Step 2: To a solution of 1-chloro-3-(difluoromethoxy)-5-nitro-benzene (1.70 g, 7.60 mmol, 1.00 eq) in methanol (10.0 mL) and water (10.0 mL) were added iron powder (2.12 g, 38.0 mmol, 5.00 eq) and ammonium chloride (2.03 g, 38.0 mmol, 5.00 eq). The reaction was stirred at 80° C. for 2 h. The mixture was concentrated under reduced pressure to give a residue. The residue was diluted with saturated sodium bicarbonate solution (20.0 mL) and extracted with ethyl acetate (3×20.0 mL). The combined organic layers were washed with brine (20.0 mL), dried over anhydrous sodium sulfate, filtered, and concentrated under reduced pressure to afford 3-chloro-5-(difluoromethoxy)aniline.

Compound 24: General procedure A with variant ii) was used for the preparation from compound VI employing 3-(8-oxa-3-azabicyclo[3.2.1]octan-3-ylmethyl)-5-chloro-4-methylaniline.

¹H NMR (400 MHz, DMSO-d₆) δ=10.98 (s, 1H), 10.47 (br d, J=2.4 Hz, 1H), 10.40 (br s, 1H), 7.87 (d, J=1.2 Hz, 1H), 7.76 (br s, 1H), 7.63 (s, 1H), 7.60-7.48 (m, 2H), 5.10 (dd, J=5.2, 13.2 Hz, 1H), 4.42 (br d, J=17.6 Hz, 4H), 4.27 (br s, 2H), 3.24 (br d, J=10.8 Hz, 2H), 3.11 (br s, 2H), 3.04 (br t, J=7.6 Hz, 2H), 2.95-2.87 (m, 1H), 2.73 (br t, J=7.6 Hz, 2H), 2.63-2.58 (m, 1H), 2.46-2.40 (m, 1H), 2.37 (s, 3H), 2.24 (br d, J=7.2 Hz, 2H), 2.05-1.97 (m, 1H), 1.96-1.81 (m, 2H). MS (ESI) m/z 565.4 [M+H]⁺

Step 1: To a solution of (3-chloro-2-methyl-5-nitro-phenyl)methanol (2.00 g, 9.92 mmol, 1.00 eq) in dichloromethane (30.0 mL) was added thionyl chloride (3.60 mL, 49.6 mmol, 5.00 eq) at 25° C. The reaction was stirred at 25° C. for 12 h. The reaction was quenched by addition of ice water (20.0 mL) at 0° C., and the aqueous layer was extracted with ethyl acetate (3×20.0 mL). The combined organic layers were washed with brine (30.0 mL), dried over anhydrous sodium sulfate, filtered, and concentrated under reduced pressure to afford 1-chloro-3-(chloromethyl)-2-methyl-5-nitrobenzene.

Step 2: To a solution of 1-chloro-3-(chloromethyl)-2-methyl-5-nitro-benzene (1.40 g, 6.36 mmol, 1.00 eq), 8-oxa-3-azabicyclo[3.2.1]octane (1.14 g, 7.63 mmol, 1.20 eq, HCl) and potassium carbonate (2.64 g, 19.1 mmol, 3.00 eq) in acetonitrile (20.0 mL) was added potassium iodide (106 mg, 0.64 mmol, 0.10 eq) at 25° C. The reaction was stirred at 80° C. for 12 h. The mixture was filtered and concentrated under reduced pressure to give a residue. The residue was purified by silica gel column chromatography (petroleum ether/ethyl acetate=10/1 to 2/1) to afford 3-(3-chloro-2-methyl-5-nitrobenzyl)-8-oxa-3-azabicyclo[3.2.1]octane.

Step 3: To a solution of 3-(3-chloro-2-methyl-5-nitrobenzyl)-8-oxa-3-azabicyclo[3.2.1]octane (1.00 g, 3.37 mmol, 1.00 eq) in methanol (20.0 mL) were added iron powder (941 mg, 16.9 mmol, 5.00 eq), ammonium chloride (901 mg, 16.9 mmol, 5.00 eq) and water (5.00 mL) at 25° C. The reaction was stirred at 80° C. for 2 h. The mixture was concentrated under reduced pressure to give a residue. Sodium bicarbonate (20.0 mL) was added, and the aqueous layer was extracted with ethyl acetate (3×20.0 mL). The combined organic layers were washed with brine (20.0 mL), dried over anhydrous sodium sulfate, filtered, and concentrated under reduced pressure to afford 3-(8-oxa-3-azabicyclo[3.2.1]octan-3-ylmethyl)-5-chloro-4-methylaniline.

### Compound 25

Step 1: To a solution of tert-butyl 3-((3-amino-5-chlorophenoxy)methyl)pyrrolidine-1-carboxylate (310 mg, 0.95 mmol, 1.50 eq), O-(7-azabenzotriazol-1-yl)-N,N,N,N-tetramethyluroniumhexafluorophosphate (360 mg, 0.95 mmol, 1.50 eq) and N,N-diisopropylethylamine (0.33 mL, 1.90 mmol, 3.00 eq) in dimethylformamide (3.00 mL) was added 3-(2-(2,6-dioxopiperidin-3-yl)-3-oxoisoindolin-5-yl)propanoic acid (200 mg, 632 μmol, 1.00 eq). The reaction was stirred at 25° C. for 2 h. The mixture was filtered, and the filtrate was purified by reversed phase preparative HPLC to afford tert-butyl 3-((3-chloro-5-(3-(2-(2,6-dioxopiperidin-3-yl)-3-oxoisoindolin-5-yl)propanamido)phenoxy)methyl)pyrrolidine-1-carboxylate.

Step 2: A solution of tert-butyl 3-((3-chloro-5-(3-(2-(2,6-dioxopiperidin-3-yl)-3-oxoisoindolin-5-yl)propanamido)phenoxy)methyl)pyrrolidine-1-carboxylate (300 mg, 0.48 mmol, 1.00 eq) in hydrochloric acid/ethyl acetate (4 M, 5.00 mL, 41.7 eq) was stirred at 25° C. for 1 h. The mixture was concentrated under reduced pressure to give a residue. The residue was purified by reversed phase preparative HPLC to afford Compound 25.

¹H NMR (400 MHz, DMSO-d₆) δ=10.99 (s, 1H), 10.32 (s, 1H), 9.32 (br s, 2H), 7.62 (s, 1H), 7.52 (d, J=0.9 Hz, 2H), 7.28 (td, J=1.7, 16.3 Hz, 2H), 6.72 (t, J=2.0 Hz, 1H), 5.10 (dd, J=5.1, 13.3 Hz, 1H), 4.49-4.38 (m, 1H), 4.33-4.24 (m, 1H), 4.04-3.96 (m, 2H), 3.37-3.12 (m, 3H), 3.06-2.97 (m, 3H), 2.94-2.84 (m, 1H), 2.76-2.66 (m, 3H), 2.64-2.55 (m, 1H), 2.45-2.35 (m, 1H), 2.14-1.96 (m, 2H), 1.82-1.66 (m, 1H). MS (ESI) m/z 525.0 [M+H]⁺

Step 1: To a solution of tert-butyl 3-(hydroxymethyl)pyrrolidine-1-carboxylate (3.00 g, 14.9 mmol, 1.00 eq) and triethylamine (5.19 mL, 37.3 mmol, 2.50 eq) in dichloromethane (30.0 mL) at 0° C. was added methylsulfamoyl chloride (1.50 mL, 19.4 mmol, 1.30 eq) dropwise over 2 min under nitrogen atmosphere. The reaction was then stirred at 25° C. for 2 h. The mixture was diluted with ethyl acetate (100 mL) and water (150 mL). The organic layer was separated, and the aqueous layer was extracted with ethyl acetate (3×100 mL). The combined organic layers were dried over sodium sulfate, filtered, and concentrated under reduced pressure to afford tert-butyl 3-(((methylsulfonyl)oxy)methyl)pyrrolidine-1-carboxylate.

Step 2: A solution of tert-butyl 3-(((methylsulfonyl)oxy)methyl) pyrrolidine-1-carboxylate (3.00 g, 10.8 mmol, 1.00 eq), 3-chloro-5-nitrophenol (2.05 g, 11.8 mmol, 1.10 eq) and caesium carbonate (10.5 g, 32.2 mmol, 3.00 eq) in dimethylformamide (30.0 mL) was stirred at 80° C. for 12 h. The mixture was diluted with ethyl acetate (100 mL) and water (100 mL). The organic layer was separated, and the aqueous layer was extracted with ethyl acetate (3×80.0 mL). The combined organic layers were dried over sodium sulfate, filtered, and concentrated under reduced pressure to give a residue. The residue was purified by silica gel column chromatography (petroleum ether/ethyl acetate=3/1) to afford tert-butyl 3-((3-chloro-5-nitrophenoxy)methyl)pyrrolidine-1-carboxylate.

Step 3: A solution of tert-butyl 3-((3-chloro-5-nitrophenoxy)methyl)pyrrolidine-1-carboxylate (2.00 g, 5.61 mmol, 1.00 eq), iron powder (2.19 g, 39.2 mmol, 7.00 eq) and ammonium chloride (2.10 g, 39.2 mmol, 7.00 eq) in methanol (30.0 mL) and water (30.0 mL) was stirred at 80° C. for 2 h. The mixture was filtered over Celite, and the filtrate was concentrated under reduced pressure to afford tert-butyl 3-((3-amino-5-chlorophenoxy)methyl)pyrrolidine-1-carboxylate.

Compound 26: General procedure A with variant i) was used for the preparation from compound VI employing 2,3-dihydrobenzofuran-6-amine.

¹H NMR (400 MHz, DMSO-d₆) δ=10.15 (s, 1H), 8.99 (s, 1H), 6.79 (s, 1H), 6.73-6.62 (m, 2H), 6.35-6.21 (m, 2H), 6.10 (dd, J=1.7, 7.9 Hz, 1H), 4.27 (dd, J=5.1, 13.3 Hz, 1H), 3.66 (t, J=8.7 Hz, 2H), 3.61-3.54 (m, 1H), 3.51-3.40 (m, 1H), 2.30-2.22 (m, 2H), 2.18 (br t, J=7.5 Hz, 2H), 2.07 (ddd, J=5.4, 13.6, 17.5 Hz, 1H), 1.87-1.72 (m, 3H), 1.72-1.69 (m, 1H), 1.63-1.47 (m, 1H), 1.21-1.12 (m, 1H). MS (ESI) m/z 434.2 [M+H]⁺

Compound 27: General procedure A with variant i) was used for the preparation from compound VI employing 1-phenylpiperazine.

¹H NMR (400 MHz, DMSO-d₆) δ=10.98 (s, 1H), 7.64 (s, 1H), 7.59-7.43 (m, 2H), 7.35-7.09 (m, 2H), 6.93 (br d, J=8.3 Hz, 2H), 6.81 (br t, J=7.2 Hz, 1H), 5.11 (dd, J=5.0, 13.2 Hz, 1H), 4.40 (br d, J=17.0 Hz, 1H), 4.28 (br d, J=17.1 Hz, 1H), 3.59 (br d, J=4.0 Hz, 4H), 3.11-3.02 (m, 4H), 2.98-2.87 (m, 3H), 2.74 (br t, J=7.5 Hz, 2H), 2.62 (br s, 1H), 2.43-2.36 (m, 1H), 2.01-1.93 (m, 1H). MS (ESI) m/z 461.4 [M+H]⁺

Compound 28: General procedure A with variant i) was used for the preparation from compound VI employing 3-(difluoromethoxy)-5-fluoro-aniline.

¹H NMR (400 MHz, DMSO-d₆) δ=11.06-10.89 (m, 1H), 10.31 (s, 1H), 7.63 (s, 1H), 7.52 (s, 2H), 7.45-7.42 (m, 1H), 7.38 (td, J=2.0, 11.2 Hz, 1H), 7.24 (d, J=3.8 Hz, 1H), 7.07-7.05 (m, 1H), 6.79 (td, J=2.2, 9.8 Hz, 1H), 5.11 (dd, J=5.2, 13.4 Hz, 1H), 4.55-4.36 (m, 1H), 4.33-4.20 (m, 1H), 3.03 (t, J=7.4 Hz, 2H), 2.97-2.85 (m, 1H), 2.71 (t, J=7.6 Hz, 2H), 2.60 (br d, J=16.8 Hz, 1H), 2.46-2.34 (m, 1H), 2.05-1.96 (m, 1H). MS (ESI) m/z 476.1 [M+H]⁺

Step 1: To a solution of methanol (3.00 mL, 74.1 mmol, 3.93 eq) in N-methyl-pyrrolidone (10.0 mL) was added sodium hydride (60% dispersion in mineral oil) (830 mg, 20.7 mmol, 1.10 eq). The reaction was stirred at 0° C. for 1 h, then 1,3-difluoro-5-nitro-benzene (3.00 g, 18.9 mmol, 1.00 eq) was added. The reaction was stirred at 25° C. for another 11 h. The reaction was quenched with 1M hydrochloric acid (40.0 mL) and extracted with ethyl acetate (3×30.0 mL). The combined organic layers were washed with brine (15.0 mL), dried over anhydrous sodium sulfate, filtered, and concentrated under reduced pressure give a residue. The residue was purified by silica gel column chromatography (petroleum ether/ethyl acetate=10/1 to 1/1) to afford 1-fluoro-3-methoxy-5-nitro-benzene.

Step 2: To a solution of 1-fluoro-3-methoxy-5-nitro-benzene (2.50 g, 14.6 mmol, 1.00 eq) in dichloromethane (15.0 mL) was added boron tribromide (11.0 g, 43.8 mmol, 4.22 mL, 3.00 eq) at −78° C. The reaction was stirred at −78° C. for 1 h, and then at 25° C. for 11 h. The reaction was quenched with methanol (30.0 mL) and concentrated under reduced pressure to give a residue. The residue was purified by silica gel column chromatography (petroleum ether/ethyl acetate=10/1 to 5/1) to afford 3-fluoro-5-nitro-phenol.

Step 3: A solution of 3-fluoro-5-nitro-phenol (0.500 g, 3.18 mmol, 1.00 eq), sodium 2-chloro-2,2-difluoro-acetate (1.46 g, 9.55 mmol, 3.00 eq) and potassium carbonate (879 mg, 6.37 mmol, 2.00 eq) in dimethylformamide (10.0 mL) and water (2.00 mL) was stirred at 100° C. for 12 h. The mixture was poured into water (20.0 mL). The aqueous layer was extracted with ethyl acetate (3×30 mL). The combined organic layers were washed with brine (30.0 mL), dried over anhydrous sodium sulfate, filtered, and concentrated under reduced pressure to give a residue. The residue was purified by silica gel column chromatography (petroleum ether/ethyl acetate=10/1 to 5/1) to afford 1-(difluoromethoxy)-3-fluoro-5-nitrobenzene.

Step 4: A mixture of 1-(difluoromethoxy)-3-fluoro-5-nitrobenzene (700 mg, 3.38 mmol, 1.00 eq), ferrous powder (566 mg, 10.1 mmol, 3.00 eq) and ammonium chloride (904 mg, 16.9 mmol, 5.00 eq) in methanol (6.00 mL) and water (3.00 mL) was stirred at 80° C. for 1 h. The mixture was filtered, and the filtrate was concentrated under reduced pressure to afford 3-(difluoromethoxy)-5-fluoro-aniline.

Compound 29: General procedure A with variant i) was used for the preparation from compound VI employing 3-(difluoromethoxy)-4-methyl-aniline.

¹H NMR (400 MHz, DMSO-d₆) δ=11.12-10.86 (m, 1H), 10.12-9.90 (m, 1H), 7.34-7.32 (m, 1H), 7.32-7.26 (m, 1H), 7.24-7.18 (m, 1H), 7.14-7.08 (m, 1H), 6.94-6.90 (m, 1H), 5.18-5.04 (m, 1H), 4.46-4.38 (m, 1H), 4.36-4.22 (m, 1H), 3.08-2.98 (m, 2H), 2.98-2.86 (m, 1H), 2.68 (br t, J=7.6 Hz, 2H), 2.64-2.58 (m, 1H), 2.44-2.36 (m, 1H), 2.00 (br dd, J=5.0, 10.4 Hz, 1H). MS (ESI) m/z 472.3 [M+H]⁺

Step 1: To a solution of 2-methyl-5-nitrophenol (5.00 g, 32.7 mmol, 1.00 eq) and sodium 2-chloro-2,2-difluoroacetate (12.4 g, 81.6 mmol, 2.50 eq) in dimethylformamide (50.0 mL) was added caesium carbonate (21.3 g, 65.3 mmol, 2.00 eq) in portions. The reaction was stirred at 100° C. for 2 h. The mixture was diluted with water (800 mL) and extracted with ethyl acetate (3×100 mL). The combined organic layers were washed with brine (80.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 2-(difluoromethoxy)-1-methyl-4-nitrobenzene.

Step 2: To a solution of 2-(difluoromethoxy)-1-methyl-4-nitrobenzene (4.85 g, 23.8 mmol, 1.00 eq) and ammonium chloride (6.39 g, 119 mmol, 5.00 eq) in methanol (40.0 mL) and water (40.0 mL) was added iron powder (4.00 g, 71.6 mmol, 3.00 eq) in portions. The reaction was stirred at 80° C. for 2 h. The mixture was filtered, and the filtrate was concentrated under reduced pressure to give a residue. Water (100 mL) was added, and the mixture was extracted with ethyl acetate (3×50.0 mL). The combined organic layers were washed with brine (20 mL), dried over anhydrous sodium sulfate, filtered, and concentrated to afford 3-(difluoromethoxy)-4-methylaniline.

Compound 30: General procedure A with variant i) was used for the preparation from compound VI employing 3-(difluoromethoxy)-4-fluoroaniline.

¹H NMR (400 MHz, DMSO-d₆) δ=10.98 (s, 1H), 10.13 (s, 1H), 7.72 (dd, J=2.0, 7.2 Hz, 1H), 7.62 (s, 1H), 7.51 (d, J=1.0 Hz, 2H), 7.40-7.39 (m, 1H), 7.38 (s, 1H), 7.38-7.35 (m, 1H), 7.35-7.29 (m, 1H), 7.20 (s, 1H), 7.02 (s, 1H), 5.10 (dd, J=5.2, 13.2 Hz, 1H), 4.44-4.37 (m, 1H), 4.32-4.24 (m, 1H), 3.02 (t, J=7.6 Hz, 2H), 2.95-2.86 (m, 1H), 2.70-2.66 (m, 2H), 2.61-2.58 (m, 1H), 2.42-2.31 (m, 1H), 2.04-1.95 (m, 1H). MS (ESI) m/z 476.1 [M+H]⁺

Step 1: To a solution of 2-fluoro-5-nitrophenol (500 mg, 3.18 mmol, 1.00 eq) and methyl 2-chloro-2,2-difluoroacetate (2.43 g, 15.9 mmol, 5.00 eq) in dimethylformamide (20.0 mL) and water (3.00 mL) was added potassium carbonate (880 mg, 6.37 mmol, 2.00 eq). The reaction was stirred at 100° C. for 12 h. Water (50.0 mL) was added, and the aqueous layer was extracted with ethyl acetate (3×20.0 mL). The organic layers were gathered, dried over anhydrous sodium sulfate, filtered, and concentrated under reduced pressure to give a residue. The residue was purified by silica gel column chromatography (petroleum ether/ethyl acetate=20/1 to 10/1) to afford 2-(difluoromethoxy)-1-fluoro-4-nitrobenzene.

Step 2: To a solution of 2-(difluoromethoxy)-1-fluoro-4-nitrobenzene (400 mg, 1.93 mmol, 1.00 eq) in methanol (9.00 mL) and water (3.00 mL) were added ammonium chloride (516 mg, 9.66 mmol, 5.00 eq) and ferrous powder (539 mg, 9.66 mmol, 5.00 eq). The reaction was stirred at 80° C. for 2 h. The mixture was filtered and concentrated under reduced pressure to afford 3-(difluoromethoxy)-4-fluoroaniline. It was used to the next step without further purification.

Compound 31: General procedure A with variant i) was used for the preparation from compound VI employing 3-chloro-5-(1-methylpyrrolidin-3-yl)oxy-aniline.

¹H NMR (400 MHz, DMSO-d₆) δ=10.99 (s, 1H), 10.28 (br d, J=10.5 Hz, 1H), 7.62 (s, 1H), 7.52 (s, 2H), 7.35-7.24 (m, 2H), 6.78-6.73 (m, 1H), 5.20-5.06 (m, 2H), 4.46-4.38 (m, 1H), 4.31-4.25 (m, 1H), 3.99-3.69 (m, 3H), 3.39-3.15 (m, 2H), 3.09-3.01 (m, 2H), 2.93-2.82 (m, 4H), 2.77-2.68 (m, 2H), 2.64-2.59 (m, 1H), 2.43-2.34 (m, 1H), 2.31-2.17 (m, 1H), 2.05-1.97 (m, 1H). MS (ESI) m/z 525.2 [M+H]⁺

Step 1: To a solution of 3-chloro-5-nitro-phenol (5.00 g, 28.8 mmol, 1.00 eq), tert-butyl 3-hydroxypyrrolidine-1-carboxylate (5.93 g, 31.7 mmol, 1.10 eq) and triphenylphosphine (8.31 g, 31.7 mmol, 1.10 eq) in tetrahydrofuran (2.00 mL) was added diisopropyl azodiformate (6.16 mL, 31.7 mmol, 1.10 eq) at 0° C. The reaction was stirred at 25° C. for 12 h. The mixture was concentrated under reduced pressure to give a residue. The residue was purified by reversed phase preparative HPLC to afford tert-butyl 3-(3-chloro-5-nitrophenoxy)pyrrolidine-1-carboxylate.

Step 2: To a solution of tert-butyl 3-(3-chloro-5-nitro-phenoxy)pyrrolidine-1-carboxylate (2.00 g, 5.83 mmol, 1.00 eq) in ethyl acetate (10.0 mL) was added hydrochloric acid in ethyl acetate (4 M, 20.0 mL, 13.7 eq). The reaction was stirred at 25° C. for 1 h. The mixture was concentrated under reduced pressure to afford 3-(3-chloro-5-nitrophenoxy)pyrrolidine.

Step 3: To a solution of 3-(3-chloro-5-nitrophenoxy)pyrrolidine (300 mg, 1.24 mmol, 1.00 eq) in methanol (2.00 mL) were added paraformaldehyde 37% purity (2.00 mL, 26.8 mmol, 21.7 eq) and acetic acid (0.71 mL, 1.24 mmol, 1.00 eq). The reaction was stirred at 25° C. for 0.5 h. Then sodium cyanoborohydride (777 mg, 12.3 mmol, 10.0 eq) was added. The reaction was stirred at 25° C. for another 11.5 h. The mixture was concentrated under reduced pressure to give a residue. The residue was poured into water (60.0 mL) and stirred for 30 min. The aqueous layer was extracted with ethyl acetate (3×30.0 mL). The combined organic layers were washed with brine (80.0 mL), dried over anhydrous sodium sulfate, filtered, and concentrated under reduced pressure to give a residue. The residue was purified by silica gel column chromatography (petroleum ether/ethyl acetate=1/0 to 1/2) to afford 3-(3-chloro-5-nitrophenoxy)-1-methylpyrrolidine.

Step 4: A mixture of 3-(3-chloro-5-nitro-phenoxy)-1-methyl-pyrrolidine (250 mg, 974 μmol, 1.00 eq), iron powder (163 mg, 2.92 mmol, 3.00 eq) and ammonium chloride (260 mg, 4.87 mmol, 5.00 eq) in methyl alcohol (4.00 mL) and water (2.00 mL) was stirred at 80° C. for 1 h. The mixture was filtered, and the filtrate was concentrated under reduced pressure to give a residue. Water (100 mL) was added, and the mixture was stirred for 10 min. The aqueous layer was extracted with ethyl acetate (3×30.0 mL). The combined organic layers were washed with brine (80.0 mL), dried over anhydrous sodium sulfate, filtered, and concentrated under reduced pressure to afford 3-chloro-5-(1-methylpyrrolidin-3-yl)oxy-aniline.

Compound 32: General procedure A with variant iii) was used for the preparation from compound VI employing 3-ethoxy-4-methylaniline.

¹H NMR (400 MHz, DMSO-d₆) δ=10.98 (s, 1H), 9.82 (s, 1H), 7.63 (s, 1H), 7.52 (s, 2H), 7.26 (s, 1H), 7.01 (s, 2H), 5.11 (dd, J=5.2, 13.2 Hz, 1H), 4.45-4.38 (m, 1H), 4.33-4.25 (m, 1H), 3.97 (q, J=6.8 Hz, 2H), 3.02 (br t, J=7.6 Hz, 2H), 2.96-2.86 (m, 1H), 2.65 (br t, J=7.6 Hz, 2H), 2.62-2.57 (m, 1H), 2.43-2.34 (m, 1H), 2.08 (s, 3H), 2.03-1.96 (m, 1H), 1.35 (t, J=7.2 Hz, 3H). MS (ESI) m/z 450.3 [M+H]⁺

Step 1: To a solution of 2-methyl-5-nitrophenol (1.00 g, 6.53 mmol, 1.00 eq) and iodoethane (0.57 mL, 7.18 mmol, 1.10 eq) in dimethylformamide (10.0 mL) was added potassium carbonate (2.71 g, 19.6 mmol, 3.00 eq) under nitrogen. The reaction was stirred at 40° C. for 2 h under nitrogen atmosphere. The mixture was poured into water (20.0 mL) and extracted with ethyl acetate (3×20.0 mL). The combined organic layers were gathered, washed with brine (10.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 2-ethoxy-1-methyl-4-nitrobenzene.

Step 2: To a solution of 2-ethoxy-1-methyl-4-nitrobenzene (0.900 g, 4.97 mmol, 1.00 eq) in methanol (10.0 mL) and water (10.0 mL) were added iron powder (1.39 g, 24.8 mmol, 5.00 eq) and ammonium chloride (1.33 g, 24.8 mmol, 5.00 eq). The reaction was stirred at 80° C. for 2 h. The mixture was concentrated under reduced pressure to give a slurry. The slurry was poured into saturated sodium bicarbonate solution (20.0 mL) and extracted with ethyl acetate (3×20.0 mL). The combined organic layers were washed with brine (20.0 mL), dried over anhydrous sodium sulfate, filtered, and concentrated under reduced pressure to afford 3-ethoxy-4-methylaniline.

Compound 33: General procedure A with variant i) was used for the preparation from compound VI employing 3-methoxy-4-methylaniline.

¹H NMR (400 MHz, DMSO-d₆) δ=10.98 (s, 1H), 9.84 (s, 1H), 7.64 (s, 1H), 7.52 (s, 2H), 7.26 (s, 1H), 7.08-6.98 (m, 2H), 5.12 (dd, J=5.2, 13.2 Hz, 1H), 4.42 (d, J=17.2 Hz, 1H), 4.30 (d, J=17.2 Hz, 1H), 3.74 (s, 3H), 3.34 (s, 2H), 3.10-2.86 (m, 3H), 2.72-2.56 (m, 4H), 2.48-2.24 (m, 2H), 2.08 (s, 3H), 2.06-1.92 (m, 1H). MS (ESI) m/z 436.3 [M+H]⁺

Compound 34: General procedure A with variant iii) was used for the preparation from compound VI employing 6-phenylpyridin-3-amine.

¹H NMR (400 MHz, DMSO-d₆) δ=10.98 (s, 1H), 10.26 (s, 1H), 8.79 (d, J=2.4 Hz, 1H), 8.14 (dd, J=2.4, 8.8 Hz, 1H), 8.08-8.00 (m, 2H), 7.92 (d, J=8.4 Hz, 1H), 7.66 (s, 1H), 7.57-7.51 (m, 2H), 7.50-7.44 (m, 2H), 7.43-7.37 (m, 1H), 5.11 (dd, J=5.2, 13.2 Hz, 1H), 4.47-4.39 (m, 1H), 4.35-4.25 (m, 1H), 3.07 (br t, J=7.6 Hz, 2H), 2.91 (ddd, J=5.2, 13.6, 17.2 Hz, 1H), 2.79-2.72 (m, 2H), 2.62 (br d, J=2.0 Hz, 1H), 2.47-2.36 (m, 1H), 2.06-1.94 (m, 1H). MS (ESI) m/z 469.2 [M+H]⁺

Compound 35: General procedure A with variant ii) was used for the preparation from compound VI employing 3-(difluoromethoxy)-4-methyl-5-(morpholinomethyl)aniline.

¹H NMR (400 MHz, DMSO-d₆) δ=10.99 (s, 1H), 10.64 (br d, J=2.0 Hz, 1H), 10.37 (s, 1H), 7.68 (d, J=1.6 Hz, 1H), 7.63 (s, 1H), 7.58 (s, 1H), 7.57-7.50 (m, 2H), 7.13 (t, J=72 Hz, 1H), 5.09 (dd, J=5.2, 13.2 Hz, 1H), 4.45-4.40 (m, 1H), 4.37 (s, 2H), 4.32-4.25 (m, 1H), 3.96-3.89 (m, 2H), 3.86-3.79 (m, 2H), 3.34-3.27 (m, 2H), 3.23 (br s, 2H), 3.03 (br t, J=7.2 Hz, 2H), 2.91 (ddd, J=5.2, 13.6, 17.6 Hz, 1H), 2.77-2.69 (m, 2H), 2.61 (br d, J=16.8 Hz, 1H), 2.43-2.34 (m, 1H), 2.29 (s, 3H), 2.06-1.95 (m, 1H). MS (ESI) m/z 571.4 [M+H]⁺

Step 1: To a solution of 2-methyl-5-nitro-benzoic acid (10.0 g, 55.2 mmol, 1.00 eq) in sulfuric acid (20.0 mL) was added N-Iodosuccinimide (14.9 g, 66.3 mmol, 1.20 eq). The reaction was stirred at 60° C. for 2 h. The mixture was diluted with ice water (200 mL) and the resulting precipitate was collected by filtration. The filter cake was washed with water (100 mL) and dried under vacuum to afford 3-iodo-2-methyl-5-nitro-benzoic acid.

Step 2: To a solution of 3-iodo-2-methyl-5-nitro-benzoic acid (5.00 g, 16.3 mmol, 1.00 eq), copper iodide (310 mg, 1.63 mmol, 0.100 eq), and quinolin-8-ol (0.56 mL, 3.26 mmol, 0.200 eq) in water (3.00 mL) and dimethylsulfoxide (3.00 mL) was added potassium hydroxide (3.65 g, 65.1 mmol, 4.00 eq). The reaction was stirred at 100° C. for 12 h. The mixture was diluted with water (50.0 mL) and extracted with ethyl acetate (2×50.0 mL). The combined organic layers were washed with water (50.0 mL), dried over anhydrous sodium sulfate, filtered, and concentrated under reduced pressure to afford 3-hydroxy-2-methyl-5-nitro-benzoic acid.

Step 3: To a solution of 3-hydroxy-2-methyl-5-nitro-benzoic acid (3.20 g, 16.2 mmol, 1.00 eq) and morpholine (1.71 mL, 19.5 mmol, 1.20 eq) in dichloromethane (100 mL) were added triethylamine (2.26 mL, 16.2 mmol, 1.00 eq) and O-(7-azabenzotriazol-1-yl)-N,N,N,N-tetramethyluroniumhexafluorophosphate (7.41 g, 19.5 mmol, 1.20 eq) at 20° C. The reaction was stirred at 20° C. for 12 h. The mixture was concentrated under reduced pressure to afford a residue. The residue was diluted with water (100 mL) and extracted with ethyl acetate (100 mL). The organic layer was washed with water (100 mL), dried over anhydrous sodium sulfate, filtered, and the filtrate was concentrated under reduced pressure to give a residue. The residue was purified by silica gel column chromatography (petroleum ether/ethyl acetate=1/1 to 0/1) to afford (3-hydroxy-2-methyl-5-nitro-phenyl)-morpholino-methanone.

Step 4: A solution of (3-hydroxy-2-methyl-5-nitro-phenyl)-morpholino-methanone (2.00 g, 7.51 mmol, 1.00 eq), potassium carbonate (2.08 g, 15.0 mmol, 2.00 eq) and sodium 2-chloro-2,2-difluoroacetate (4.58 g, 30.0 mmol, 4.00 eq) in dimethylformamide (24.0 mL) and water (3.00 mL) was stirred at 100° C. for 12 h. The mixture was diluted with water (150 mL) and extracted with ethyl acetate (3×100 mL). The combined organic layers were washed with brine (50.0 mL), dried over sodium sulfate, filtered, and concentrated under reduced pressure to give a residue. The residue was purified by silica gel column chromatography (petroleum ether/ethyl acetate=3/1) to afford (3-(difluoromethoxy)-2-methyl-5-nitrophenyl)(morpholino)methanone.

Step 5: To a solution of (3-(difluoromethoxy)-2-methyl-5-nitrophenyl)(morpholino)methanone (1.70 g, 5.38 mmol, 1.00 eq) in tetrahydrofuran (3.00 mL) was added borane dimethyl sulfide complex (10 M, 1.08 mL, 2.00 eq) dropwise at 0° C. under nitrogen atmosphere. The reaction was stirred at 60° C. for 4 h. The reaction was quenched by addition with methanol (5.00 mL) and the solvents were removed under reduced pressure to afford 4-(3-(difluoromethoxy)-2-methyl-5-nitrobenzyl)morpholine.

Step 6: A solution of 4-(3-(difluoromethoxy)-2-methyl-5-nitrobenzyl)morpholine (1.50 g, 4.96 mmol, 1.00 eq), iron powder (1.94 g, 34.7 mmol, 7.00 eq) and ammonium chloride (1.86 g, 34.7 mmol, 7.00 eq) in methanol (5.00 mL) and water (5.00 mL) was stirred at 80° C. for 2 h. The mixture was filtered, and the filtrate was concentrated under reduced pressure to afford 3-(difluoromethoxy)-4-methyl-5-(morpholinomethyl)aniline.

Compound 36: General procedure A with variant i) was used for the preparation from compound VI employing 5-methoxy-6-methyl-pyridin-3-amine.

¹H NMR (400 MHz, DMSO-d₆) δ=10.99 (s, 1H), 10.10 (s, 1H), 8.18 (d, J=1.9 Hz, 1H), 8.14 (s, 1H), 7.68 (d, J=1.9 Hz, 1H), 7.64 (s, 1H), 7.53 (d, J=1.0 Hz, 2H), 5.11 (dd, J=5.0, 13.3 Hz, 1H), 4.47-4.37 (m, 1H), 4.34-4.22 (m, 1H), 3.79 (s, 3H), 3.04 (t, J=7.4 Hz, 2H), 2.97-2.87 (m, 1H), 2.76-2.66 (m, 2H), 2.65-2.56 (m, 1H), 2.47-2.33 (m, 1H), 2.29 (s, 3H), 2.05-1.95 (m, 1H). MS (ESI) m/z 437.1 [M+H]⁺

Step 1: To a solution of 2-chloro-3-methoxy-5-nitro-pyridine (3.00 g, 15.9 mmol, 1.00 eq), methylboronic acid (1.90 g, 31.8 mmol, 2.00 eq) and potassium carbonate (6.60 g, 47.7 mmol, 3.00 eq) in dioxane (30.0 mL) was added tetrakis[triphenylphosphine]palladium(0) (1.84 g, 1.59 mmol, 0.10 eq) at 25° C. The reaction was stirred at 110° C. for 12 h under nitrogen. The mixture was concentrated under reduced pressure to give a residue. The residue was purified by silica gel column chromatography (petroleum ether/ethyl acetate=1/0 to 30/1) to afford 3-methoxy-2-methyl-5-nitropyridine.

Step 2: A mixture of 3-methoxy-2-methyl-5-nitropyridine (500 mg, 2.97 mmol, 1.00 eq), iron powder (498 mg, 8.92 mmol, 3.00 eq), and ammonium chloride (795 mg, 14.9 mmol, 5.00 eq) in methanol (4.00 mL) and water (2.00 mL) was stirred at 80° C. for 1 h. The mixture was filtered, and the filtrate was concentrated under reduced pressure to give a residue. The residue was added to water (80.0 mL) and stirred for 10 min. The solution was extracted with ethyl acetate (3×50.0 mL). The combined organic layers were washed with brine (80.0 mL), dried over anhydrous sodium sulfate, filtered, and concentrated under reduced pressure to afford 5-methoxy-6-methyl-pyridin-3-amine.

Compound 37: General procedure A with variant i) was used for the preparation from compound VI employing 3-chloro-5-((1-methylpyrrolidin-3-yl)methoxy)aniline.

¹H NMR (400 MHz, DMSO-d₆) δ=11.00 (s, 1H), 10.10 (s, 1H), 7.68-7.56 (m, 1H), 7.52 (s, 2H), 7.28 (br s, 1H), 7.22 (s, 1H), 6.74 (s, 1H), 5.10 (br dd, J=4.8, 13.2 Hz, 1H), 4.42 (br d, J=17.2 Hz, 1H), 4.30 (br d, J=17.0 Hz, 1H), 4.06-3.88 (m, 2H), 3.58 (br s, 2H), 3.16 (br s, 2H), 3.04 (br t, J=7.0 Hz, 2H), 2.98-2.90 (m, 1H), 2.84 (s, 4H), 2.70 (br t, J=7.4 Hz, 2H), 2.62 (br d, J=17.2 Hz, 1H), 2.44-2.32 (m, 1H), 2.18 (br dd, J=7.2, 13.2 Hz, 1H), 2.06-1.94 (m, 1H), 1.80 (br dd, J=6.8, 13.4 Hz, 1H). MS (ESI) m/z 539.3 [M+H]⁺

Step 1: To a solution of tert-butyl 3-(hydroxymethyl)pyrrolidine-1-carboxylate (3.00 g, 14.9 mmol, 1.00 eq) and triethylamine (3.77 g, 37.3 mmol, 5.19 mL, 2.50 eq) in dichloromethane (30.0 mL) at 0° C. was added methylsulfamoyl chloride (1.50 mL, 19.4 mmol, 1.30 eq) dropwise under nitrogen atmosphere. The reaction was stirred at 25° C. for 2 h. The mixture was diluted with ethyl acetate (100 mL) and water (150 mL). The organic layer was separated, and the aqueous layer was extracted with ethyl acetate (3×100 mL). The organic layers were gathered, dried over sodium sulfate, filtered, and concentrated under reduced pressure to afford tert-butyl 3-(((methylsulfonyl)oxy)methyl)pyrrolidine-1-carboxylate.

Step 2: To a solution of tert-butyl 3-(((methylsulfonyl)oxy)methyl) pyrrolidine-1-carboxylate (3.00 g, 10.8 mmol, 1.00 eq) in dimethylformamide (30.0 mL) was added 3-chloro-5-nitrophenol (2.05 g, 11.8 mmol, 1.10 eq) and cesium carbonate (10.5 g, 32.2 mmol, 3.00 eq). The reaction was stirred at 80° C. for 12 h. The mixture was diluted with ethyl acetate (100 mL) and water (100 mL). The organic layer was separated, and the aqueous layer was extracted with ethyl acetate (3×80.0 mL). The organic layers were gathered, dried over sodium sulfate, filtered, and concentrated under reduced pressure to give a residue. The residue was purified by silica gel column chromatography (petroleum ether/ethyl acetate=3/1) to afford tert-butyl 3-((3-chloro-5-nitrophenoxy)methyl) pyrrolidine-1-carboxylate.

Step 3: To a solution of tert-butyl 3-((3-chloro-5-nitrophenoxy)methyl)pyrrolidine-1-carboxylate (1.10 g, 3.08 mmol, 1.00 eq) in ethyl acetate (5.00 mL) was added hydrochloric acid in ethyl acetate (4 M, 10 mL). The reaction was stirred at 25° C. for 2 h. The mixture was concentrated under reduced pressure to afford 3-((3-chloro-5-nitrophenoxy) methyl)pyrrolidine.

Step 4: To a solution of 3-((3-chloro-5-nitrophenoxy)methyl)pyrrolidine (1.50 g, 5.84 mmol, 1.00 eq) in 2,2,2-trifluoroethanol (10.0 mL) was added paraformaldehyde (0.80 mL, 29.2 mmol, 5.00 eq). The reaction was stirred at 60° C. for 0.5 h. Sodium borohydride (442 mg, 11.7 mmol, 2.00 eq) was added in portions, and the reaction was stirred at 60° C. for 1 h. The reaction was quenched with saturated ammonium chloride solution (10.0 mL) and concentrated under reduced pressure to give a residue. The residue was purified by reversed phase preparative HPLC to afford 3-((3-chloro-5-nitrophenoxy)methyl)-1-methylpyrrolidine.

Step 5: To a solution of 3-((3-chloro-5-nitrophenoxy)methyl)-1-methylpyrrolidine (1.20 g, 4.43 mmol, 1.00 eq) in methanol (6.00 mL) and water (6.00 mL) was added ammonium chloride (1.66 g, 31.0 mmol, 7.00 eq) and iron powder (1.73 g, 31.0 mmol, 7.00 eq). The reaction was stirred at 80° C. for 2 h. The mixture was filtered over Celite, and the filtrate was concentrated under reduced pressure to afford 3-chloro-5-((1-methylpyrrolidin-3-yl)methoxy)aniline.

Compound 38: General procedure A with variant i) was used for the preparation from compound VI employing 4-cyclopropylaniline.

¹H NMR (400 MHz, DMSO-d₆) δ=11.00 (br s, 1H), 9.84 (s, 1H), 7.64 (s, 1H), 7.52 (s, 2H), 7.48-7.42 (m, J=8.4 Hz, 2H), 7.04-6.94 (m, J=8.4 Hz, 2H), 5.12 (dd, J=5.0, 13.2 Hz, 1H), 4.2 (br d, J=17.0 Hz, 1H), 4.30 (d, J=17.2 Hz, 1H), 3.04 (br t, J=7.4 Hz, 2H), 2.98-2.84 (m, 1H), 2.66 (br t, J=7.6 Hz, 2H), 2.58 (br d, J=1.6 Hz, 1H), 2.48-2.32 (m, 1H), 2.06-1.96 (m, 1H), 1.90-1.80 (m, 1H), 0.96-0.82 (m, 2H), 0.66-0.54 (m, 2H). MS (ESI) m/z 432.2 [M+H]⁺

Compound 39: General procedure A with variant i) was used for the preparation from compound VI employing 3-chloro-4-methyl-5-(2-morpholinoethoxy)aniline.

¹H NMR (400 MHz, DMSO-d₆) δ=11.00 (s, 1H), 10.06 (s, 1H), 9.73 (br s, 1H), 8.13 (s, 1H), 7.62 (s, 1H), 7.47-7.55 (m, 2H), 7.23-7.38 (m, 2H), 5.09 (dd, J=13.2, 5.2 Hz, 1H), 4.41 (d, J=17.2 Hz, 1H), 4.28 (br d, J=17.2 Hz, 3H), 3.99 (br s, 2H), 3.44-3.81 (m, 6H), 3.11-3.28 (m, 2H), 3.02 (br t, J=7.2 Hz, 2H), 2.85-2.96 (m, 1H), 2.68 (br t, J=7.2 Hz, 2H), 2.60 (br d, J=16.8 Hz, 1H), 2.31-2.42 (m, 1H), 2.17 (s, 3H), 1.93-2.03 (m, 1H). MS (ESI) m/z 569.2 [M+H]⁺

Step 1: A solution of 2-chloro-1-methyl-4-nitrobenzene (12.1 mL, 58.3 mmol, 1.00 eq) and N-iodosuccinimide (14.4 g, 64.1 mmol, 1.10 eq) in sulfuric acid (100 mL) was stirred at 60° C. for 2 h. The reaction was quenched by addition of ice water (200 mL) at 0° C. The aqueous layer was extracted with ethyl acetate (3×50.0 mL). The combined organic layers were washed with brine (100 mL), dried over sodium sulfate, filtered, and concentrated under reduced pressure to give a residue. The residue was purified by silica gel column chromatography (petroleum ether/ethyl acetate=1/0) to afford 1-chloro-3-iodo-2-methyl-5-nitrobenzene.

Step 2: A solution of 1-chloro-3-iodo-2-methyl-5-nitrobenzene (7.80 g, 26.2 mmol, 1.00 eq), potassium hydroxide (4.41 g, 78.7 mmol, 3.00 eq), tris(dibenzylidenethyl acetatecetone)dipalladium(0) (1.20 g, 1.31 mmol, 0.05 eq) and 2-di-tert-butylphosphino-2′,4′,6′-triisopropylbiphenyl (557 mg, 1.31 mmol, 0.050 eq) in dioxane (80.0 mL) and water (16.0 mL) was stirred at 80° C. for 12 h under nitrogen atmosphere. The mixture was acidified to pH˜4 and concentrated under reduced pressure to give a residue. Brine (200 mL) was added, and the aqueous layer was extracted with ethyl acetate (3×50.0 mL). The combined organic layers were dried over sodium sulfate, filtered, and concentrated under reduced pressure to give a residue. The residue was purified by silica gel column chromatography (petroleum ether/ethyl acetate=1/0 to 17/3) to afford 3-chloro-2-methyl-5-nitrophenol.

Step 3: A suspension of 3-chloro-2-methyl-5-nitrophenol (500 mg, 2.67 mmol, 1.00 eq), 4-(2-chloroethyl) morpholine (479 mg, 3.20 mmol, 1.20 eq), potassium carbonate (553 mg, 4.00 mmol, 1.50 eq) and potassium iodide (133 mg, 0.80 mmol, 0.30 eq) in dimethylformamide (5.00 mL) was stirred at 80° C. for 1 h. The mixture was poured into brine (50.0 mL) and extracted with ethyl acetate (3×15.0 mL). The combined organic layers were dried over sodium sulfate, filtered, and concentrated under reduced pressure to afford 4-(2-(3-chloro-2-methyl-5-nitrophenoxy)ethyl)morpholine.

Step 4: To a solution of 4-(2-(3-chloro-2-methyl-5-nitrophenoxy)ethyl)morpholine (570 mg, 1.90 mmol, 1.00 eq) in methanol (5.00 mL) and water (1.00 mL) were added iron powder (529 mg, 9.48 mmol, 5.00 eq) and ammonium chloride (507 mg, 9.48 mmol, 5.00 eq). The reaction was stirred at 80° C. for 12 h under nitrogen. The mixture was filtered, and the filtrate was concentrated under reduced pressure to give a residue. The residue was treated with brine (5.00 mL) and saturated aqueous sodium bicarbonate (5.00 mL). The aqueous layer was extracted with ethyl acetate (3×10.0 mL), dried over sodium sulfate, filtered, and concentrated under reduced pressure to afford 3-chloro-4-methyl-5-(2-morpholinoethoxy)aniline.

### Compound 40

Step 1: To a solution of 3-(2-(2,6-dioxopiperidin-3-yl)-3-oxoisoindolin-5-yl)propanoic acid (120 mg, 0.38 mmol, 1.00 eq), tert-butyl 3-(5-amino-3-chloro-2-methylphenoxy)pyrrolidine-1-carboxylate (149 mg, 0.45 mmol, 1.20 eq) and 0-(7-azabenzotriazol-1-yl)-N,N,N,N-tetramethyluroniumhexafluorophosphate (173 mg, 0.45 mmol, 1.20 eq) in dimethylformamide (2.00 mL) was added N,N-diisopropylethylamine (0.20 mL, 1.14 mmol, 3.00 eq) at 25° C. The reaction was stirred at 25° C. for 12 h. Water (10.0 mL) was added, and the resulting precipitate was collected by filtration. The filter cake was triturated with water (15.0 mL) at 25° C. for 10 min, then lyophilized to afford tert-butyl 3-(3-chloro-5-(3-(2-(2,6-dioxopiperidin-3-yl)-3-oxoisoindolin-5-yl)propanamido)-2-methylphenoxy)pyrrolidine-1-carboxylate.

Step 2: A solution of tert-butyl 3-(3-chloro-5-(3-(2-(2,6-dioxopiperidin-3-yl)-3-oxoisoindolin-5-yl)propanamido)-2-methylphenoxy)pyrrolidine-1-carboxylate (200 mg, 0.32 mmol, 1.00 eq) and hydrochloric acid/ethyl acetate (5.00 mL) in ethyl acetate (10.0 mL) was stirred at 25° C. for 12 h. The mixture was concentrated under reduced pressure to give a residue. The residue was purified by reversed phase preparative HPLC to afford N-(3-chloro-4-methyl-5-(pyrrolidin-3-yloxy)phenyl)-3-(2-(2,6-dioxopiperidin-3-yl)-3-oxoisoindolin-5-yl)propanamide.

¹H NMR (400 MHz, DMSO-d₆) δ=10.99 (s, 1H), 10.21 (s, 1H), 9.58-9.39 (m, 2H), 7.62 (s, 1H), 7.52 (s, 2H), 7.39 (s, 1H), 7.24 (d, J=1.2 Hz, 1H), 5.11 (dd, J=5.2, 13.2 Hz, 1H), 5.03 (br s, 1H), 4.46-4.38 (m, 1H), 4.33-4.23 (m, 1H), 3.51-3.45 (m, 1H), 3.40-3.32 (m, 2H), 3.30-3.22 (m, 1H), 3.03 (br t, J=7.2 Hz, 2H), 2.91 (ddd, J=5.6, 13.6, 17.6 Hz, 1H), 2.69 (br t, J=7.2 Hz, 2H), 2.60 (br d, J=17.6 Hz, 1H), 2.45-2.36 (m, 1H), 2.27-2.19 (m, 1H), 2.15 (s, 4H), 2.04-1.97 (m, 1H). MS (ESI) m/z 525.4 [M+1]⁺

Step 1: To a solution of tert-butyl 3-hydroxypyrrolidine-1-carboxylate (3.00 g, 16.0 mmol, 1.00 eq) and triethylamine (5.58 mL, 40.0 mmol, 2.50 eq) in dichloromethane (30.0 mL) was added methanesulfonyl chloride (1.74 mL, 22.4 mmol, 1.40 eq) dropwise at 0° C. The reaction was stirred at 25° C. for 2 h. The mixture was diluted with water (150 mL) and extracted with ethyl acetate (3×80.0 mL). The combined organic layers were washed with brine (60.0 mL), dried over sodium sulfate, filtered, and concentrated under reduced pressure to afford tert-butyl 3-((methylsulfonyl)oxy)pyrrolidine-1-carboxylate.

Step 2: A solution of 3-chloro-2-methyl-5-nitrophenol (260 mg, 1.39 mmol, 1.00 eq), tert-butyl 3-((methylsulfonyl)oxy)pyrrolidine-1-carboxylate (441 mg, 1.66 mmol, 1.20 eq) and potassium carbonate (575 mg, 4.16 mmol, 3.00 eq) in dimethylformamide (10.0 mL) was stirred at 80° C. for 4 h. The mixture was poured into water (20.0 mL) and extracted with ethyl acetate (3×20.0 mL). The combined organic layers were washed with brine (10.0 mL), dried over anhydrous sodium sulfate, filtered, and concentrated under reduced pressure to give a residue. The residue was purified by silica gel column chromatography (petroleum ether/ethyl acetate=1/0 to 5/1) to afford tert-butyl 3-(3-chloro-2-methyl-5-nitrophenoxy)pyrrolidine-1-carboxylate.

Step 3: To a mixture of tert-butyl 3-(3-chloro-2-methyl-5-nitrophenoxy)pyrrolidine-1-carboxylate (430 mg, 1.21 mmol, 1.00 eq), iron powder (337 mg, 6.03 mmol, 5.00 eq) and ammonium chloride (322 g, 6.03 mmol, 5.00 eq) in methanol (10.0 mL) was added water (10.0 mL) at 25° C. The reaction was stirred at 80° C. for 2 h. The mixture was concentrated under reduced pressure to give a residue. The residue was added to saturated sodium bicarbonate (20.0 mL) and extracted with ethyl acetate (3×20.0 mL). The combined organic layers were washed with brine (20.0 mL), dried over anhydrous sodium sulfate, filtered, and concentrated under reduced pressure to afford tert-butyl 3-(5-amino-3-chloro-2-methylphenoxy)pyrrolidine-1-carboxylate.

Compound 41: General procedure A with variant i) was used for the preparation from compound VI employing 6-(tert-butyl)pyridin-3-amine.

¹H NMR (400 MHz, DMSO-d₆) δ=10.99 (s, 1H), 10.88-10.58 (m, 1H), 9.07-8.88 (m, 1H), 8.39-8.21 (m, 1H), 7.97-7.72 (m, 1H), 7.64 (s, 1H), 7.53 (s, 2H), 5.10 (dd, J=5.2, 13.2 Hz, 1H), 4.46-4.39 (m, 1H), 4.34-4.26 (m, 1H), 3.07 (t, J=7.2 Hz, 2H), 2.91 (m, 1H), 2.79 (d, J=7.2 Hz, 2H), 2.61 (d, J=16.4 Hz, 1H), 2.46-2.35 (m, 1H), 2.05-1.95 (m, 1H), 1.47-1.34 (m, 9H). MS (ESI) m/z 449.2 [M+H]⁺

Compound 42: General procedure A with variant i) was used for the preparation from compound VI employing 3-fluoro-4-methyl-5-(morpholinomethyl)aniline.

¹H NMR (400 MHz, DMSO-d₆) δ=10.98 (s, 1H), 10.01 (s, 1H), 8.13 (s, 1H), 7.62 (s, 1H), 7.55-7.49 (m, 3H), 7.15 (s, 1H), 5.10 (dd, J=5.0, 13.2 Hz, 1H), 4.45-4.36 (m, 1H), 4.32-4.25 (m, 1H), 3.60-3.52 (m, 4H), 3.39 (s, 2H), 3.01 (br t, J=7.5 Hz, 2H), 2.96-2.85 (m, 1H), 2.66 (br t, J=7.5 Hz, 2H), 2.59 (br d, J=18.1 Hz, 1H), 2.45-2.38 (m, 1H), 2.35 (br s, 4H), 2.14 (d, J=1.2 Hz, 3H), 2.03-1.94 (m, 1H). MS (ESI) m/z 523.3 [M+H]⁺

Step 1: To a solution of 3-fluoro-2-methylbenzoic acid (10.0 g, 64.9 mmol, 1.00 eq) in sulfuric acid (100 mL) was added potassium nitrate (7.22 g, 71.4 mmol, 1.10 eq) in portions at 0° C. The reaction was stirred at 0° C. for 1 h. The mixture was poured into water (100 mL) and the resulting precipitate was collected by filtration. The filter cake was dried under vacuum, then added to water (100 mL) and extracted with ethyl acetate (3×80.0 mL). The combined organic layers were washed with brine (60.0 mL), dried over sodium sulfate, filtered, and concentrated under reduced pressure to afford 3-fluoro-2-methyl-5-nitrobenzoic acid.

Step 2: To a solution of 3-fluoro-2-methyl-5-nitrobenzoic acid (11.0 g, 55.2 mmol, 1.00 eq) in tetrahydrofuran (100 mL) was added borane dimethyl sulfide complex (10 M, 11.0 mL, 2.00 eq) at 0° C. The reaction was stirred at 25° C. for 12 h. The mixture was poured into methanol (200 mL) and concentrated under reduced pressure to give a residue. Water (150 mL) was added, and the pH was adjusted to pH=10 by addition of 15% sodium hydroxide solution. The aqueous layer was extracted with ethyl acetate (3×80.0 mL). The combined organic layers were washed with brine (50.0 mL), dried over sodium sulfate, filtered, and concentrated under reduced pressure to give a residue. The residue was purified by silica gel column chromatography (petroleum ether/ethyl acetate=4/1) to afford (3-fluoro-2-methyl-5-nitrophenyl)methanol.

Step 3: To a solution of (3-fluoro-2-methyl-5-nitrophenyl)methanol (870 mg, 4.70 mmol, 1.00 eq) in dichloromethane (10.0 mL) was added thionyl chloride (1.70 mL, 23.5 mmol, 5.00 eq) dropwise at 0° C. The reaction was stirred at 25° C. for 12 h. The mixture was concentrated under reduced pressure to afford 1-(chloromethyl)-3-fluoro-2-methyl-5-nitrobenzene.

Step 4: To a solution of 1-(chloromethyl)-3-fluoro-2-methyl-5-nitrobenzene (950 mg, 4.67 mmol, 1.00 eq) and triethylamine (1.62 mL, 11.7 mmol, 2.50 eq) in acetonitrile (10.0 mL) was added morpholine (0.51 mL, 5.83 mmol, 1.25 eq) dropwise. The reaction was stirred at 25° C. for 12 h. The mixture was concentrated under reduced pressure to give a residue. The residue was diluted with water (80.0 mL) and extracted with ethyl acetate (3×60.0 mL). The combined organic layers were washed with brine (40.0 mL), dried over sodium sulfate, filtered, and concentrated under reduced pressure to give a residue. The residue was purified by silica gel column chromatography (petroleum ether/ethyl acetate=4/1) to afford 4-(3-fluoro-2-methyl-5-nitrobenzyl)morpholine.

Step 5: To a solution of 4-(3-fluoro-2-methyl-5-nitrobenzyl)morpholine (1.00 g, 3.93 mmol, 1.00 eq) and ammonium chloride (1.05 g, 19.7 mmol, 5.00 eq) in methanol (8.00 mL) and water (2.00 mL) was added iron powder (1.10 g, 19.7 mmol, 5.00 eq) in portions. The reaction was stirred at 80° C. for 2 h. The mixture was filtered, and the filtrate was diluted with water (80.0 mL) and extracted with ethyl acetate (3×60.0 mL). The combined organic layers were washed with brine (40.0 mL), dried over sodium sulfate, filtered, and concentrated under reduced pressure to afford 3-fluoro-4-methyl-5-(morpholinomethyl)aniline.

Compound 43: General procedure A with variant i) was used for the preparation from compound VI employing 3-chloro-5-ethyl-4-methylaniline.

¹H NMR (400 MHz, DMSO-d₆) δ=10.97 (br s, 1H), 9.96 (s, 1H), 7.69-7.60 (m, 2H), 7.51 (s, 2H), 7.21 (d, J=1.6 Hz, 1H), 5.09 (dd, J=5.1, 13.2 Hz, 1H), 4.45-4.36 (m, 1H), 4.33-4.22 (m, 1H), 3.01 (br t, J=7.5 Hz, 2H), 2.96-2.84 (m, 1H), 2.66 (br t, J=7.5 Hz, 2H), 2.61 (br s, 1H), 2.60-2.55 (m, 2H), 2.38 (dt, J=9.0, 13.2 Hz, 1H), 2.22 (s, 3H), 2.04-1.94 (m, 1H), 1.12 (t, J=7.5 Hz, 3H). MS (ESI) m/z 468.2 [M+H]⁺

Step 1: To a solution of 2-chloro-1-methyl-4-nitrobenzene (0.60 mL, 2.91 mmol, 1.00 eq) in sulfuric acid (5.00 mL, 98% purity) was added N-iodosuccinimide (787 mg, 3.50 mmol, 1.20 eq) in portions. The reaction was stirred at 60° C. for 1 h. The mixture was quenched with sodium carbonate (10%, 100 mL) and extracted with ethyl acetate (3×30.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 1-chloro-3-iodo-2-methyl-5-nitrobenzene.

Step 2: To a solution of 1-chloro-3-iodo-2-methyl-5-nitrobenzene (760 mg, 2.55 mmol, 1.00 eq) in toluene (15.0 mL) were added diisopropylethylamine (1.34 mL, 7.66 mmol, 3.00 eq), [1,1′-bis(diphenylphosphino)ferrocene]dichloropalladium(II) (187 mg, 256 μmol, 0.10 eq) and potassium vinyltrifluoroborate (685 mg, 5.11 mmol, 2.00 eq) in portions under nitrogen. The reaction was stirred at 110° C. for 12 h. The mixture was concentrated under reduced pressure to give a residue, which was purified by silica gel column chromatography (petroleum ether/ethyl acetate=1/0 to 10/1) to afford 1-chloro-2-methyl-5-nitro-3-vinylbenzene.

Step 3: To a solution of 1-chloro-2-methyl-5-nitro-3-vinylbenzene (370 mg, 1.87 mmol, 1.00 eq) in tetrahydrofuran (6.00 mL) and ethyl acetate (6.00 mL) were added zinc chloride (12.7 μL, 271 μmol, 0.140 eq) and palladium on activated carbon (10%) (wetted with ca. 55% water) (50.0 mg) in portions under H₂ (15 Psi). The reaction was stirred at 25° C. for 1 h. The mixture was filtered, and the filtrate was concentrated under reduced pressure to give a residue. The residue was purified by silica gel column chromatography (petroleum ether/ethyl acetate=20/1 to 5/1) to afford 3-chloro-5-ethyl-4-methylaniline.

Compound 44: General procedure A with variant i) was used for the preparation from compound VI employing 4-(2-pyridyl)aniline.

¹H NMR (400 MHz, DMSO-d₆) δ=10.98 (s, 1H), 10.10 (s, 1H), 8.64 (d, J=4.6 Hz, 1H), 8.06 (s, 1H), 8.02 (s, 1H), 7.96-7.84 (m, 2H), 7.76-7.62 (m, 3H), 7.58-7.50 (m, 2H), 7.36-7.30 (m, 1H), 5.12 (dd, J=5.2, 13.4 Hz, 1H), 4.42 (d, J=17.2 Hz, 1H), 4.30 (d, J=17.2 Hz, 1H), 3.12-3.04 (m, 1H), 3.12-3.02 (m, 1H), 2.96-2.88 (m, 1H), 2.96-2.82 (m, 1H), 2.76-2.68 (m, 2H), 2.60 (br d, J=17.8 Hz, 1H), 2.50-2.34 (m, 1H), 2.06-1.96 (m, 1H). MS (ESI) m/z 469.4 [M+H]⁺

Step 1: To a solution of (4-nitrophenyl)boronic acid (1.41 g, 8.44 mmol, 1.00 eq) and 2-bromopyridine (1.20 mL, 12.7 mmol, 1.50 eq) in ethanol (35.0 mL) were added potassium carbonate (2.33 g, 16.9 mmol, 2.00 eq) and tetrakis[triphenylphosphine]palladium(0) (1.95 g, 1.69 mmol, 0.20 eq) in one portion. The reaction was stirred at 90° C. under nitrogen for 12 h. The mixture was diluted with water (30.0 mL) and exacted with ethyl acetate (3×30 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1 to 2/1) to afford 2-(4-nitrophenyl)pyridine.

Step 2: To a solution of 2-(4-nitrophenyl)pyridine (600 mg, 3.00 mmol, 1.00 eq) in methanol (6.00 mL) and water (3.00 mL) were added ferrous powder (502 mg, 8.99 mmol, 3.00 eq) and ammonium chloride (802 mg, 15.0 mmol, 5.00 eq). The reaction was stirred at 80° C. for 2 h. The mixture was filtered, and the filtrate was concentrated under reduced pressure to give a residue. The residue was diluted with water (30.0 mL) and exacted with ethyl acetate (3×30.0 mL). The combined organic layers were washed with brine (2×10.0 mL), dried over sodium sulfate, filtered, and concentrated under reduced pressure to afford 4-(2-pyridyl)aniline.

Compound 45: General procedure A with variant iv) was used for the preparation from compound VI employing 4-(tert-butyl)aniline.

¹H NMR (400 MHz, DMSO-d₆) δ=10.97 (br s, 1H), 9.83 (s, 1H), 7.62 (s, 1H), 7.51 (s, 2H), 7.47 (d, J=8.4 Hz, 2H), 7.28 (d, J=8.4 Hz, 2H), 5.14-5.06 (m, 1H), 4.45-4.36 (m, 1H), 4.32-4.24 (m, 1H), 3.02 (br t, J=7.4 Hz, 2H), 2.96-2.84 (m, 1H), 2.65 (br t, J=7.4 Hz, 2H), 2.59 (br d, J=17.4 Hz, 2H), 2.45-2.31 (m, 1H), 2.05-1.94 (m, 1H), 1.26-1.26 (m, 1H), 1.24 (s, 9H). MS (ESI) m/z 448.4 [M+H]⁺

Compound 46: General procedure A with variant iii) was used for the preparation from compound VI employing 5-ethyl-6-methylpyridin-3-amine.

¹H NMR (400 MHz, DMSO-d₆) δ=10.98 (s, 1H), 9.98 (s, 1H), 8.43 (d, J=2.4 Hz, 1H), 7.76 (d, J=2.4 Hz, 1H), 7.63 (s, 1H), 7.52 (s, 2H), 5.11 (dd, J=5.2, 13.2 Hz, 1H), 4.46-4.37 (m, 1H), 4.33-4.25 (m, 1H), 3.03 (br t, J=7.6 Hz, 2H), 2.96-2.87 (m, 1H), 2.72-2.66 (m, 2H), 2.57 (q, J=7.6 Hz, 3H), 2.45-2.40 (m, 1H), 2.39 (s, 3H), 2.04-1.96 (m, 1H), 1.15 (t, J=7.6 Hz, 3H). MS (ESI) m/z 435.3 [M+H]⁺

Step 1: To a solution of 3-bromo-2-methyl-5-nitropyridine (900 mg, 4.15 mmol, 1.00 eq), ethylboronic acid (919 mg, 12.4 mmol, 3.00 eq) and potassium carbonate (1.72 g, 12.4 mmol, 3.00 eq) in dioxane (10.0 mL) was added tetrakis[triphenylphosphine]palladium(0) (479 mg, 415 μmol, 0.100 eq) under nitrogen. The reaction was stirred at 110° C. for 12 h under nitrogen atmosphere. The mixture was concentrated under reduced pressure to give a residue. The residue was purified by silica gel column chromatography (petroleum ether/ethyl acetate=20/1 to 5/1) to afford 3-ethyl-2-methyl-5-nitropyridine.

Step 2: A mixture of 3-ethyl-2-methyl-5-nitropyridine (600 mg, 3.61 mmol, 1.00 eq), iron powder (605 mg, 10.8 mmol, 3.00 eq) and ammonium chloride (579 mg, 10.8 mmol, 3.00 eq) in methanol (10.0 mL) and water (10.0 mL) was stirred at 80° C. for 2 h. The mixture was concentrated under reduced pressure to give a residue. The residue was added to a saturated sodium bicarbonate solution (20.0 mL) and extracted with ethyl acetate (3×20.0 mL). The combined organic layers were washed with brine (20.0 mL), dried over anhydrous sodium sulfate, filtered, and concentrated under reduced pressure to afford 5-ethyl-6-methylpyridin-3-amine.

Compound 47: General procedure A with variant i) was used for the preparation from compound VI employing 6-ethyl-5-methyl-pyridin-3-amine.

¹H NMR (400 MHz, DMSO-d₆) δ=10.98 (s, 1H), 10.82-10.66 (m, 1H), 8.88 (d, J=1.6 Hz, 1H), 8.15 (s, 1H), 7.63 (s, 1H), 7.55-7.50 (m, 2H), 5.10 (dd, J=5.1, 13.3 Hz, 1H), 4.49-4.38 (m, 1H), 4.33-4.23 (m, 1H), 3.06 (br t, J=7.2 Hz, 2H), 2.95-2.87 (m, 3H), 2.80 (br t, J=7.3 Hz, 2H), 2.63 (br s, 1H), 2.43 (s, 3H), 2.40-2.33 (m, 1H), 2.04-1.95 (m, 1H), 1.23 (t, J=7.6 Hz, 3H). MS (ESI) m/z 435.1 [M+H]⁺

Step 1: To a solution of 2-chloro-3-methyl-5-nitropyridine (2.00 g, 11.6 mmol, 1.00 eq), ethylboronic acid (2.14 g, 29.0 mmol, 2.50 eq) and potassium carbonate (4.81 g, 34.8 mmol, 3.00 eq) in dioxane (20.0 mL) was added tetrakis[triphenylphosphine]palladium(O) (1.34 g, 1.16 mmol, 0.100 eq) at 20° C. The reaction was stirred at 110° C. under nitrogen for 12 h. The mixture was concentrated under reduced pressure to give a residue. The residue was purified by silica gel column chromatography (petroleum ether/ethyl acetate=1/0 to 20/1) to afford 2-ethyl-3-methyl-5-nitropyridine.

Step 2: A mixture of 2-ethyl-3-methyl-5-nitropyridine (800 mg, 4.81 mmol, 1.00 eq), iron powder (806 mg, 14.4 mmol, 3.00 eq) and ammonium chloride (1.29 g, 24.0 mmol, 5.00 eq) in methanol (8.00 mL) and water (4.00 mL) was stirred at 80° C. for 1 h. The mixture was filtered, and the filtrate was concentrated under reduced pressure to give a residue. The residue was added to water (100 mL) and the solution was stirred for 10 min, then extracted with ethyl acetate (3×50.0 mL). The combined organic layers were washed with brine (100 mL), dried over anhydrous sodium sulfate, filtered, and concentrated under reduced pressure to afford 6-ethyl-5-methylpyridin-3-amine.

Compound 48: General procedure A with variant i) was used for the preparation from compound VI employing 7,8-dihydro-5H-pyrano[4,3-b]pyridin-3-amine.

¹H NMR (400 MHz, DMSO-d₆) δ=11.00 (s, 1H), 10.11 (s, 1H), 8.44 (s, 1H), 7.77 (d, J=2.0 Hz, 1H), 7.62 (s, 1H), 7.51 (s, 2H), 5.10 (dd, J=5.2, 13.2 Hz, 1H), 4.67 (s, 2H), 4.46-4.37 (m, 1H), 4.32-4.24 (m, 1H), 3.94 (t, J=5.8 Hz, 2H), 3.02 (t, J=7.2 Hz, 2H), 2.96-2.85 (m, 1H), 2.79 (t, J=5.8 Hz, 2H), 2.72-2.67 (m, 2H), 2.59 (d, J=18.2 Hz, 1H), 2.44-2.36 (m, 1H), 2.04-1.93 (m, 1H). MS (ESI) m/z 449.2 [M+H]⁺

Compound 49: General procedure A with variant i) was used for the preparation from compound VI employing 6,7-dihydro-5H-cyclopenta[b]pyridin-3-amine.

¹H NMR (400 MHz, DMSO-d₆) δ=11.02 (s, 1H), 10.42 (br s, 1H), 8.70 (br s, 1H), 8.14 (s, 1H), 8.10-8.02 (m, 1H), 7.64 (s, 1H), 7.54 (s, 2H), 5.12 (dd, J=5.0, 13.4 Hz, 1H), 4.42 (d, J=17.2 Hz, 1H), 4.28 (d, J=17.2 Hz, 1H), 3.10-3.02 (m, 3H), 2.76 (br t, J=7.4 Hz, 2H), 2.60 (br d, J=17.8 Hz, 2H), 2.48-2.32 (m, 2H), 2.14 (td, J=7.8, 15.2 Hz, 3H), 2.06-1.96 (m, 1H). MS (ESI) m/z 433.3 [M−H]⁺

Step 1: To a solution of 3-nitro-6,7-dihydro-5H-cyclopenta[b]pyridine (600 mg, 3.65 mmol, 1.00 eq) in methanol (6.00 mL) and water (3.00 mL) were added ferrous powder (612 mg, 11.0 mmol, 3.00 eq) and ammonium chloride (977 mg, 18.3 mmol, 5.00 eq). The reaction was stirred at 80° C. for 2 h. The mixture was filtered, and the filtrate was concentrated under reduced pressure to give a residue. Water (30.0 mL) was added to the residue, and the mixture was exacted with ethyl acetate (3×30.0 mL). The combined organic layers were washed with brine (2×10.0 mL), dried over sodium sulfate, filtered, and concentrated under reduced pressure to afford 6,7-dihydro-5H-cyclopenta[b]pyridin-3-amine.

Compound 50: General procedure A with variant ii) was used for the preparation from compound VI employing 3-chloro-4-methyl-5-((1-methylpyrrolidin-3-yl)oxy)aniline.

¹H NMR (400 MHz, DMSO-d₆) δ=11.10-11.01 (m, 1H), 11.01 (s, 1H), 10.72-10.62 (m, 1H), 10.20 (d, J=3.2 Hz, 1H), 7.63 (s, 1H), 7.53 (s, 2H), 7.45-7.33 (m, 1H), 7.26-7.13 (m, 1H), 5.14-4.99 (m, 2H), 4.45-4.39 (m, 1H), 4.31 (s, 1H), 4.19 (br s, 1H), 3.98-3.92 (m, 1H), 3.76 (br dd, J=5.2, 12.4 Hz, 1H), 3.71-3.61 (m, 1H), 3.43-3.37 (m, 1H), 3.30-3.24 (m, 1H), 3.24-3.11 (m, 1H), 3.03 (br t, J=7.6 Hz, 2H), 2.96-2.89 (m, 2H), 2.89-2.84 (m, 2H), 2.69 (br t, J=7.6 Hz, 2H), 2.62-2.58 (m, 1H), 2.40 (br dd, J=4.4, 13.2 Hz, 1H), 2.27-2.20 (m, 1H), 2.17 (d, J=4.0 Hz, 3H), 2.11-2.05 (m, 1H), 2.03-1.96 (m, 1H). MS (ESI) m/z 539.3 [M+H]⁺

Step 1: A solution of tert-butyl 3-(3-chloro-2-methyl-5-nitrophenoxy)pyrrolidine-1-carboxylate (described in example 44) (730 mg, 2.05 μmol, 1.00 eq) and hydrochloric acid/ethyl acetate (10.0 mL) in ethyl acetate (20.0 mL) was stirred at 25° C. for 4 h. The mixture was concentrated under reduced pressure to give a residue. The residue was diluted with saturated sodium bicarbonate (50.0 mL) and extracted with ethyl acetate (3×20.0 mL). The combined organic layers were washed with brine (20.0 mL), dried over anhydrous sodium sulfate, filtered, and concentrated under reduced pressure to afford 3-(3-chloro-2-methyl-5-nitrophenoxy)pyrrolidine.

Step 2: To a solution of 3-(3-chloro-2-methyl-5-nitrophenoxy)pyrrolidine (400 mg, 1.56 mmol, 1.00 eq) and formaldehyde 37% (12.0 mL, 161 mmol, 103 eq) in methanol (3.00 mL) was added acetic acid (0.09 mL, 1.56 mmol, 1.00 eq) at 25° C. The reaction was stirred at 25° C. for 0.5 h. Then sodium cyanoborohydride (979 mg, 15.6 mmol, 10.0 eq) was added, and the reaction was stirred at 25° C. for another 1.5 h. The mixture was concentrated under reduced pressure to give a residue. The residue was diluted with water (50.0 mL) and extracted with ethyl acetate (3×20.0 mL). The combined organic layers were washed with brine (20.0 mL), dried over sodium sulfate, filtered, and concentrated under reduced pressure to give a residue. The residue was purified by reversed phase preparative HPLC to afford 3-(3-chloro-2-methyl-5-nitrophenoxy)-1-methylpyrrolidine.

Step 3: To a mixture of 3-(3-chloro-2-methyl-5-nitrophenoxy)-1-methylpyrrolidine (250 mg, 923 μmol, 1.00 eq), iron powder (258 mg, 4.62 mmol, 5.00 eq) and ammonium chloride (247 g, 4.62 mmol, 5.00 eq) in methanol (5.00 mL) was added water (5.00 mL) at 25° C. The reaction was stirred at 80° C. for 2 h. The mixture was concentrated under reduced pressure to give a residue. The residue was poured into saturated sodium bicarbonate (20.0 mL) and extracted with ethyl acetate (3×20.0 mL). The combined organic layers were washed with brine (20.0 mL), dried over anhydrous sodium sulfate, filtered, and concentrated under reduced pressure to afford 3-chloro-4-methyl-5-((1-methylpyrrolidin-3-yl)oxy)aniline.

### Compound 51

Step 1: To a solution of 3-(2-(2,6-dioxopiperidin-3-yl)-3-oxoisoindolin-5-yl)propanoic acid (167 mg, 0.53 mmol, 1.00 eq), tert-butyl 3-((5-amino-3-chloro-2-methylphenoxy)methyl) pyrrolidine-1-carboxylate (180 mg, 0.53 mmol, 1.00 eq) and N,N-diisopropylethylamine (0.28 mL, 1.58 mmol, 3.00 eq) in dimethylformamide (1.00 mL) was added 0-(7-Azabenzotriazol-1-yl)-N,N,N′,N′-tetramethyluronium hexafluorophosphate (301 mg, 0.79 mmol, 1.50 eq) in portions. The reaction was stirred at 25° C. for 12 h. The mixture was diluted with water (50.0 mL) and extracted with ethyl acetate (3×30.0 mL). The combined organic layers were washed with brine (20.0 mL), dried over anhydrous sodium sulfate, filtered, and concentrated under reduced pressure to afford tert-butyl 3-((3-chloro-5-(3-(2-(2,6-dioxopiperidin-3-yl)-3-oxoisoindolin-5-yl)propanamido)-2-methylphenoxy)methyl)pyrrolidine-1-carboxylate.

Step 2: A solution of tert-butyl 3-((3-chloro-5-(3-(2-(2,6-dioxopiperidin-3-yl)-3-oxoisoindolin-5-yl)propanamido)-2-methylphenoxy)methyl)pyrrolidine-1-carboxylate (300 mg, 0.47 mmol, 1.00 eq) in hydrochloric acid/ethyl acetate (3.00 M, 5.00 mL) was stirred at 25° C. for 1 h. The mixture was concentrated under reduced pressure to give a residue, which was purified by reversed phase preparative HPLC to afford Compound 51.

¹H NMR (400 MHz, DMSO-d₆) δ=10.98 (s, 1H), 10.15 (s, 1H), 9.17 (br s, 2H), 7.62 (s, 1H), 7.52 (s, 2H), 7.37-7.20 (m, 2H), 5.10 (dd, J=5.1, 13.3 Hz, 1H), 4.47-4.37 (m, 1H), 4.33-4.23 (m, 1H), 4.02-3.93 (m, 2H), 3.39-3.33 (m, 1H), 3.30-3.24 (m, 1H), 3.21-3.14 (m, 1H), 3.07-2.99 (m, 3H), 2.96-2.87 (m, 1H), 2.80-2.73 (m, 1H), 2.72-2.67 (m, 2H), 2.60 (br d, J=18.2 Hz, 1H), 2.47-2.34 (m, 1H), 2.15 (s, 3H), 2.14-2.06 (m, 1H), 2.04-1.96 (m, 1H), 1.85-1.73 (m, 1H). MS (ESI) m/z 539.3 [M+H]⁺

Step 1: To a solution of 2-chloro-1-methyl-4-nitrobenzene (10.0 g, 58.3 mmol, 12.1 mL, 1.00 eq) in sulfuric acid (100 mL) was added N-iodosuccinimide (14.4 g, 64.1 mmol, 1.10 eq) in portions. The reaction was stirred at 60° C. for 2 h. The mixture was poured into water (300 mL) and extracted with ethyl acetate (3×80.0 mL). The combined organic layers were washed with brine (50 mL) and dried over anhydrous sodium sulfate, filtered, and concentrated under reduced pressure to afford 1-chloro-3-iodo-2-methyl-5-nitrobenzene.

Step 2: To a solution of 1-chloro-3-iodo-2-methyl-5-nitrobenzene (7.00 g, 23.5 mmol, 1.00 eq) and potassium hydroxide (3.96 g, 70.6 mmol, 3.00 eq) in dioxane (70.0 mL) and water (10.0 mL) were added tris(dibenzylideneacetone)dipalladium(0) (2.15 g, 2.35 mmol, 0.10 eq) and 2-di-tert-butylphosphino-2,4,6-triisopropylbiphenyl (999 mg, 2.35 mmol, 0.10 eq) under nitrogen. The reaction was stirred at 80° C. for 12 h. The mixture was diluted with water (300 mL) and extracted with ethyl acetate (3×80.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 3-chloro-2-methyl-5-nitrophenol.

Step 3: To a solution of 3-chloro-2-methyl-5-nitrophenol (1.00 g, 5.33 mmol, 1.00 eq) and tert-butyl 3-(((methylsulfonyl)oxy)methyl)pyrrolidine-1-carboxylate (1.64 g, 5.86 mmol, 1.10 eq) in dimethylformamide (10.0 mL) was added potassium carbonate (2.21 g, 15.9 mmol, 3.00 eq) in portions. The reaction was stirred at 80° C. for 12 h. The mixture was filtered, and the filtrate was concentrated under reduced pressure to give a residue. The residue was purified by reversed phase preparative HPLC to afford tert-butyl 3-((3-chloro-2-methyl-5-nitrophenoxy)methyl) pyrrolidine-1-carboxylate.

Step 4: To a solution of tert-butyl 3-((3-chloro-2-methyl-5-nitrophenoxy)methyl)pyrrolidine-1-carboxylate (400 mg, 1.08 mmol, 1.00 eq) and ammonium chloride (289 mg, 5.39 mmol, 5.00 eq) in methanol (4.00 mL) and water (4.00 mL) was added iron powder (181 mg, 3.24 mmol, 3.00 eq) in portions. The reaction was stirred at 80° C. for 2 h. The mixture was filtered, and the filtrate was concentrated under reduced pressure to give a residue. The residue was diluted with water (100 mL) and extracted with ethyl acetate (3×30.0 mL). The combined organic layers were washed with brine (20.0 mL), dried over anhydrous sodium sulfate, filtered, and concentrated under reduced pressure to afford tert-butyl 3-((5-amino-3-chloro-2-methylphenoxy)methyl)pyrrolidine-1-carboxylate.

Compound 52: General procedure A with variant i) was used for the preparation from compound VI employing 2,6-dimethylpyridin-4-amine.

¹H NMR (400 MHz, DMSO-d₆) δ=14.86 (br s, 1H), 11.54 (br s, 1H), 11.62-11.32 (m, 1H), 11.0 (s, 1H), 7.78-7.72 (m, 2H), 7.64 (s, 1H), 7.58-7.50 (m, 2H), 5.10 (dd, J=5.2, 13.4 Hz, 1H), 4.42 (d, J=17.2 Hz, 1H), 4.30 (d, J=17.2 Hz, 1H), 3.44-3.32 (m, 26H), 3.12-3.00 (m, 2H), 2.98-2.86 (m, 3H), 2.68 (br d, J=1.6 Hz, 1H), 2.62 (s, 6H), 2.58-2.52 (m, 1H), 2.40 (dt, J=9.0, 13.4 Hz, 1H), 2.04-1.96 (m, 1H). MS (ESI) m/z 421.3 [M+H]⁺

Compound 53: General procedure A with variant i) was used for the preparation from compound VI employing 4-methyl-3-(morpholinomethyl)-5-(trifluoromethoxy)aniline.

¹H NMR (400 MHz, DMSO-d₆) δ=10.68 (br s, 1H), 9.96 (br s, 1H), 8.12 (s, 1H), 7.70 (br s, 1H), 7.64 (s, 2H), 7.58-7.46 (m, 2H), 5.04 (dd, J=5.2, 13.0 Hz, 1H), 4.46-4.30 (m, 2H), 3.88-3.58 (m, 4H), 3.10-3.02 (m, 4H), 2.94-2.84 (m, 2H), 2.78-2.58 (m, 7H), 2.58-2.52 (m, 2H), 2.44-2.40 (m, 1H), 2.32-2.18 (m, 3H), 2.10-1.98 (m, 1H). MS (ESI) m/z 589.5 [M+H]⁺

Step 1: To a solution of 2-methyl-5-nitro-benzoic acid (10.0 g, 55.2 mmol, 1.00 eq) in sulfuric acid (20.0 mL) was added N-Iodosuccinimide (14.9 g, 66.3 mmol, 1.20 eq). The reaction was stirred at 60° C. for 2 h. The mixture was diluted with ice water (200 mL) and filtered. The filter cake was washed with water (100 mL) and dried under vacuum to afford 3-iodo-2-methyl-5-nitro-benzoic acid.

Step 2: To a solution of 3-iodo-2-methyl-5-nitro-benzoic acid (5.00 g, 16.3 mmol, 1.00 eq), copper iodide (310 mg, 1.63 mmol, 0.10 eq) and quinolin-8-ol (563 μL, 3.26 mmol, 0.20 eq) in water (3.00 mL) and dimethylsulfoxide (3.00 mL) was added a solution of potassium hydroxide (3.65 g, 65.1 mmol, 4.00 eq). The reaction was stirred at 100° C. for 12 h. The mixture was diluted with water (50.0 mL) and extracted with ethyl acetate (2×50.0 mL). The combined organic layers were washed with water (50.0 mL), dried over anhydrous sodium sulfate, filtered, and concentrated under reduced pressure to afford 3-hydroxy-2-methyl-5-nitro-benzoic acid. It was used directly in the next step.

Step 3: To a solution of 3-hydroxy-2-methyl-5-nitro-benzoic acid (3.20 g, 16.2 mmol, 1.00 eq) and morpholine (1.71 mL, 19.5 mmol, 1.20 eq) in dichloromethane (100 mL) were added triethylamine (2.26 mL, 16.2 mmol, 1.00 eq) and O-(7-azabenzotriazol-1-yl)-N,N,N,N-tetramethyluroniumhexafluorophosphate (7.41 g, 19.5 mmol, 1.20 eq) at 20° C. The reaction was stirred at 20° C. for 12 h. The mixture was concentrated under reduced pressure to give a residue. The residue was diluted with water (100 mL) and extracted with ethyl acetate (100 mL). The organic layer was washed with water (100 mL), dried over anhydrous sodium sulfate, filtered, and the filtrate was concentrated under reduced pressure to give a residue. The residue was purified by silica gel column chromatography (petroleum ether/ethyl acetate=1/1 to 0/1) to afford (3-hydroxy-2-methyl-5-nitro-phenyl)-morpholino-methanone.

Step 4: To a solution of (3-hydroxy-2-methyl-5-nitro-phenyl)-morpholino-methanone (1.30 g, 4.88 mmol, 1.00 eq), silver trifluoromethanesulfonate (6.27 g, 24.4 mmol, 5.00 eq), 1-(chloromethyl)-4-fluoro-1,4-diazoniabicyclo[2.2.2]octane bis(tetrafluoroborate) (3.46 g, 9.77 mmol, 2.00 eq), N-fluorobenzenesulfonimide (3.08 g, 9.77 mmol, 2.00 eq) and caesium fluoride (4.45 g, 29.3 mmol, 1.08 mL, 6.00 eq) in toluene (130 mL) were added trimethyl(trifluoromethyl)silane (3.47 g, 24.4 mmol, 5.00 eq) and 2-fluoropyridine (2.10 mL, 24.4 mmol, 5.00 eq) under nitrogen. The reaction was stirred at 20° C. for 12 h under nitrogen. The mixture was filtered, and the filtrate was concentrated under reduced pressure to give a residue. The residue was diluted with water (100 mL) and extracted with ethyl acetate (100 mL). The organic layer was washed with water (20.0 mL), dried over anhydrous sodium sulfate, filtered, and the filtrate was concentrated under reduced pressure to give a residue. The residue was purified by silica gel column chromatography (petroleum ether/ethyl acetate=1/0 to 2/1) to afford (2-methyl-5-nitro-3-(trifluoromethoxy)phenyl)(morpholino)methanone.

Step 5: To a solution of (2-methyl-5-nitro-3-(trifluoromethoxy)phenyl)-morpholino-methanone (900 mg, 2.69 mmol, 1.00 eq) in tetrahydrofuran (15.0 mL) was added borane dimethyl sulfide complex (10.0 M, 539 μL, 2.00 eq) at 0° C. The reaction was stirred at 60° C. for 30 min. The mixture was quenched with methanol (2.00 mL) and concentrated under reduced pressure to give a residue. The residue was purified by reversed phase preparative HPLC to afford 4-(2-methyl-5-nitro-3-(trifluoromethoxy)benzyl)morpholine.

Step 6: To a solution of 4-(2-methyl-5-nitro-3-(trifluoromethoxy)benzyl)morpholine (400 mg, 1.25 mmol, 1.00 eq) in methanol (5.00 mL) and water (5.00 mL) was added iron powder (488 mg, 8.74 mmol, 7.00 eq) and ammonium chloride (468 mg, 8.74 mmol, 7.00 eq). The reaction was stirred at 80° C. for 2 h. The mixture was diluted with saturated sodium carbonate (1.00 mL) and extracted with ethyl acetate (2×10.0 mL). The combined organic layers were washed with water (5.00 mL), dried over anhydrous sodium sulfate, filtered, and the filtrate was concentrated under reduced pressure to give a residue. The residue was purified by silica gel column chromatography (petroleum ether/ethyl acetate=3/1 to 1/1) to afford 4-methyl-3-(morpholinomethyl)-5-(trifluoromethoxy)aniline.

Compound 54: General procedure A with variant iv) was used for the preparation from compound VI employing 2-ethylpyridin-4-amine.

¹H NMR (400 MHz, DMSO-d₆) δ=10.98 (s, 1H), 10.26 (s, 1H), 8.34-8.30 (m, 1H), 8.29 (d, J=5.5 Hz, 1H), 7.62 (s, 1H), 7.51 (d, J=0.9 Hz, 2H), 7.42 (d, J=1.8 Hz, 1H), 7.38-7.33 (m, 1H), 5.13-5.07 (m, 1H), 4.45-4.37 (m, 1H), 4.32-4.25 (m, 1H), 3.02 (t, J=7.4 Hz, 2H), 2.96-2.84 (m, 1H), 2.76-2.68 (m, 2H), 2.68-2.63 (m, 2H), 2.63-2.55 (m, 1H), 2.45-2.31 (m, 1H), 2.04-1.95 (m, 1H), 1.18 (t, J=7.6 Hz, 3H). MS (ESI) m/z 421.3 [M+H]⁺

Compound 55: General procedure A with variant i) was used for the preparation from compound VI employing 4-(3-methyloxetan-3-yl)aniline.

¹H NMR (400 MHz, DMSO-d₆) δ=10.99 (s, 1H), 9.92 (s, 1H), 7.63 (s, 1H), 7.57-7.50 (m, 4H), 7.19-7.15 (m, 2H), 5.11 (dd, J=5.1, 13.3 Hz, 1H), 4.76 (d, J=5.6 Hz, 2H), 4.51 (d, J=5.6 Hz, 2H), 4.45-4.38 (m, 1H), 4.32-4.25 (m, 1H), 3.03 (t, J=7.5 Hz, 2H), 2.95-2.84 (m, 1H), 2.67 (t, J=7.5 Hz, 2H), 2.64-2.58 (m, 1H), 2.45-2.34 (m, 1H), 2.04-1.95 (m, 1H), 1.60 (s, 3H). MS (ESI) m/z 462.1 [M+H]⁺

Step 1: To a solution of diethyl 2-methylmalonate (13.6 g, 78.0 mmol, 13.3 mL, 1.10 eq) in dimethylformamide (80.0 mL) was added sodium hydride (60% dispersion in mineral oil) (3.40 g, 85.0 mmol, 1.20 eq) slowly at 0° C. The reaction was stirred at 0° C. for 0.5 h, then 1-fluoro-4-nitrobenzene (10.0 g, 70.8 mmol, 7.52 mL, 1.00 eq) was added. The mixture was stirred at 25° C. for 3 h. Water (200 mL) was added, and the mixture was extracted with ethyl acetate (3×50.0 mL). The combined organic layers were dried over sodium sulfate, filtered, and concentrated under reduced pressure to give a residue. The residue was purified by silica gel column chromatography (petroleum ether/ethyl acetate=10/1 to 8/1) to afford diethyl 2-methyl-2-(4-nitrophenyl)malonate.

Step 2: To a solution of diethyl 2-methyl-2-(4-nitrophenyl)malonate (7.00 g, 23.7 mmol, 1.00 eq) in tetrahydrofuran (15.0 mL) was added lithium aluminium hydride (953 mg, 25.1 mmol, 1.06 eq) slowly at 0° C. under nitrogen. The reaction was stirred at 0° C. for 3 h. The mixture was then partitioned between dichloromethane and 1 M hydrochloric acid. The organic layer was separated, and the aqueous layer was extracted with dichloromethane (3×50.0 mL). The combined organic layers were concentrated under reduced pressure to give a residue. The residue was purified by silica gel column chromatography (petroleum ether/ethyl acetate=10/1 to 0/1) to afford 2-methyl-2-(4-nitrophenyl)propane-1,3-diol.

Step 3: To a solution of 2-methyl-2-(4-nitrophenyl)propane-1,3-diol (200 mg, 947 μmol, 1.00 eq) in tetrahydrofuran (10.0 mL) was added n-butyllithium (2.5 M in hexane, 0.46 mL, 1.21 eq) and tosyl chloride (271 mg, 1.42 mmol, 1.50 eq) at 0° C. The reaction was stirred at 25° C. for 1 h. After cooling to 0° C., n-butyllithium (2.5 M in hexane, 0.46 mL, 1.21 eq) was added. The reaction was stirred at 65° C. for another 2 h. The reaction was quenched with saturated ammonium chloride (10 mL) and extracted with ethyl acetate (50.0 mL). The organic layer was separated and concentrated under reduced pressure to give a residue. The residue was purified by silica gel column chromatography (petroleum ether/ethyl acetate=3/1) to afford 3-methyl-3-(4-nitrophenyl)oxetane.

Step 4: To a solution of 3-methyl-3-(4-nitrophenyl)oxetane (56.0 mg, 290 μmol, 1.00 eq) in ethyl acetate (5.00 mL) was added wet palladium on carbon (10% weight on C) (20.0 mg). The reaction was stirred at 25° C. for 2 h under hydrogen (15.0 Psi). The mixture was filtered, and the filtrate was concentrated under reduced pressure to afford 4-(3-methyloxetan-3-yl)aniline.

Compound 56: General procedure A with variant i) was used for the preparation from compound VI employing 3-fluoro-5-(trifluoromethoxy)aniline.

¹H NMR (400 MHz, DMSO-d₆) δ=10.98 (br s, 1H), 10.40 (s, 1H), 7.62 (s, 1H), 7.52 (s, 2H), 7.50-7.44 (m, 2H), 7.02 (br d, J=9.0 Hz, 1H), 5.10 (dd, J=5.1, 13.3 Hz, 1H), 4.47-4.37 (m, 1H), 4.33-4.24 (m, 1H), 3.03 (t, J=7.5 Hz, 2H), 2.95-2.85 (m, 1H), 2.71 (t, J=7.5 Hz, 2H), 2.64-2.58 (m, 1H), 2.45-2.35 (m, 1H), 2.05-1.94 (m, 1H). MS (ESI) m/z 494.1 [M+H]⁺

Compound 57: General procedure A with variant i) was used for the preparation from compound VI employing 2-ethyl-6-methylpyridin-4-amine.

¹H NMR (400 MHz, DMSO-d₆) δ=10.98 (s, 1H), 10.42-10.29 (m, 1H), 8.13 (s, 1H), 7.62 (s, 1H), 7.51 (s, 2H), 7.33 (br d, J=18.1 Hz, 2H), 5.10 (dd, J=5.1, 13.3 Hz, 1H), 4.45-4.36 (m, 1H), 4.35-4.23 (m, 1H), 3.02 (br t, J=7.3 Hz, 2H), 2.94-2.87 (m, 1H), 2.74 (br t, J=7.3 Hz, 2H), 2.71-2.65 (m, 2H), 2.59 (br d, J=16.8 Hz, 1H), 2.41 (s, 3H), 2.39-2.30 (m, 1H), 2.04-1.95 (m, 1H), 1.19 (t, J=7.6 Hz, 3H). MS (ESI) m/z 435.2 [M+H]⁺

Step 1: To a solution of 2-chloro-6-methyl-4-nitropyridine (3.00 g, 17.4 mmol, 1.00 eq), ethylboronic acid (3.85 g, 52.2 mmol, 3.00 eq) and potassium carbonate (7.21 g, 52.2 mmol, 3.00 eq) in dioxane (30.0 mL) was added tetrakis(triphenylphosphine)palladium(0) (2.01 g, 1.74 mmol, 0.10 eq) under nitrogen atmosphere. The reaction was stirred at 110° C. for 12 h. The mixture was concentrated under reduced pressure to give residue. The residue was purified by silica gel column chromatography (petroleum ether/ethyl acetate=10/1) to afford 2-ethyl-6-methyl-4-nitropyridine.

Step 2: To a solution of 2-ethyl-6-methyl-4-nitropyridine (1.50 g, 9.03 mmol, 1.00 eq) and ammonium chloride (2.41 g, 45.1 mmol, 5.00 eq) in methanol (12.0 mL) and water (3.00 mL) was added iron powder (2.52 g, 45.1 mmol, 5.00 eq) in portions. The reaction was stirred at 80° C. for 2 h. The mixture was filtered, and the filtrate was concentrated under reduced pressure to give a residue. The residue was diluted with saturated sodium bicarbonate (150 mL) and extracted with ethyl acetate (3×80.0 mL). The combined organic layers were washed with brine (60.0 mL), dried over sodium sulfate, filtered, and concentrated under reduced pressure to give a residue. The residue was purified by reversed phase preparative HPLC to afford 2-ethyl-6-methylpyridin-4-amine.

Compound 58: General procedure A with variant i) was used for the preparation from compound VI employing 4-(1-methylcyclopropyl)aniline.

¹H NMR (400 MHz, DMSO-d₆) δ=10.98 (s, 1H), 9.84 (s, 1H), 7.63 (s, 1H), 7.51 (s, 2H), 7.46 (d, J=8.6 Hz, 2H), 7.13 (d, J=8.6 Hz, 2H), 5.10 (dd, J=5.0, 13.3 Hz, 1H), 4.46-4.36 (m, 1H), 4.34-4.23 (m, 1H), 3.02 (br t, J=7.5 Hz, 2H), 2.97-2.84 (m, 1H), 2.65 (br t, J=7.6 Hz, 2H), 2.58 (br s, 1H), 2.46-2.35 (m, 1H), 2.04-1.95 (m, 1H), 1.34 (s, 3H), 0.80-0.74 (m, 2H), 0.74-0.67 (m, 2H). MS (ESI) m/z 446.3 [M+H]⁺

Step 1: To freshly distilled dichloromethane (50.0 mL) was added diethylzinc (1 M in toluene, 40.6 mL, 4.00 eq). The solution was cooled to −40° C., and diiodomethane (40.6 mL, 4.00 eq) in dichloromethane (10.0 mL) was added slowly. The mixture was stirred at −40° C. for 30 min, then trifluoroacetic acid (0.15 mL, 2.03 mmol, 0.20 eq) and N,N-dimethylacetamide (1.05 mL, 10.1 mmol, 1.00 eq) in dichloromethane (10.0 mL) were added. The reaction was stirred at −15° C. for 0.5 h, then 1-bromo-4-(prop-1-en-2-yl)benzene (2.00 g, 10.1 mmol, 1.00 eq) in dichloromethane (10.0 mL) was added at 0° C. The mixture was stirred at 25° C. for 12 h. The reaction was quenched with ice-water (50.0 mL) at 0° C. The organic layer was separated and concentrated under reduced pressure to give a residue. The residue was purified by silica gel column chromatography (petroleum ether 100%) to afford 1-bromo-4-(1-methylcyclopropyl)benzene.

Step 2: To a solution of 1-bromo-4-(1-methylcyclopropyl)benzene (2.00 g, 9.47 mmol, 1.00 eq) (crude) in tert-amyl alcohol (100 mL) were added tert-butyl carbamate (2.00 g, 17.1 mmol, 1.80 eq), methanesulfonato(2-di-tbutylphosphino-2,4,6-tri-ipropyl-1,1-biphenyl)(2-amino-1,1-biphenyl-2-yl)palladium(II) (600 mg, 755 μmol, 0.0800 eq) and sodium tert-butoxide (2 M in tetrahydrofuran, 14.0 mL, 2.96 eq). The reaction was stirred at 90° C. for 3 h under nitrogen. The mixture was diluted with ethyl acetate (200 mL) and water (200 mL). The organic layer was separated and concentrated under reduced pressure to give a residue. The residue was purified by reversed phase preparative HPLC to afford tert-butyl (4-(1-methylcyclopropyl)phenyl) carbamate.

Step 3: To a solution of tert-butyl (4-(1-methylcyclopropyl)phenyl) carbamate (520 mg, 2.10 mmol, 1.00 eq) in ethyl acetate (10.0 mL) was added hydrogen chloride/ethyl acetate (4 M, 10 mL, 19.0 eq). The reaction was stirred at 25° C. for 1 h. The mixture was concentrated under reduced pressure to afford 4-(1-methylcyclopropyl)aniline.

### Compound 59

Step 1: To a solution of 3-(2-(2,6-dioxopiperidin-3-yl)-3-oxoisoindolin-5-yl)propanoic acid (100 mg, 0.32 mmol, 1.00 eq) and 4-(trifluoromethoxy)pyridin-2-amine (68 mg, 0.38 mmol, 1.20 eq) in pyridine (0.50 mL) was added phosphorus oxychloride (59 μL, 0.63 mmol, 2.00 eq) dropwise. The reaction was stirred at 50° C. for 2 h. Dimethylsulfoxide (2.00 mL) was added, and the mixture was filtered. The filtrate was purified by reversed phase preparative HPLC to afford Compound 59.

¹H NMR (400 MHz, DMSO-d₆) δ=11.02-10.86 (m, 2H), 8.41 (d, J=5.6 Hz, 1H), 8.09 (s, 1H), 7.63 (s, 1H), 7.51 (s, 2H), 7.10 (dd, J=1.1, 5.6 Hz, 1H), 5.10 (dd, J=5.0, 13.3 Hz, 1H), 4.45-4.37 (m, 1H), 4.32-4.25 (m, 1H), 3.02 (br t, J=7.5 Hz, 2H), 2.94-2.87 (m, 1H), 2.82-2.77 (m, 2H), 2.59 (br d, J=15.0 Hz, 1H), 2.44-2.34 (m, 1H), 2.03-1.95 (m, 1H). MS (ESI) m/z 477.2 [M+H]⁺

Compound 60: General procedure A with variant i) was used for the preparation from compound VI employing 4-fluoro-3-(trifluoromethoxy)aniline.

¹H NMR (400 MHz, DMSO-d₆) δ=10.98 (s, 1H), 10.26 (s, 1H), 7.94 (br d, J=6.4 Hz, 1H), 7.64 (s, 1H), 7.54-7.48 (m, 3H), 7.48-7.42 (m, 1H), 5.12 (dd, J=5.2, 13.4 Hz, 1H), 4.42 (d, J=17.2 Hz, 1H), 4.30 (d, J=17.2 Hz, 1H), 3.04 (br t, J=7.6 Hz, 2H), 2.30-2.86 (m, 1H), 2.70 (t, J=7.6 Hz, 2H), 2.64-2.58 (m, 1H), 2.46-2.32 (m, 1H), 2.08-1.94 (m, 1H). MS (ESI) m/z 494.3 [M+H]⁺

Compound 61: General procedure A with variant i) was used for the preparation from compound VI employing 3-chloro-4-methyl-5-((1-methylpyrrolidin-3-yl)methoxy)aniline.

¹H NMR (400 MHz, DMSO-d₆) δ=10.99 (s, 1H), 10.41 (br s, 1H), 10.10 (s, 1H), 7.62 (s, 1H), 7.52 (s, 2H), 7.35-7.21 (m, 2H), 5.10 (dd, J=5.0, 13.3 Hz, 1H), 4.46-4.37 (m, 1H), 4.33-4.25 (m, 1H), 4.07-3.94 (m, 2H), 3.73 (br dd, J=4.6, 6.8 Hz, 1H), 3.27-3.19 (m, 1H), 3.14-3.06 (m, 1H), 3.03 (br t, J=7.3 Hz, 2H), 2.96-2.87 (m, 2H), 2.84 (t, J=5.3 Hz, 3H), 2.71-2.65 (m, 2H), 2.61 (br d, J=14.7 Hz, 1H), 2.46-2.37 (m, 1H), 2.37-2.21 (m, 1H), 2.15 (s, 3H), 2.14-2.05 (m, 1H), 2.00 (tdd, J=2.6, 5.1, 10.0 Hz, 1H), 1.97-1.70 (m, 1H). MS (ESI) m/z 553.3 [M+H]⁺

Step 1: To a solution of 2-chloro-1-methyl-4-nitrobenzene (10.0 g, 58.3 mmol, 12.1 mL, 1.00 eq) in sulfuric acid (100 mL) was added N-iodosuccinimide (14.4 g, 64.1 mmol, 1.10 eq) in portions. The reaction was stirred at 60° C. for 2 h. The mixture was poured into water (300 mL) and extracted with ethyl acetate (3×80.0 mL). The combined organic layers were washed with brine (50 mL) and dried over anhydrous sodium sulfate, filtered, and concentrated under reduced pressure to afford 1-chloro-3-iodo-2-methyl-5-nitrobenzene.

Step 2: To a solution of 1-chloro-3-iodo-2-methyl-5-nitrobenzene (7.00 g, 23.5 mmol, 1.00 eq) and potassium hydroxide (3.96 g, 70.6 mmol, 3.00 eq) in dioxane (70.0 mL) and water (10.0 mL) were added tris(dibenzylideneacetone)dipalladium(0) (2.15 g, 2.35 mmol, 0.10 eq) and 2-di-tert-butylphosphino-2,4,6-triisopropylbiphenyl (999 mg, 2.35 mmol, 0.10 eq) under nitrogen. The reaction was stirred at 80° C. for 12 h. The mixture was diluted with water (300 mL) and extracted with ethyl acetate (3×80.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 3-chloro-2-methyl-5-nitrophenol.

Step 3: To a solution of 3-chloro-2-methyl-5-nitrophenol (1.00 g, 5.33 mmol, 1.00 eq) and tert-butyl 3-(((methylsulfonyl)oxy)methyl)pyrrolidine-1-carboxylate (1.64 g, 5.86 mmol, 1.10 eq) in dimethylformamide (10.0 mL) was added potassium carbonate (2.21 g, 15.9 mmol, 3.00 eq) in portions. The reaction was stirred at 80° C. for 12 h. The mixture was filtered, and the filtrate was concentrated under reduced pressure to give a residue. The residue was purified by reversed phase preparative HPLC to afford tert-butyl 3-((3-chloro-2-methyl-5-nitrophenoxy)methyl) pyrrolidine-1-carboxylate.

Step 4: A mixture of tert-butyl 3-((3-chloro-2-methyl-5-nitrophenoxy)methyl)pyrrolidine-1-carboxylate (1.26 g, 3.40 mmol, 1.00 eq) in hydrochloric acid/ethyl acetate (4.0 M, 1.13 mL) was stirred at 25° C. for 1 h. The mixture was concentrated under reduced pressure to afford 3-((3-chloro-2-methyl-5-nitrophenoxy)methyl)pyrrolidine (crude, HCl).

Step 5: To a solution of 3-((3-chloro-2-methyl-5-nitrophenoxy)methyl)pyrrolidine (900 mg, 3.32 mmol, 1.00 eq) in 2,2,2-trifluoroethanol (10.0 mL) was added paraformaldehyde (0.46 mL, 16.6 mmol, 5.00 eq). The reaction was stirred at 60° C. for 0.5 h. Then sodium borohydride (252 mg, 6.65 mmol, 2.00 eq) was added in portions, and the reaction was stirred at 60° C. for 1 h. The mixture was diluted with water (100 mL) and extracted with ethyl acetate (3×30.0 mL). The combined organic layers were washed with brine (20.0 mL), dried over anhydrous sodium sulfate, filtered, and concentrated under reduced pressure to afford 3-((3-chloro-2-methyl-5-nitrophenoxy)methyl)-1-methylpyrrolidine.

Step 6: To a solution of 3-((3-chloro-2-methyl-5-nitrophenoxy)methyl)-1-methylpyrrolidine (850 mg, 2.99 mmol, 1.00 eq) and ammonium chloride (798 mg, 14.9 mmol, 5.00 eq) in methanol (5.00 mL) and water (5.00 mL) was added iron powder (500 mg, 8.96 mmol, 3.00 eq) in portions. The reaction was stirred at 80° C. for 2 h. The mixture was concentrated under reduced pressure to give a residue. The residue was purified by reversed phase preparative HPLC to afford 3-chloro-4-methyl-5-((1-methylpyrrolidin-3-yl)methoxy)aniline.

### Compound 62

Step 1: To a solution of 3-(2-(2,6-dioxopiperidin-3-yl)-3-oxoisoindolin-5-yl)propanoic acid (100 mg, 316 μmol, 1.00 eq) in dimethylformamide (2.00 mL) were added 3-(trifluoromethyl)isothiazol-5-amine (80.0 mg, 474 μmol, 1.50 eq), 2,4,6-tripropyl-1,3,5,2,4,6-trioxatriphosphinane 2,4,6-trioxide (0.28 mL, 301 mg, 474 μmol, 1.50 eq, 50% purity) and N,N-diisopropylethylamine (0.17 mL, 0.95 mmol, 3.00 eq) at 25° C. The reaction was stirred at 25° C. for 16 h. The mixture was diluted with water (30.0 mL) and extracted with ethyl acetate (2×30.0 mL). The combined organic layers were washed with water (30.0 mL) and brine (10.0 mL), dried over anhydrous sodium sulfate, filtered, and concentrated under reduced pressure to give a residue. The residue was purified by reversed phase preparative HPLC to afford Compound 62.

¹H NMR (400 MHz, DMSO-d₆) δ=10.97 (br d, J=1.6 Hz, 1H), 7.64 (s, 1H), 7.52 (s, 2H), 7.17 (s, 1H), 5.10 (dd, J=4.4, 13.5 Hz, 1H), 4.46-4.25 (m, 2H), 3.13-3.05 (m, 2H), 2.95-2.84 (m, 3H), 2.63 (br s, 1H), 2.42-2.36 (m, 1H), 2.06-1.95 (m, 1H). MS (ESI) m/z 467.0 [M+H]⁺

Step 1: To a solution of potassium tert-butoxide (1 M, 112 mL, 1.60 eq) was added a solution of ethyl 2,2,2-trifluoroacetate (9.71 mL, 70.3 mmol, 1.00 eq) in acetonitrile (4.26 mL, 80.9 mmol, 1.15 eq) slowly at 0° C. The reaction was stirred at 20° C. for 24 h. The pH was adjusted to pH=6-7 with a 1 M hydrochloric acid solution, and the mixture was extracted with ethyl acetate (2×200 mL). The combined organic layers were washed with brine (200 mL), dried over anhydrous sodium sulfate, filtered, and concentrated under reduced pressure to afford 4,4,4-trifluoro-3-oxobutanenitrile. It was used in the next step directly.

Step 2: A solution of 4,4,4-trifluoro-3-oxobutanenitrile (8.00 g, 58.3 mmol, 1.00 eq), ammonium formate (11.0 g, 175 mmol, 3.00 eq) and acetic acid (0.33 mL, 5.84 mmol, 0.10 eq) in toluene (50.0 mL) was heated to 140° C. in a Dean-Stark trap for 3 h. The mixture was concentrated under reduced pressure to afford (Z)-3-amino-4,4,4-trifluorobut-2-enenitrile. It was used in the next step directly.

Step 3: To a solution of (Z)-3-amino-4,4,4-trifluorobut-2-enenitrile (8.00 g, 58.8 mmol, 1.00 eq) in dimethylformamide (30.0 mL) were added magnesium chloride (5.60 g, 58.8 mmol, 1.00 eq) and sodium hydrosulfide (6.59 g, 117 mmol, 2.00 eq) in portions while maintaining the temperature between 0-25° C. The reaction was stirred for 16 h at 25° C. The mixture was concentrated under reduced pressure to afford (Z)-3-amino-4,4,4-trifluorobut-2-enethioamide. It was used in the next step directly.

Step 4: To an ice-cold mixture of (Z)-3-amino-4,4,4-trifluorobut-2-enethioamide (10.0 g, 58.7 mmol, 1.00 eq) in pyridine (60.0 mL) was added hydrogen peroxide 30% (12.3 mL, 128 mmol, 2.18 eq) at 0° C. The reaction was stirred at 25° C. for 16 h. The mixture was quenched with saturated sodium sulfite solution (100 mL) and extracted with ethyl acetate (2×80.0 mL). The combined organic layers were washed with brine (80.0 mL), dried over anhydrous sodium sulfate, filtered, and concentrated under reduced pressure to give a residue. The residue was purified by silica gel column chromatography to afford 3-(trifluoromethyl)isothiazol-5-amine.

Compound 63: General procedure A with variant i) was used for the preparation from compound VI employing 4-(1,1-difluoroethyl)aniline.

¹H NMR (400 MHz, DMSO-d₆) δ=10.97 (s, 1H), 10.11 (s, 1H), 7.71-7.60 (m, 3H), 7.54-7.43 (m, 4H), 5.10 (dd, J=5.1, 13.4 Hz, 1H), 4.44-4.35 (m, 1H), 4.32-4.22 (m, 1H), 3.03 (br t, J=7.5 Hz, 2H), 2.97-2.83 (m, 1H), 2.70 (br t, J=7.6 Hz, 2H), 2.59 (br d, J=17.4 Hz, 1H), 2.42-2.31 (m, 1H), 2.05-1.98 (m, 1H), 1.93 (t, J=18.7 Hz, 3H). MS (ESI) m/z 434.4 [M−21]⁺

Compound 64: General procedure A with variant iv) was used for the preparation from compound VI employing 6-methyl-5-(trifluoromethoxy)pyridin-3-amine.

¹H NMR (400 MHz, DMSO-d₆) δ=10.98 (s, 1H), 10.58 (s, 1H), 8.61 (d, J=2.1 Hz, 1H), 8.22 (s, 1H), 7.63 (s, 1H), 7.53 (d, J=0.9 Hz, 2H), 5.17-5.06 (m, 1H), 4.42 (br d, J=17.2 Hz, 2H), 4.33-4.24 (m, 1H), 3.05 (br t, J=7.5 Hz, 2H), 2.98-2.86 (m, 1H), 2.79-2.71 (m, 2H), 2.62 (br s, 1H), 2.43 (s, 3H), 2.41-2.31 (m, 1H), 2.05-1.95 (m, 1H). MS (ESI) m/z 491.2 [M+H]⁺

Step 1: To a solution of 2-methyl-5-nitropyridin-3-amine (10.0 g, 65.3 mmol, 1.00 eq) in sulfuric acid (2.50 M, 107 mL, 4.10 eq) was added sodium nitrite (5.41 g, 78.4 mmol, 1.20 eq) dissolved in water (20.0 mL) dropwise at 0° C. The reaction was stirred at 0° C. for 0.5 h. Then sulfuric acid (1 M, 53.6 mL, 0.820 eq) was added dropwise, and the reaction was stirred at 70° C. for 1 h. The mixture was diluted with water (200 mL) and extracted with ethyl acetate (3×50.0 mL). The combined organic layers were washed brine (40.0 mL), dried over sodium sulfate, filtered, and concentrated under reduced pressure to give a residue. The residue was purified by silica gel column chromatography (petroleum ether/ethyl acetate=10/1 to 2/1) to afford 2-methyl-5-nitropyridin-3-ol.

Step 2: To a solution of 2-methyl-5-nitropyridin-3-ol (800 mg, 5.19 mmol, 1.00 eq) in dimethylformamide (8.00 mL) was added sodium hydride (60% dispersion in mineral oil) (415 mg, 10.4 mmol, 2.00 eq) in portions at 0° C. The reaction was stirred at 0° C. for 0.5 h. Then dibromodifluoromethane (0.96 mL, 10.4 mmol, 2.00 eq) was added dropwise at 0° C., and the reaction was stirred at 25° C. for 2 h. The mixture was quenched with an ammonium chloride solution (100 mL) and extracted with ethyl acetate (3×50.0 mL). The combined organic layers were washed with brine (40.0 mL), dried over sodium sulfate, filtered, and concentrated under reduced pressure to give a residue. The residue was purified by silica gel column chromatography (petroleum ether/ethyl acetate=10/1 to 5/1) to afford 3-(bromodifluoromethoxy)-2-methyl-5-nitropyridine.

Step 3: To a solution of 3-(bromodifluoromethoxy)-2-methyl-5-nitropyridine (180 mg, 636 μmol, 1.00 eq) in dichloromethane (3.00 mL) was added silver tetrafluoroborate (186 mg, 954 μmol, 1.50 eq) in portions. The reaction was stirred at 25° C. for 1 h. The mixture was filtered, and the filtrate was concentrated under reduced pressure to afford 2-methyl-5-nitro-3-(trifluoromethoxy)pyridine.

Step 4: A mixture of 2-methyl-5-nitro-3-(trifluoromethoxy)pyridine (100 mg, 450 μmol, 1.00 eq) and palladium on carbon (10% weight on C) (10.0 mg) in methanol (300 mL) was stirred at 25° C. for 2 h under hydrogen atmosphere (15 psi). The mixture was filtered over Celite, and the filtrate was concentrated under reduced pressure to afford 6-methyl-5-(trifluoromethoxy)pyridin-3-amine.

Compound 65: General procedure A with variant iv) was used for the preparation from compound VI employing 3-ethylisothiazol-5-amine.

¹H NMR (400 MHz, DMSO-d₆) δ=11.82 (s, 1H), 10.99 (s, 1H), 7.63 (s, 1H), 7.54-7.47 (m, 2H), 6.67 (s, 1H), 5.11 (dd, J=4.9, 13.3 Hz, 1H), 4.47-4.37 (m, 1H), 4.34-4.24 (m, 1H), 3.11-3.01 (m, 2H), 2.94-2.85 (m, 1H), 2.84-2.78 (m, 2H), 2.69-2.59 (m, 3H), 2.39 (br dd, J=4.1, 12.9 Hz, 1H), 2.05-1.95 (m, 1H), 1.18 (t, J=7.6 Hz, 3H). MS (ESI) m/z 427.2 [M+H]⁺

Step 1: To a solution of 3-oxopentanenitrile (2.00 g, 20.6 mmol, 1.00 eq) in toluene (60.0 mL) were added ammonium formate (4.00 g, 63.4 mmol, 3.08 eq) and acetic acid (0.12 mL, 2.10 mmol, 0.10 eq). The reaction was stirred at 130° C. for 12 h with a Dean-Stark trap. After cooling to room temperature, the mixture was filtered, and the filtrate was concentrated under reduced pressure to afford (Z)-3-aminopent-2-enenitrile.

Step 2: To a solution of thioacetamide (2.80 g, 37.3 mmol, 1.99 eq) and (Z)-3-aminopent-2-enenitrile (1.80 g, 18.7 mmol, 1.00) in dioxane (10.0 mL) was added hydrochloric acid/dioxane (4 M, 20.0 mL, 4.27 eq). The reaction was stirred at 20° C. for 16 h. The mixture was poured into ammonium hydroxide (28%, 20.0 mL) at 0° C., and then diluted with ethyl acetate (200 mL) and water (200 mL). The organic layer was separated and concentrated under reduced pressure to afford (Z)-3-aminopent-2-enethioamide.

Step 3: To a solution of (Z)-3-aminopent-2-enethioamide (2.20 g, 16.9 mmol, 1.00 eq) in methanol (20.0 mL) was added hydrogen peroxide 30% (6.19 mL, 64.3 mmol, 3.81 eq). The reaction was stirred at 20° C. for 2 h. The mixture was quenched with 10% aqueous sodium sulfite, then diluted with water (50.0 mL) and ethyl acetate (100 mL). The organic layer was separated and concentrated under reduced pressure. The residue was purified by column chromatography on silica gel (petroleum ether/ethyl acetate=3/1) to give 3-ethylisothiazol-5-amine.

Compound 66: General procedure A with variant i) was used for the preparation from compound VI employing 3-(difluoromethoxy)-5-(morpholinomethyl)aniline.

¹H NMR (400 MHz, DMSO-d₆) δ=10.99 (s, 1H), 10.94-10.70 (m, 1H), 10.36 (s, 1H), 7.67 (s, 1H), 7.63 (s, 1H), 7.53 (s, 2H), 7.49 (s, 1H), 7.23 (d, J=73.2 Hz, 1H), 7.18 (br s, 1H), 5.10 (dd, J=5.1, 13.3 Hz, 1H), 4.40 (s, 1H), 4.31 (br s, 3H), 3.94 (br d, J=11.5 Hz, 2H), 3.74 (br s, 2H), 3.24 (br d, J=12.1 Hz, 2H), 3.15-3.08 (m, 2H), 3.04 (br t, J=7.3 Hz, 2H), 2.96-2.87 (m, 1H), 2.74 (br t, J=7.3 Hz, 2H), 2.62-2.58 (m, 1H), 2.40 (br dd, J=4.5, 13.0 Hz, 1H), 2.04-1.96 (m, 1H). MS (ESI) m/z 557.3 [M+H]⁺

Step 1: To a solution of 3-hydroxy-5-nitrobenzoic acid (1.80 g, 9.83 mmol, 1.00 eq) in dimethylformamide (10.0 mL) were added morpholine (0.86 mL, 9.83 mmol, 1.00 eq), 0-(7-azabenzotriazol-1-yl)-N,N,N′,N′-tetramethyluronium hexafluorophosphate (7.45 g, 19.6 mmol, 2.00 eq) and diisopropylethylamine (5.16 mL, 29.6 mmol, 3.00 eq). The reaction was stirred at 25° C. for 2 h. The mixture was extracted with ethyl acetate/water (200 ml/100 ml). The organic layer was collected, and the solvents were partly removed under reduced pressure to give a concentrated solution. The solution was purified by reversed phase preparative HPLC to afford (3-hydroxy-5-nitrophenyl)(morpholino)methanone.

Step 2: To a solution of (3-hydroxy-5-nitrophenyl)(morpholino)methanone (1.77 g, 7.02 mmol, 1.00 eq) in dimethylformamide (15.0 mL) were added (2-chloro-2,2-difluoro-acetyl)oxysodium (2.67 g, 17.5 mmol, 2.50 eq) and cesium carbonate (4.57 g, 14.0 mmol, 2.00 eq). The reaction was stirred at 100° C. for 2 h. The mixture was extracted with water/ethyl acetate (100 ml/100 ml). The organic layer was collected, and the solvents were partly removed under reduced pressure to give a concentrated solution. The solution was purified by reversed phase preparative HPLC to afford (3-(difluoromethoxy)-5-nitrophenyl)(morpholino)methanone.

Step 3: To a solution of (3-(difluoromethoxy)-5-nitrophenyl)(morpholino)methanone (1.39 g, 4.60 mmol, 1.00 eq) in tetrahydrofuran (5.00 mL) was added borane dimethyl sulfide complex (2 M in THF) (0.92 mL, 2.00 eq) at 25° C. The mixture was stirred at 25° C. for 0.5 h, then at 60° C. for 1.5 h. Methanol (5.00 mL) was added, and the mixture was extracted with water/ethyl acetate (50.0 ml/50.0 ml). The organic layer was collected, and the solvents were partly removed under reduced pressure to give a concentrated solution. The solution was purified by reversed phase preparative HPLC to afford 4-(3-(difluoromethoxy)-5-nitrobenzyl)morpholine.

Step 4: To a solution of 4-(3-(difluoromethoxy)-5-nitrobenzyl)morpholine (795 mg, 2.76 mmol, 1.00 eq) in methanol (15.0 mL) and water (5.00 mL) were added iron power (770 mg, 13.8 mmol, 5.00 eq) and ammonium chloride (1.18 g, 22.0 mmol, 8.00 eq). The reaction was stirred at 80° C. for 2 h. The mixture was filtered, and the filtrate was concentrated under reduced pressure to give a concentrated solution. The solution was extracted with ethyl acetate/saturated sodium bicarbonate (40.0 ml/10.0 ml). The organic layer was collected, dried over sodium sulfate, filtered, and concentrated under reduced pressure to afford 3-(difluoromethoxy)-5-(morpholinomethyl)aniline.

### Compound 67

Step 1: To a solution of methyl 2-methyl-5-nitrobenzoate (10.0 g, 51.2 mmol, 1.00 eq) in trichloromethane (100 mL) was added N-bromosuccinimide (9.12 g, 51.2 mmol, 1.00 eq) and benzoyl peroxide (2.48 g, 10.3 mmol, 0.200 eq) in portions. The mixture was stirred at 90° C. for 12 h under nitrogen. The mixture was concentrated to give a crude product. The crude product was purified by silica gel chromatography (petroleum ether/ethyl acetate=1/0 to 5/1) to give methyl 2-(bromomethyl)-5-nitrobenzoate. ¹H NMR (400 MHz, CDCl₃) δ=8.75 (d, J=2.4 Hz, 1H), 8.27 (dd, J=2.5, 8.5 Hz, 1H), 7.62 (d, J=8.5 Hz, 1H), 4.93 (s, 2H), 3.94 (s, 3H).

Step 2: To a solution of methyl 2-(bromomethyl)-5-nitrobenzoate (4.50 g, 16.4 mmol, 1.00 eq) and 3-aminopiperidine-2,6-dione (2.70 g, 16.4 mmol, 1.00 eq, HCl) in acetonitrile (50.0 mL) was added N,N-diisopropylethylamine (6.37 g, 49.3 mmol, 8.58 mL, 3.00 eq) dropwise. The mixture was stirred at 90° C. for 12 h. The mixture was concentrated to give crude product. The crude product was triturated with ethyl acetate/water=2/1 (30.0 mL) and filtered. The filter cake was dried to give 3-(6-nitro-1-oxoisoindolin-2-yl)piperidine-2,6-dione. ¹H NMR (400 MHz, DMSO-d₆) δ=11.05 (s, 1H), 8.63-8.31 (m, 2H), 7.93 (d, J=8.3 Hz, 1H), 5.17 (dd, J=5.1, 13.3 Hz, 1H), 4.76-4.39 (m, 2H), 2.99-2.86 (m, 1H), 2.62 (br d, J=17.6 Hz, 1H), 2.48-2.36 (m, 1H), 2.09-2.00 (m, 1H). MS (ESI) m/z 290.2 [M+H]⁺

Step 3: To a solution of 3-(6-nitro-1-oxoisoindolin-2-yl)piperidine-2,6-dione (2.62 g, 9.06 mmol, 1.00 eq) in dimethyl formamide (3.00 mL) and tetrahydrofuran (25.0 mL) was added palladium/carbon (500 mg 10% purity) in portions. The mixture was stirred at 20° C. for 1 h under hydrogen (15 psi). The mixture was filtered and the filtrate was concentrated to give crude product. The crude product was triturated with water (20.0 mL) and filtered. The filter cake was dried to give 3-(6-amino-1-oxoisoindolin-2-yl)piperidine-2,6-dione. ¹H NMR (400 MHz, DMSO-d₆) δ=10.95 (s, 1H), 7.22 (d, J=8.1 Hz, 1H), 6.91-6.75 (m, 2H), 5.34 (s, 2H), 5.04 (dd, J=5.1, 13.2 Hz, 1H), 4.29-4.22 (m, 1H), 4.16-4.10 (m, 1H), 2.93 (br d, J=5.4 Hz, 0.5H), 2.89-2.83 (m, 0.5H), 2.59 (br d, J=19.2 Hz, 1H), 2.36 (dq, J=4.5, 13.2 Hz, 1H), 1.98 (dtd, J=2.2, 5.2, 12.6 Hz, 1H). (The H NMR comes from the pilot run). MS (ESI) m/z 260.2 [M+H]⁺

Step 4: To a solution of 3-(6-amino-1-oxoisoindolin-2-yl)piperidine-2,6-dione (1.68 g, 6.48 mmol, 1.00 eq) and tert-butyl 2-bromoacetate (1.26 g, 6.48 mmol, 958 uL, 1.00 eq) in dimethyl formamide (15.0 mL) was added N,N-diisopropylethylamine (2.51 g, 19.4 mmol, 3.39 mL, 3.00 eq) dropwise. The mixture was stirred at 60° C. for 12 h. The mixture was concentrated to give crude product. The crude product was purified by reversed-phase HPLC (0.1% FA condition) to give tert-butyl 2-((2-(2,6-dioxopiperidin-3-yl)-3-oxoisoindolin-5-yl)amino)acetate. ¹H NMR (400 MHz, DMSO-d₆) δ=10.96 (s, 1H), 7.29 (d, J=8.3 Hz, 1H), 6.87 (dd, J=2.3, 8.3 Hz, 1H), 6.78 (d, J=2.1 Hz, 1H), 6.28 (t, J=6.4 Hz, 1H), 5.06 (dd, J=5.1, 13.3 Hz, 1H), 4.32-4.24 (m, 1H), 4.20-4.12 (m, 1H), 3.84 (d, J=6.5 Hz, 2H), 2.96-2.84 (m, 1H), 2.59 (br dd, J=2.1, 15.5 Hz, 1H), 2.44-2.34 (m, 1H), 1.98 (dtd, J=2.3, 5.2, 12.6 Hz, 1H), 1.42 (s, 9H). MS (ESI) m/z 374.2 [M+H]⁺

Step 5: To a mixture of tert-butyl 2-((2-(2,6-dioxopiperidin-3-yl)-3-oxoisoindolin-5-yl)amino)acetate (700 mg, 1.87 mmol, 1.00 eq) in dichloromethane (7.00 mL) was added trifluoroacetic acid (4.62 g, 40.5 mmol, 3.00 mL, 21.6 eq) dropwise. The mixture was stirred at 25° C. for 12 h. The mixture was concentrated to give a crude product. The crude product was triturated with ethyl acetate (20.0 mL) and filtered. The filter cake was dried to give 2-((2-(2,6-dioxopiperidin-3-yl)-3-oxoisoindolin-5-yl)amino)acetic acid. MS (ESI) m/z 318.0 [M+H]⁺

Step 6: To a solution of 2-((2-(2,6-dioxopiperidin-3-yl)-3-oxoisoindolin-5-yl)amino)acetic acid (250 mg, 788 umol, 1.00 eq), 3-chloro-4-methyl-5-(morpholinomethyl)aniline (247 mg, 1.02 mmol, 1.30 eq) and triethylamine (319 mg, 3.15 mmol, 439 uL, 4.00 eq) in dimethyl formamide (3.00 mL) was added 1-(3-dimethylaminopropyl)-3-ethylcarbodiimide hydrochloride (302 mg, 1.58 mmol, 2.00 eq) and 6-chloro-1-hydroxibenzotriazol (213 mg, 1.58 mmol, 2.00 eq) in portions. The mixture was stirred at 25° C. for 12 h. The mixture was filtered. The filtrate was purified by prep-HPLC and prep-HPLC then lyophilized to give N-(3-chloro-4-methyl-5-(morpholinomethyl)phenyl)-2-((2-(2,6-dioxopiperidin-3-yl)-3-oxoisoindolin-5-yl)amino)acetamide. ¹H NMR (400 MHz, DMSO-d₆) δ=10.95 (s, 1H), 10.13 (s, 1H), 7.80 (d, J=2.1 Hz, 1H), 7.40 (d, J=2.1 Hz, 1H), 7.31 (d, J=8.3 Hz, 1H), 6.94 (dd, J=2.3, 8.3 Hz, 1H), 6.82 (d, J=2.1 Hz, 1H), 6.35 (t, J=6.2 Hz, 1H), 5.06 (dd, J=5.1, 13.3 Hz, 1H), 4.33-4.23 (m, 1H), 4.20-4.12 (m, 1H), 3.94 (d, J=6.1 Hz, 2H), 3.56 (br t, J=4.4 Hz, 4H), 3.42 (s, 2H), 2.98-2.83 (m, 1H), 2.59 (td, J=1.9, 15.4 Hz, 1H), 2.40-2.32 (m, 5H), 2.30 (s, 3H), 2.03-1.91 (m, 1H). MS (ESI) m/z 540.1 [M+H]⁺

### Compound 68

Step 1: To a solution of methyl 5-hydroxy-2-methyl-benzoate (2.00 g, 12.0 mmol, 1.00 eq) in dichloromethane (20.0 mL) was added imidazole (1.64 g, 24.1 mmol, 2.00 eq) and tert-butyl-chloro-dimethyl-silane (2.72 g, 18.1 mmol, 2.21 mL, 1.50 eq). The mixture was stirred at 20° C. for 12 h. The reaction mixture was diluted with water (40.0 mL) and extracted with dichloromethane (3×100 mL). The combined organic layers were washed with brine (2×100 mL), dried over magnesium sulfate, filtered and concentrated under reduced pressure to give a residue. The residue was purified by column chromatography (SiO₂, petroleum ether/ethyl acetate=1/0) and then concentrated under reduced pressure to give methyl 5-((tert-butyldimethylsilyl)oxy)-2-methylbenzoate. ¹H NMR (400 MHz, CDCl₃) δ=7.38 (d, J=2.8 Hz, 1H), 7.09 (d, J=8.2 Hz, 1H), 6.89 (dd, J=2.8, 8.3 Hz, 1H), 3.89 (s, 3H), 2.51 (s, 3H), 0.99 (s, 9H), 0.20 (s, 6H).

Step 2: To a solution of methyl 5-((tert-butyldimethylsilyl)oxy)-2-methylbenzoate (3.00 g, 10.7 mmol, 1.00 eq) in trichloromethane (60.0 mL) was added benzoyl peroxide (518 mg, 2.14 mmol, 0.200 eq) and N-Bromosuccinimide (1.90 g, 10.7 mmol, 1.00 eq). The mixture was stirred at 90° C. for 12 h under nitrogen. The reaction mixture was filtered and concentrated under reduced pressure to give a residue. The residue was purified by column chromatography (SiO₂, petroleum ether/ethyl acetate=1/0 to 10/1) to give methyl 2-(bromomethyl)-5-((tert-butyldimethylsilyl)oxy)benzoate.

¹H NMR (400 MHz, CDCl₃) δ=7.42 (d, J=2.6 Hz, 1H), 7.32 (d, J=8.4 Hz, 1H), 6.95 (dd, J=2.8, 8.3 Hz, 1H), 4.93 (s, 2H), 3.94 (s, 3H), 1.00 (s, 9H), 0.23 (s, 6H).

Step 3: To a solution of methyl 2-(bromomethyl)-5-((tert-butyldimethylsilyl)oxy)benzoate (1.70 g, 4.73 mmol, 1.00 eq) in acetonitrile (30.0 mL) was added N,N-diisopropylethylamine (1.83 g, 14.2 mmol, 2.47 mL, 3.00 eq) and 3-aminopiperidine-2,6-dione (606 mg, 3.68 mmol, 1.00 eq, hydrochloric acid). The mixture was stirred at 90° C. for 12 h. The mixture was filtered and concentrated under reduced pressure. The residue was purified by prep-HPLC and then lyophilized to give 3-(6-((tert-butyldimethylsilyl)oxy)-1-oxoisoindolin-2-yl)piperidine-2,6-dione. ¹H NMR (400 MHz, DMSO-d₆) δ=10.99 (s, 1H), 7.49 (d, J=8.2 Hz, 1H), 7.19-7.03 (m, 2H), 5.09 (dd, J=5.0, 13.3 Hz, 1H), 4.44-4.19 (m, 2H), 2.95-2.85 (m, 1H), 2.61 (br s, 1H), 2.38 (br dd, J=4.2, 13.2 Hz, 1H), 2.05-1.95 (m, 1H), 0.96 (s, 9H), 0.21 (s, 6H).

Step 4: To a solution of 3-(6-((tert-butyldimethylsilyl)oxy)-1-oxoisoindolin-2-yl)piperidine-2,6-dione (4.00 g, 10.7 mmol, 1.00 eq) in tetrahydrofuran (40.0 mL) was added tetra-n-butylammoniumfluoride trihydrate (3.37 g, 10.7 mmol, 1.00 eq). The mixture was stirred at 25° C. for 2 h. The reaction mixture was diluted with ammonium chloride (40.0 mL) and extracted with ethyl acetate (3×100 mL). The combined organic layers were washed with brine (2×40.0 mL), dried over magnesium sulfate, filtered and concentrated under reduced pressure to give a residue. The residue was purified by prep-HPLC and then lyophilized to give 3-(6-hydroxy-1-oxoisoindolin-2-yl)piperidine-2,6-dione. ¹H NMR (400 MHz, DMSO-d₆) δ=10.97 (s, 1H), 9.84 (s, 1H), 7.39 (d, J=8.2 Hz, 1H), 7.08-6.97 (m, 2H), 5.06 (dd, J=5.2, 13.2 Hz, 1H), 4.40-4.11 (m, 2H), 2.95-2.84 (m, 1H), 2.59 (br d, J=17.2 Hz, 1H), 2.44-2.33 (m, 1H), 1.99 (ddd, J=2.6, 5.2, 10.1 Hz, 1H).

Step 5: To a solution of 3-(6-hydroxy-1-oxo-isoindolin-2-yl)piperidine-2,6-dione (160 mg, 615 umol, 1.00 eq) and tert-butyl 2-bromoacetate (120 mg, 615 umol, 90.9 uL, 1.00 eq) in dimethylformamide (2.00 mL) was added potassium iodide (10.2 mg, 61.5 umol, 0.100 eq) and potassium carbonate (255 mg, 1.84 mmol, 3.00 eq). The mixture was stirred at 60° C. for 12 h. The reaction mixture was diluted with dimethylformamide (50.0 mL) and filtered to give a mixture. The filtrate was purified by prep-HPLC then lyophilized to give tert-butyl 2-((3-oxo-2-(6-oxopiperidin-3-yl)isoindolin-5-yl)oxy)acetate. MS (ESI) m/z 319.0 [M+H]⁺

Step 6: To a solution of tert-butyl 2-((3-oxo-2-(6-oxopiperidin-3-yl)isoindolin-5-yl)oxy)acetate (60.0 mg, 160 umol, 1.00 eq) in dichloromethane (0.600 mL) was added trifluoroacetic acid (924 mg, 8.10 mmol, 0.600 mL, 50.6 eq). The mixture was stirred at 25° C. for 2 h. The reaction mixture was concentrated under reduced pressure to remove dichloromethane (0.600 mL), filtered and concentrated under reduced pressure to give a residue. The residue was purified by prep-HPLC and further purification by prep-HPLC then lyophilized to give 2-((2-(2,6-dioxopiperidin-3-yl)-3-oxoisoindolin-5-yl)oxy)acetic acid. ¹H NMR (400 MHz, DMSO-d₆) δ=10.98 (s, 1H), 7.51 (d, J=8.4 Hz, 1H), 7.20 (dd, J=2.4, 8.3 Hz, 1H), 7.15 (d, J=2.4 Hz, 1H), 5.10 (dd, J=5.2, 13.3 Hz, 1H), 4.76 (s, 2H), 4.41-4.21 (m, 2H), 2.91 (ddd, J=5.4, 13.6, 17.5 Hz, 1H), 2.59 (br d, J=18.4 Hz, 1H), 2.45-2.33 (m, 1H), 2.05-1.95 (m, 1H). MS (ESI) m/z 319.1 [M+H]⁺

Step 7: To a solution of 2-((2-(2,6-dioxopiperidin-3-yl)-3-oxoisoindolin-5-yl)oxy)acetic acid (200 mg, 628 umol, 1.00 eq) and 3-chloro-4-methyl-5-(morpholinomethyl)aniline (166 mg, 691 umol, 1.10 eq) in dimethylformamide (3.00 mL) was added 1-(3-dimethylaminopropyl)-3-ethylcarbodiimide hydrochloride (241 mg, 1.26 mmol, 2.00 eq), N,N-diisopropylethylamine (244 mg, 1.89 mmol, 328 uL, 3.00 eq) and 1-hydroxybenzotriazole (170 mg, 1.26 mmol, 2.00 eq). The mixture was stirred at 25° C. for 12 h. The reaction mixture was diluted with dimethylformamide (2.00 mL) and filtered to give a mixture. The filtrate was purified by prep-HPLC, then lyophilized to give N-(3-chloro-4-methyl-5-(morpholinomethyl)phenyl)-2-((2-(2,6-dioxopiperidin-3-yl)-3-oxoisoindolin-5-yl)oxy)acetamide. ¹H NMR (400 MHz, DMSO-d₆) δ=10.98 (s, 1H), 10.20 (s, 1H), 8.15 (s, 1H), 7.81 (d, J=2.2 Hz, 1H), 7.55 (d, J=8.2 Hz, 1H), 7.46 (d, J=2.0 Hz, 1H), 7.32-7.27 (m, 2H), 5.10 (dd, J=5.2, 13.3 Hz, 1H), 4.80 (s, 2H), 4.44-4.22 (m, 2H), 3.58-3.54 (m, 4H), 3.43 (s, 2H), 2.91 (ddd, J=5.4, 13.6, 17.5 Hz, 1H), 2.59 (br dd, J=1.8, 15.7 Hz, 1H), 2.42-2.34 (m, 5H), 2.30 (s, 3H), 2.04-1.96 (m, 1H). MS (ESI) m/z 541.2 [M+H]⁺

### Compound 69

To a solution of 2-((2-(2,6-dioxopiperidin-3-yl)-3-oxoisoindolin-5-yl)oxy)acetic acid (200 mg, 628 umol, 1.00 eq) and 3-chloro-4-methylaniline (97.9 mg, 691 umol, 1.10 eq) in dimethylformamide (2.00 mL) was added 1-hydroxybenzotriazole (170 mg, 1.26 mmol, 2.00 eq), N,N-diisopropylethylamine (244 mg, 1.89 mmol, 328 uL, 3.00 eq) and 1-(3-dimethylaminopropyl)-3-ethylcarbodiimide hydrochloride (241 mg, 1.26 mmol, 2.00 eq). The mixture was stirred at 25° C. for 12 h. The reaction mixture was diluted with dimethylformamide (2.00 mL) filtered and to give a mixture. The filtrate was purified by prep-HPLC and lyophilized to give N-(3-chloro-4-methylphenyl)-2-((2-(2,6-dioxopiperidin-3-yl)-3-oxoisoindolin-5-yl)oxy)acetamide. ¹H NMR (400 MHz, DMSO-d₆) δ=10.97 (br s, 1H), 10.21 (s, 1H), 7.82 (d, J=2.0 Hz, 1H), 7.55 (d, J=8.2 Hz, 1H), 7.46 (dd, J=2.0, 8.3 Hz, 1H), 7.37-7.23 (m, 3H), 5.10 (dd, J=5.2, 13.3 Hz, 1H), 4.81 (s, 2H), 4.43-4.22 (m, 2H), 2.98-2.84 (m, 1H), 2.62-2.56 (m, 1H), 2.45-2.34 (m, 1H), 2.27 (s, 3H), 2.05-1.95 (m, 1H). MS (ESI) m/z 442.1 [M+H]⁺

Protocol for Fluorescent Polarization Assay

Compound activity was monitored in a fluorescence polarization (FP) homogeneous assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Unless otherwise stated, all reagents were purchased from Sigma Aldrich. Enzymatic reactions were conducted in Perkin-Elmer Black 384 well ProxiPlate Plus (catalogue no. 6008269) in 10 μL total volume. Full length wild-type cereblon CRBN (80.0 nM, 10 μL) was incubated in assay buffer containing 20 mM HEPES (pH 8.0), 150 NaCl, 0.5 mM TCEP and 0.05% Tween 20 in the presence or absence of compound (300 nL). Inhibitors were stored as 10 mM DMSO stocks in an inert environment (low humidity, dark, low oxygen, room temperature) using the Storage Pod System. Compounds and DMSO were dispensed using the Echo E5XX (Labcyte Inc. USA) to give concentrations from 300 to 0.937 or 3000 to 9.3 nM in a 12 data point curve. Mutant YWAA CRBN (80.0 nM, 10 μL) which does not interact with the fluorescent probe was used as a negative control for the assay. Following incubation at room temperature for 30 min, the assay was initiated by dispensing the probe to a final concentration of 5 nM (2.5 nL of a 20 μM stock) using the Echo E5XX. FP was measured after a period of 12 hours using a Pherastar plate reader (BMG Labtech, Germany) exciting at 590 nm and measuring the amount of parallel and perpendicular light at 675 nm. The FP signal was subsequently normalized to the no-compound control (i.e., DMSO). Analysis and IC50 values were derived using Dotmatics (Dotmatics UK) software.

TABLE 2
IC50 values determined in the fluorescence polarization
assay indicating the cereblon binding

| | rFP | | rFP | | rFP |
|---|---|---|---|---|---|
| Compound | IC50 | Compound | IC50 | Compound | IC50 |
| 1 | A | 2 | A | 3 | A |
| 4 | B | 5 | C | 6 | D |
| 7 | B | 8 | B | 9 | B |
| 10 | B | 11 | D | 12 | B |
| 13 | D | 14 | B | 15 | C |
| 16 | A | 17 | B | 18 | D |
| 19 | B | 20 | B | 21 | D |
| 22 | B | 23 | D | 24 | C |
| 25 | B | 26 | B | 27 | D |
| 28 | B | 29 | C | 30 | B |
| 31 | B | 32 | B | 33 | B |
| 34 | A | 35 | B | 36 | C |
| 37 | B | 38 | A | 39 | A |
| 40 | A | 41 | C | 42 | C |
| 43 | C | 44 | B | 45 | B |
| 46 | B | 47 | B | 48 | C |
| 49 | C | 50 | A | 51 | A |
| 52 | D | 53 | B | 54 | B |
| 55 | C | 56 | D | 57 | A |
| 58 | C | 59 | B | 60 | D |
| 61 | A | 62 | | 63 | A |
| 64 | C | 65 | D | 66 | D |
| 67 | A | 68 | A | 69 | B |

Table 2 assigns each compound a code indicating the ability for cereblon binding by means of their IC50 values: A, B, C or D. According to the code, A represents an IC50 value of ≤500 nM, B represents an IC50 value>500 nM and ≤1100 nM, C represents an IC50 value of >1100 nM and ≤1700 nM and D represents an IC50 value of >1700 nM.

In some embodiments, the disclosure is directed to compounds with an IC50 value of less than 1100 nM, i.e. directed to compounds 1, 2, 3, 4, 7, 8, 9, 10, 12, 14, 16, 17, 19, 20, 22, 25, 26, 28, 30, 31, 32, 33, 34, 35, 37, 38, 39, 40, 44, 45, 46, 47, 50, 51, 53, 54, 57, 59, 61 and 63.

### Example 4: Compound Binding by Immunofluorescence Assay

The representative compounds were tested in an immunofluorescence assay for their activity to bind to degrade GSPT1. CAL-Si cells were purchased from DSMZ (cat. Number ACC302), sub-cultured in 90% Dulbecco's MEM (4.5 g/L glucose, Gibco 11965)+10% 10 heat inactivated FBS (BioConcept, 2-01F136I) and incubated at 37° C., 5% CO₂. For the assay, imaging microtiterplate Cell Carrier 96 Ultra (Perkin Elmer 6055302) were pre-coated with Fibronectin (Sigma F085, 30 μl at 0.2 μg/ml) in PBS (100 μl, Gibco 14190) for 45 min at room temperature, rinsed with PBS and CAL-Si cells (30K cells/well) were plated and let to adhere overnight. Cells were treated with compounds typically using a serial dilution ranging from 30 μM to 0.1 nM for 6 hours. Compounds were stored at 10 mM DMSO stocks. Vehicle (DMSO), positive (CC-885, 10 μM) and rescue controls (positive control plus 0.2 μM bortezomib) were also included at this stage. Cells were subsequently rinsed with PBS and fixed in 10% Formalin solution (50 μl, Sigma HT5011)) for 20 mins at room temperature. Following three consecutive PBS washes (100 μl), cells were permeabilized in 0.1% Triton X-100 in PBS (Sigma 93443, 50 μl) for 15 mins at room temperature. Following three further PBS washes, 50p blocking buffer (1% BSA, Sigma A4503, in PBS) was added for 45 min for signal-to-noise reduction.

Primary antibody (human GSPT1, Sigma HPA052488) was diluted in blocking buffer (dil.1/300, 35 μl/well) and incubated with the cells overnight at 4° C. After three PBS washes, Alexa-fluor 488 coupled secondary antibodies (Invitrogen, A32731, dil.1/1000), Alexa-fluor 647-Phalloidin (Invitrogen, A22287, dil.1/200) and DAPI (Thermo, #62248, dil.1/1000) were diluted in blocking buffer and incubated with the samples for 2 hours at room temperature. After three final PBS washes, samples were conserved in 100p PBS in the dark, until measurement. Image acquisition was performed on the Operetta High-Content Imager (Perkin-Elmer). Fluorescence intensity of Alexa-Fluor 488 (GSPT1), Alexa-Fluor 647 (Actin) and DAPI (Nucleus) were measured. For the determination of GSPT1 DC₅₀ values, a custom algorithm implemented in the PerkinElmer image analysis software Harmony-Acapella® was developed. After user-defined setting of adjustment parameters, the analysis was run identically without human intervention for all image fields. DAPI staining of the nuclei was used to determine the location of cells using standard nuclei detection modules. Segmentation artifacts were removed by threshold-based filters for area, roundness and intensity. The outline of the cells was determined analogously from the sum of the normalized, smoothed DAPI and Actin channel, starting from each nucleus.

The Alexa-Fluor 488 (GSPT1) signal intensity in each cell was finally measured, in order to obtain a Mean intensity per cell. GSPT1 degradation (DC₅₀) was calculated after normalization to controls and data import in CDD vault Database, using non-linear regression.

TABLE 3
Activity for GSPT1 degradation

| Compound | Code | Compound | Code | Compound | Code |
|---|---|---|---|---|---|
| 1 | A | 2 | A | 3 | B |
| 4 | C | 5 | B | 6 | C |
| 7 | A | 8 | B | 9 | C |
| 10 | C | 11 | A | 12 | C |
| 13 | C | 14 | C | 15 | C |
| 16 | C | 17 | C | 18 | C |
| 19 | C | 20 | B | 21 | C |
| 22 | B | 23 | A | 24 | B |
| 25 | C | 26 | C | 27 | C |
| 28 | B | 29 | A | 30 | B |
| 31 | B | 32 | B | 33 | B |
| 34 | A | 35 | A | 36 | C |
| 37 | C | 38 | C | 39 | B |
| 40 | C | 41 | A | 42 | A |
| 43 | A | 44 | A | 45 | A |
| 46 | B | 47 | B | 48 | C |
| 49 | C | 50 | B | 51 | C |
| 52 | C | 53 | A | 54 | C |
| 55 | C | 56 | B | 57 | C |
| 58 | B | 59 | C | 60 | B |
| 61 | C | 62 | | 63 | B |
| 64 | B | 65 | B | 66 | B |
| 67 | C | 68 | C | 69 | C |

Table 3 assigns each compound a code indicating the ability for GSPT1 degradation: A, B or C. According to the code, A represents a DC50 value of ≤30 nM, B represents a DC50 value>30 nM and ≤300 nM and C represents a DC50 value of >300 nM.

In some embodiments, the compounds of any of formula I to X exhibit a DC50 value of 30 nM or less, i.e. compounds with code A. In some embodiments the compound is selected from the group consisting of 1, 22, 7, 11, 23, 29, 34, 35, 41, 42, 43, 44, 45 and 53:

| | DC50 | | DC50 | | DC50 |
|---|---|---|---|---|---|
| Compound | [nM] | Compound | [nM] | Compound | [nM] |
| 1 | 16 | 2 | 6 | 7 | 25 |
| 11 | 17 | 23 | 27 | 29 | 6 |
| 34 | 13 | 35 | 12 | 41 | 26 |
| 42 | 30 | 43 | 26 | 44 | 27 |
| 45 | 22 | 53 | 22 | | |

### Example 5: CK1alpha/Ikaros/Aiolos/ZFP91 Selectivity Determination by Western Blot Assay

MM1S cells were purchased from ATCC (cat. Number CRL-2974), sub-cultured in 9000 RPMI 1640 with 10% FBS, supplemented with Ix P/S and incubated at 37° C., 500 CO2. Compounds were stored as 10 mM DMSO stock. For the assay, MM1S cells (3 million cells/well) were plated in 6-well plates and incubated over night. Cells were treated with respective compounds using a serial dilution: 0.3 μM, 3 μM and 30 μM as well as a vehicle only (DMSO) control for 6 hours. Media with suspension cells was subsequently transferred to 15 mL conical tubes, wells rinsed twice with ice-cold PBS and merged with cell suspension in respective 15 mL conical tube. Cells were spinned down, supernatant aspirated, pellets resuspended in ice-cold PBS and transferred to microtubes. Cells were spinned down, supernatant aspirated and pellets resuspended in 120 μL RIPA lysis buffer supplemented with protease and phosphatase inhibitors. Cell lysates were incubated on ice for 20 minutes followed by centrifugation at >20,000×g for 5 min. Supernatants were transferred to fresh microtubes and stored at −80° C. Total protein concentration was determined using a BCA assay with a BSA standard curve and concentration of all samples was adjusted to 1 mg/mL. 25 μL 4×LDS sample buffer supplemented with 100 mM DTT was added to 75 μL sample. Samples were centrifuged (8,000×g, 1 min) and incubated at 95° C. for 5 min followed by another centrifugation step (8,000×g, 1 min). 20 μL of each sample was loaded on a 4-12% gel alongside a protein molecular weight marker. Gels were run in the presence of MOPS buffer at 80 Volts for 30 min, followed by 120 Volts for 1.5 h and proteins subsequently transferred onto nitrocellulose membranes at 20 Volts for 7 min using an iBlot2 Gel Transfer Device. Membranes were then cut horizontally into two pieces, covering 80-50 kDa and 50-25 kDa. Blocking the membranes was performed by gently shaking in 5% (w/v) skim milk in TSB-T for 1 hr at room temperature. All primary antibodies were used at a 1/1000 in 5% (w/v) BSA dissolved in TBST and incubated with membranes over night at 4° C. After three washes with 1×TBST for 5 min, HRP-coupled secondary antibodies diluted in 5% (w/v) BSA/TBST (Goat Anti-Rb IgG, dil. 1/10,000; Goat Anti-Mouse IgG, dil. 1/5000) were added for 1 hr at room temperature. After three washes with 1×TBST (5 minutes each), membranes were incubated with ECL reagent for 1 min at room temperature. Chemiluminescence signals were then detected using a LAS-4000 system with default settings and signals quantified using Image Studio Lite software (version 5.2). Membrane parts previously incubated with antibody against CK1alpha were stripped off antibodies by incubating with stripping buffer for 30 minutes followed by three washed with TBST (5 min each), blocking with 5% (w/v) skim milk for 1 hr, and incubation with primary antibody against GAPDH overnight at 4° C. Subsequent washes, incubation with secondary antibody and signal acquisition were performed as described above.

TABLE 4
Selectivity for relevant Zincfinger proteins:

| Compound | IKZF1 | IKZF3 | CK1alpha | ZFP91 | GSPT1 |
|---|---|---|---|---|---|
| 1 | A | A | B | B | F |
| 2 | A | C | C | C | F |

Table 4 assigns each compound a code indicating the ability for the degradation of IKZF1, IKZF3, CK1alpha and ZFP91: A, B, C, D, E or F. According to the code, A represents no degradation observed at 30 μM, B represents trace degradation at 30 μM (below 20%), C represents weak degradation at 30 μM (below 50%), D represents degradation at 30 μM (>90%), E represents degradation at 3 μM (>90%) and F represents degradation at 0.3 μM (>90%).

1. A compound of formula I 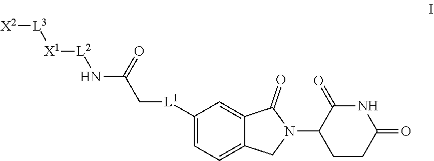
or a pharmaceutically acceptable salt or stereoisomer thereof,
wherein
X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of OH, halogen, linear or branched C₁₋₆ alkyl, CF₃, CHF₂, CMeF₂, —O—(CH₂)₂—OMe, OCF₃, OCHF₂, —CN, NH₂, C₁₋₆ alkoxy, and —C₁₋₆ alkylhydroxy;
or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₆ alkoxy, and C₁₋₄ alkylhydroxy;
X² is selected from the group consisting of hydrogen, C₃₋₆ cycloalkyl, —C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is selected from —CH₂—, O and NH;
L² is selected from covalent bond, and linear or branched C₁₋₆ alkyl; and
L³ is selected from covalent bond, linear or branched C₁₋₆ alkyl, —O—, and —C₁₋₄ alkoxy.

2. The compound of claim 1 or pharmaceutically acceptable salts or stereoisomers thereof, wherein X¹ is selected from the group consisting of linear or branched C₁₋₆ alkyl, C₃₋₆ cycloalkyl, C₆ aryl, 5-10 membered heteroaryl, and 4-6 membered heterocycloalkyl, wherein X¹ is unsubstituted or substituted with one or more substituents independently selected from the group consisting of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, and C₁₋₄ alkoxy; or X¹ together with the N atom of the amide forms a 4-8 membered heterocycloalkyl, which is unsubstituted or substituted with one or more substituents independently selected from the group consisting of halogen, linear or branched C₁₋₄ alkyl, CF₃, CHF₂, CMeF₂, —O—(CH₂)₂—OMe, OCF₃, OCHF₂, —CN, NH₂, —C₁₋₄ alkylhydroxy, and C₁₋₆ alkoxy.

3. The compound of claim 1 or 2, wherein X² is selected from the group consisting of H, C₃₋₆ cycloalkyl, C₆ aryl, 5-10 membered heteroaryl, and 4-8 membered heterocycloalkyl, wherein X² is unsubstituted or substituted with one or more substituents independently selected from the group consisting of linear or branched C₁₋₄ alkyl, —C₁₋₄ alkoxy, NMe₂, halogen, CF₃, CHF₂, CMeF₂, OCF₃, and OCHF₂.

4. The compound of any one of the preceding claims, wherein L¹ is —CH₂— and/or L² is selected from a covalent bond and —CH₂— and/or L³ is selected from the group consisting of a covalent bond, —CH₂— —O—CH₂—, —O—CH₂—CH₂— and —O—.

5. The compound of claim 1, wherein the compound is of formula II 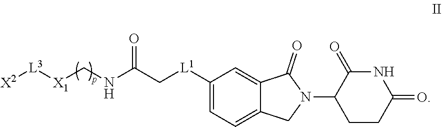
or a pharmaceutically acceptable salt or stereoisomer thereof,
wherein
X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, CF₃, CHF₂, —O—CHF₂, —O—(CH₂)₂—OMe, OCF₃, —CN, NH₂, C₁₋₆ alkoxy and C₁₋₆ alkylhydroxy;
or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₄ alkylhydroxy, and C₁₋₆ alkoxy;
X² is selected from the group consisting of hydrogen, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L¹ is selected from —CH₂—, O and NH;
L³ is selected from the group consisting of a covalent bond, linear or branched C₁₋₆ alkyl, —O—, and —C₁₋₄ alkoxy; and
p is 0, or 1.

6. The compound of any of the preceding claims, wherein the compound is, of formula III, IV, or V: 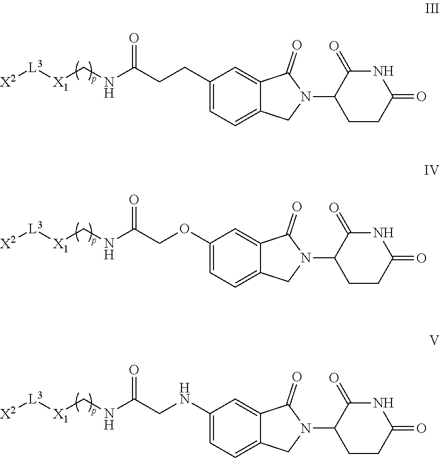
or a pharmaceutically acceptable salt or stereoisomer thereof,
wherein
X¹ is selected from the group consisting of linear or branched C₁₋₆ alkyl, C₃₋₆ cycloalkyl, C₆₋₁₀ aryl, 5-10 membered heteroaryl, and 4-8 membered heterocycloalkyl, wherein X¹ is unsubstituted or substituted with one or more substituents independently selected from the group consisting of halogen, linear or branched C₁₋₆ alkyl, CF₃, CHF₂, —O—CHF₂, —O—(CH₂)₂—OMe, OCF₃, —CN, NH₂, C₁₋₆ alkoxy and C₁₋₆ alkylhydroxy;
or X¹ together with the N atom of the amide forms a 4-8 membered heterocycloalkyl, which is unsubstituted or substituted with one or more substituents independently selected from the group consisting of halogen, linear or branched —C₁₋₆ alkyl, CF₃, CHF₂, CMeF₂, —O—(CH₂)₂—OMe, OCF₃, OCHF₂, —CN, NH₂, C₁₋₄ alkylhydroxy, and C₁₋₆ alkoxy;
X² is selected from the group consisting of hydrogen, C₃₋₆ cycloalkyl, C₆₋₁₀ aryl, 5-10 membered heteroaryl, and 4-8 membered heterocycloalkyl, wherein X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
L³ is selected from the group consisting of a covalent bond, linear or branched C₁₋₆ alkyl, —O—, and —C₁₋₄ alkoxy; and
p is 0 or 1.

7. The compound of claim 1, wherein the compound is of formula VI, VII or VIII: 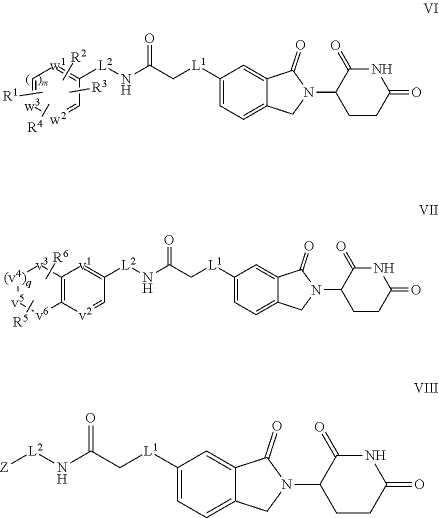
or a pharmaceutically acceptable salt or stereoisomer thereof,
wherein
each of w¹, w², and w³ is independently selected from the group consisting of C, N, S, and O, with the proviso that at least one of w¹, w², and w³ is C;
one or two of v³, v⁴, v⁵, and v⁶ is independently selected from C and O and the remaining of v³, v⁴, v⁵, and v⁶ are C;
each of v¹, and v² is independently selected from C and N;
L¹ is selected from —CH₂—, O and NH;
L² is selected from the group consisting of a covalent bond, and linear or branched C₁₋₆ alkyl;
each of R¹, R², R³, and R⁴ is independently selected from the group consisting of hydrogen, linear or branched —C₁₋₆ alkyl, —C₁₋₆ alkoxy, CF₃, CHF₂, CMeF₂, —O—(CH₂)₂—OMe, OCF₃, OCHF₂, —CN, NH₂, —C₁₋₆ alkylhydroxy, halogen, such as F, Cl, Br, e.g. F or Cl, and a group of formula -L³-X², wherein L³ is selected from the group consisting of a covalent bond, linear or branched C₁₋₆ alkyl, —O—, and —C₁₋₄ alkoxy, and X² is selected from the group consisting of C_{3≢}cycloalkyl, C₆-10 aryl, 5-10 membered heteroaryl, and 4-8 membered heterocycloalkyl, wherein
X² is unsubstituted or substituted with one or more substituents independently selected from the group consisting of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and C₁₋₄ alkylhydroxy;
each of R⁵, and R⁶ is independently selected from the group consisting of H, linear or branched C₁₋₄ alkyl, and halogen, preferably F, or Cl, or more preferably F;
Z is selected from the group consisting of linear or branched —C₁₋₆ alkyl, —C₃₋₆ cycloalkyl, —C₁₋₄ alkoxy, and 4-6 membered heterocycloalkyl, wherein Z is unsubstituted or substituted with a substituent selected from the group consisting of with C₁₋₄ alkyl, C₆ aryl, C₆ aryloxy, 6 membered heteroaryl and CF₃; or Z together with the N atom of the amide forms a 4-6 membered heterocycloalkyl, which is unsubstituted or substituted with a substituent selected from the group consisting of C₁₋₄ alkyl, C₆ aryl, C₆ aryloxy, 6 membered heteroaryl and CF₃;
q is 0 or 1; and
m is 0 or 1.

8. The compound of any of the preceding claims, wherein the compound is of formula X 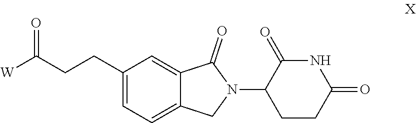
or a pharmaceutically acceptable salt or stereoisomer thereof,
wherein
W is selected from the group consisting of:

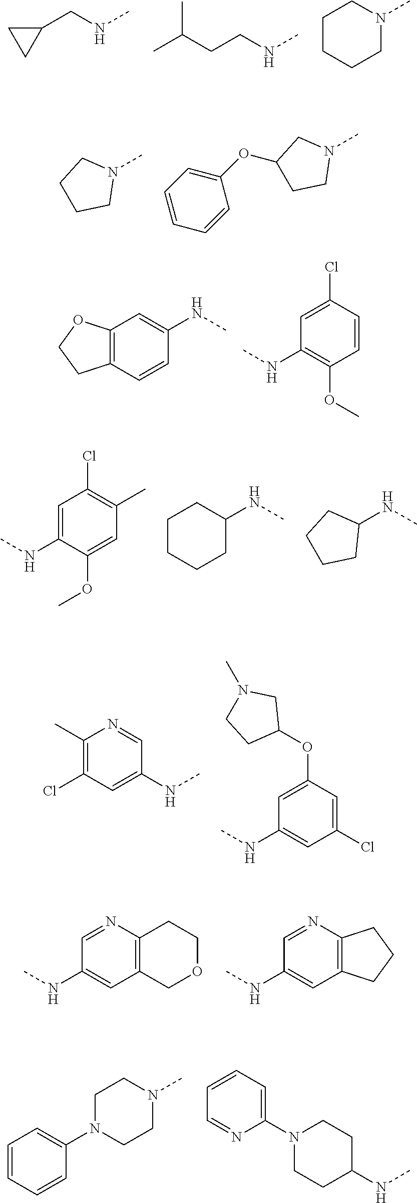

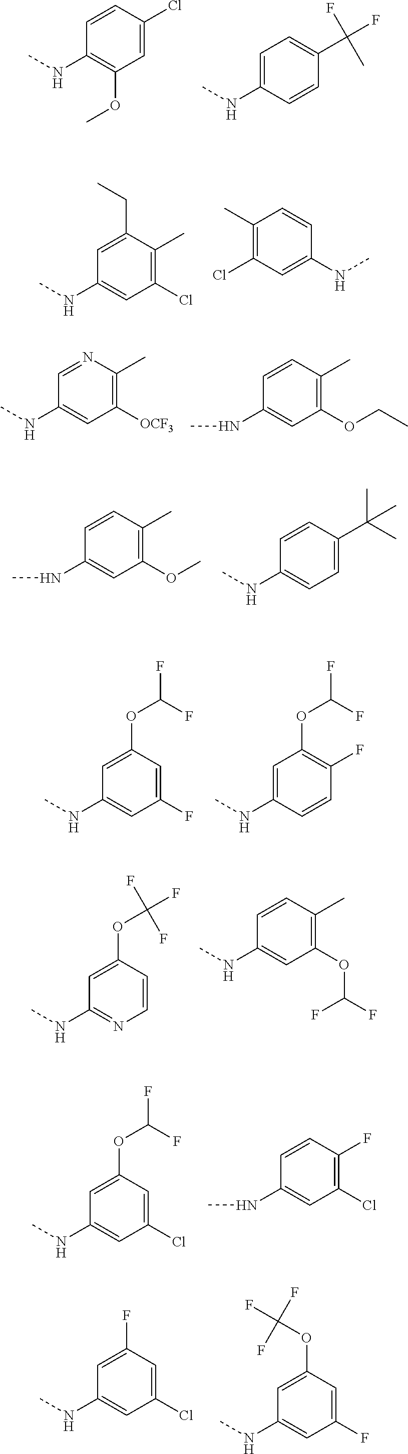

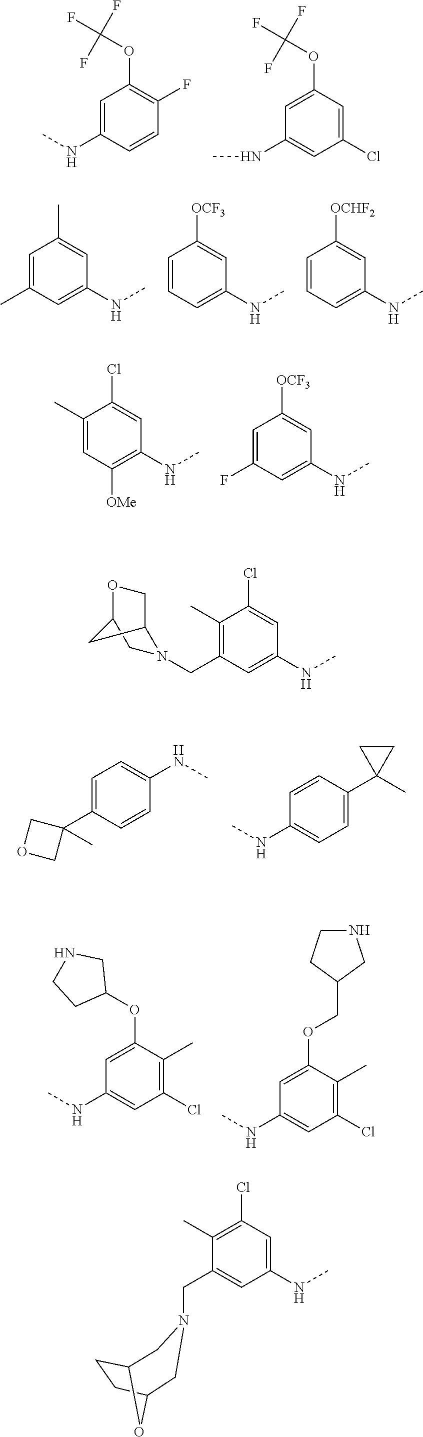

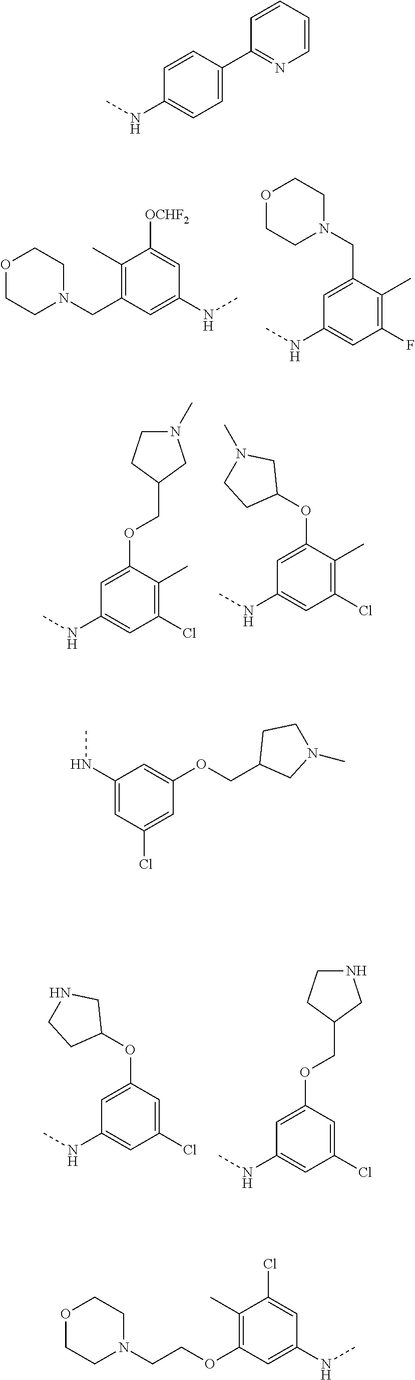

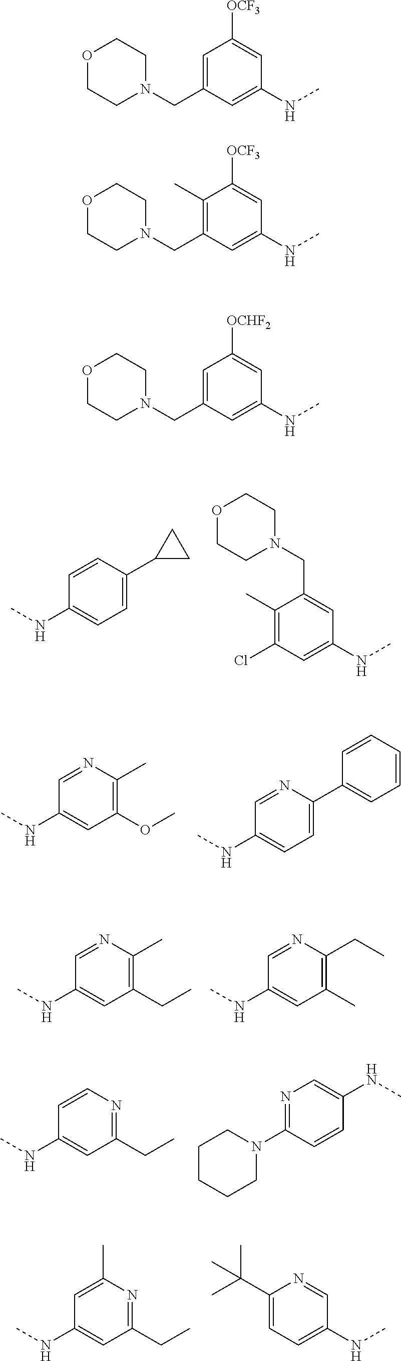

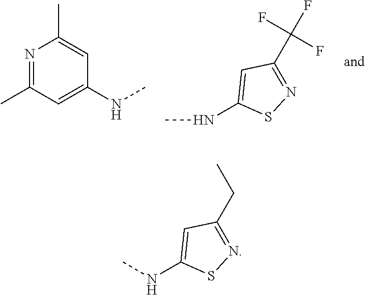

9. A compound selected from the group consisting of 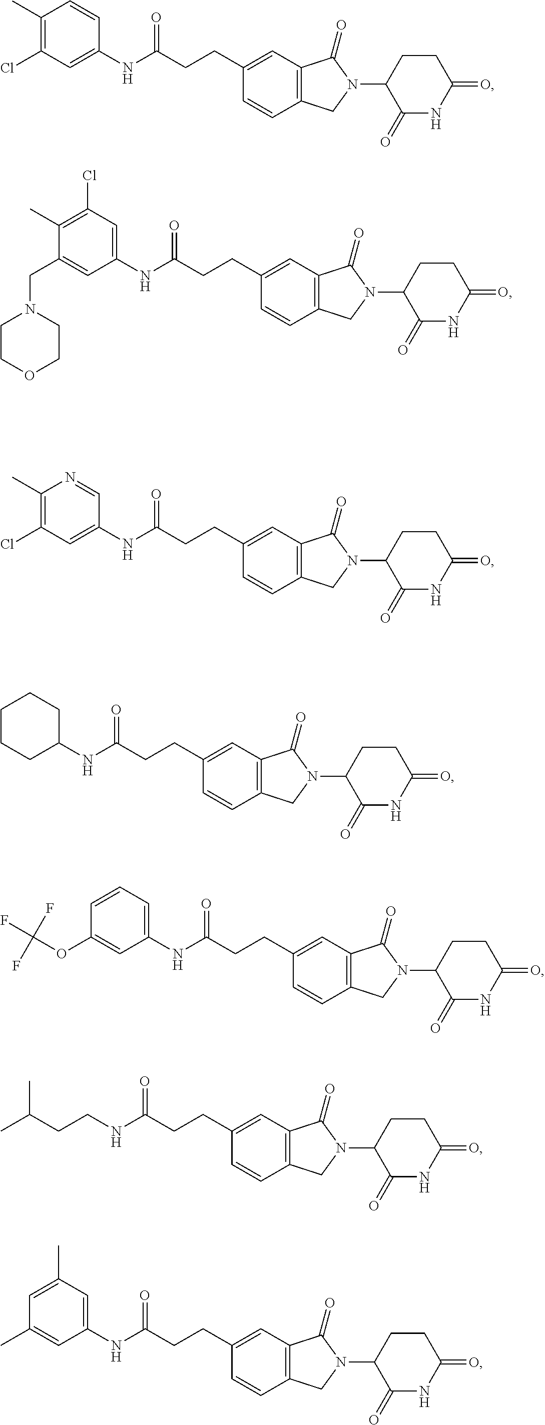 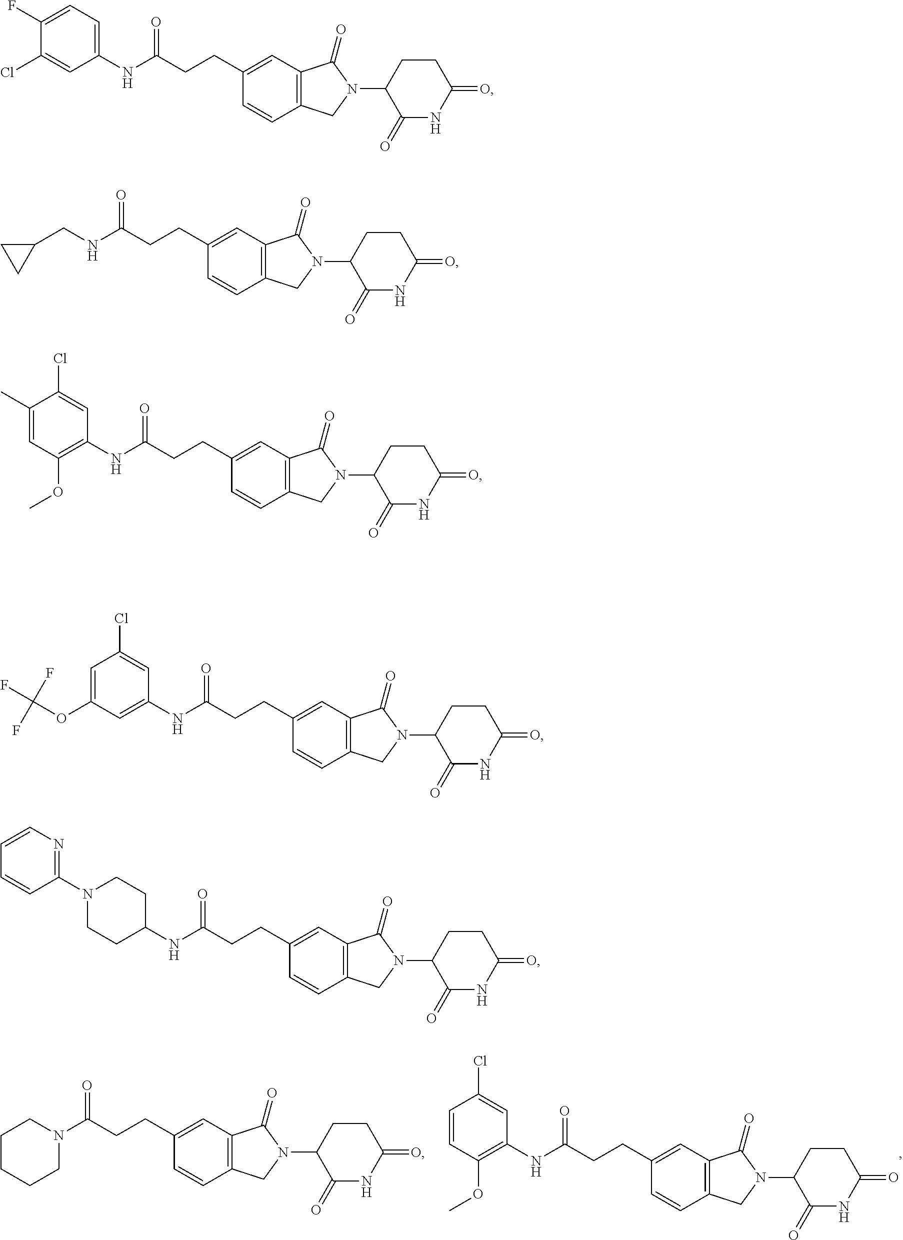 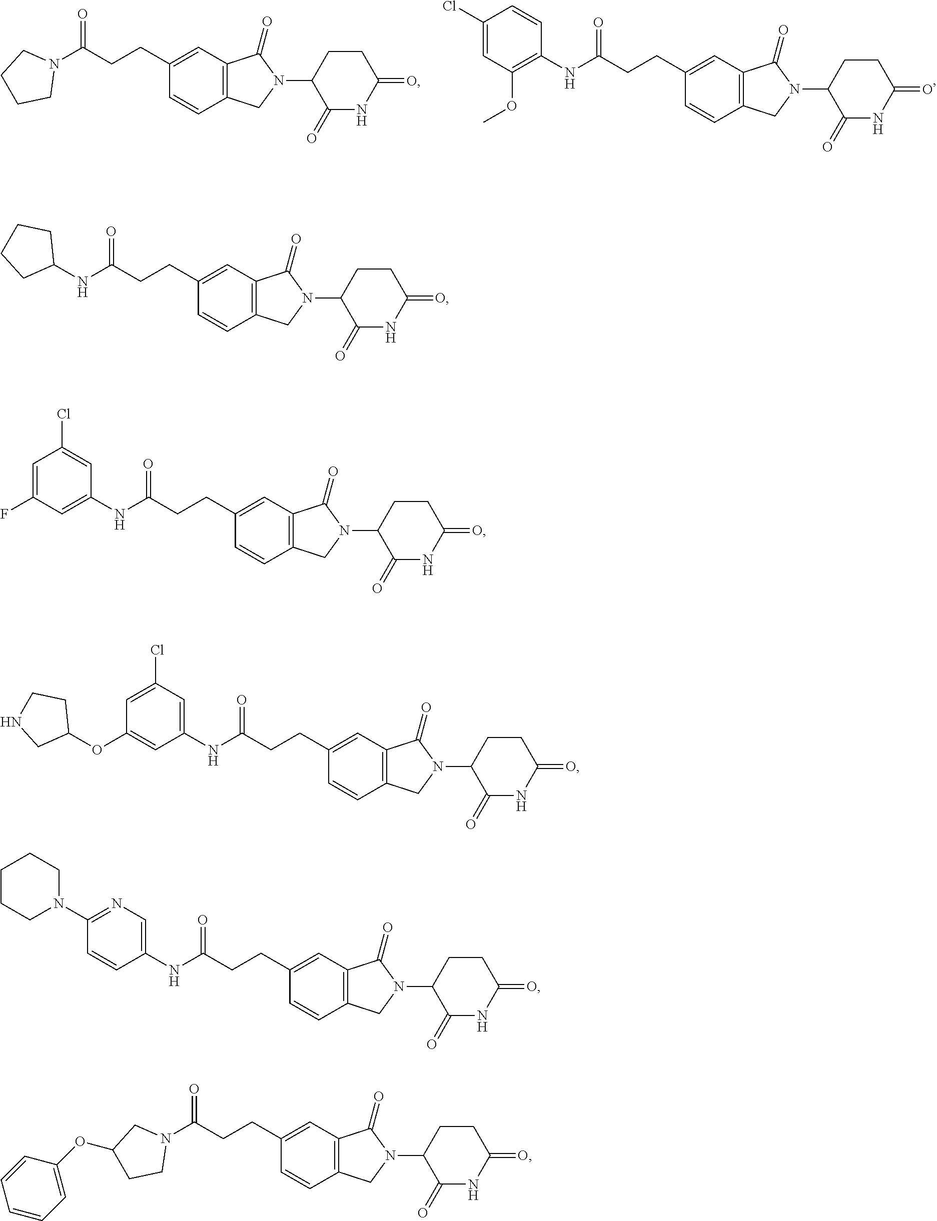 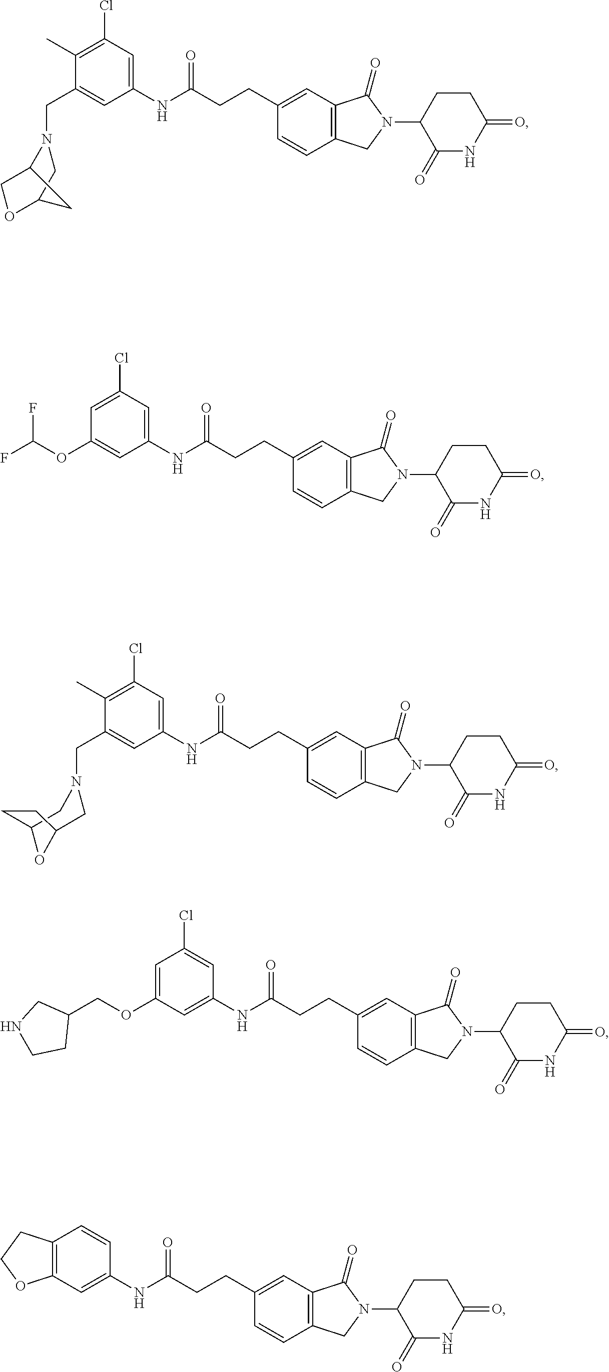 [SMILES: Cc1c(Cl)cc(NC(=O)CCc2ccc3c(c2)C(=O)N(C2CCC(=O)NC2=O)C3)cc1CN1CC2CC1CO2 ; O=C1CCC(N2Cc3ccc(CCC(=O)Nc4cc(Cl)cc(OC(F)F)c4)cc3C2=O)C(=O)N1 ; Cc1c(Cl)cc(NC(=O)CCc2ccc3c(c2)C(=O)N(C2CCC(=O)NC2=O)C3)cc1CN1CC2CCC(C1)O2 ; O=C1CCC(N2Cc3ccc(CCC(=O)Nc4cc(Cl)cc(OCC5CCNC5)c4)cc3C2=O)C(=O)N1 ; O=C1CCC(N2Cc3ccc(CCC(=O)Nc4ccc5c(c4)OCC5)cc3C2=O)C(=O)N1] 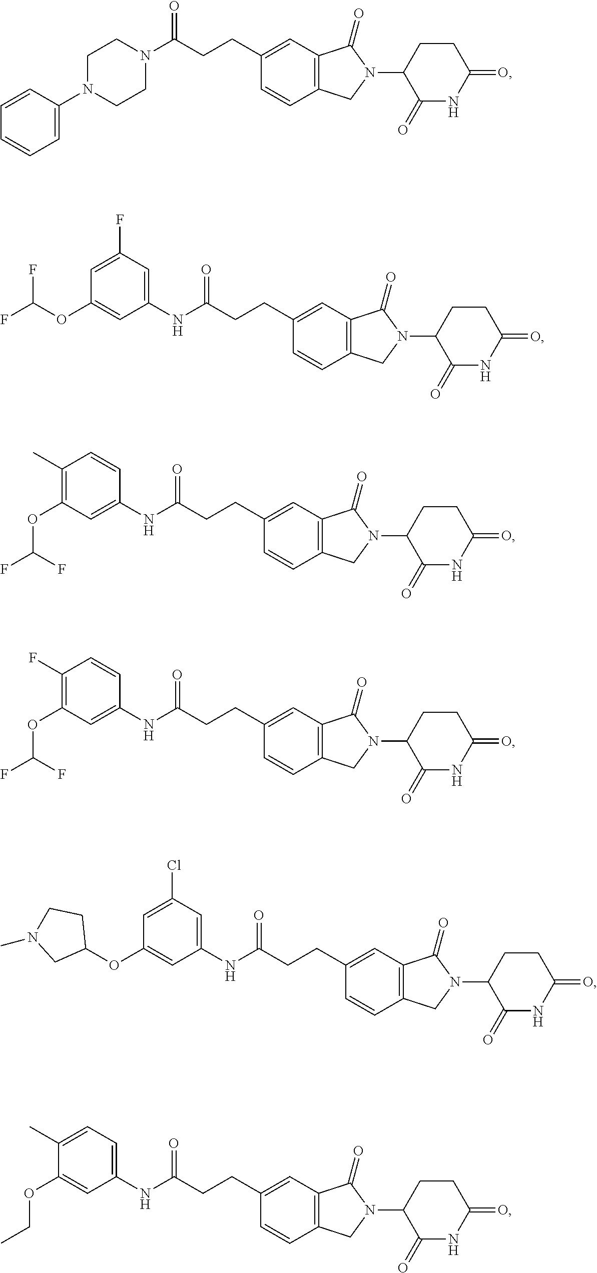 [SMILES: O=C1CCC(N2Cc3ccc(CCC(=O)N4CCN(c5ccccc5)CC4)cc3C2=O)C(=O)N1 ; O=C1CCC(N2Cc3ccc(CCC(=O)Nc4cc(F)cc(OC(F)F)c4)cc3C2=O)C(=O)N1 ; Cc1ccc(NC(=O)CCc2ccc3c(c2)C(=O)N(C2CCC(=O)NC2=O)C3)cc1OC(F)F ; O=C1CCC(N2Cc3ccc(CCC(=O)Nc4ccc(F)c(OC(F)F)c4)cc3C2=O)C(=O)N1 ; CN1CCC(Oc2cc(Cl)cc(NC(=O)CCc3ccc4c(c3)C(=O)N(C3CCC(=O)NC3=O)C4)c2)C1 ; CCOc1cc(NC(=O)CCc2ccc3c(c2)C(=O)N(C2CCC(=O)NC2=O)C3)ccc1C] 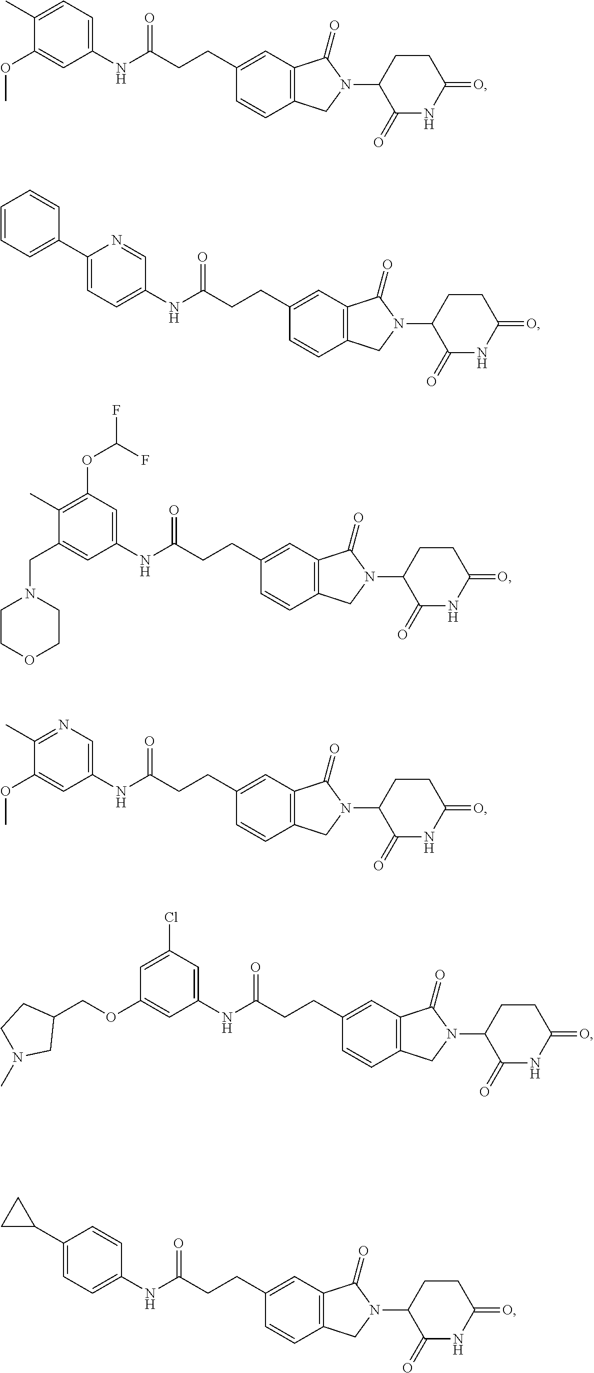 [SMILES: COc1cc(NC(=O)CCc2ccc3c(c2)C(=O)N(C2CCC(=O)NC2=O)C3)ccc1C ; O=C1CCC(N2Cc3ccc(CCC(=O)Nc4ccc(-c5ccccc5)nc4)cc3C2=O)C(=O)N1 ; Cc1c(CN2CCOCC2)cc(NC(=O)CCc2ccc3c(c2)C(=O)N(C2CCC(=O)NC2=O)C3)cc1OC(F)F ; COc1cc(NC(=O)CCc2ccc3c(c2)C(=O)N(C2CCC(=O)NC2=O)C3)cnc1C ; CN1CCC(COc2cc(Cl)cc(NC(=O)CCc3ccc4c(c3)C(=O)N(C3CCC(=O)NC3=O)C4)c2)C1 ; O=C1CCC(N2Cc3ccc(CCC(=O)Nc4ccc(C5CC5)cc4)cc3C2=O)C(=O)N1] 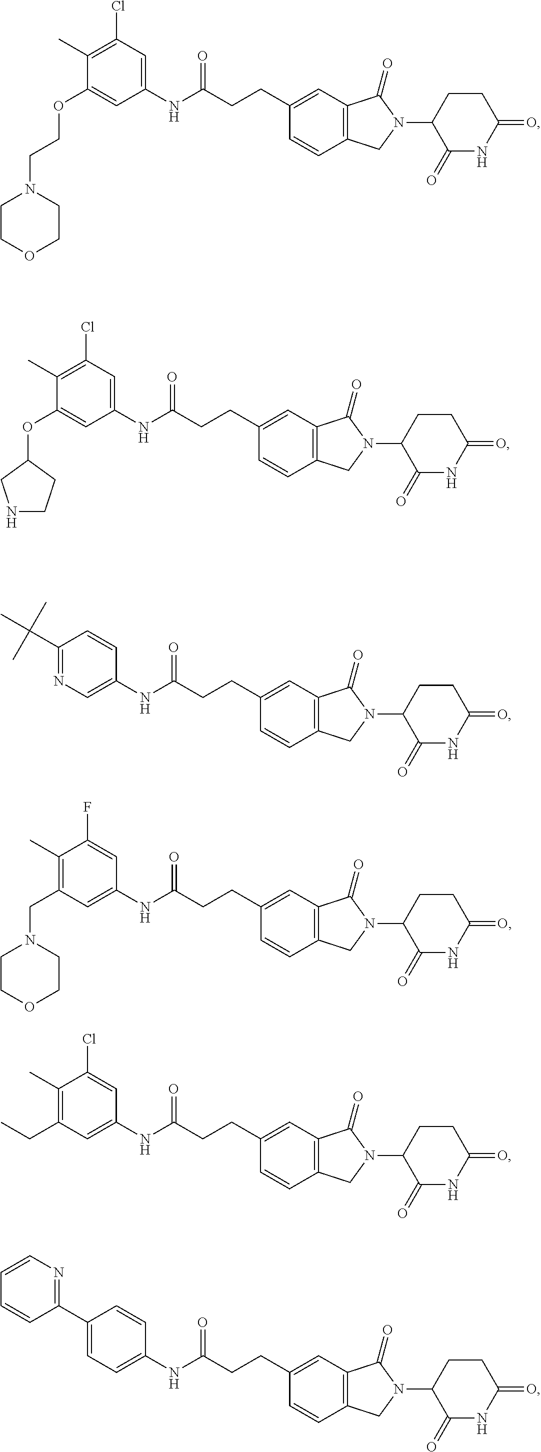 [SMILES: Cc1c(Cl)cc(NC(=O)CCc2ccc3c(c2)C(=O)N(C2CCC(=O)NC2=O)C3)cc1OCCN1CCOCC1 ; Cc1c(Cl)cc(NC(=O)CCc2ccc3c(c2)C(=O)N(C2CCC(=O)NC2=O)C3)cc1OC1CCNC1 ; CC(C)(C)c1ccc(NC(=O)CCc2ccc3c(c2)C(=O)N(C2CCC(=O)NC2=O)C3)cn1 ; Cc1c(F)cc(NC(=O)CCc2ccc3c(c2)C(=O)N(C2CCC(=O)NC2=O)C3)cc1CN1CCOCC1 ; CCc1cc(NC(=O)CCc2ccc3c(c2)C(=O)N(C2CCC(=O)NC2=O)C3)cc(Cl)c1C ; O=C1CCC(N2Cc3ccc(CCC(=O)Nc4ccc(-c5ccccn5)cc4)cc3C2=O)C(=O)N1] 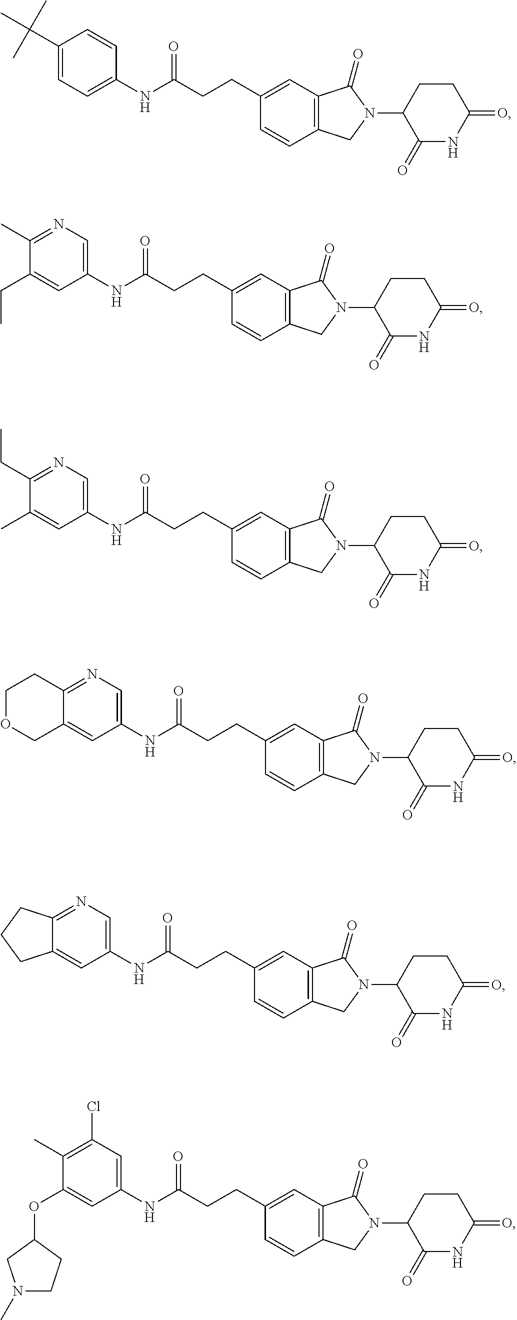 [SMILES: CC(C)(C)c1ccc(NC(=O)CCc2ccc3c(c2)C(=O)N(C2CCC(=O)NC2=O)C3)cc1 ; CCc1cc(NC(=O)CCc2ccc3c(c2)C(=O)N(C2CCC(=O)NC2=O)C3)cnc1C ; CCc1ncc(NC(=O)CCc2ccc3c(c2)C(=O)N(C2CCC(=O)NC2=O)C3)cc1C ; O=C1CCC(N2Cc3ccc(CCC(=O)Nc4cnc5c(c4)COCC5)cc3C2=O)C(=O)N1 ; O=C1CCC(N2Cc3ccc(CCC(=O)Nc4cnc5c(c4)CCC5)cc3C2=O)C(=O)N1 ; Cc1c(Cl)cc(NC(=O)CCc2ccc3c(c2)C(=O)N(C2CCC(=O)NC2=O)C3)cc1OC1CCN(C)C1] 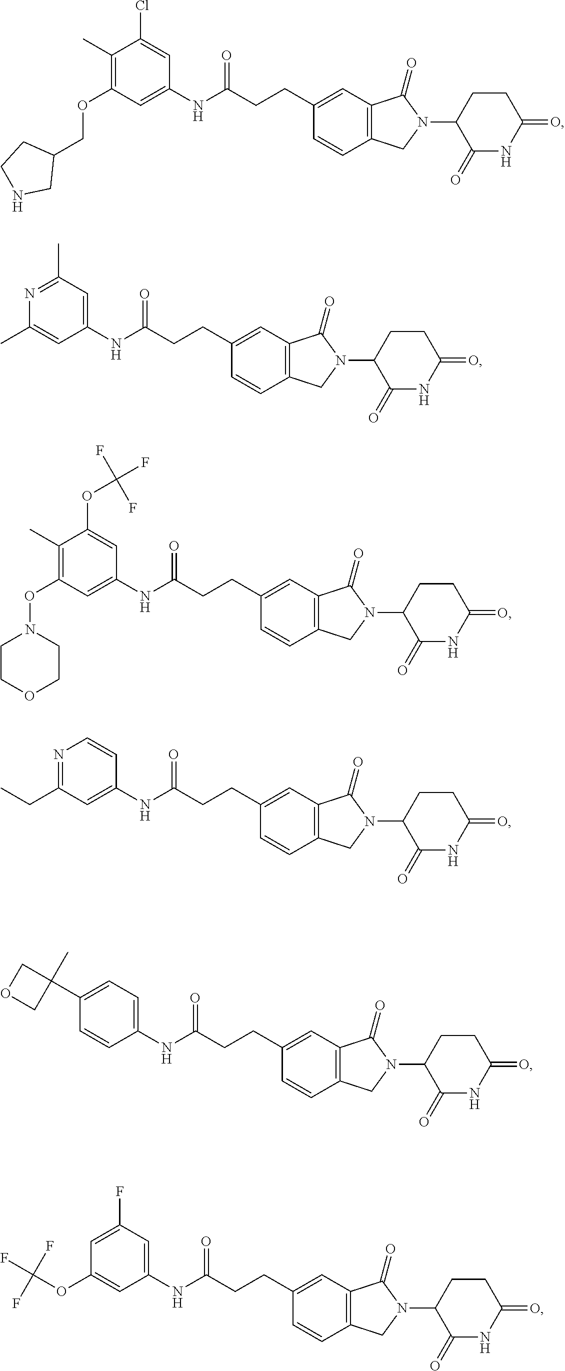 [SMILES: Cc1c(Cl)cc(NC(=O)CCc2ccc3c(c2)C(=O)N(C2CCC(=O)NC2=O)C3)cc1OCC1CCNC1 ; Cc1cc(NC(=O)CCc2ccc3c(c2)C(=O)N(C2CCC(=O)NC2=O)C3)cc(C)n1 ; Cc1c(ON2CCOCC2)cc(NC(=O)CCc2ccc3c(c2)C(=O)N(C2CCC(=O)NC2=O)C3)cc1OC(F)(F)F ; CCc1cc(NC(=O)CCc2ccc3c(c2)C(=O)N(C2CCC(=O)NC2=O)C3)ccn1 ; CC1(c2ccc(NC(=O)CCc3ccc4c(c3)C(=O)N(C3CCC(=O)NC3=O)C4)cc2)COC1 ; O=C1CCC(N2Cc3ccc(CCC(=O)Nc4cc(F)cc(OC(F)(F)F)c4)cc3C2=O)C(=O)N1] 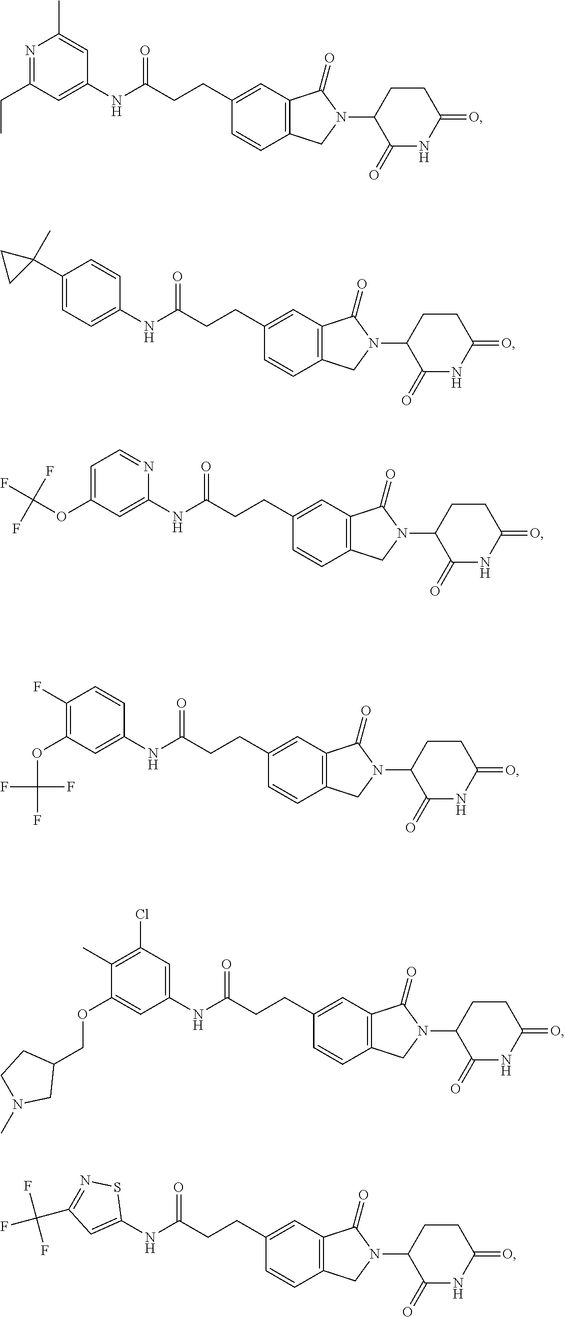 [SMILES: CCc1cc(NC(=O)CCc2ccc3c(c2)C(=O)N(C2CCC(=O)NC2=O)C3)cc(C)n1 ; CC1(c2ccc(NC(=O)CCc3ccc4c(c3)C(=O)N(C3CCC(=O)NC3=O)C4)cc2)CC1 ; O=C1CCC(N2Cc3ccc(CCC(=O)Nc4cc(OC(F)(F)F)ccn4)cc3C2=O)C(=O)N1 ; O=C1CCC(N2Cc3ccc(CCC(=O)Nc4ccc(F)c(OC(F)(F)F)c4)cc3C2=O)C(=O)N1 ; Cc1c(Cl)cc(NC(=O)CCc2ccc3c(c2)C(=O)N(C2CCC(=O)NC2=O)C3)cc1OCC1CCN(C)C1 ; O=C1CCC(N2Cc3ccc(CCC(=O)Nc4cc(C(F)(F)F)ns4)cc3C2=O)C(=O)N1] 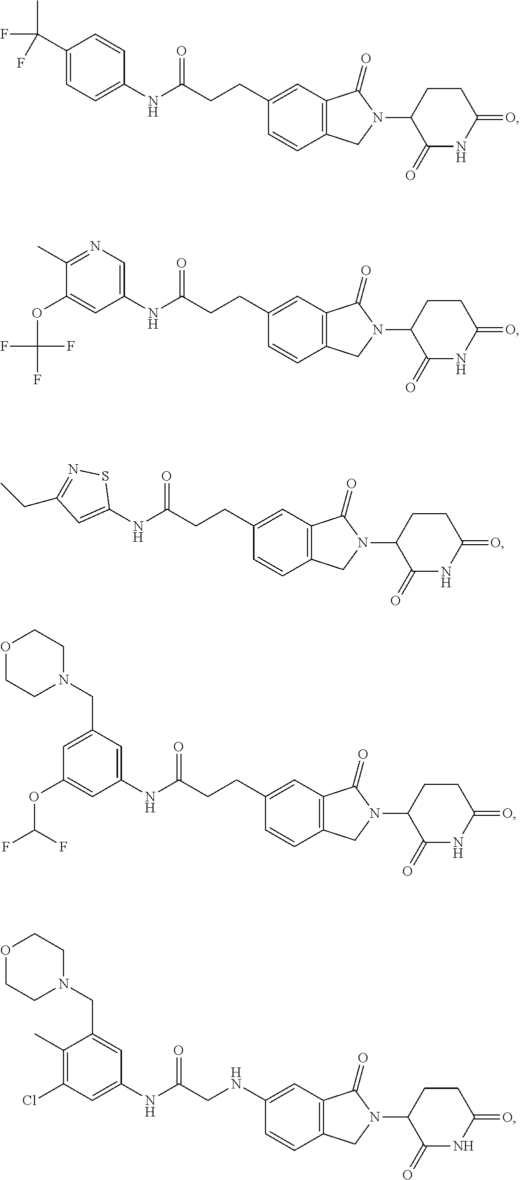 [SMILES: CC(F)(F)c1ccc(NC(=O)CCc2ccc3c(c2)C(=O)N(C2CCC(=O)NC2=O)C3)cc1 ; Cc1ncc(NC(=O)CCc2ccc3c(c2)C(=O)N(C2CCC(=O)NC2=O)C3)cc1OC(F)(F)F ; CCc1cc(NC(=O)CCc2ccc3c(c2)C(=O)N(C2CCC(=O)NC2=O)C3)sn1 ; O=C1CCC(N2Cc3ccc(CCC(=O)Nc4cc(CN5CCOCC5)cc(OC(F)F)c4)cc3C2=O)C(=O)N1 ; Cc1c(Cl)cc(NC(=O)CNc2ccc3c(c2)C(=O)N(C2CCC(=O)NC2=O)C3)cc1CN1CCOCC1] 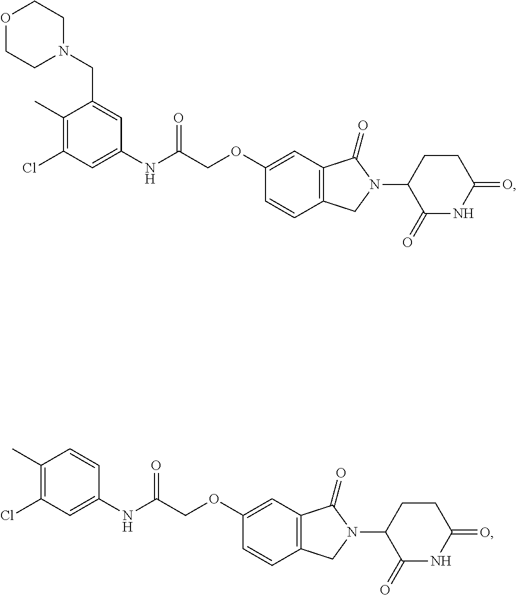 [SMILES: Cc1c(Cl)cc(NC(=O)COc2ccc3c(c2)C(=O)N(C2CCC(=O)NC2=O)C3)cc1CN1CCOCC1 ; Cc1ccc(NC(=O)COc2ccc3c(c2)C(=O)N(C2CCC(=O)NC2=O)C3)cc1Cl]
and pharmaceutically acceptable salts and stereoisomers thereof.

10. A composition comprising a compound according to any one of claims 1-9 or pharmaceutically acceptable salts or stereoisomers thereof.

11. The composition of claim 10, further comprising a pharmaceutically acceptable carrier.

12. The composition of claim 10 or 11, further comprising a second therapeutically active agent.

13. A compound according to any one of claims 1 to 9 or a composition of any one of claims 10-12 for use in therapy.

14. A compound according to any one of claims 1 to 9 or a composition of any one of claims 10-12 for use in the treatment of a disease associated with GSPT1.

15. A compound according to any one of claims 1 to 9 or a composition of any one of claims 10-12 for use in the treatment of cancer, in particular lung cancer, breast cancer and neuroendocrine cancer.

16. A use of a compound according to any one of claims 1 to 9 or a composition according to any one of claims 10-12 in the treatment of a disease associated with GSPT1, such as cancer, in particular lung cancer, breast cancer and neuroendocrine cancer, comprising administering to a subject a therapeutically-effective amount of the composition.

17. A method of treating cancer, in particular lung cancer, breast cancer and neuroendocrine cancer, in a subject, comprising administering to a subject a therapeutically effective amount of a compound according to any one of claims 1 to 9 or a composition of any one of claims 10 to 12.

18. A method of treating a Myc-driven cancer in a subject in need thereof, comprising administering to the subject a therapeutically effective amount of the compound of claim 1.

19. The method of claim 18, wherein the Myc-driven cancer is a Myc-driven lung cancer.

20. The method of claim 18, wherein the Myc-driven cancer is a Myc-driven small cell lung cancer.

21. The method of claim 20, wherein the Myc-driven small cell lung cancer is a high L-Myc small cell lung cancer.

22. The method of claim 18, wherein the Myc-driven cancer is a Myc-driven non-small cell lung cancer.

23. The method of claim 22, wherein the Myc-driven non-small cell lung cancer is a high N-Myc non-small cell lung cancer.

24. The method of claim 18, comprising orally administering the compound to the subject.

25. A method of degrading GSPT1 in a subject suffering from cancer, comprising administering to the subject a therapeutically effective amount of the compound of claim 1.

26. The method of claim 25, wherein the cancer is a Myc-driven cancer.

27. The method of claim 26, wherein the Myc-driven cancer is a Myc-driven lung cancer.

28. The method of claim 26, wherein the Myc-driven cancer is a Myc-driven small cell lung cancer.

29. The method of claim 28, wherein the Myc-driven small cell lung cancer is a high L-Myc small cell lung cancer.

30. The method of claim 25, wherein the Myc-driven cancer is a Myc-driven non-small cell lung cancer.

31. The method of claim 30, wherein the Myc-driven non-small cell lung cancer is a high N-Myc non-small cell lung cancer.

32. The method of claim 25, comprising orally administering the compound to the subject.

33. A method of reducing the level of GSPT1 in a subject suffering from cancer, comprising administering to the subject a therapeutically effective amount of the compound of claim 1.

34. The method of claim 33, wherein the cancer is a Myc-driven cancer.

35. The method of claim 34, wherein the Myc-driven cancer is a Myc-driven lung cancer.

36. The method of claim 34, wherein the Myc-driven cancer is a Myc-driven small cell lung cancer.

37. The method of claim 36, wherein the Myc-driven small cell lung cancer is a high L-Myc small cell lung cancer.

38. The method of claim 34, wherein the Myc-driven cancer is a Myc-driven non-small cell lung cancer.

39. The method of claim 38, wherein the Myc-driven non-small cell lung cancer is a high N-Myc non-small cell lung cancer.

40. The method of claim 33, comprising orally administering the compound to the subject.
